(19) United States
(12) Patent Application Publication
(10) Pub. No.: US 2025/0214960 A1
(43) Pub. Date: Jul. 3, 2025

(54) COMPOUNDS HAVING ACTIVITY OF DEGRADING GSPT1 AND APPLICATION THEREOF

(71) Applicants: SHANGHAI MEIZER PHARMACEUTICALS CO., LTD., Shanghai (CN); FUDAN UNIVERSITY, Shanghai (CN)

(72) Inventors: Yonghui WANG, Shanghai (CN); Yongzhi SHU, Shanghai (CN); Yunpeng ZHAO, Shanghai (CN); Qiong XIE, Shanghai (CN); Zhendong CHEN, Shanghai (CN); Xinqiao SHI, Shanghai (CN)

(21) Appl. No.: 18/707,314

(22) PCT Filed: Nov. 4, 2022
(86) PCT No.: PCT/CN2022/129952

§ 371 Date: May 3, 2024

(30) Foreign Application Priority Data
Nov. 5, 2021 (CN) 202111306535.8

(51) Int. Cl. C07D 401/04; A61K 31/454; A61K 31/5377; A61P 35/00; C07D 401/14; C07D 405/14; C07D 413/14

(52) U.S. Cl. CPC C07D 401/04; A61K 31/454; A61K 31/5377; A61P 35/00; C07D 401/14; C07D 405/14; C07D 413/14

(57) ABSTRACT

Disclosed in the present application are compounds having the activity of degrading GSPT1 and an application thereof. The compounds disclosed in the present application have the activity of degrading GSPT1, and can be used for preparing drugs for treating GSPT1 activity-related diseases.

### TECHNICAL FIELD

The present embodiment relates to the field of chemical pharmaceuticals, particularly to a class of compounds having activity of degrading GSPT1 and application thereof.

### BACKGROUND

Although a large number of protein targets related to disease occurrence and development have been discovered, due to the fact that about 70% of these proteins do not have suitable small molecule binding sites (inavailable targets for drug), traditional small molecule drugs are difficult to effectively target and regulate the physiological functions of these proteins, and even antibody drugs have little effect on these target proteins. Another way to regulate abnormal protein function is by regulating protein synthesis or degradation, such as using small interfering RNA (siRNA), antisense oligonucleotides, or gene editing techniques to knock out or silence target protein genes.

These nucleic acid-based techniques interfere with the transcription and translation processes of target proteins, thereby affecting their synthesis. The limitation of this type of technology is its poor stability and low bioavailability in human body, which greatly limits its widespread application. Therefore, regulating the degradation of target proteins has become a very promising strategy.

Ubiquitin-proteasome system (UPS) is an important physiological mechanism by which the body selectively degrades abnormal proteins. In short, the target protein is ubiquitinated through the cascade catalytic activity of various enzymes within the cell, which in turn is recognized and degraded by proteasomes. The ubiquitation step is divided into three steps: 1) activation: Ubiquitin first binds to adenosine triphosphate to form a ubiquitin adenylate complex. Ubiquitin is then separated from adenosine phosphate, and its carboxyl end is connected to the thiol group on the cysteine residue of E1 ubiquitin activating enzyme through a thioester bond. 2) Combination: E1 ubiquitin activating enzyme transfers activated ubiquitin to E2 ubiquitin binding enzyme through thioesterification reaction. 3) Connection: E3 ubiquitin ligase labels ubiquitin bound on E2 to the target protein, connecting the glycine on the carboxyl end of ubiquitin to the lysine portion on the target protein through an isomeric peptide bond. Through the catalysis of three enzymes, the ubiquitination cascade reaction ultimately forms the target protein polyubiquitin chain, which is transported to the proteasome for degradation.

E3 ligases can specifically recognize target protein substrates. Currently, E3 ligases are mainly divided into HECT (homologous to E6AP C terminus) family and RING finger family. CRL4^{CRBN} E3 ligases belong to RING-finger family, which is a protein complex composed of multiple subunits, including the substrate protein recognition module Cereblon (gene: CRBN), the E2 ubiquitin binding enzyme recognition module (RING domain), and the connecting part between them (Cullin protein). CRBN directly binds to substrates throughout the protein complex, controlling the substrate specificity of the entire ubiquitination process.

After years of research by scientists, thalidomide and its analogues have become effective therapeutic drugs for hematological malignancies. Research has shown that these compounds can target and bind to Cereblon, thereby controlling the specific recognition of substrate proteins by CRL4^{CRBN} E3 ubiquitin ligase, ubiquitination, and ultimately degradation by proteasome systems.

Thalidomide and its derivatives (IMiDs: immunomodulatory drugs; CELMoDs: Cereblon E3 ubiquitin ligase regulatory drugs) are also known as molecular glues.

It is interesting that though the existing IMiDs and CELMoDs have very similar structures, they exhibit different degradation functions. For example, both pomalidomide and lenalidomide can degrade zinc finger transcription factors 1 and 3 (IKZF1/3), but only lenalidomide can degrade casein kinase 1α (CK1α), which indicates that minor changes in molecular structure can significantly alter the substrate specificity of E3 ligases.

The degradation of zinc finger transcription factor 1/3 (IKZF1/3) can be used to treat multiple myeloma, while casein kinase 1α (CK1α) may be an effective target for 5q myelodysplastic syndrome. The existing compounds can selectively induce the degradation of GSPT1 by acting on the CRL4^{CRBN} E3 ligase and have been shown to have extensive anti-AML (acute myeloid leukemia) activity.

In summary, as an important target for anti-tumor and immunomodulatory drugs, CRBN has been proven to have clear therapeutic effects in various hematological malignancies such as multiple myeloma and chronic lymphocytic leukemia, skin diseases such as leprosy nodular erythema, and autoimmune diseases such as systemic lupus erythematosus. Lenalidomide is mainly used to treat multiple myeloma and myelodysplastic syndrome, but its effect is not ideal for other indications, and amine drugs have many adverse reactions (especially peripheral neuropathy). Therefore, the development of new structurally novel compounds as CRL4^{CRBN} E3 ubiquitin ligase modulators further enhances the therapeutic effect of tumors, reduces drug toxicity and side effects, and expands the clinical demand for new indications of amine drugs, which has significant research value and practical significance.

### SUMMARY OF INVENTION

The present invention is to provide a class of compounds having activity of degrading GSPT1 and application thereof.

To resolve the aforementioned technical problems, the present invention provides compounds as shown in Formula I, or a pharmaceutically acceptable salt, solvent, or stereoisomer thereof:

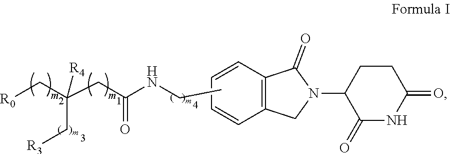

wherein,
R₀ is NR₁R₂ or OR₂;
R₁ is selected from: H, C₁₋₈ alkyl, C₂₋₈ alkenyl, C₂₋₈ alkynyl, C₃₋₁₀ cycloalkyl, 3- to 8-membered heterocyclic groups, aryl and heteroaryl;
R₂ is selected from: H, C₁₋₈ alkyl, C₂₋₈ alkenyl, C₂₋₈ alkynyl, C₃₋₁₀ cycloalkyl, 3- to 8-membered heterocyclic groups, aryl, heteroaryl, (CH₂)ₙC(O)R₅, C(O)(CHR₆)ₙR₅, C(O)(CHR₆)ₙOR₅, S(O)₂R₅, C(O)C(O)R₅; wherein R₅ is independently selected from: H, C₁₋₈ alkyl, C₂₋₈ alkenyl, C₂₋₈ alkynyl, C₃₋₁₀ cycloalkyl, 3- to 8-membered heterocyclic groups, aryl, heteroaryl, NR₇R₈; R₇ is selected from: H, C₁₋₈ alkyl, R₈ is selected from: H, C₁₋₈ alkyl, C₂₋₈ alkenyl, C₂₋₈ alkynyl, C₃₋₁₀ cycloalkyl, 3- to 8-membered heterocyclic groups, aryl, heteroaryl, alkyl carbonyl, alkoxycarbonyl, or R₇ and R₈ can be connected into a ring; R₆ is independently selected from: H, C₁₋₈ alkyl, C₂₋₈ alkenyl, C₂₋₈ alkynyl, C₃₋₁₀ cycloalkyl, 3- to 8-membered heterocyclic groups, aryl, heteroaryl, n is independently 0 or 1;
R₃ is selected from: C₁₋₈ alkyl, C₂₋₈ alkenyl, C₂₋₈ alkynyl, C₃₋₁₀ cycloalkyl, 3- to 8-membered heterocyclic groups, aryl, heteroaryl, C(O)R₉; wherein, R₉ is selected from: C₁₋₈ alkyl, C₂₋₈ alkenyl, C₂₋₈ alkynyl, C₃₋₁₀ cycloalkyl, 3- to 8-membered heterocyclic groups, aryl, heteroaryl, NH₂; or R₂ and R₃ can be connected into a ring (Preferably, the 3- to 20-membered ring is either non-substituted or substituted by 1-3 substituents, which can be saturated, unsaturated or aromatic ring, or a single or fused ring);
R₄ is selected from: H, C₁₋₈ alkyl, C₂₋₈ alkenyl, C₂₋₈ alkynyl, C₃₋₁₀ cycloalkyl, 3- to 8-membered heterocyclic groups, aryl, heteroaryl; or R₃ and R₄ can be connected into a ring (Preferably, the 3- to 20-membered ring is either non-substituted or substituted by 1-3 substituents, which can be saturated, unsaturated carbon ring, or a heterocyclic ring);
Wherein, each of the above-mentioned alkyl, alkenyl, alkynyl, cycloalkyl, heterocyclic, aryl, and heteroaryl groups can be optionally and independently substituted by 1-3 substituents, the substituents are independently halogens, C₁₋₄ alkyl, C₂₋₄ alkenyl, C₂₋₄ alkynyl, C₃₋₈ cycloalkyl, 3- to 8-membered heterocyclic groups, aryl, heteroaryl, single or multi-halogenated C₁₋₄ alkyl (like —CF₃), alkoxy, alkyl carbonyl, alkoxycarbonyl, CN, OH, NH₂, or NO₂;
m₁ is 0, 1 or 2;
m₂ is 0, 1 or 2;
m₃ is 0, 1 or 2;
m₄ is 0, 1 or 2.

In another embodiment, R₀ is OR₂; R₂ is C(O) OR₅, R₅ is selected from: C₁₋₈ alkyl, C₃₋₈ cycloalkyl, 3- to 8-membered heterocyclic groups.

In another embodiment, R₀ is NR₁R₂; R₁ is selected from: H, C₁₋₈ alkyl; R₂ is selected from: C₁₋₈ alkyl, (CH₂)ₙC(O)R₅, C(O)(CHR₆)ₙR₅, C(O)(CHR₆)ₙOR₅, S(O)₂R₅, C(O)C(O)R₅, wherein, the R₅ is independently selected from: H, C₁₋₈ alkyl, C₃₋₈ cycloalkyl, 3- to 8-membered heterocyclic groups, aryl, heteroaryl, NR₇R₈; R₇ is selected from: H, C₁₋₈ alkyl; R₈ is selected from: H, C₁₋₈ alkyl, C₃₋₁₀ cycloalkyl, 3- to 8-membered heterocyclic groups, aryl, heteroaryl, alkyl carbonyl, alkoxycarbonyl, the R₆ is independently selected from: H, C₁₋₈ alkyl, C₃₋₈ cycloalkyl, 3- to 8-membered heterocyclic groups, aryl, heteroaryl, and n is independently 0 or 1.

In another embodiment, R₀ is NR₁R₂; R₁ is selected from: H, C₁₋₈ alkyl; R₂ is selected from: C₁₋₈ alkyl, (CH₂)ₙC(O)R₅, C(O)(CHR₆)ₙR₅, C(O)(CHR₆)ₙOR₅, S(O)₂R₅, C(O)C(O)R₅, wherein, the R₅ is independently selected from: H, C₁₋₈ alkyl, C₃₋₈ cycloalkyl, 3- to 8-membered heterocyclic groups, aryl, heteroaryl, NR₇R₈; R₇ is selected from: H, C₁₋₈ alkyl; R₈ is selected from: H, C₁₋₈ alkyl, C₃₋₁₀ cycloalkyl, 3- to 8-membered heterocyclic groups, aryl, heteroaryl, alkyl carbonyl, alkoxycarbonyl, the R₆ is independently selected from: H, C₁₋₈ alkyl, C₃₋₈ cycloalkyl, aryl, and n is independently 0 or 1.

In another embodiment, R₀ is NR₁R₂; R₁ is selected from: H, C₁₋₄ alkyl; R₂ is C₁₋₈ alkyl.

In another embodiment, R₀ is NR₁R₂; R₁ is selected from: H, C₁₋₄ alkyl; R₂ is (CH₂)ₙC(O)R₅, R₅ is selected from: C₁₋₈ alkyl, C₃₋₈ cycloalkyl, aryl, NR₇R₈; R₇ is H, R₈ is selected from: C₁₋₈ alkyl, C₃₋₁₀ cycloalkyl, aryl, arylalkyl (such as benzyl), n is 0 or 1.

In another embodiment, R₀ is NR₁R₂; R₁ is selected from: H, C₁₋₄ alkyl; R₂ is C(O)(CHR₆)ₙR₅, R₅ is selected from: C₁₋₈ alkyl, C₃₋₈ cycloalkyl, aryl, NR₇R₈; R₇ is H, R₈ is selected from: C₁₋₈ alkyl, C₃₋₁₀ cycloalkyl, aryl, aryl alkyl (such as benzyl); R₆ is selected from: H, C₁₋₈ alkyl, C₃₋₈ cycloalkyl, aryl, n is 0 or 1.

In another embodiment, R₀ is NR₁R₂; R₁ is selected from: H, C₁₋₄ alkyl; R₂ is C(O)(CHR₆)ₙR₅, R₅ is selected from: NR₇R₈; R₇ is selected from: H, C₁₋₄ alkyl; R₈ is selected from: C₁₋₈ alkyl, alkyl carbonyl and alkoxycarbonyl; R₆ is independently selected from: H, C₁₋₈ alkyl, C₃₋₈ cycloalkyl, aryl, n is 0 or 1.

In another embodiment, R₀ is NR₁R₂; R₁ is selected from: H, C₁₋₄ alkyl; R₂ is C(O)(CHR₆)ₙOR₅, R₅ is selected from: C₁₋₈ alkyl, C₃₋₈ cycloalkyl, 3- to 8-membered heterocyclic groups, aryl, heteroaryl; R₆ is selected from: H, C₁₋₄ alkyl, n is 0 or 1.

In another embodiment, R₀ is NR₁R₂; R₁ is selected from: H, C₁₋₄ alkyl; R₂ is S(O)₂R₅, R₅ is selected from: C₁₋₈ alkyl, C₃₋₈ cycloalkyl, aryl (preferably alkyl substituted phenyl).

In another embodiment, R₀ is NR₁R₂; R₁ is selected from: H, C₁₋₄ alkyl; R₂ is C(O)C(O)R₅, R₅ is selected from: aryl, heteroaryl.

In another embodiment, the structure of the compound is shown in Formula I′:

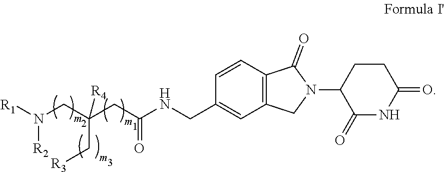

In another embodiment, when R₁ is H, R₂ is not H.

In another embodiment, the structure of the compound is shown in Formula I″:

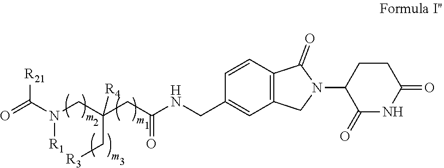

Wherein, R₂₁ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl, OR₂₂; wherein, R₂₂ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl;
Wherein, each of the above-mentioned alkyl, alkenyl, alkynyl, cycloalkyl, heterocyclic, aryl, and heteroaryl groups can be optionally and independently substituted by 1-3 substituents, the substituents are independently halogens, C₁₋₄ alkyl, C₂₋₄ alkenyl, C₂₋₄ alkynyl, C₃₋₈ cycloalkyl, 3- to 8-membered heterocyclic groups, aryl, heteroaryl, CN, OH, NH₂, or NO₂.

In another embodiment, m₁ is 0, m₂ is 0, and m₃ is 0.

In another embodiment, m₁ is 1 or 2, m₂ is 0, and m₃ is 0.

In another embodiment, m₁ is 0, m₂ is 1 or 2, and m₃ is 0.

In another embodiment, m₁ is 0, m₂ is 0, and m₃ is 1 or 2.

In another embodiment, R₁ is selected from: H or C₁₋₄ alkyl.

In another embodiment, R₂ is selected from: C(O)R₅ or C(O)OR₅; wherein, R₅ is selected from: C₁₋₆ alkyl, C₂₋₆ alkenyl, C₂₋₆ alkynyl, C₃₋₆ cycloalkyl, 3- to 8-membered heterocyclic groups, aryl, and heteroaryl; wherein, each of the above-mentioned C₁₋₆ alkyl, C₂₋₆ alkenyl, C₂₋₆ alkynyl, C₃₋₆ cycloalkyl, 3- to 8-membered heterocyclic groups, aryl, heteroaryl can be optionally and independently substituted by 1-3 substituents, the substituents are independently halogens, C₁₋₄ alkyl, C₂₋₄ alkenyl, C₂₋₄ alkynyl, C₃₋₈ cycloalkyl, 3- to 8-membered heterocyclic groups, aryl, heteroaryl, CN, OH, NH₂, or NO₂.

In another embodiment, R₃ is selected from: C₁₋₈ alkyl, C₂₋₈ alkenyl, C₂₋₈ alkynyl, C₃₋₈ cycloalkyl, 3- to 8-membered heterocyclic groups, aryl, and heteroaryl: wherein, each of the above-mentioned C₁₋₈ alkyl, C₂₋₈ alkenyl, C₃₋₈ cycloalkyl, 3- to 8-membered heterocyclic groups, aryl, heteroaryl can be optionally and independently substituted by 1-3 substituents, the substituents are independently halogens, C₁₋₄ alkyl, C₂₋₄ alkenyl, C₂₋₄ alkynyl, C₃₋₈ cycloalkyl, 3- to 8-membered heterocyclic groups, aryl, heteroaryl, CN, OH, NH₂, or NO₂.

In another embodiment, R₃ is selected from: C₁₋₈ alkyl, C₃₋₈ cycloalkyl, 3- to 8-membered heterocyclic groups, aryl, wherein, each of the above-mentioned C₁₋₈ alkyl, C₃₋₈ cycloalkyl, aryl can be optionally and independently substituted by 1-3 substituents, the substituents are independently halogens, C₁₋₄ alkyl, single or multi-halogenated C₁₋₄ alkyl (like CF₃), alkoxy, C₃₋₈ cycloalkyl, aryl, OH.

In another embodiment, R₃ is phenyl which is substituted by 1-3 substituents, the substituents are independently halogens, C₁₋₄ alkyl, single or multi-halogenated C₁₋₄ alkyl (like CF₃), alkoxy, OH.

In another embodiment, R₄ is H; or, R₃ and R₄ are connected into 3- to 12-membered heterocyclic groups which is unsubstituted or substituted by 1-3 substituents, saturated, unsaturated, or aromatic carbon ring or heterocycles; the above-mentioned 1-3 substituents are independently halogens, C₁₋₄ alkyl, C₂₋₄ alkenyl, C₂₋₄ alkynyl, C₃₋₈ cycloalkyl, 3- to 8-membered heterocyclic groups, aryl, heteroaryl, CN, OH, NH₂, or NO₂.

In another embodiment, the compound is not:

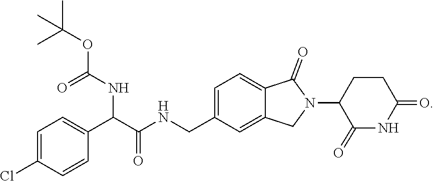 [SMILES: CC(C)(C)OC(=O)NC(C(=O)NCc1ccc2c(c1)CN(C1CCC(=O)NC1=O)C2=O)c1ccc(Cl)cc1]

In another embodiment, the compound is not:

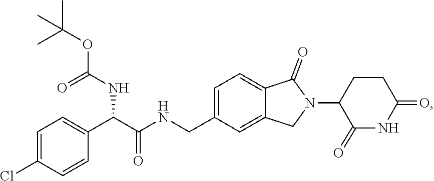 [SMILES: CC(C)(C)OC(=O)N[C@H](C(=O)NCc1ccc2c(c1)CN(C1CCC(=O)NC1=O)C2=O)c1ccc(Cl)cc1]

4-150.

In a preferred embodiment, the compound structure is shown in Formula II,

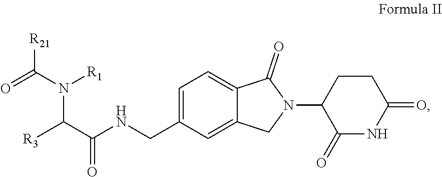

Wherein,
R₁ is selected from: H, C₁₋₈ alkyl, C₂₋₈ alkenyl, C₂₋₈ alkynyl, C₃₋₈ cycloalkyl, 3- to 8-membered heterocyclic groups, aryl and heteroaryl;
R₃ is selected from: C₁₋₈ alkyl, C₂₋₈ alkenyl, C₂₋₈ alkynyl, C₃₋₈ cycloalkyl, 3- to 8-membered heterocyclic groups, aryl and heteroaryl;
R₂₁ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl, OR₂₂; wherein, R₂₂ is selected from: C₁₋₈ alkyl, C₂₋₈ alkenyl, C₂₋₈ alkynyl, C₃₋₈ cycloalkyl, 3- to 8-membered heterocyclic groups, aryl and heteroaryl;
Wherein, each of the above-mentioned alkyl, alkenyl, alkynyl, cycloalkyl, heterocyclic groups, aryl, and heteroaryl can be optionally and independently substituted by 1-3 substituents, the substituents are independently halogens, C₁₋₄ alkyl, C₂₋₄ alkenyl, C₂₋₄ alkynyl, C₃₋₈ cycloalkyl, 3- to 8-membered heterocyclic groups, aryl, heteroaryl, CN, OH, NH₂, or NO₂.

In another embodiment, R₁ is selected from: H, and C₁₋₄ alkyl group without or with 1 to 3 substituents.

In another embodiment, R₃ is selected from: C₁₋₄ alkyl without or with 1 to 3 substituents, C₃₋₈ cycloalkyl without or with 1 to 3 substituents and aryl without or with 1 to 3 substituents.

In another embodiment, R₂₁ is selected from: C₁₋₄ alkyl without or with 1 to 3 substituents, C₃₋₆ cycloalkyl without or with 1 to 3 substituents and aryl without or with 1 to 3 substituents, and OR₂₂; wherein, R₂₂ is selected from: C₁₋₄ alkyl without or with 1 to 3 substituents, C₃₋₈ cycloalkyl without or with 1 to 3 substituents, aryl without or with 1 to 3 substituents, and heteroaryl without or with 1 to 3 substituents.

In some preferred embodiments, the compound is selected from any of the following groups:

| Compound | |
|---|---|
| No. | Structure |
| 4-151 | 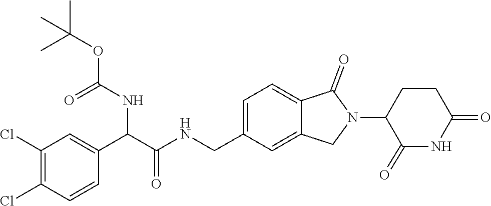 [SMILES: CC(C)(C)OC(=O)NC(C(=O)NCc1ccc2c(c1)CN(C1CCC(=O)NC1=O)C2=O)c1ccc(Cl)c(Cl)c1] |
| 5-77 | 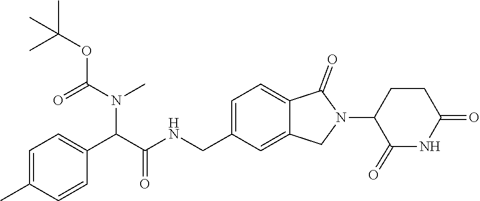 [SMILES: Cc1ccc(C(C(=O)NCc2ccc3c(c2)CN(C2CCC(=O)NC2=O)C3=O)N(C)C(=O)OC(C)(C)C)cc1] |
| 4-166 | 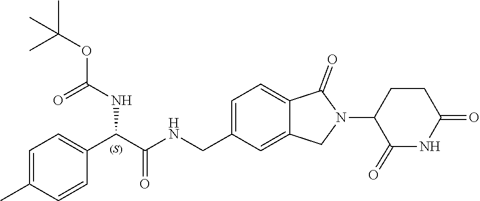 |
| 4-135 | 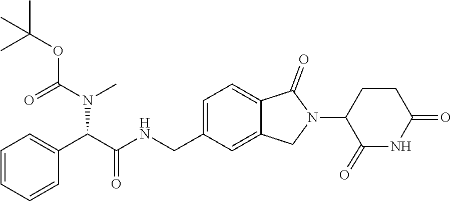 [SMILES: CN(C(=O)OC(C)(C)C)[C@H](C(=O)NCc1ccc2c(c1)CN(C1CCC(=O)NC1=O)C2=O)c1ccccc1] |
| 5-5 | 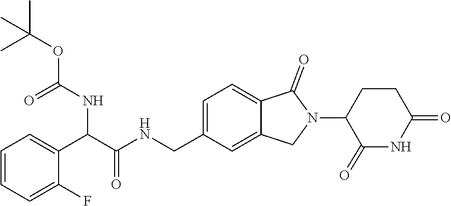 [SMILES: CC(C)(C)OC(=O)NC(C(=O)NCc1ccc2c(c1)CN(C1CCC(=O)NC1=O)C2=O)c1ccccc1F] |
| 4-123 | 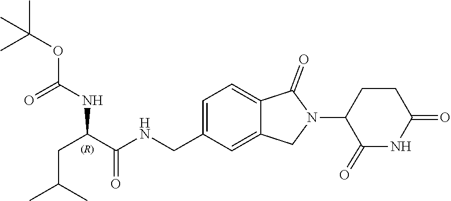 |
| 4-87 | 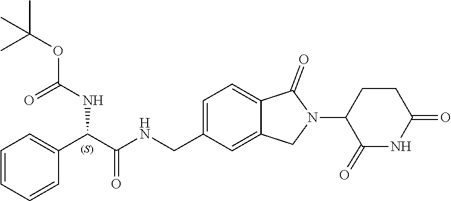 |
| 5-57 | 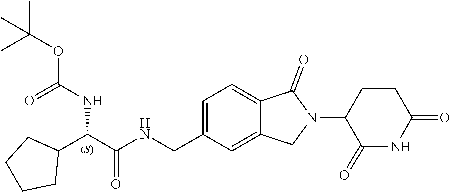 |
| 4-150 | 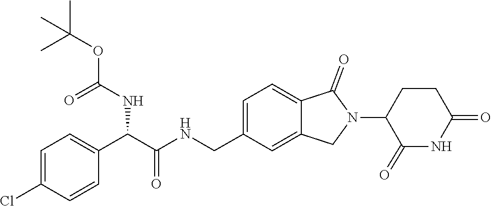 [SMILES: CC(C)(C)OC(=O)N[C@H](C(=O)NCc1ccc2c(c1)CN(C1CCC(=O)NC1=O)C2=O)c1ccc(Cl)cc1] |
| 5-56 | 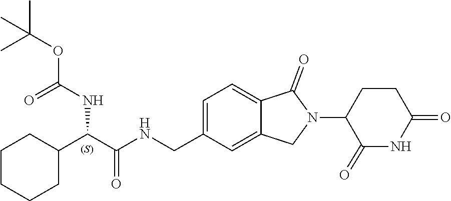 |
| 5-7 | 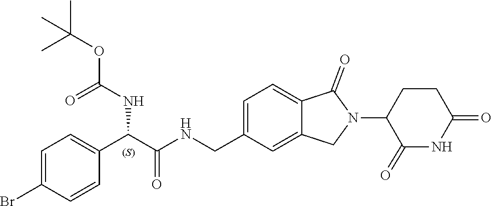 |
| 4-159 | 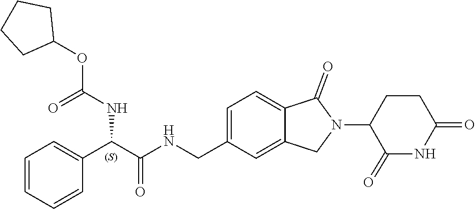 |
| 4-152 | 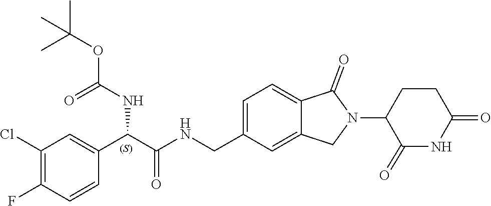 |
| 4-160 | 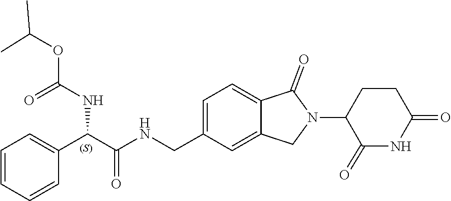 |
| 5-17 | 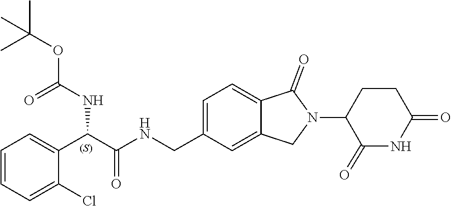 |
| 4-175 | 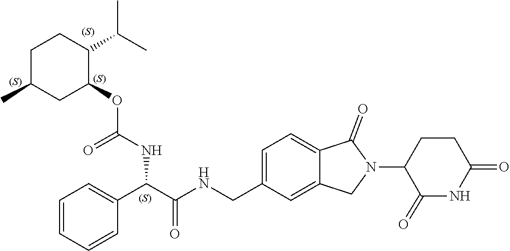 |
| 1-89 | 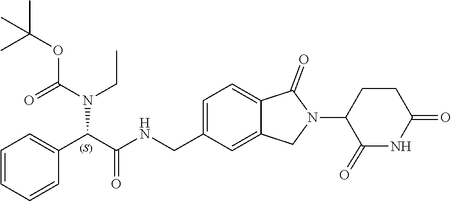 |
| 5-36 | 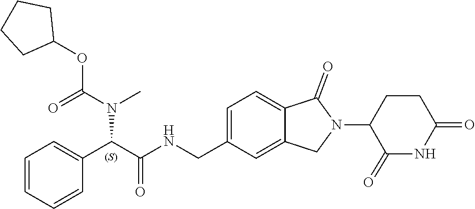 |
| 5-31 | 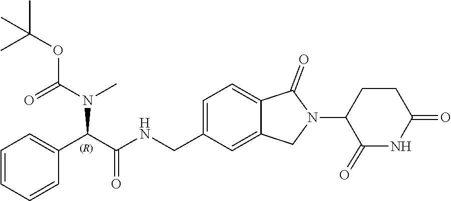 |
| 5-29 | 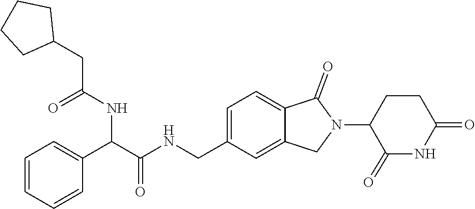 [SMILES: O=C1CCC(N2Cc3cc(CNC(=O)C(NC(=O)CC4CCCC4)c4ccccc4)ccc3C2=O)C(=O)N1] |
| 5-75 | 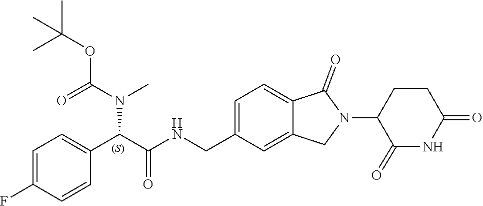 |
| 5-50 | 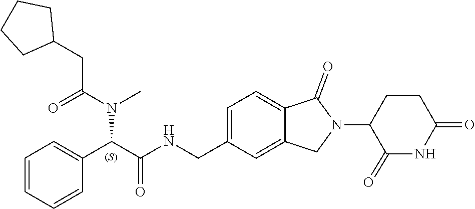 |
| 5-76 | 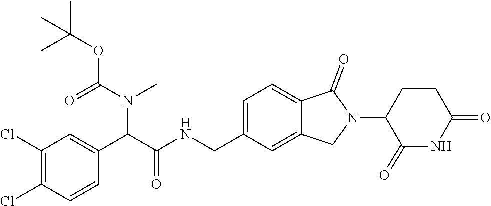 [SMILES: CN(C(=O)OC(C)(C)C)C(C(=O)NCc1ccc2c(c1)CN(C1CCC(=O)NC1=O)C2=O)c1ccc(Cl)c(Cl)c1] |
| 5-30 | 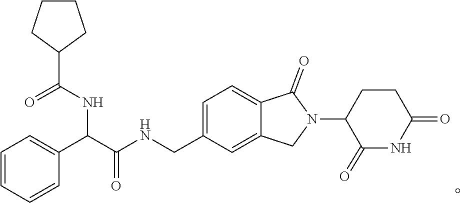 [SMILES: O=C1CCC(N2Cc3cc(CNC(=O)C(NC(=O)C4CCCC4)c4ccccc4)ccc3C2=O)C(=O)N1] |
| 6-01 | 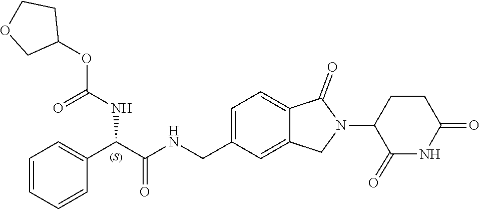 |
| 6-02 | 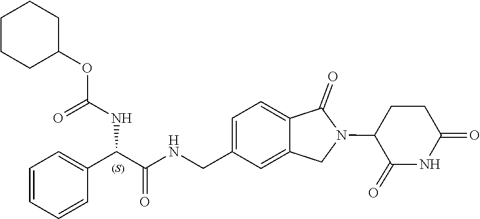 |
| 6-03 | 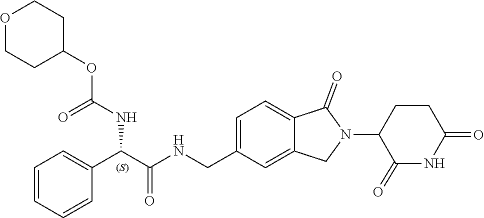 |
| 4-175b | 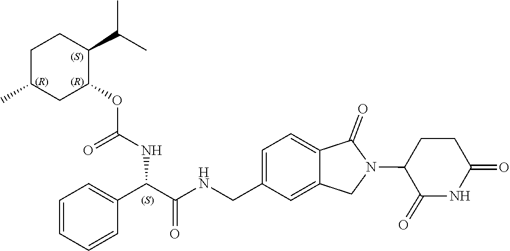 |
| 5-17b | 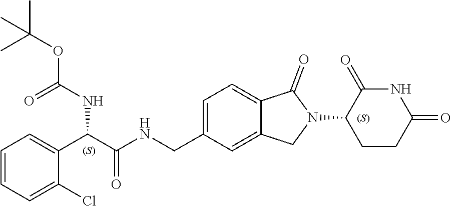 |

In a preferred embodiment, the compound structure is shown in Formula III,

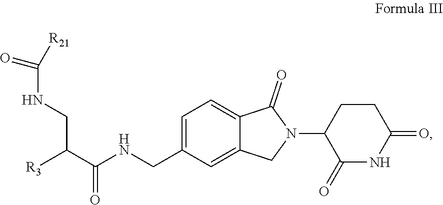

Wherein,
R₃ is selected from: C₁₋₈ alkyl, C₂₋₈ alkenyl, C₂₋₈ alkynyl, C₃₋₈ cycloalkyl, 3- to 8-membered heterocyclic groups, aryl and heteroaryl;
R₂₁ is selected from: C₁₋₈ alkyl, C₂₋₈ alkenyl, C₂₋₈ alkynyl, C₃₋₈ cycloalkyl, 3- to 8-membered heterocyclic groups, aryl and heteroaryl, OR₂₂; wherein, R₂₂ is selected from: C₁₋₈ alkyl, C₂₋₈ alkenyl, C₂₋₈ alkynyl, C₃₋₈ cycloalkyl, 3- to 8-membered heterocyclic groups, aryl and heteroaryl;
Wherein, each of the above-mentioned alkyl, alkenyl, alkynyl, cycloalkyl, heterocyclic groups, aryl, heteroaryl can be optionally and independently substituted by 1-3 substituents, the substituents are independently halogens, C₁₋₄ alkyl, C₂₋₄ alkenyl, C₂₋₄ alkynyl, C₃₋₈ cycloalkyl, 3- to 8-membered heterocyclic groups, aryl, heteroaryl, CN, OH, NH₂, or NO₂.

In another embodiment, R₃ is selected from: C₁₋₄ alkyl without or with 1 to 3 substituents, C₃₋₈ cycloalkyl without or with 1 to 3 substituents and aryl without or with 1 to 3 substituents.

In another embodiment, R₂₁ is OR₂₂; wherein, R₂₂ is selected from: C₁₋₄ alkyl without or with 1 to 3 substituents, C₃₋₈ cycloalkyl without or with 1 to 3 substituents and aryl without or with 1 to 3 substituents, heteroaryl without or with 1 to 3 substituents.

In some preferred embodiments, the compound is selected from any of the following groups:

| Compound | |
|---|---|
| No. | Structure |
| 5-55 | 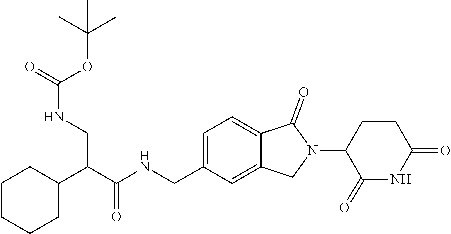 [SMILES: CC(C)(C)OC(=O)NCC(C(=O)NCc1ccc2c(c1)CN(C1CCC(=O)NC1=O)C2=O)C1CCCCC1] |
| 5-10 | 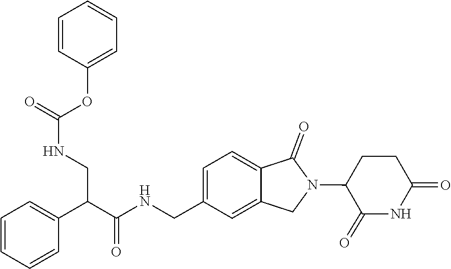 [SMILES: O=C1CCC(N2Cc3cc(CNC(=O)C(CNC(=O)Oc4ccccc4)c4ccccc4)ccc3C2=O)C(=O)N1] |
| 4-131 | 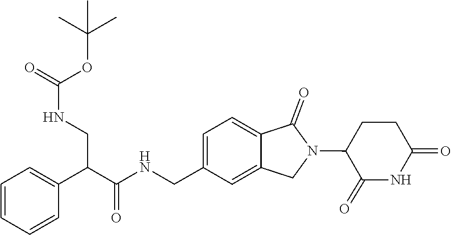 [SMILES: CC(C)(C)OC(=O)NCC(C(=O)NCc1ccc2c(c1)CN(C1CCC(=O)NC1=O)C2=O)c1ccccc1] |
| 5-41 | 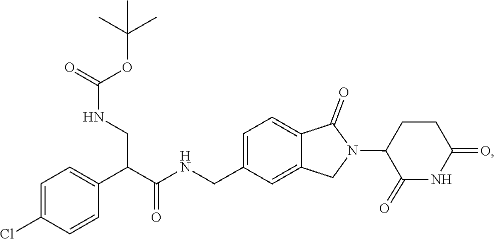 [SMILES: CC(C)(C)OC(=O)NCC(C(=O)NCc1ccc2c(c1)CN(C1CCC(=O)NC1=O)C2=O)c1ccc(Cl)cc1] |
| | and |
| 5-33 | 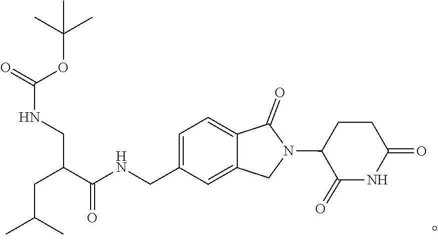 [SMILES: CC(C)CC(CNC(=O)OC(C)(C)C)C(=O)NCc1ccc2c(c1)CN(C1CCC(=O)NC1=O)C2=O] |

In a preferred embodiment, the compound structure is shown in Formula IV,

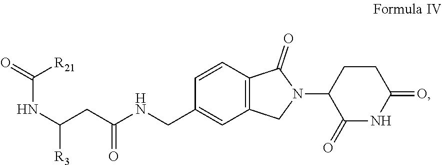

Wherein,
R₃ is selected from: C₁₋₈ alkyl, C₂₋₈ alkenyl, C₂₋₈ alkynyl, C₃₋₈ cycloalkyl, 3- to 8-membered heterocyclic groups, aryl and heteroaryl;
R₂₁ is selected from: C₁₋₈ alkyl, C₂₋₈ alkenyl, C₂₋₈ alkynyl, C₃₋₈ cycloalkyl, 3- to 8-membered heterocyclic groups, aryl and heteroaryl, OR₂₂; wherein, R₂₂ is selected from: C₁₋₈ alkyl, C₂₋₈ alkenyl, C₂₋₈ alkynyl, C₃₋₈ cycloalkyl, 3- to 8-membered heterocyclic groups, aryl and heteroaryl;
Wherein, each of the above-mentioned alkyl, alkenyl, alkynyl, cycloalkyl, heterocyclic groups, aryl, heteroaryl can be optionally and independently substituted by 1-3 substituents, the substituents are independently halogens, C₁₋₄ alkyl, C₂₋₄ alkenyl, C₂₋₄ alkynyl, C₃₋₈ cycloalkyl, 3- to 8-membered heterocyclic groups, aryl, heteroaryl, CN, OH, NH₂, or NO₂.

In another embodiment, R₃ is selected from: C₁₋₄ alkyl without or with 1 to 3 substituents, C₃₋₈ cycloalkyl without or with 1 to 3 substituents and aryl without or with 1 to 3 substituents.

In another embodiment, R₂₁ is OR₂₂; wherein, R₂₂ is selected from: C₁₋₄ alkyl without or with 1 to 3 substituents, C₃₋₈ cycloalkyl without or with 1 to 3 substituents and aryl without or with 1 to 3 substituents, heteroaryl without or with 1 to 3 substituents.

In some preferred embodiments, the compound is selected from any of the following groups:

| Compound No. | Structure |
|---|---|
| 5-97 | 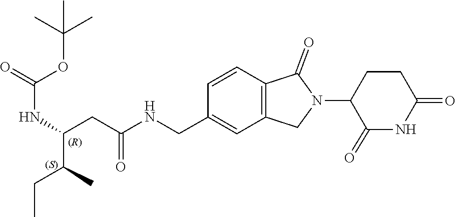 |
| 4-130 | 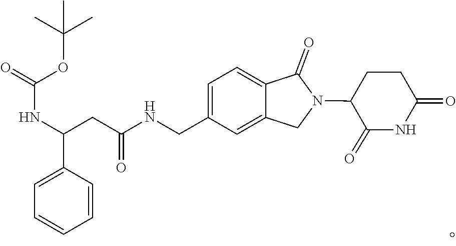 [SMILES: CC(C)(C)OC(=O)NC(CC(=O)NCc1ccc2c(c1)CN(C1CCC(=O)NC1=O)C2=O)c1ccccc1] |
| 5-99 | 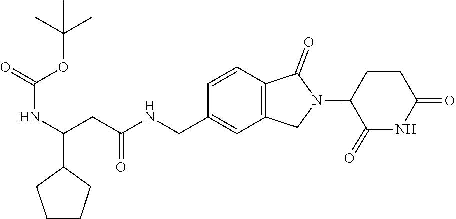 [SMILES: CC(C)(C)OC(=O)NC(CC(=O)NCc1ccc2c(c1)CN(C1CCC(=O)NC1=O)C2=O)C1CCCC1] |

In a preferred embodiment, the compound structure is shown in Formula V,

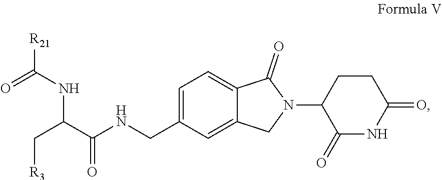

Wherein,
R₃ is selected from: C₁₋₈ alkyl, C₂₋₈ alkenyl, C₂₋₈ alkynyl, C₃₋₈ cycloalkyl, 3- to 8-membered heterocyclic groups, aryl and heteroaryl;
R₂₁ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl, OR₂₂; wherein, R₂₂ is selected from: C₁₋₈ alkyl, C₂₋₈ alkenyl, C₂₋₈ alkynyl, C₃₋₈ cycloalkyl, 3- to 8-membered heterocyclic groups, aryl and heteroaryl;
Wherein, each of the above-mentioned alkyl, alkenyl, alkynyl, cycloalkyl, heterocyclic groups, aryl, heteroaryl can be optionally and independently substituted by 1-3 substituents, the substituents are independently halogens, C₁₋₄ alkyl, C₂₋₄ alkenyl, C₂₋₄ alkynyl, C₃₋₈ cycloalkyl, 3- to 8-membered heterocyclic groups, aryl, heteroaryl, CN, OH, NH₂, or NO₂.

In another embodiment, R₃ is selected from: C₁₋₄ alkyl without or with 1 to 3 substituents, C₃₋₈ cycloalkyl without or with 1 to 3 substituents and aryl without or with 1 to 3 substituents.

In another embodiment, R₂₁ is OR₂₂; wherein, R₂₂ is selected from: C₁₋₄ alkyl without or with 1 to 3 substituents, C₃₋₈ cycloalkyl without or with 1 to 3 substituents and aryl without or with 1 to 3 substituents, heteroaryl without or with 1 to 3 substituents.

In some preferred embodiments, the compound is selected from any of the following groups:

| Compound | |
|---|---|
| No. | Structure |
| 5-39 | 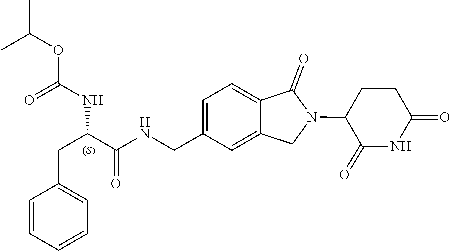 |
| 5-46 | 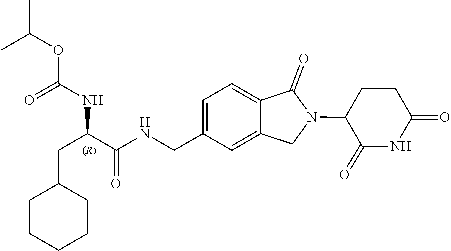 |
| 5-22 | 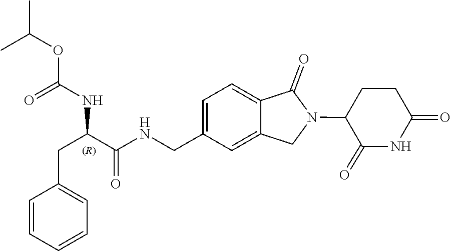 |
| 5-37 | 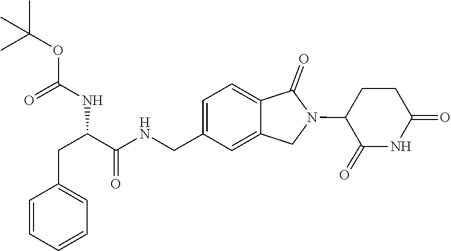 [SMILES: CC(C)(C)OC(=O)N[C@@H](Cc1ccccc1)C(=O)NCc1ccc2c(c1)CN(C1CCC(=O)NC1=O)C2=O] |
| 4-132 | 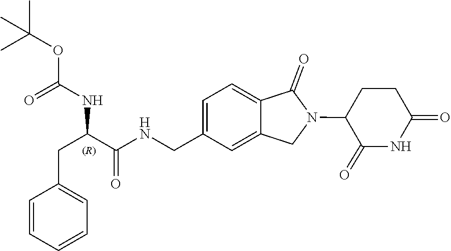 |
| 5-83 | 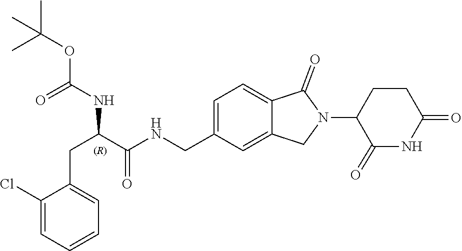 |
| 5-92 | 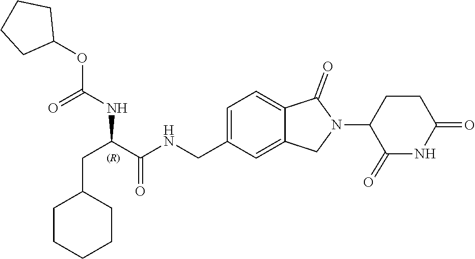 |
| 5-93 | 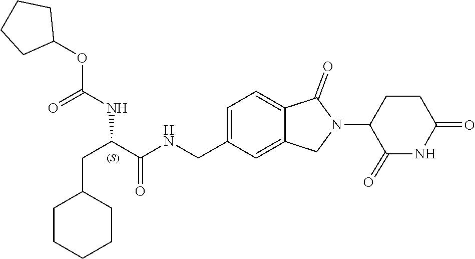 |
| 5-94 | 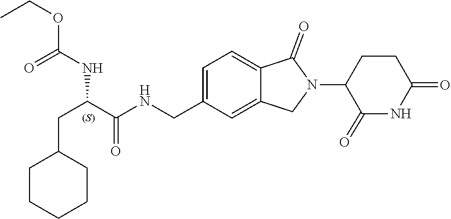 |

Those skilled in the art should understand that in the general formula compound of the present invention, the prerequisite for selecting each functional group is that can form a stable chemical structure.

In the second aspect, the invention provide a pharmaceutical composition comprising an effective dose of a compound of the first aspect of the present invention or a pharmaceutically acceptable salt, prodrug thereof, and a pharmaceutically acceptable carrier.

In another preferred example, the effective dose refers to the therapeutic or inhibitory effective dose, preferably 0.01˜99.99%.

In another preferred example, the drug combination further comprises one or more anti-tumor agents.

In another preferred example, the drug combination is used to degrade GSPT1 or inhibit its activity.

In another preferred example, the drug combination is used for treating diseases related to GSPT1 overexpression.

In the third aspect, provided is a use of a compound as described in the first aspect of the present invention for:
(a) Preparation of drugs for treating diseases related to GSPT1 activity or expression levels;
(b) Preparation of GSPT1 targeted inhibitors or degradation agents;
(c) Non therapeutic inhibition or degradation of GSPT1 in vitro;
(d) Non therapeutic inhibition of tumor cell proliferation in vitro; and/or
(e) Treating diseases related to GSPT1 activity or expression levels.

In another preferred example, said diseases include tumors, etc.

In the fourth aspect, a method for inhibiting or degrading GSPT1 is provided, comprising the steps of administering an effective dose of a compound of the first aspect of the present invention or a pharmaceutically acceptable salt thereof to the subject of action, or administering an effective dose of a pharmaceutical composition as described in the second aspect of the present invention to the subject of action.

In another preferred example, said inhibition is a non therapeutic inhibition in vitro.

In another preferred example, when an effective dose of compound of Formula I as described in the first aspect of the present invention or a pharmaceutically acceptable salt thereof is applied to the subject of action, the dose is 0.001-500 nmol/L, preferably 0.01-200 nmol/L.

In the fifth aspect, a method for treating diseases related to GSPT1 is provided, comprising: administrating a therapeutic effective dose of compound of Formula I as described in the first aspect of the present invention, or a pharmaceutical composition as described in the second aspect of the present invention to the treated subject.

In another preferred example, the subject is a mammal; preferably human.

In another preferred example, the diseases related to GSPT1 are tumors.

In the sixth aspect, a method for inhibiting tumor cells in vitro is provided, comprising: administrating a compound of Formula I as described in the first aspect of the present invention or a pharmaceutical composition as described in the second aspect of the present invention to tumor cells in an effective inhibitory dose.

In another preferred example, said tumor cells overexpress GSPT1 protein.

It should be understood that within the scope of the present invention, the above-mentioned technical features of the present invention and the specific technical features described below (such as embodiments) can be combined with each other to form new or preferred technical solutions. Due to space limitations, we will not elaborate on each one here.

### EMBODIMENT

After extensive and in-depth research, the inventor prepared a class of compounds with the structure shown in Formula I and found that they had GSPT1 inhibition and degradation activity. And the compounds exhibit inhibitory and degradation effects on GSPT1 protein at extremely low concentrations. Therefore, they can be used to treat diseases related to GSPT1 activity or expression, such as tumors. On this basis, the present invention has been completed.

### Compounds of the Present Invention

In a preferred embodiment of the present invention, the present invention provides a compound as shown in Formula I, or a pharmaceutically acceptable salt thereof:

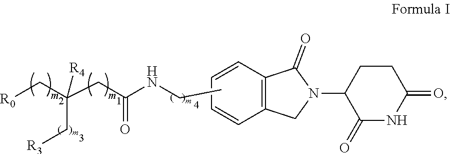

Wherein:
R₀ is NR₁R₂ or OR₂;
R₁ is selected from: H, C₁₋₈ alkyl, C₂₋₈ alkenyl, C₂₋₈ alkynyl, C₃₋₁₀ cycloalkyl, 3- to 8-membered heterocyclic groups, aryl and heteroaryl;
R₂ is selected from: H, C₁₋₈ alkyl, C₂₋₈ alkenyl, C₂₋₈ alkynyl, C₃₋₁₀ cycloalkyl, 3- to 8-membered heterocyclic groups, aryl, heteroaryl, (CH₂)ₙC(O)R₅, C(O)(CHR₆)ₙR₅, C(O)(CHR₆)ₙOR₅, S(O)₂R₅, C(O)C(O)R₅; wherein R₅ is independently selected from: H, C₁₋₈ alkyl, C₂₋₈ alkenyl, C₂₋₈ alkynyl, C₃₋₁₀ cycloalkyl, 3- to 8-membered heterocyclic groups, aryl, heteroaryl, NR₇R₈; R₇ is selected from: H, C₁₋₈ alkyl; R₈ is selected from: H, C₁₋₈ alkyl, C₂₋₈ alkenyl, C₂₋₈ alkynyl, C₃₋₁₀ cycloalkyl, 3- to 8-membered heterocyclic groups, aryl, heteroaryl, alkyl carbonyl, alkoxycarbonyl, or R₇ and R₈ are connected into a ring (preferably a 3- to 20-membered ring that is non substituted or substituted by 1-3 substituents, which can be saturated, unsaturated, or aromatic, and can be a single or fused ring); R₆ is independently selected from: H, C₁₋₈ alkyl, C₂₋₈ alkenyl, C₂₋₈ alkynyl, C₃₋₈ cycloalkyl, 3- to 8-membered heterocyclic groups, aryl, heteroaryl, n is independently 0 or 1;
R₃ is selected from: C₁₋₈ alkyl, C₂₋₈ alkenyl, C₂₋₈ alkynyl, C₃₋₁₀ cycloalkyl, 3- to 8-membered heterocyclic groups, aryl, heteroaryl, C(O)R₉; wherein, R₉ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl, NH₂; or R₂ and R₃ are connected into a ring (preferably a 3- to 20-membered ring that is non substituted or substituted by 1-3 substituents, which can be saturated, unsaturated, or aromatic, and can be a single or fused ring);
R₄ is selected from: H, C₁₋₈ alkyl, C₂₋₈ alkenyl, C₂₋₈ alkynyl, C₃₋₁₀ cycloalkyl, 3- to 8-membered heterocyclic groups, aryl, heteroaryl; or R₃ and R₄ are connected into a ring (preferably, the 3- to 20-membered ring is either non substituted or substituted by 1-3 substituents, which can be saturated, unsaturated carbon ring, or a heterocyclic ring);
Wherein, each of the above-mentioned alkyl, alkenyl, alkyne, cycloalkyl, heterocyclic, aryl, and heteroaryl groups can be optionally and independently substituted by 1-3 substituents, the substituents are independently halogens, C₁₋₄ alkyl, C₂₋₄ alkenyl, C₂₋₄ alkynyl, C₃₋₈ cycloalkyl, 3- to 8-membered heterocyclic groups, aryl, heteroaryl, single or multi halogenated C₁₋₄ alkyl (like-CF₃), alkoxy, alkyl carbonyl, alkoxycarbonyl, CN, OH, NH₂, or NO₂;
m₁ is 0, 1 or 2;
m₂ is 0, 1 or 2;
m₃ is 0, 1 or 2;
m₄ is 0, 1 or 2.

In another embodiment, the structure of the compound is shown in Formula I′:

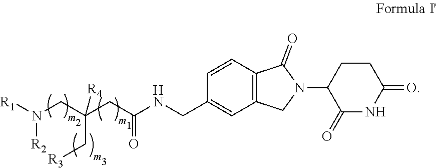

In another embodiment, when R₁ is H, R₂ is not H. The inventor found that when R₁ and R₂ in formula I′ are both hydrogen, the degradation activity of the compound towards GSPT1 protein is extremely poor, with a DC₅₀ much greater than 10 μM.

In another embodiment, the structure of the compound is shown in Formula II:

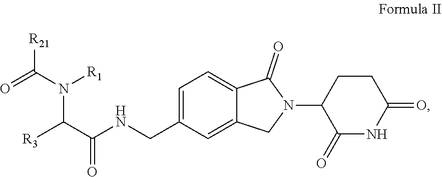

Wherein,
R₁ is selected from: H, C₁₋₈ alkyl, C₂₋₈ alkenyl, C₂₋₈ alkynyl, C₃₋₈ cycloalkyl, 3- to 8-membered heterocyclic groups, aryl, heteroaryl;
R₃ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl;
R₂₁ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl, OR₂₂; wherein, R₂₂ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl;
Wherein, each of the above-mentioned alkyl, alkenyl, alkyne, cycloalkyl, heterocyclic, aryl, and heteroaryl groups can be optionally and independently substituted by 1-3 substituents, the substituents are independently halogens, C₁₋₄ alkyl, C₂₋₄ alkenyl, C₂₋₄ alkynyl, C₃₋₈ cycloalkyl, 3- to 8-membered heterocyclic groups, aryl, heteroaryl, CN, OH, NH₂, or NO₂.

In another preferred embodiment, the structure of the compound is shown in Formula III:

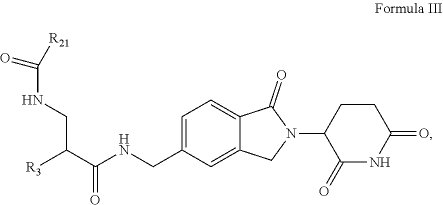

Wherein,
R₃ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl;
R₂₁ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl, OR₂₂; wherein, R₂₂ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl;
Wherein, each of the above-mentioned alkyl, alkenyl, alkyne, cycloalkyl, heterocyclic, aryl, and heteroaryl groups can be optionally and independently substituted by 1-3 substituents, the substituents are independently halogens, C₁₋₄ alkyl, C₂₋₄ alkenyl, C₂₋₄ alkynyl, C₃₋₈ cycloalkyl, 3- to 8-membered heterocyclic groups, aryl, heteroaryl, CN, OH, NH₂, or NO₂.

In another embodiment, the structure of the compound is shown in Formula IV:

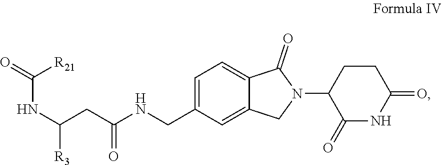

Wherein,
R₃ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl;
R₂₁ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl, OR₂₂; wherein, R₂₂ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl;
Wherein, each of the above-mentioned alkyl, alkenyl, alkyne, cycloalkyl, heterocyclic, aryl, and heteroaryl groups can be optionally and independently substituted by 1-3 substituents, the substituents are independently halogens, C₁₋₄ alkyl, C₂₋₄ alkenyl, C₂₋₄ alkynyl, C₃₋₈ cycloalkyl, 3- to 8-membered heterocyclic groups, aryl, heteroaryl, CN, OH, NH₂, or NO₂.

In another embodiment, the structure of the compound is shown in Formula V:

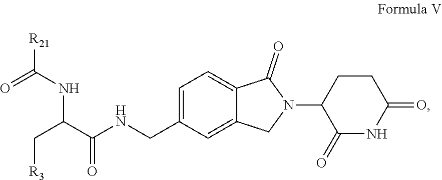

Wherein,
R₃ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl;
R₂₁ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl, OR₂₂; wherein, R₂₂ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl;
Wherein, each of the above-mentioned alkyl, alkenyl, alkyne, cycloalkyl, heterocyclic, aryl, and heteroaryl groups can be optionally and independently substituted by 1-3 substituents, the substituents are independently halogens, C₁₋₄ alkyl, C₂₋₄ alkenyl, C₂₋₄ alkynyl, C₃₋₈ cycloalkyl, 3- to 8-membered heterocyclic groups, aryl, heteroaryl, CN, OH, NH₂, or NO₂.

Preferably, the compounds of the present invention are selected from:

| No. | Structure |
|---|---|
| 4-85 | 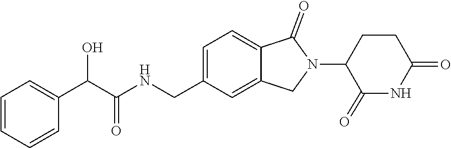 [SMILES: O=C1CCC(N2Cc3cc(CNC(=O)C(O)c4ccccc4)ccc3C2=O)C(=O)N1] |
| 5-15-2 | 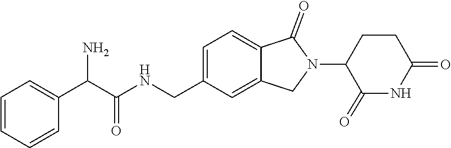 [SMILES: NC(C(=O)NCc1ccc2c(c1)CN(C1CCC(=O)NC1=O)C2=O)c1ccccc1] |
| 4-87-2 | 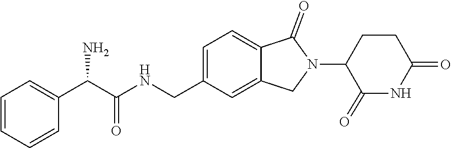 [SMILES: N[C@H](C(=O)NCc1ccc2c(c1)CN(C1CCC(=O)NC1=O)C2=O)c1ccccc1] |
| 4-122-2 | 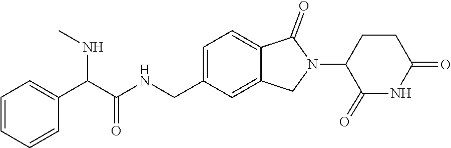 [SMILES: CNC(C(=O)NCc1ccc2c(c1)CN(C1CCC(=O)NC1=O)C2=O)c1ccccc1] |
| 4-112 | 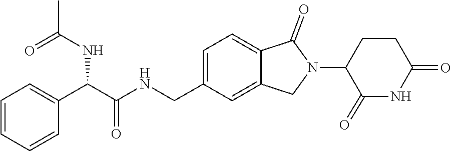 [SMILES: CC(=O)N[C@H](C(=O)NCc1ccc2c(c1)CN(C1CCC(=O)NC1=O)C2=O)c1ccccc1] |
| 4-111 | 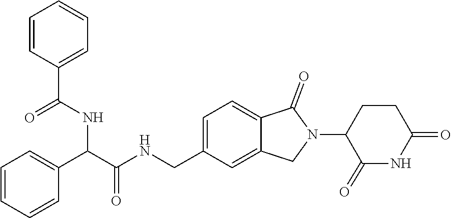 [SMILES: O=C1CCC(N2Cc3cc(CNC(=O)C(NC(=O)c4ccccc4)c4ccccc4)ccc3C2=O)C(=O)N1] |
| 4-121 | 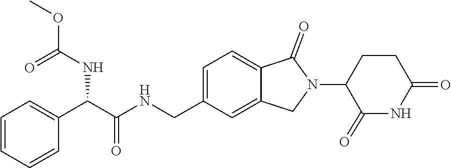 [SMILES: COC(=O)N[C@H](C(=O)NCc1ccc2c(c1)CN(C1CCC(=O)NC1=O)C2=O)c1ccccc1] |
| 4-125 | 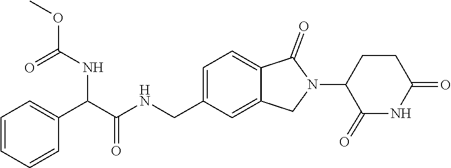 [SMILES: COC(=O)NC(C(=O)NCc1ccc2c(c1)CN(C1CCC(=O)NC1=O)C2=O)c1ccccc1] |
| 4-138 | 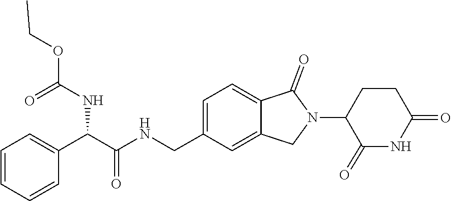 [SMILES: CCOC(=O)N[C@H](C(=O)NCc1ccc2c(c1)CN(C1CCC(=O)NC1=O)C2=O)c1ccccc1] |
| 4-160 | 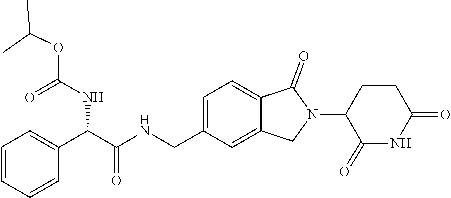 [SMILES: CC(C)OC(=O)N[C@H](C(=O)NCc1ccc2c(c1)CN(C1CCC(=O)NC1=O)C2=O)c1ccccc1] |
| 4-87 | 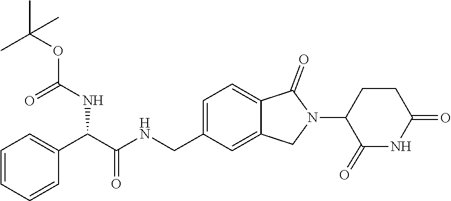 [SMILES: CC(C)(C)OC(=O)N[C@H](C(=O)NCc1ccc2c(c1)CN(C1CCC(=O)NC1=O)C2=O)c1ccccc1] |
| 4-108 | 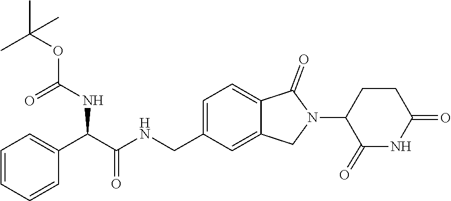 [SMILES: CC(C)(C)OC(=O)N[C@@H](C(=O)NCc1ccc2c(c1)CN(C1CCC(=O)NC1=O)C2=O)c1ccccc1] |
| 4-127 | 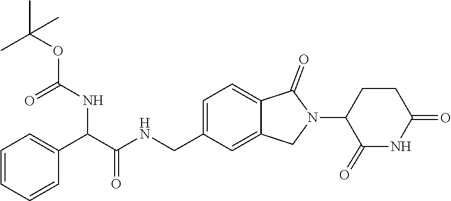 [SMILES: CC(C)(C)OC(=O)NC(C(=O)NCc1ccc2c(c1)CN(C1CCC(=O)NC1=O)C2=O)c1ccccc1] |
| 4-159 | 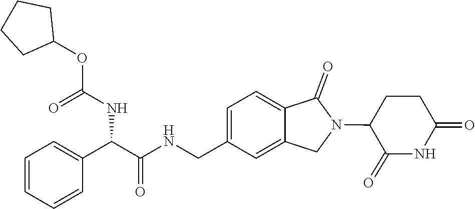 [SMILES: O=C1CCC(N2Cc3cc(CNC(=O)[C@@H](NC(=O)OC4CCCC4)c4ccccc4)ccc3C2=O)C(=O)N1] |
| 4-175 | 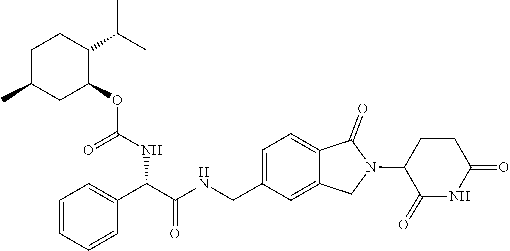 |
| 4-195 | 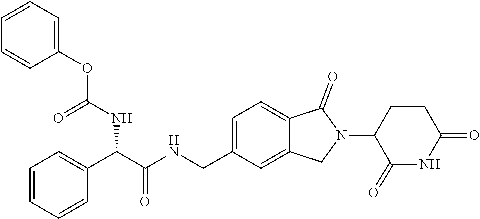 [SMILES: O=C1CCC(N2Cc3cc(CNC(=O)[C@@H](NC(=O)Oc4ccccc4)c4ccccc4)ccc3C2=O)C(=O)N1] |
| 4-139 | 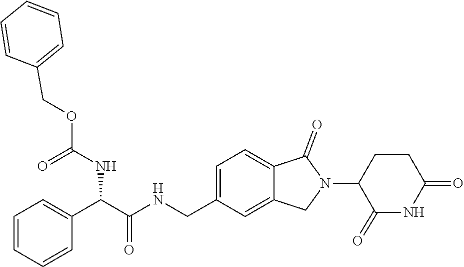 [SMILES: O=C1CCC(N2Cc3cc(CNC(=O)[C@@H](NC(=O)OCc4ccccc4)c4ccccc4)ccc3C2=O)C(=O)N1] |
| 4-184 | 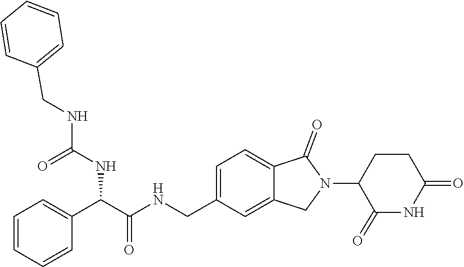 [SMILES: O=C1CCC(N2Cc3cc(CNC(=O)[C@@H](NC(=O)NCc4ccccc4)c4ccccc4)ccc3C2=O)C(=O)N1] |
| 4-182 | 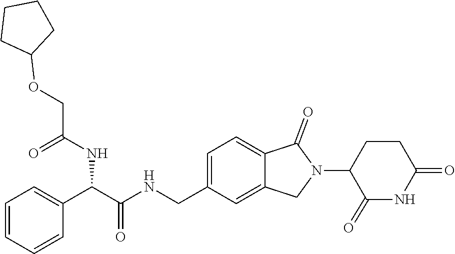 [SMILES: O=C1CCC(N2Cc3cc(CNC(=O)[C@@H](NC(=O)COC4CCCC4)c4ccccc4)ccc3C2=O)C(=O)N1] |
| 4-172 | 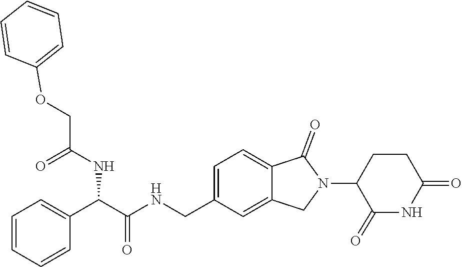 [SMILES: O=C1CCC(N2Cc3cc(CNC(=O)[C@@H](NC(=O)COc4ccccc4)c4ccccc4)ccc3C2=O)C(=O)N1] |
| 5-16 | 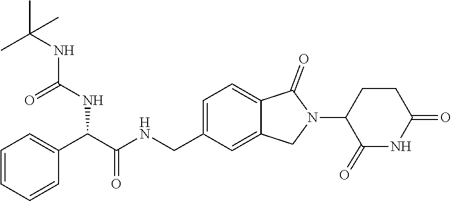 [SMILES: CC(C)(C)NC(=O)N[C@H](C(=O)NCc1ccc2c(c1)CN(C1CCC(=O)NC1=O)C2=O)c1ccccc1] |
| 4-126 | 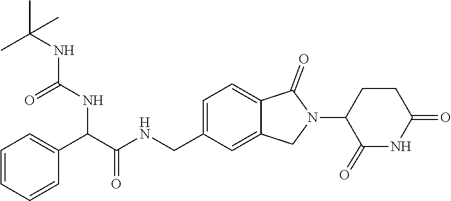 [SMILES: CC(C)(C)NC(=O)NC(C(=O)NCc1ccc2c(c1)CN(C1CCC(=O)NC1=O)C2=O)c1ccccc1] |
| 4-129 | 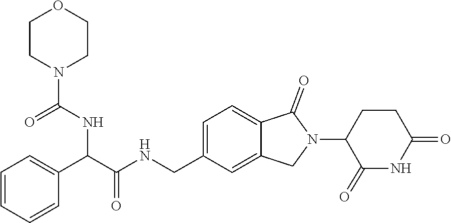 [SMILES: O=C1CCC(N2Cc3cc(CNC(=O)C(NC(=O)N4CCOCC4)c4ccccc4)ccc3C2=O)C(=O)N1] |
| 4-158 | 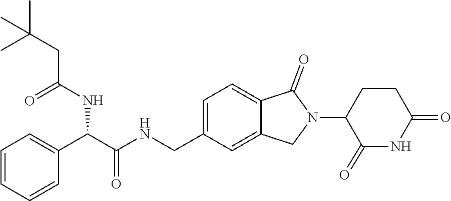 [SMILES: CC(C)(C)CC(=O)N[C@H](C(=O)NCc1ccc2c(c1)CN(C1CCC(=O)NC1=O)C2=O)c1ccccc1] |
| 4-165 | 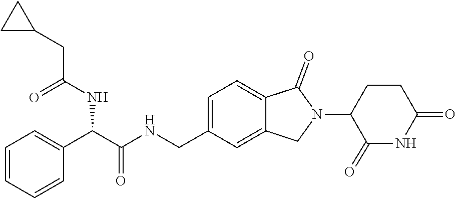 [SMILES: O=C1CCC(N2Cc3cc(CNC(=O)[C@@H](NC(=O)CC4CC4)c4ccccc4)ccc3C2=O)C(=O)N1] |
| 4-176 | 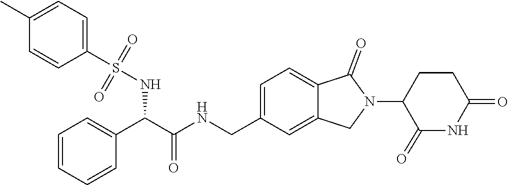 [SMILES: Cc1ccc(S(=O)(=O)N[C@H](C(=O)NCc2ccc3c(c2)CN(C2CCC(=O)NC2=O)C3=O)c2ccccc2)cc1] |
| 4-177 | 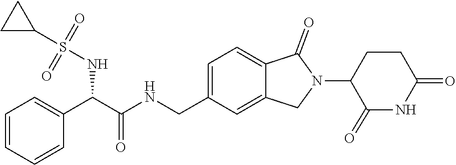 [SMILES: O=C1CCC(N2Cc3cc(CNC(=O)[C@@H](NS(=O)(=O)C4CC4)c4ccccc4)ccc3C2=O)C(=O)N1] |
| 4-197 | 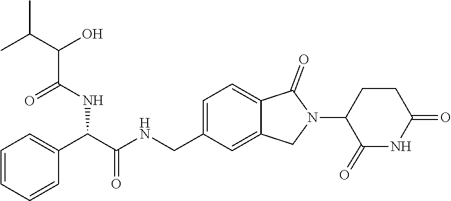 [SMILES: CC(C)C(O)C(=O)N[C@H](C(=O)NCc1ccc2c(c1)CN(C1CCC(=O)NC1=O)C2=O)c1ccccc1] |
| 4-192 | 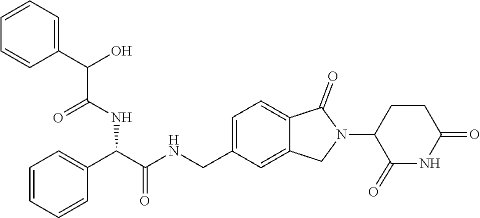 [SMILES: O=C1CCC(N2Cc3cc(CNC(=O)[C@@H](NC(=O)C(O)c4ccccc4)c4ccccc4)ccc3C2=O)C(=O)N1] |
| 4-199 | 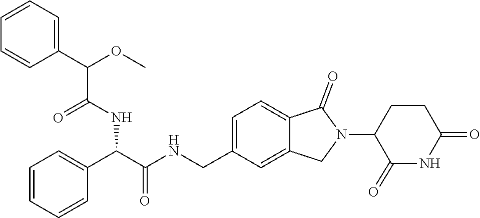 [SMILES: COC(C(=O)N[C@H](C(=O)NCc1ccc2c(c1)CN(C1CCC(=O)NC1=O)C2=O)c1ccccc1)c1ccccc1] |
| 4-198 | 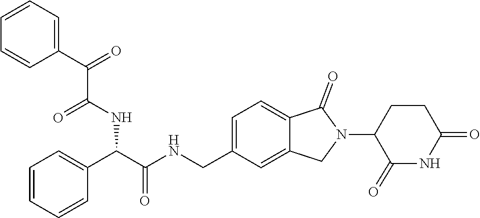 [SMILES: O=C1CCC(N2Cc3cc(CNC(=O)[C@@H](NC(=O)C(=O)c4ccccc4)c4ccccc4)ccc3C2=O)C(=O)N1] |
| 4-196-2 | 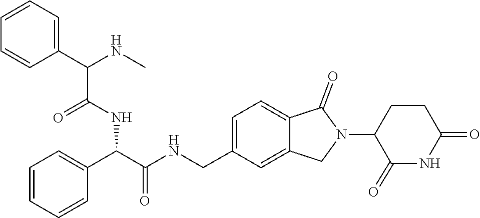 [SMILES: CNC(C(=O)N[C@H](C(=O)NCc1ccc2c(c1)CN(C1CCC(=O)NC1=O)C2=O)c1ccccc1)c1ccccc1] |
| 4-193 | 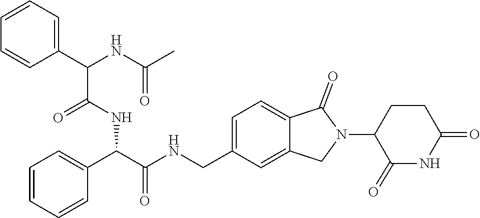 [SMILES: CC(=O)NC(C(=O)N[C@H](C(=O)NCc1ccc2c(c1)CN(C1CCC(=O)NC1=O)C2=O)c1ccccc1)c1ccccc1] |
| 4-109 | 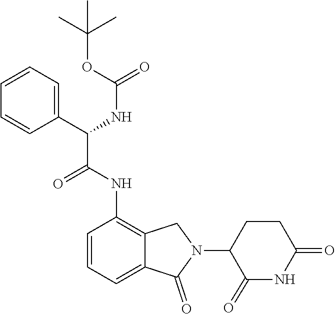 [SMILES: CC(C)(C)OC(=O)N[C@H](C(=O)Nc1cccc2c1CN(C1CCC(=O)NC1=O)C2=O)c1ccccc1] |
| 4-118 | 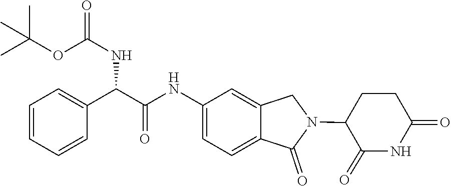 [SMILES: CC(C)(C)OC(=O)N[C@H](C(=O)Nc1ccc2c(c1)CN(C1CCC(=O)NC1=O)C2=O)c1ccccc1] |
| 4-153 | 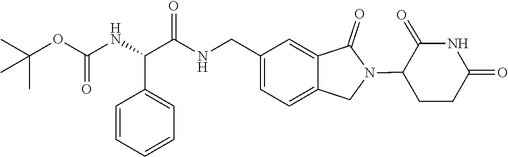 [SMILES: CC(C)(C)OC(=O)N[C@H](C(=O)NCc1ccc2c(c1)C(=O)N(C1CCC(=O)NC1=O)C2)c1ccccc1] |
| 4-167 | 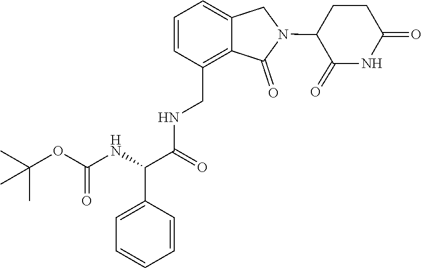 [SMILES: CC(C)(C)OC(=O)N[C@H](C(=O)NCc1cccc2c1C(=O)N(C1CCC(=O)NC1=O)C2)c1ccccc1] |
| 4-117 | 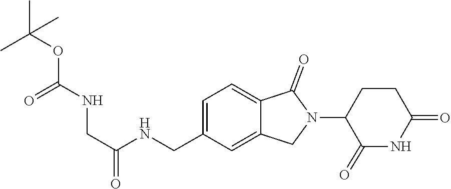 [SMILES: CC(C)(C)OC(=O)NCC(=O)NCc1ccc2c(c1)CN(C1CCC(=O)NC1=O)C2=O] |
| 4-123 | 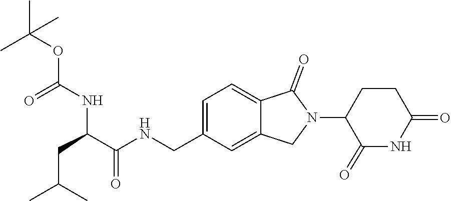 [SMILES: CC(C)C[C@@H](NC(=O)OC(C)(C)C)C(=O)NCc1ccc2c(c1)CN(C1CCC(=O)NC1=O)C2=O] |
| 5-57 | 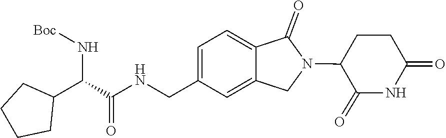 [SMILES: CC(C)(C)OC(=O)N[C@H](C(=O)NCc1ccc2c(c1)CN(C1CCC(=O)NC1=O)C2=O)C1CCCC1] |
| 5-56 | 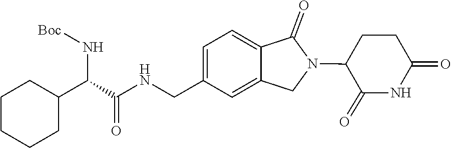 [SMILES: CC(C)(C)OC(=O)N[C@H](C(=O)NCc1ccc2c(c1)CN(C1CCC(=O)NC1=O)C2=O)C1CCCCC1] |
| 4-124 | 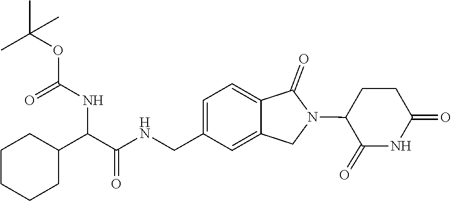 [SMILES: CC(C)(C)OC(=O)NC(C(=O)NCc1ccc2c(c1)CN(C1CCC(=O)NC1=O)C2=O)C1CCCCC1] |
| 4-133 | 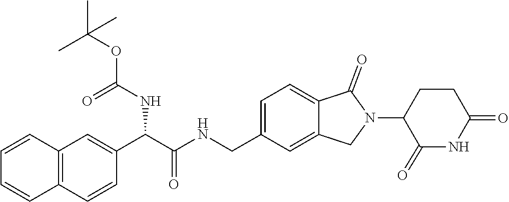 [SMILES: CC(C)(C)OC(=O)N[C@H](C(=O)NCc1ccc2c(c1)CN(C1CCC(=O)NC1=O)C2=O)c1ccc2ccccc2c1] |
| 4-134 | 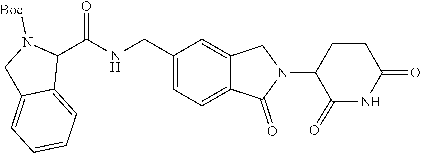 [SMILES: CC(C)(C)OC(=O)N1Cc2ccccc2C1C(=O)NCc1ccc2c(c1)CN(C1CCC(=O)NC1=O)C2=O] |
| 5-32 | 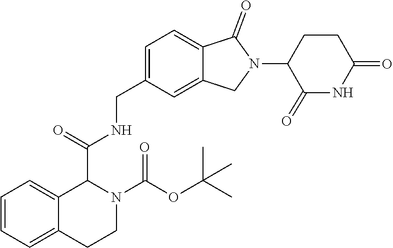 [SMILES: CC(C)(C)OC(=O)N1CCc2ccccc2C1C(=O)NCc1ccc2c(c1)CN(C1CCC(=O)NC1=O)C2=O] |
| 4-136 | 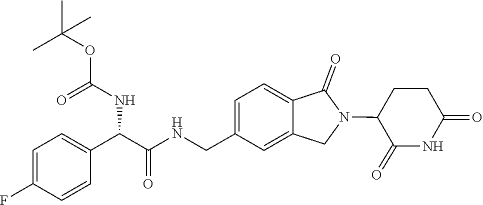 [SMILES: CC(C)(C)OC(=O)N[C@H](C(=O)NCc1ccc2c(c1)CN(C1CCC(=O)NC1=O)C2=O)c1ccc(F)cc1] |
| 5-5 | 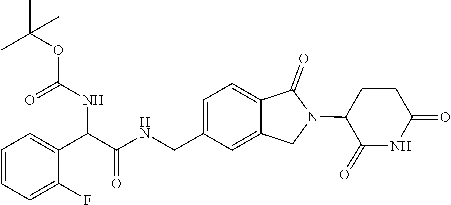 [SMILES: CC(C)(C)OC(=O)NC(C(=O)NCc1ccc2c(c1)CN(C1CCC(=O)NC1=O)C2=O)c1ccccc1F] |
| 4-200 | 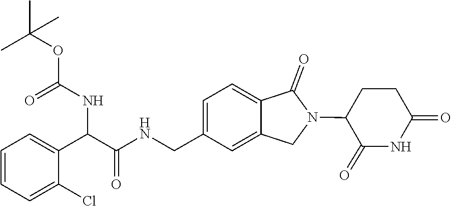 [SMILES: CC(C)(C)OC(=O)NC(C(=O)NCc1ccc2c(c1)CN(C1CCC(=O)NC1=O)C2=O)c1ccccc1Cl] |
| 5-17 | 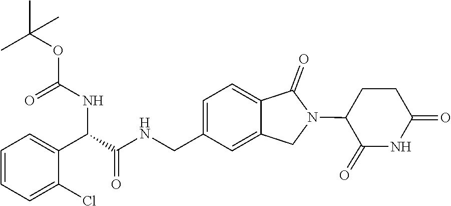 [SMILES: CC(C)(C)OC(=O)N[C@H](C(=O)NCc1ccc2c(c1)CN(C1CCC(=O)NC1=O)C2=O)c1ccccc1Cl] |
| 4-140 | 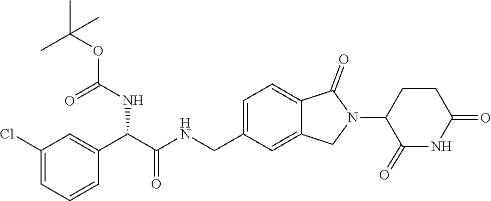 [SMILES: CC(C)(C)OC(=O)N[C@H](C(=O)NCc1ccc2c(c1)CN(C1CCC(=O)NC1=O)C2=O)c1cccc(Cl)c1] |
| 4-150 | 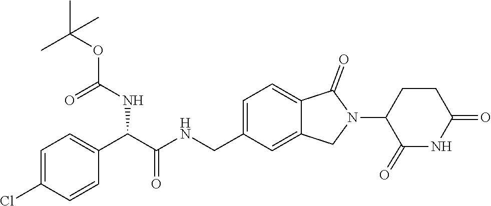 [SMILES: CC(C)(C)OC(=O)N[C@H](C(=O)NCc1ccc2c(c1)CN(C1CCC(=O)NC1=O)C2=O)c1ccc(Cl)cc1] |
| 5-7 | 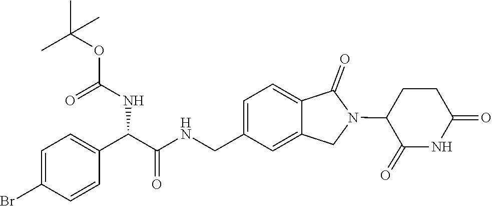 [SMILES: CC(C)(C)OC(=O)N[C@H](C(=O)NCc1ccc2c(c1)CN(C1CCC(=O)NC1=O)C2=O)c1ccc(Br)cc1] |
| 4-152 | 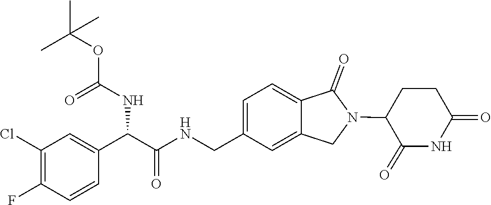 [SMILES: CC(C)(C)OC(=O)N[C@H](C(=O)NCc1ccc2c(c1)CN(C1CCC(=O)NC1=O)C2=O)c1ccc(F)c(Cl)c1] |
| 4-151 | 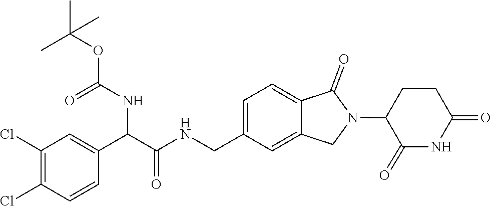 [SMILES: CC(C)(C)OC(=O)NC(C(=O)NCc1ccc2c(c1)CN(C1CCC(=O)NC1=O)C2=O)c1ccc(Cl)c(Cl)c1] |
| 5-6 | 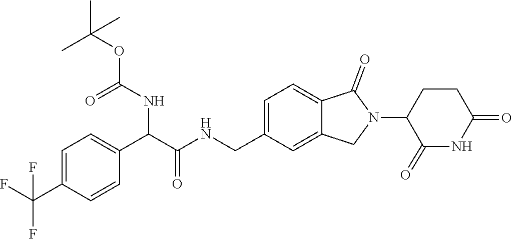 [SMILES: CC(C)(C)OC(=O)NC(C(=O)NCc1ccc2c(c1)CN(C1CCC(=O)NC1=O)C2=O)c1ccc(C(F)(F)F)cc1] |
| 5-13 | 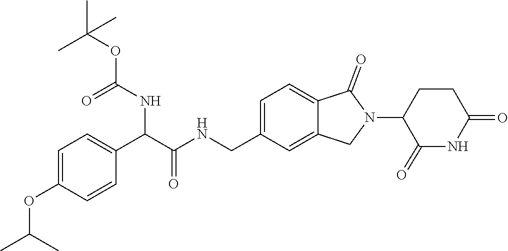 [SMILES: CC(C)Oc1ccc(C(NC(=O)OC(C)(C)C)C(=O)NCc2ccc3c(c2)CN(C2CCC(=O)NC2=O)C3=O)cc1] |
| 4-137 | 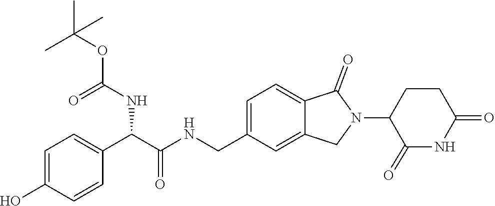 [SMILES: CC(C)(C)OC(=O)N[C@H](C(=O)NCc1ccc2c(c1)CN(C1CCC(=O)NC1=O)C2=O)c1ccc(O)cc1] |
| 4-166 | 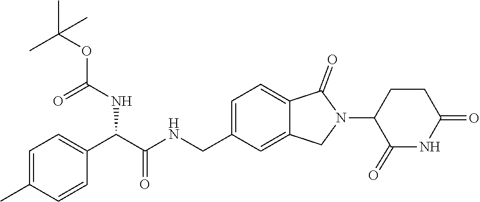 [SMILES: Cc1ccc([C@H](NC(=O)OC(C)(C)C)C(=O)NCc2ccc3c(c2)CN(C2CCC(=O)NC2=O)C3=O)cc1] |
| 4-130 | 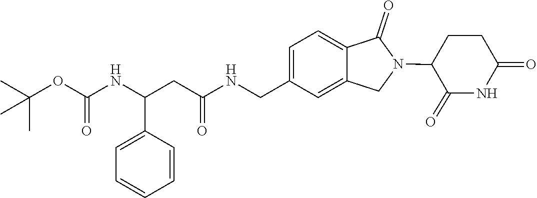 [SMILES: CC(C)(C)OC(=O)NC(CC(=O)NCc1ccc2c(c1)CN(C1CCC(=O)NC1=O)C2=O)c1ccccc1] |
| 4-131 | 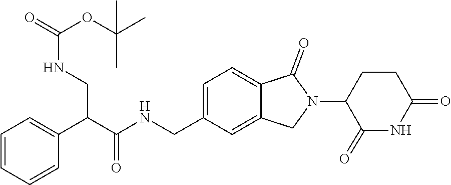 [SMILES: CC(C)(C)OC(=O)NCC(C(=O)NCc1ccc2c(c1)CN(C1CCC(=O)NC1=O)C2=O)c1ccccc1] |
| 4-201 | 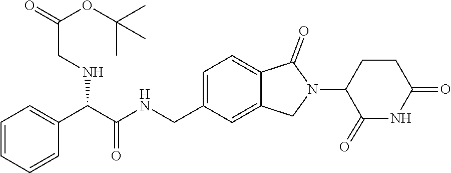 [SMILES: CC(C)(C)OC(=O)CN[C@H](C(=O)NCc1ccc2c(c1)CN(C1CCC(=O)NC1=O)C2=O)c1ccccc1] |
| 5-39 | 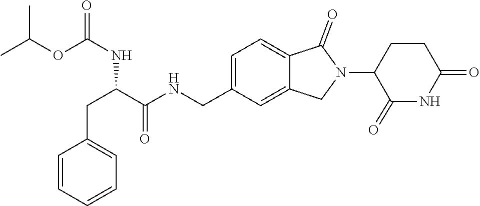 [SMILES: CC(C)OC(=O)N[C@@H](Cc1ccccc1)C(=O)NCc1ccc2c(c1)CN(C1CCC(=O)NC1=O)C2=O] |
| 5-37 | 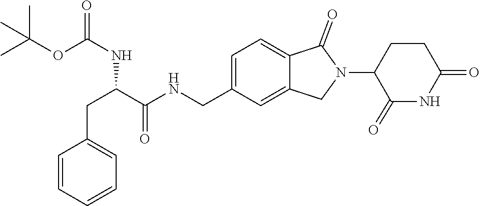 [SMILES: CC(C)(C)OC(=O)N[C@@H](Cc1ccccc1)C(=O)NCc1ccc2c(c1)CN(C1CCC(=O)NC1=O)C2=O] |
| 5-22 | 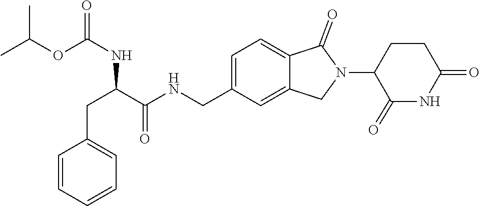 [SMILES: CC(C)OC(=O)N[C@H](Cc1ccccc1)C(=O)NCc1ccc2c(c1)CN(C1CCC(=O)NC1=O)C2=O] |
| 4-132 | 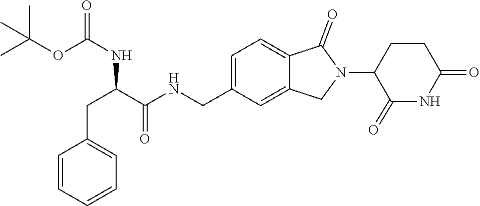 [SMILES: CC(C)(C)OC(=O)N[C@H](Cc1ccccc1)C(=O)NCc1ccc2c(c1)CN(C1CCC(=O)NC1=O)C2=O] |
| 4-171 | 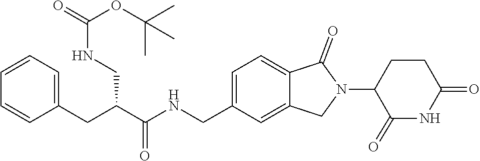 [SMILES: CC(C)(C)OC(=O)NC[C@@H](Cc1ccccc1)C(=O)NCc1ccc2c(c1)CN(C1CCC(=O)NC1=O)C2=O] |
| 4-186 | 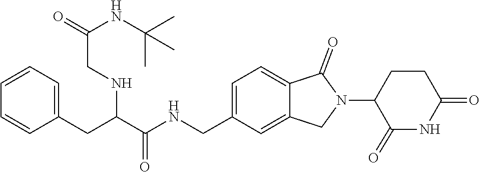 [SMILES: CC(C)(C)NC(=O)CNC(Cc1ccccc1)C(=O)NCc1ccc2c(c1)CN(C1CCC(=O)NC1=O)C2=O] |
| 4-187 | 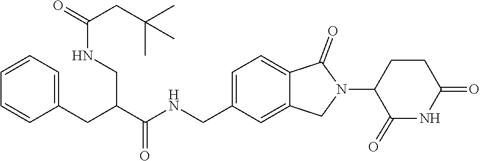 [SMILES: CC(C)(C)CC(=O)NCC(Cc1ccccc1)C(=O)NCc1ccc2c(c1)CN(C1CCC(=O)NC1=O)C2=O] |
| 4-188 | 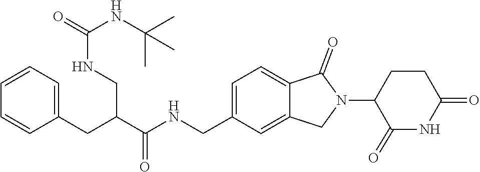 [SMILES: CC(C)(C)NC(=O)NCC(Cc1ccccc1)C(=O)NCc1ccc2c(c1)CN(C1CCC(=O)NC1=O)C2=O] |
| 4-189 | 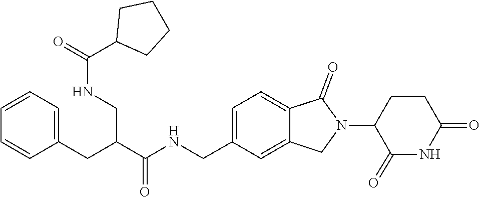 [SMILES: O=C1CCC(N2Cc3cc(CNC(=O)C(CNC(=O)C4CCCC4)Cc4ccccc4)ccc3C2=O)C(=O)N1] |
| 5-8-2 | 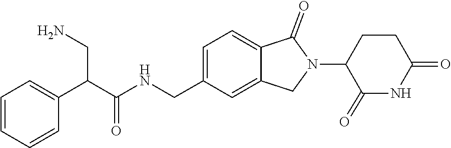 [SMILES: NCC(C(=O)NCc1ccc2c(c1)CN(C1CCC(=O)NC1=O)C2=O)c1ccccc1] |
| 5-2 | 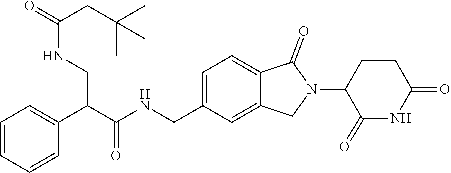 [SMILES: CC(C)(C)CC(=O)NCC(C(=O)NCc1ccc2c(c1)CN(C1CCC(=O)NC1=O)C2=O)c1ccccc1] |
| 5-3 | 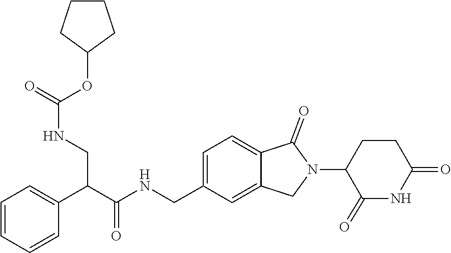 [SMILES: O=C1CCC(N2Cc3cc(CNC(=O)C(CNC(=O)OC4CCCC4)c4ccccc4)ccc3C2=O)C(=O)N1] |
| 5-9 | 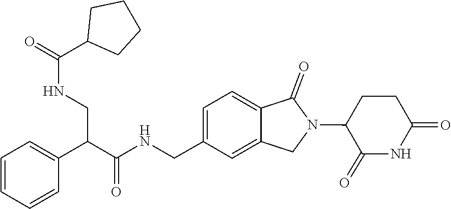 [SMILES: O=C1CCC(N2Cc3cc(CNC(=O)C(CNC(=O)C4CCCC4)c4ccccc4)ccc3C2=O)C(=O)N1] |
| 5-10 | 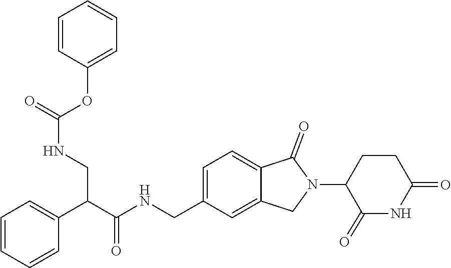 [SMILES: O=C1CCC(N2Cc3cc(CNC(=O)C(CNC(=O)Oc4ccccc4)c4ccccc4)ccc3C2=O)C(=O)N1] |
| 5-11 | 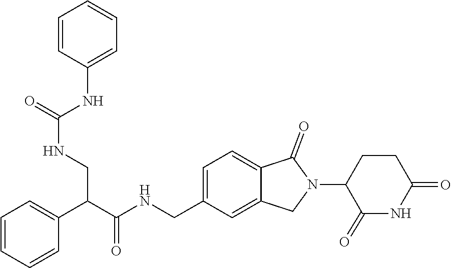 [SMILES: O=C1CCC(N2Cc3cc(CNC(=O)C(CNC(=O)Nc4ccccc4)c4ccccc4)ccc3C2=O)C(=O)N1] |
| 5-12 | 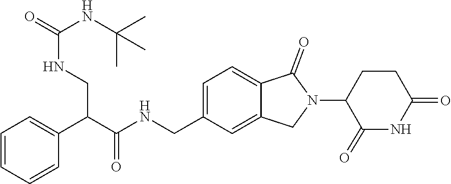 [SMILES: CC(C)(C)NC(=O)NCC(C(=O)NCc1ccc2c(c1)CN(C1CCC(=O)NC1=O)C2=O)c1ccccc1] |
| 5-24 | 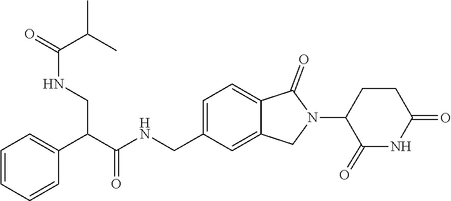 [SMILES: CC(C)C(=O)NCC(C(=O)NCc1ccc2c(c1)CN(C1CCC(=O)NC1=O)C2=O)c1ccccc1] |
| 5-23 | 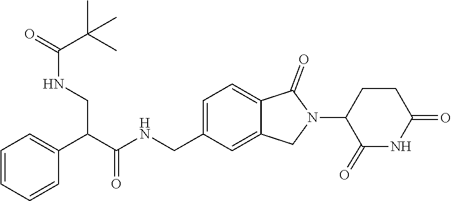 [SMILES: CC(C)(C)C(=O)NCC(C(=O)NCc1ccc2c(c1)CN(C1CCC(=O)NC1=O)C2=O)c1ccccc1] |
| 5-25 | 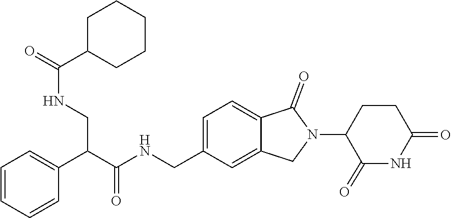 [SMILES: O=C1CCC(N2Cc3cc(CNC(=O)C(CNC(=O)C4CCCCC4)c4ccccc4)ccc3C2=O)C(=O)N1] |
| 5-26 | 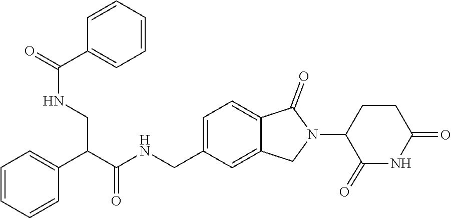 [SMILES: O=C1CCC(N2Cc3cc(CNC(=O)C(CNC(=O)c4ccccc4)c4ccccc4)ccc3C2=O)C(=O)N1] |
| 5-29 | 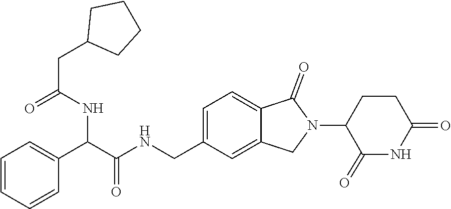 [SMILES: O=C1CCC(N2Cc3cc(CNC(=O)C(NC(=O)CC4CCCC4)c4ccccc4)ccc3C2=O)C(=O)N1] |
| 5-30 | 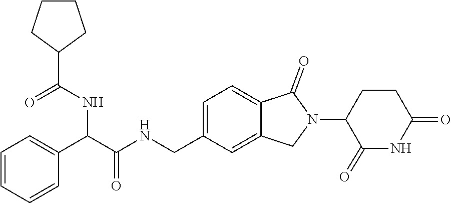 [SMILES: O=C1CCC(N2Cc3cc(CNC(=O)C(NC(=O)C4CCCC4)c4ccccc4)ccc3C2=O)C(=O)N1] |
| 5-33 | 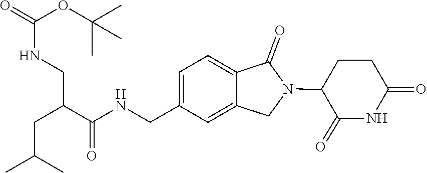 [SMILES: CC(C)CC(CNC(=O)OC(C)(C)C)C(=O)NCc1ccc2c(c1)CN(C1CCC(=O)NC1=O)C2=O] |
| 5-38 | 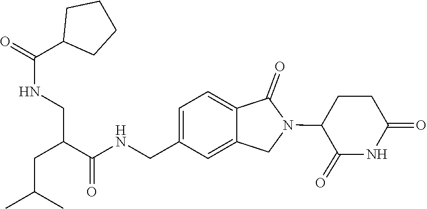 [SMILES: CC(C)CC(CNC(=O)C1CCCC1)C(=O)NCc1ccc2c(c1)CN(C1CCC(=O)NC1=O)C2=O] |
| 5-41 | 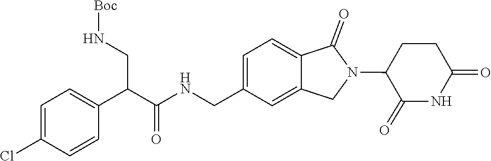 [SMILES: CC(C)(C)OC(=O)NCC(C(=O)NCc1ccc2c(c1)CN(C1CCC(=O)NC1=O)C2=O)c1ccc(Cl)cc1] |
| 5-43 | 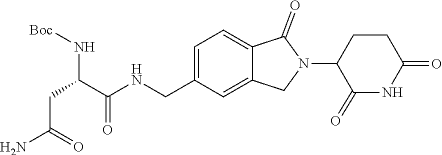 [SMILES: CC(C)(C)OC(=O)N[C@@H](CC(N)=O)C(=O)NCc1ccc2c(c1)CN(C1CCC(=O)NC1=O)C2=O] |
| 5-46 | 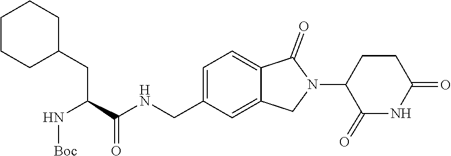 [SMILES: CC(C)(C)OC(=O)N[C@@H](CC1CCCCC1)C(=O)NCc1ccc2c(c1)CN(C1CCC(=O)NC1=O)C2=O] |
| 5-47 | 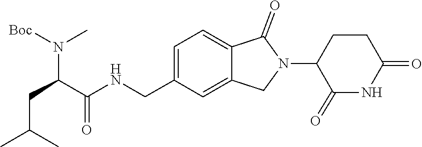 [SMILES: CC(C)C[C@H](C(=O)NCc1ccc2c(c1)CN(C1CCC(=O)NC1=O)C2=O)N(C)C(=O)OC(C)(C)C] |
| 5-52 | 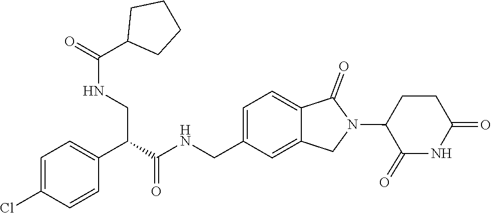 [SMILES: O=C1CCC(N2Cc3cc(CNC(=O)[C@@H](CNC(=O)C4CCCC4)c4ccc(Cl)cc4)ccc3C2=O)C(=O)N1] |
| 5-53 | 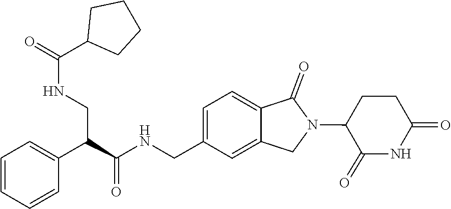 |
| 5-54 | 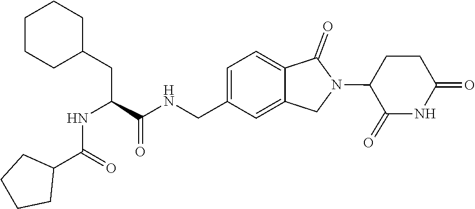 [SMILES: O=C1CCC(N2Cc3cc(CNC(=O)[C@H](CC4CCCCC4)NC(=O)C4CCCC4)ccc3C2=O)C(=O)N1] |
| 5-55 | 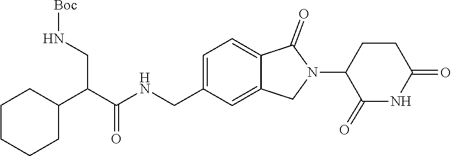 [SMILES: CC(C)(C)OC(=O)NCC(C(=O)NCc1ccc2c(c1)CN(C1CCC(=O)NC1=O)C2=O)C1CCCCC1] |
| 5-58 | 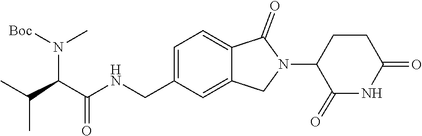 [SMILES: CC(C)[C@H](C(=O)NCc1ccc2c(c1)CN(C1CCC(=O)NC1=O)C2=O)N(C)C(=O)OC(C)(C)C] |
| 5-59 | 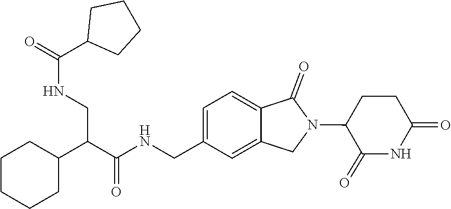 [SMILES: O=C1CCC(N2Cc3cc(CNC(=O)C(CNC(=O)C4CCCC4)C4CCCCC4)ccc3C2=O)C(=O)N1] |
| 5-69 | 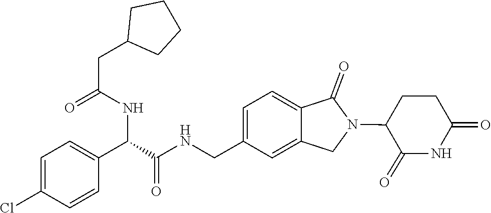 [SMILES: O=C1CCC(N2Cc3cc(CNC(=O)[C@@H](NC(=O)CC4CCCC4)c4ccc(Cl)cc4)ccc3C2=O)C(=O)N1] |
| 5-70 | 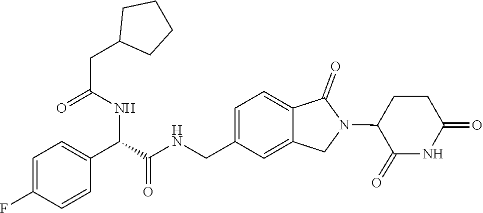 [SMILES: O=C1CCC(N2Cc3cc(CNC(=O)[C@@H](NC(=O)CC4CCCC4)c4ccc(F)cc4)ccc3C2=O)C(=O)N1] |
| 4-135 | 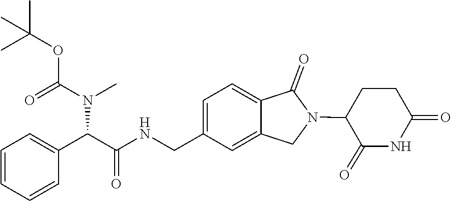 [SMILES: CN(C(=O)OC(C)(C)C)[C@H](C(=O)NCc1ccc2c(c1)CN(C1CCC(=O)NC1=O)C2=O)c1ccccc1] |
| 5-31 | 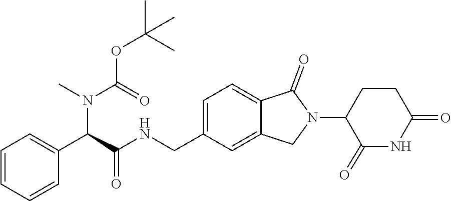 [SMILES: CN(C(=O)OC(C)(C)C)[C@@H](C(=O)NCc1ccc2c(c1)CN(C1CCC(=O)NC1=O)C2=O)c1ccccc1] |
| 5-34 | 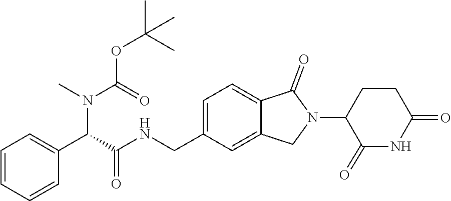 [SMILES: CN(C(=O)OC(C)(C)C)[C@H](C(=O)NCc1ccc2c(c1)CN(C1CCC(=O)NC1=O)C2=O)c1ccccc1] |
| 5-35 | 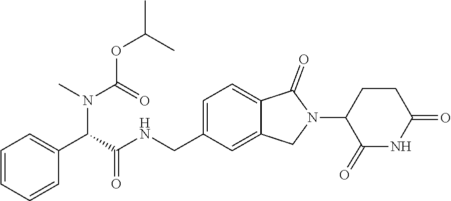 [SMILES: CC(C)OC(=O)N(C)[C@H](C(=O)NCc1ccc2c(c1)CN(C1CCC(=O)NC1=O)C2=O)c1ccccc1] |
| 5-36 | 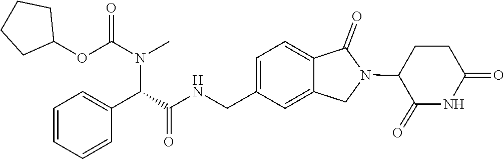 [SMILES: CN(C(=O)OC1CCCC1)[C@H](C(=O)NCc1ccc2c(c1)CN(C1CCC(=O)NC1=O)C2=O)c1ccccc1] |
| 5-40 | 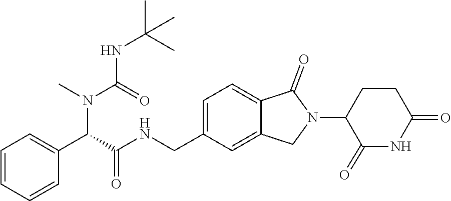 [SMILES: CN(C(=O)NC(C)(C)C)[C@H](C(=O)NCc1ccc2c(c1)CN(C1CCC(=O)NC1=O)C2=O)c1ccccc1] |
| 5-75 | 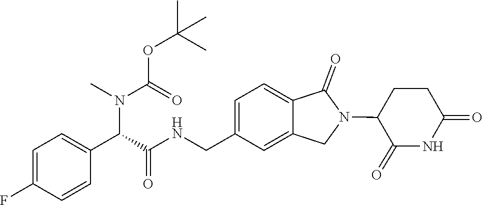 [SMILES: CN(C(=O)OC(C)(C)C)[C@H](C(=O)NCc1ccc2c(c1)CN(C1CCC(=O)NC1=O)C2=O)c1ccc(F)cc1] |
| 5-76 | 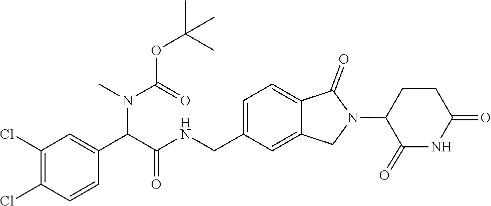 [SMILES: CN(C(=O)OC(C)(C)C)C(C(=O)NCc1ccc2c(c1)CN(C1CCC(=O)NC1=O)C2=O)c1ccc(Cl)c(Cl)c1] |
| 5-77 | 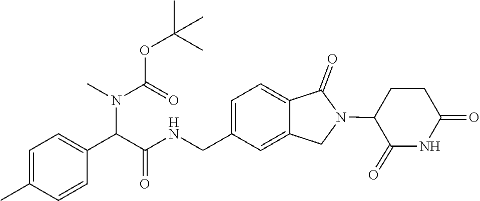 [SMILES: Cc1ccc(C(C(=O)NCc2ccc3c(c2)CN(C2CCC(=O)NC2=O)C3=O)N(C)C(=O)OC(C)(C)C)cc1] |
| 5-78 | 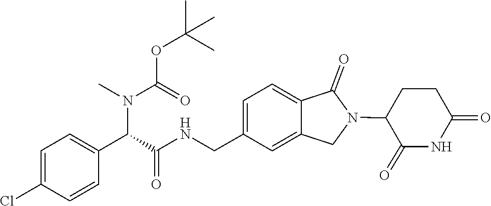 [SMILES: CN(C(=O)OC(C)(C)C)[C@H](C(=O)NCc1ccc2c(c1)CN(C1CCC(=O)NC1=O)C2=O)c1ccc(Cl)cc1] |
| 5-79 | 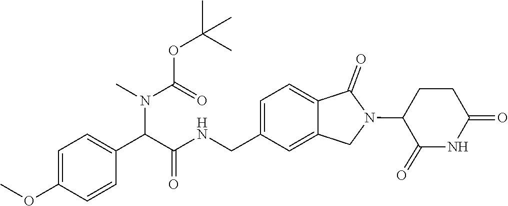 [SMILES: COc1ccc(C(C(=O)NCc2ccc3c(c2)CN(C2CCC(=O)NC2=O)C3=O)N(C)C(=O)OC(C)(C)C)cc1] |
| 1-86 | 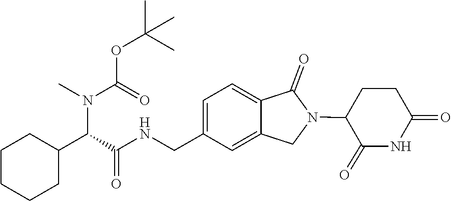 [SMILES: CN(C(=O)OC(C)(C)C)[C@H](C(=O)NCc1ccc2c(c1)CN(C1CCC(=O)NC1=O)C2=O)C1CCCCC1] |
| 1-89 | 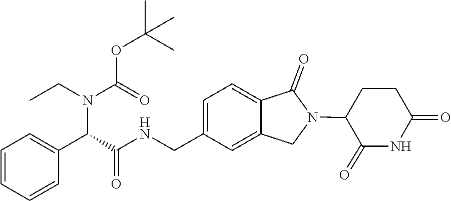 [SMILES: CCN(C(=O)OC(C)(C)C)[C@H](C(=O)NCc1ccc2c(c1)CN(C1CCC(=O)NC1=O)C2=O)c1ccccc1] |
| 5-50 | 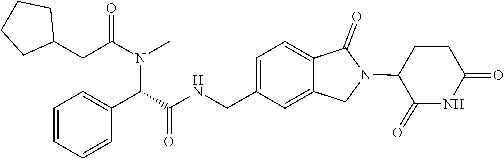 [SMILES: CN(C(=O)CC1CCCC1)[C@H](C(=O)NCc1ccc2c(c1)CN(C1CCC(=O)NC1=O)C2=O)c1ccccc1] |
| 5-49 | 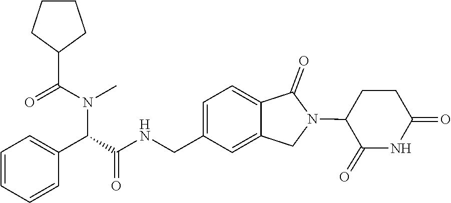 [SMILES: CN(C(=O)C1CCCC1)[C@H](C(=O)NCc1ccc2c(c1)CN(C1CCC(=O)NC1=O)C2=O)c1ccccc1] |
| 5-88 | 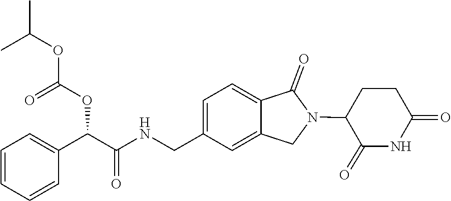 [SMILES: CC(C)OC(=O)O[C@H](C(=O)NCc1ccc2c(c1)CN(C1CCC(=O)NC1=O)C2=O)c1ccccc1] |
| 5-82 | 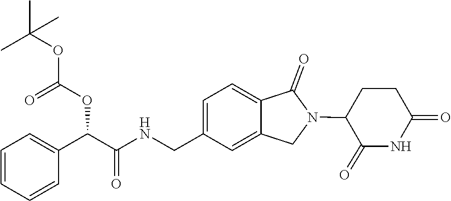 [SMILES: CC(C)(C)OC(=O)O[C@H](C(=O)NCc1ccc2c(c1)CN(C1CCC(=O)NC1=O)C2=O)c1ccccc1] |
| 5-87 | 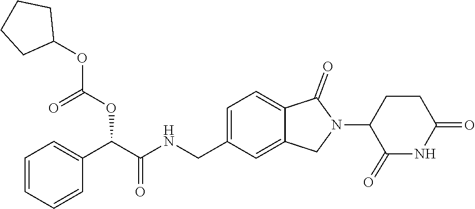 [SMILES: O=C1CCC(N2Cc3cc(CNC(=O)[C@@H](OC(=O)OC4CCCC4)c4ccccc4)ccc3C2=O)C(=O)N1] |
| 5-83 | 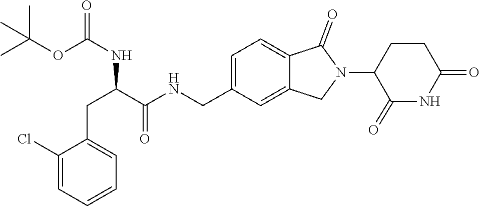 [SMILES: CC(C)(C)OC(=O)N[C@H](Cc1ccccc1Cl)C(=O)NCc1ccc2c(c1)CN(C1CCC(=O)NC1=O)C2=O] |
| 5-85 | 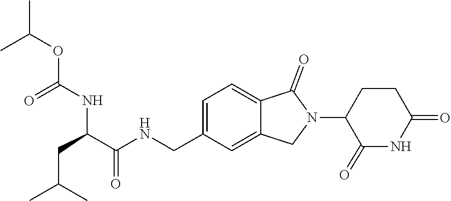 [SMILES: CC(C)C[C@@H](NC(=O)OC(C)C)C(=O)NCc1ccc2c(c1)CN(C1CCC(=O)NC1=O)C2=O] |
| 5-84 | 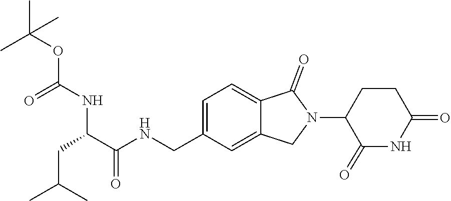 [SMILES: CC(C)C[C@H](NC(=O)OC(C)(C)C)C(=O)NCc1ccc2c(c1)CN(C1CCC(=O)NC1=O)C2=O] |
| 5-92 | 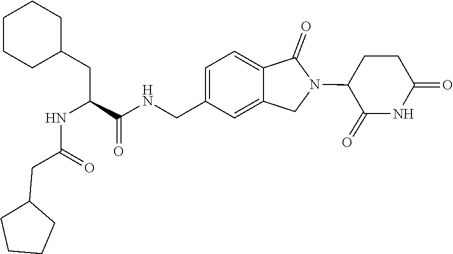 [SMILES: O=C1CCC(N2Cc3cc(CNC(=O)[C@H](CC4CCCCC4)NC(=O)CC4CCCC4)ccc3C2=O)C(=O)N1] |
| 5-93 | 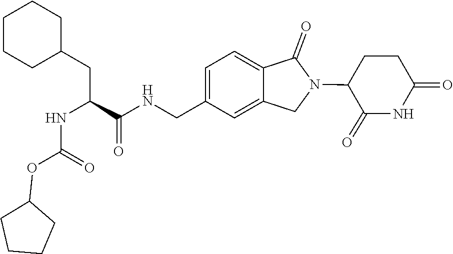 [SMILES: O=C1CCC(N2Cc3cc(CNC(=O)[C@H](CC4CCCCC4)NC(=O)OC4CCCC4)ccc3C2=O)C(=O)N1] |
| 5-94 | 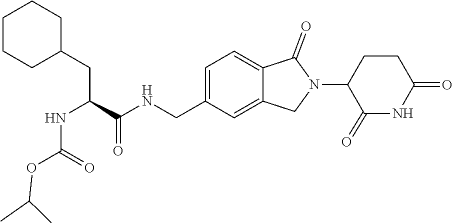 [SMILES: CC(C)OC(=O)N[C@@H](CC1CCCCC1)C(=O)NCc1ccc2c(c1)CN(C1CCC(=O)NC1=O)C2=O] |
| 5-95 | 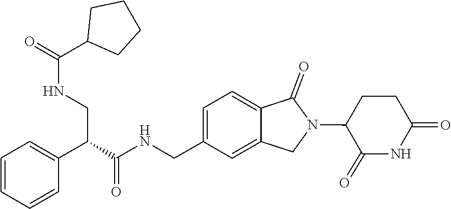 [SMILES: O=C1CCC(N2Cc3cc(CNC(=O)[C@@H](CNC(=O)C4CCCC4)c4ccccc4)ccc3C2=O)C(=O)N1] |
| 5-17b | 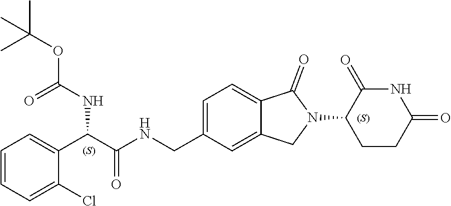 |
| 5-97 | 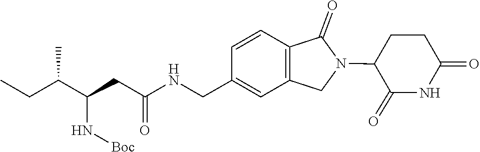 [SMILES: CC[C@H](C)[C@@H](CC(=O)NCc1ccc2c(c1)CN(C1CCC(=O)NC1=O)C2=O)NC(=O)OC(C)(C)C] |
| 5-99 | 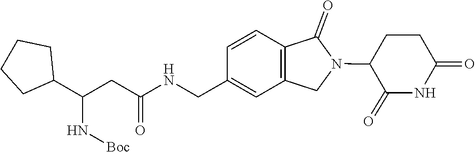 [SMILES: CC(C)(C)OC(=O)NC(CC(=O)NCc1ccc2c(c1)CN(C1CCC(=O)NC1=O)C2=O)C1CCCC1] |
| 6-01 | 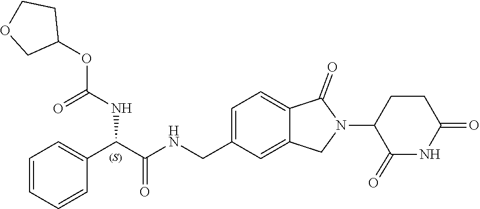 |
| 6-02 | 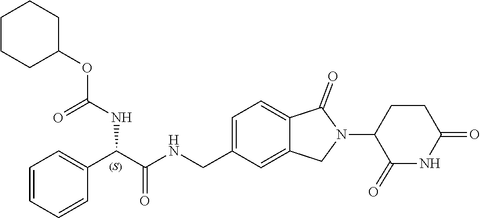 |
| 6-03 | 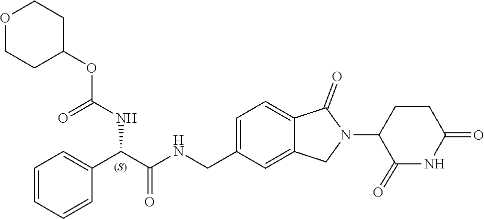 |
| 4-175b | 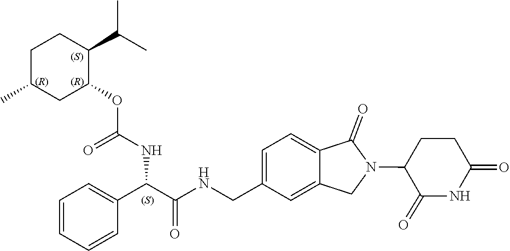 |

In other embodiments, the present invention also provides the use of compounds shown in Formula I, Formula II, Formula III, Formula IV, or Formula V of the present invention for:
(a) Preparation of drugs for treating diseases related to GSPT1 activity or expression levels;
(b) Preparation of GSPT1 targeting inhibitors or degradation agents;
(c) Non-therapeutic inhibition or degradation of GSPT1 in vitro;
(d) Non-therapeutic inhibition of tumor cell proliferation in vitro; and/or
(e) Treating diseases related to GSPT1 activity or expression levels.

In some preferred schemes, said diseases include tumors, etc.

### Term

Unless otherwise specified, the term “or” mentioned herein has the same meaning as “and/or” (referring to “or” and “and”).

Unless otherwise specified, among all compounds of the present invention, each chiral carbon atom (chiral center) can be optionally in the R configuration or S configuration, or a mixture of R configuration and S configuration.

As used herein, when used alone or as part of other substituents, the term “hydrocarbon group” refers to an alkyl, alkenyl, or alkynyl group with 1-8 carbon atoms.

As used herein, when used alone or as part of other substituents, the term “alkyl” refers to a straight chain (i.e. unbranched) or branched saturated hydrocarbon group containing only carbon atoms, or a combination of straight and branched groups. When the alkyl has a carbon atom limit (such as C₁₋₈), it refers to the alkyl group containing 1-8 carbon atoms. For example, C₁₋₈ alkyl refers to an alkyl group containing 1-8 carbon atoms, including methyl, ethyl, propyl, isopropyl, butyl, isobutyl, sec-butyl, tert-butyl, or similar groups.

As used herein, when used alone or as part of other substituents, the term “alkenyl” refers to a straight or branched carbon chain group with at least one carbon-carbon double bond. The alkenyl can be substituted or unsubstituted. When there is a limit on the number of carbon atoms in front of an alkene group (such as C₂₋₈), it refers to the alkene group containing 2-8 carbon atoms. For example, C₂₋₈ alkenyl refers to alkenyl groups containing 2-8 carbon atoms, including vinyl, propylene, 1,2-butenyl, 2,3-butenyl, butadiene, or similar groups.

As used herein, when used alone or as part of other substituents, The term “alkyneyl” refers to an aliphatic hydrocarbon group with at least one carbon-carbon triple bond. The alkyne group can be linear or branched, or a combination thereof. When the alkyne group has a limited number of carbon atoms (such as C₂₋₈ alkyne group), it refers to the alkyne group containing 2-8 carbon atoms. For example, the term “C₂₋₈ alkyne group” refers to straight or branched alkyne groups with 2-8 carbon atoms, including acetylene group, propargyl group, isopropynyl group, butyrgyl group, isobutyrgyl group, secondary butyrgyl group, tertiary butyrgyl group, or similar groups.

As used herein, when used alone or as part of other substituents, the term “cycloalkyl” refers to a cyclic group consisting of a saturated or partially saturated unit ring, a bicyclic or polycyclic (fused, bridged, or spirocyclic) ring system. When a certain cycloalkyl group has a limited number of carbon atoms (such as C₃₋₁₀), it refers to the cycloalkyl group containing 3-10 carbon atoms. In some preferred embodiments, the term “C₃₋₁₀ cycloalkyl” refers to a saturated or partially saturated monocyclic or bicyclic alkyl group with 3-10 carbon atoms, including cyclopropyl, cyclobutyl, cyclopentyl, cycloheptyl, or similar groups. “Spiral cycloalkyl” refers to a bicyclic or polycyclic group of single rings that share a carbon atom (called a spiral atom), which may contain one or more double bonds, but none of the rings have a fully conjugated π-electron system. “Fused cycloalkyl” refers to all carbon bicyclic or polycyclic groups in a system where each ring shares an adjacent pair of carbon atoms with other rings in the system. One or more rings may contain one or more double bonds, but no ring has a fully conjugated π-electron system. “Bridged cyclic alkyl” refers to an all carbon polycyclic group in which any two rings share two non-directly connected carbon atoms, which may contain one or more double bonds, but none of the rings have a fully conjugated π-electron system. The atoms contained in the cycloalkyl group are all carbon atoms. The following are some examples of cycloalkyl groups, and the present invention is not limited to the following cycloalkyl groups:

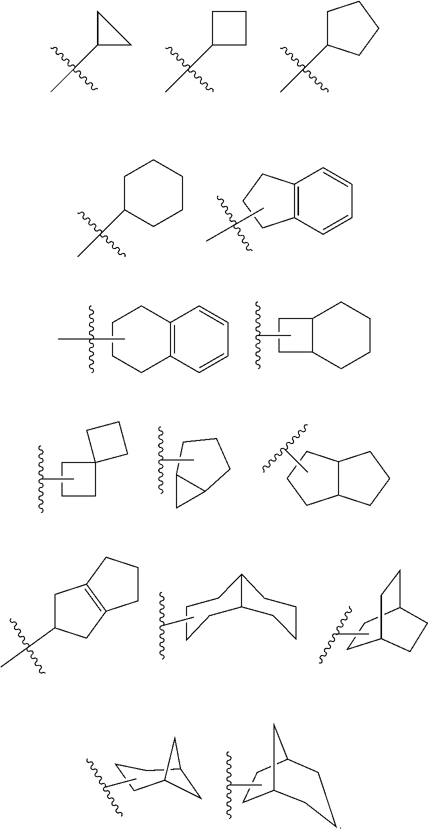

As used herein, when used alone or as part of other substituents, the term “heterocyclic group” refers to a saturated or partially unsaturated single or multi cyclic hydrocarbon substituent, where one or more (such as 1, 2, or 3) ring atoms are selected from nitrogen, oxygen, or sulfur, and the remaining ring atoms are carbon. Non-limiting embodiments of single ring heterocyclic groups include pyrrolidine, piperidine, piperazine, morpholine, thiomorpholine, and high piperazine groups. Polycyclic heterocyclic groups refer to heterocyclic groups that include helical rings, fused rings, and bridging rings. “Spiral heterocyclic group” refers to a multi ring heterocyclic group in which each ring in the system shares an atom (called Spiral atom) with other rings in the system, where one or more ring atoms are selected from nitrogen, oxygen, or sulfur, and the remaining ring atoms are carbon. “Fused heterocyclic group” refers to a multi ring heterocyclic group in a system where each ring shares an adjacent pair of atoms with other rings in the system. One or more rings may contain one or more double bonds, but no ring has a completely conjugated π-electron system, and one or more ring atoms are selected from nitrogen, oxygen, or sulfur, with the remaining ring atoms being carbon. “Bridged heterocyclic group” refers to a multi ring heterocyclic group in which any two rings share two atoms that are not directly connected. These groups may contain one or more double bonds, but none of the rings have a fully conjugated π-electron system, and one or more ring atoms are selected from nitrogen, oxygen, or sulfur, with the remaining ring atoms being carbon. If there are both saturated and aromatic rings present in the heterocyclic group (for example, saturated and aromatic rings are fused together), the point connecting to the parent ring must be on the saturated ring. Note: when the point connected to the parent is on the aromatic ring, it is called a heteroaryl group, not a heterocyclic group. The following are some examples of heterocyclic groups, and the present invention is not limited to the following heterocyclic groups:

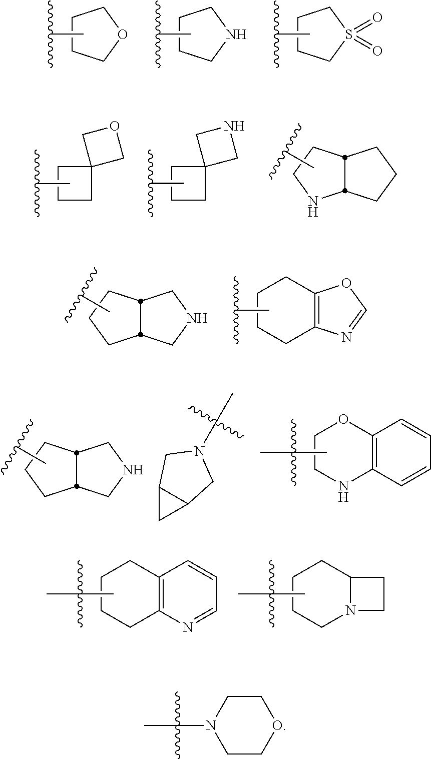

As used herein, when used alone or as part of other substituents, the term “aryl” refers to all carbon single ring or fused multi ring (i.e. ring sharing adjacent carbon atom pairs) groups with conjugated π-electron systems, such as phenyl and naphthyl. The aromatic ring can condense with other cyclic groups (including saturated and unsaturated rings), but cannot contain heteroatoms such as nitrogen, oxygen, or sulfur, and the point connecting the parent must be on the carbon atom of the ring with a conjugated π-electron system. The aryl group can be substituted or unsubstituted. The following are some examples of aryls, and the present invention is not limited to the following aryls:

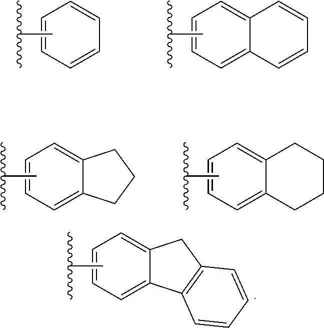

As used herein, when used alone or as part of other substituents, the term “heteroaryl” refers to a heteroaromatic group containing one to multiple heteroatoms. The heteroatoms referred to here include oxygen, sulfur, and nitrogen. For example, furan, thiophene, pyridine, pyrazole, pyrrole, N-alkylpyrrole, pyrimidine, pyrazine, imidazole, tetrazole group, etc. The heteroaromatic ring can be fused to an aromatic, heterocyclic, or cycloalkyl ring, wherein the ring connected to the parent structure is a heteroaromatic ring. Heteroaryl groups can be optionally substituted or unsubstituted. The following are some examples of heteroaryls, and the present invention is not limited to the following heteroaryls:

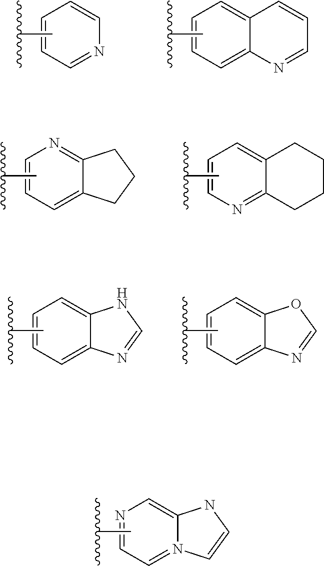

As used herein, the term “connecting into a ring” refers to two substituents being chemically bonded to form a ring structure, which can be cycloalkyl, heterocyclic, aryl, or heteroaromatic. For example, in a compound of Formula I, when the R₇ and R₈ substituents are connected to form a ring structure, the ring structure can either be a non-substituted or substituted 3-20 membered ring with 1-3 substituents or be a saturated, unsaturated, aromatic ring, or be a single or fused ring, and in addition to the N atoms connected by the R₇ and R₈ substituents, it can also contain one or more (such as 1, 2, or 3) heteroatoms selected from nitrogen, oxygen, or sulfur; when the R₂ substituent and R₃ substituent are connected to form a ring structure, the ring structure can be a 3-20 membered ring that is either non substituted or substituted by 1-3 substituents. The ring can be a saturated, unsaturated, or aromatic ring, and can be a single or fused ring; when R₃ substituents and R₄ substituents are connected to form a ring structure, the ring structure can be a 3-20 membered ring that is either non-substituted or substituted by 1-3 substituents, and can be a saturated, unsaturated carbon ring or a heterocycle.

As used herein, the term “alkoxy” or “alkyloxy” refers to an alkyl group (e.g. —O-alkyl) connected by an oxygen atom, where the alkyl group is described above. Examples of specific alkoxy groups are, such as (but not limited to) methoxy, ethoxy, propioxy, isopropoxy, butoxy, isobutyloxy, secondary butoxy, tert butoxy, or similar groups. Alkoxy can be replaced by one or more substituents, such as halogens, amino, cyano, or hydroxyl. Alkoxy can be linear or branched. When there is a carbon atom limit (such as C₁₋₈) before the alkoxy, it refers to the alkoxy containing 1-8 carbon atoms.

As used herein, the term “alkyl carbonyl” refers to a straight or branched alkyl carbonyl segment (alkyl-C(O)—). Alkyl groups can have 1-8 carbon atoms. When the alkyl carbonyl has a limited number of carbon atoms (such as C₁₋₈), it refers to the alkyl of the alkyl carbonyl containing 1-8 carbon atoms. For example, C₁₋₈ alkyl carbonyl refers to a group with a C₁₋₈ alkyl-C (O)-structure, such as methyl carbonyl, ethyl carbonyl, tert butyl carbonyl, or similar groups.

As used herein, the term “alkoxycarbonyl” refers to a straight or branched alkyl carbonyl segment (alkoxy-C═O). Alkoxy groups can have 1-8 carbon atoms. When there is a carbon atom limit (such as C₁₋₈) before the alkoxycarbonyl, it refers to the alkyl portion of the alkoxycarbonyl containing 1-8 carbon atoms. For example, C₁₋₈ alkoxycarbonyl refers to a group with a C₁₋₈ alkoxy-C═O-structure, such as methoxycarbonyl, ethoxycarbonyl, tert butoxycarbonyl, or similar groups.

As used herein, when used alone or as part of other substituents, the term “halogen” refers to F, Cl, Br, and I.

As used herein, the terms “arbitrary” or “optional” (e.g. “arbitrarily substituted”) refer to a part that is substituted or unsubstituted, and that substitution only occurs at a chemically available position. For example, H, covalent bonds, or —C(═O)— groups cannot be substituted by substituents. For the sake of convenience and conventional understanding, the terms “arbitrary substitution” or “optional substitution” only apply to sites that can be substituted by substituents, and do not include those substitutions that cannot be chemically achieved.

As used herein, the term “substitution” (with or without “arbitrary” modification) refers to the substitution of one or more hydrogen atoms on a specific functional group by a specific substituent. The specific substituent is the substituent described in the previous text, or the substituent appearing in each embodiment. Unless otherwise specified, an arbitrarily substituted group may have a substituent selected from a specific group at any substitutable site of that group, which may be the same or different at various positions. A cyclic substituent, such as a heterocyclic group, can be connected to another ring, such as a cycloalkyl group, forming a spirodicyclic system, where two rings share a common carbon atom. Those skilled in the art should understand that the expected combinations of substituents in the present invention are those that are stable or chemically achievable. The substituent, for example (but not limited to): C₁₋₈ alkyl, C₂₋₈ alkenyl, C₂₋₈ alkynyl, C₃₋₈ cycloalkyl, 3- to 12-membered heterocyclic group, aryl, heteroaryl, halogen, hydroxyl, carboxyl(-COOH), C₁₋₈ aldehydes, C₂₋₁₀ acyl, C₂₋₁₀ ester, NH₂, CN.

Those skilled in the art should understand that in the general formula compound of the present invention, the prerequisite for selecting each functional group is that the selected combination of functional groups can form a stable chemical structure.

As used herein, unless otherwise specified, the term “pharmaceutically acceptable salt” refers to a salt that is suitable for contact with the tissue of a subject (such as a person) without producing inappropriate side effects. In some embodiments, pharmaceutically acceptable salts of a compound of the present invention include salts of compounds of the present invention with acidic groups (such as potassium salts, sodium salts, magnesium salts, calcium salts) or salts of compounds of the present invention with alkaline groups (such as sulfates, hydrochloride salts, phosphates, nitrates, carbonates).

### Pharmaceutically Acceptable Salts, Solvent Complexes, Stereoisomers

As used herein, the term “pharmaceutically acceptable salt” refers to the salt formed by the compound of the present invention and pharmaceutically acceptable inorganic and organic acids, wherein preferred inorganic acids include (but are not limited to) hydrochloric acid, hydrobromic acid, phosphoric acid, nitric acid, sulfuric acid; preferred organic acids include (but are not limited to) formic acid, acetic acid, propionic acid, succinic acid, naphthalene disulfonic acid (1,5), linoleic acid, oxalic acid, tartaric acid, lactic acid, salicylic acid, benzoic acid, valeric acid, diethyl acetic acid, malonic acid, succinic acid, fumaric acid, succinic acid, adipic acid, maleic acid, malic acid, aminosulfonic acid, phenylpropanoic acid, gluconic acid, ascorbic acid, niacin, isoniacin, methanesulfonic acid, p-toluenesulfonic acid, citric acid, and amino acids.

As used herein, the term “pharmaceutically acceptable solvate” refers to a solvate formed by a compound of the present invention and a pharmaceutically acceptable solvent, wherein the pharmaceutically acceptable solvents include (but are not limited to) water, ethanol, methanol, isopropanol, tetrahydrofuran, and dichloromethane.

As used in this invention, the term “pharmaceutically acceptable stereoisomer” refers to the chiral carbon atom involved in the compound of the present invention, which can be in an R configuration, an S configuration, or a combination thereof.

### Pharmaceutical Composition and Administration Method

Due to the excellent activity of the compounds of the present invention in inhibiting or degrading GSPT1, the compounds of the present invention and their various crystal forms, pharmaceutically acceptable inorganic or organic salts, hydrates or solvate, as well as pharmaceutical compositions containing the compounds of the present invention as the main active ingredient, can be used for the treatment, prevention, and alleviation of diseases related to GSPT1 activity or expression levels.

According to the prior art, the compound of the invention can be used to treat the following diseases (but not limited to): various cancers, such as lung cancer, bladder cancer, breast cancer, stomach cancer, liver cancer, salivary adenosarcoma, ovarian cancer, prostate cancer, cervical cancer, epithelial cell cancer, multiple myeloma, pancreatic cancer, lymphoma, chronic myeloid leukemia, lymphocytic leukemia, skin T-cell lymphoma, etc.

The pharmaceutical composition of the present invention comprises a compound of the present invention or a pharmaceutically acceptable salt thereof within a safe and effective dosage range, and a pharmaceutically acceptable excipient or carrier thereof. The “safe and effective dosage” refers to the amount of compound that is sufficient to significantly improve the condition without causing serious side effects. Typically, the pharmaceutical composition contains 1-2000 mg of the compound/agent of the present invention, and more preferably, 5-200 mg of the compound/agent of the present invention. Preferably, the “one dose” is a capsule or tablet.

“Pharmaceutically acceptable carrier” refers to one or more compatible solid or liquid fillers or gel substances, which are suitable for human use and must have sufficient purity and low toxicity. “Compatibility” refers to the ability of each component in the composition to blend with the compound of the present invention and between them without significantly reducing the efficacy of the compound. Examples of pharmaceutically acceptable carrier parts include cellulose and its derivatives (such as carboxymethyl cellulose sodium, ethyl cellulose sodium, cellulose acetate, etc.), gelatin, talc, solid lubricants (such as stearic acid, magnesium stearate), calcium sulfate, vegetable oils (such as soybean oil, sesame oil, peanut oil, olive oil, etc.), polyols (such as propylene glycol, glycerol, mannitol, sorbitol, etc.), emulsifiers (such as Tween)®), wetting agents (such as sodium dodecyl sulfate), coloring agents, seasoning agents, stabilizers, antioxidants, preservatives, pyrogen-free water, etc.

There are no special restrictions on the administration methods of the compounds or drug compositions of the present invention. Representative administration methods include (but are not limited to) oral administration, intratumoral administration, rectal administration, parenteral administration (intravenous, intramuscular or subcutaneous), and local administration.

The solid dosage forms used for oral administration include capsules, tablets, pills, powders, and granules. In these solid dosage forms, the active compound is mixed with at least one conventional inert excipient (or carrier), such as sodium citrate or dicalcium phosphate, or mixed with the following components: (a) fillers or compatibilizers, such as starch, lactose, sucrose, glucose, mannitol, and silica; (b) adhesives, such as hydroxymethylcellulose, alginate, gelatin, polyvinylpyrrolidone, sucrose, and arabic gum; (c) moisturizing agents, such as glycerin; (d) disintegrants, such as agar, calcium carbonate, potato starch or cassava starch, alginic acid, certain composite silicates, and sodium carbonate; (e) slow solvents, such as paraffin; (f) absorption accelerators, such as quaternary amine compounds; (g) wetting agents, such as cetyl alcohol and monostearate glycerides; (h) adsorbents, such as kaolin; and (i) lubricants, such as talc, calcium stearate, magnesium stearate, solid polyethylene glycol, sodium dodecyl sulfate, or mixtures thereof. Capsule, tablet, and pill formulations may also include buffering agents.

Solid dosage forms such as tablets, sugar pills, capsules, pills, and granules can be prepared using coating and shell materials, such as casings and other materials well-known in the art. They can contain opaque agents, and the release of active compounds or compounds in this composition can be delayed in a certain part of the digestive tract. Examples of embedding components that can be used are polymeric substances and waxy substances. When necessary, the active compound can also form microcapsules with one or more of the above-mentioned excipients.

Liquid dosage forms for oral administration include pharmaceutically acceptable lotion, solutions, suspensions, syrups or tinctures. In addition to active compounds, liquid formulations may include inert diluents commonly used in the art, such as water or other solvents, solubilizers, and emulsifiers, such as ethanol, isopropanol, ethyl carbonate, ethyl acetate, propylene glycol, 1,3-butanediol, dimethylformamide, and oil, particularly cottonseed oil, peanut oil, corn germ oil, olive oil, castor oil, sesame oil, or mixtures of these substances.

In addition to these inert diluents, the composition may also include additives such as wetting agents, emulsifiers and suspensions, sweeteners, correctors, and spices.

In addition to active compounds, suspensions may include suspension agents, such as ethoxylated isooctadecanol, polyoxyethylene sorbitol and dehydrated sorbitol esters, microcrystalline cellulose, methanol aluminum and agar, or mixtures of these substances.

Compositions for parenteral injection may include physiologically acceptable sterile aqueous or anhydrous solutions, dispersions, suspensions or lotion, and sterile powders for reconstitution into sterile injectable solutions or dispersions. Suitable aqueous and non-aqueous carriers, diluents, solvents or excipients include water, ethanol, polyols, and their suitable mixtures.

The dosage forms of the compounds of the present invention for local administration include ointments, powders, patches, sprays, and inhalers. The active ingredients are mixed with physiologically acceptable carriers and any preservatives, buffering agents, or propellants that may be necessary under sterile conditions.

The compound of the present invention can be administered alone or in combination with other pharmaceutically acceptable compounds.

When using a pharmaceutical composition, a safe and effective amount of the compound of the present invention is administrated to mammals (such as humans) in need of treatment, where the dosage at the time of administration is the pharmaceutically recognized effective dosage. For a person weighing 60 kg, the daily dosage is usually 1-2000 mg, preferably 5-500 mg. Of course, the specific dosage should also consider factors such as the route of administration and the patient's health status, all of which are within the scope of skilled physicians.

### The Advantages of the Present Invention Include:

1. A compound as shown in Formula I is provided.
2. A GSPT1 inhibitor with a novel structure and its preparation and application is provided, which can degrade or inhibit GSPT1 at extremely low concentrations.
3. A class of pharmaceutical compositions for treating diseases related to GSPT1 activity are provided.

To clarify the purpose, technical solution, and advantages of the embodiments of the present invention, the following will further elaborate on the present invention in conjunction with specific embodiments. It should be understood that these embodiments are only used to illustrate the present invention and not to limit the scope of the present invention. The experimental methods in the following embodiments that do not specify specific conditions are usually based on conventional conditions or conditions recommended by the manufacturer. Unless otherwise specified, percentages and portions are weight percentages and weight portions. The experimental materials and reagents used in the following embodiments can be obtained from commercial channels unless otherwise specified.

Unless otherwise specified, the technical and scientific terms used herein have the same meanings as those commonly understood by ordinary technical personnel in the technical field to which this application belongs. It should be noted that the terms used herein are only intended to describe specific embodiments and are not intended to limit exemplary embodiments of this application.

### Example 1 (4-85)

### N-((2-(2,6-dioxopiperidin-3-yl)-1-oxoisoindolin-5-yl)methyl)-2-hydroxy-2-phenylacetamide

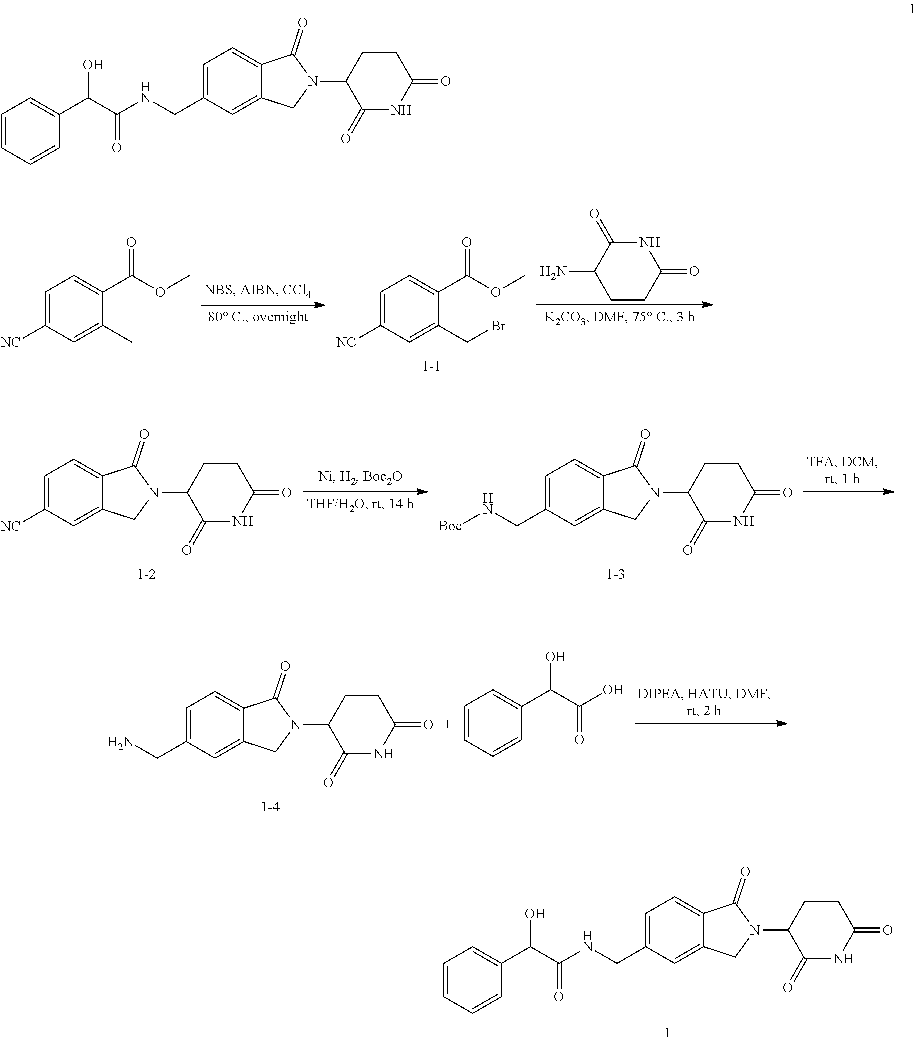

### Step 1: Preparation of methyl 2-(bromomethyl)-4-cyanobenzoate (1-1)

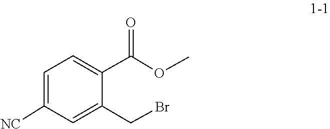

Methyl 4-cyano-2-methylbenzoate (3.0 g, 17.1 mmol), N-bromosuccinimide (4.3 g, 24.0 mmol) and azobisisobutyronitrile (0.6 g, 3.4 mmol) were dissolved in carbon tetrachloride (40 ml) under nitrogen protection, and the reaction was stirred at 80° C. overnight. The reaction solution was concentrated under reduced pressure. The residue was separated by silica gel column chromatography eluting with petroleum ether to give intermediate 1-1 (3.0 g, 70.0% yield), as a white solid.

### Step 2: Preparation of 2-(2,6-dioxopiperidin-3-yl)-1-oxoisoindoline-5-carbonitrile (1-2)

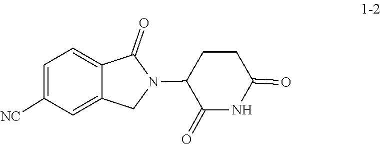 [SMILES: N#Cc1ccc2c(c1)CN(C1CCC(=O)NC1=O)C2=O]

Methyl 2-(bromomethyl)-4-cyanobenzoate (3.0 g, 11.8 mmol), 3-aminopiperidine-2,6-dione hydrochloride (1.7 g, 11.8 mmol) and anhydrous potassium carbonate (4.1 g, 35.4 mmol) were dissolved in N,N-dimethylformamide (20 ml) under nitrogen protection and the reaction was stirred at 75° C. for 3 hours. The reaction solution was concentrated under reduced pressure. The residue was stirred with water (50 ml) for 0.5 h at room temperature, filtered and dried to give 1-2 (2.5 g, 78.1% yield), as an off-white solid.

### Step 3: Preparation of tert-butyl ((2-(2,6-dioxopiperidin-3-yl)-1-oxoisoindolin-5-yl)carbamate (1-3))

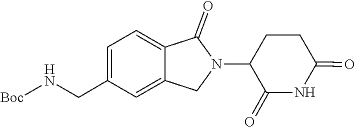 [SMILES: CC(C)(C)OC(=O)NCc1ccc2c(c1)CN(C1CCC(=O)NC1=O)C2=O]

Under the condition of hydrogen, 2-(2,6-dioxopiperidin-3-yl)-1-oxoisoindoline-5-carbonitrile (2.5 g, 9.3 mmol), Raney nickel (4 mL) and di-tert-butyl dicarbonate (3.9 g, 17.9 mmol) were dissolved in tetrahydrofuran (40 mL) and the reaction was stirred at room temperature for 14 h. Diatomite filtration was carried out, and the reaction solution was concentrated under reduced pressure, and the residue was separated by silica gel column chromatography (MeOH/DCM=1/50 elution) to give intermediates 1-3 (1.8 g, 51.4% yield), as a white solid.

### Step 4: Preparation of 3-(5-(aminomethyl)-1-oxoisoindolin-2-yl)piperidine-2,6-dione (1-4)

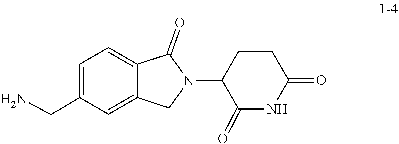 [SMILES: NCc1ccc2c(c1)CN(C1CCC(=O)NC1=O)C2=O]

Tert-butyl ((2-(2,6-dioxopiperidin-3-yl)-1-oxoisoindolin-5-yl)carbamate (1.8 g, 4.8 mmol) was dissolved in dichloromethane (10 mL), trifluoroacetic acid (5 mL) was added, and the reaction was stirred at room temperature for 1 h. The reaction solution was concentrated under reduced pressure, and the residue was separated by column chromatography on silica gel (MeOH/DCM=1/8 elution) to give intermediate 1-4 (1.3 g, 100% yield), as a white solid.

### Step 5: Preparation of N-((2-(2,6-dioxopiperidin-3-yl)-l-oxoisoindolin-5-yl)methyl)-2-hydroxy-2-phenylacetamide (1)

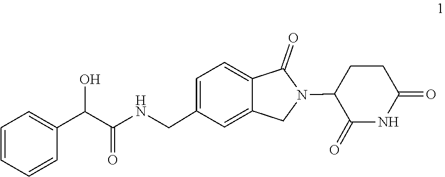 [SMILES: O=C1CCC(N2Cc3cc(CNC(=O)C(O)c4ccccc4)ccc3C2=O)C(=O)N1]

Intermediates 1-4 (38.0 mg, 0.14 mmol), 2-hydroxy-2-phenylacetic acid (21.0 mg, 0.14 mmol), N,N-diisopropylethylamine (90.3 mg, 0.70 mmol) and 2-(7-oxobenzotriazole)-N,N,N′,N′-tetramethyluronium hexafluorophosphate (69.2 mg, 0.18 mmol) was dissolved in N,N-dimethylformamide (10 mL) and reacted for 2 hours at room temperature. Water (20 mL) was added to the reaction system, extracted with ethyl acetate (30 mL×3), the organic phases were combined, washed with saturated sodium chloride solution (30 mL×3), dried with anhydrous sodium sulfate, filtered, concentrated under reduced pressure, and the residue was purified by column chromatography on silica gel (eluted with methanol/dichloromethane=1/40) to give compound 1 (42 mg, 73.7% yield), as a white solid. MS(m/z): 408.1[M+H]⁺; ¹H NMR (400 MHz, DMSO-d₆) δ11.00 (s, 1H), 8.70 (t, J=5.8 Hz, 1H), 7.64 (d, J=8.2 Hz, 1H), 7.44 (d, J=7.2 Hz, 2H), 7.40-7.32 (m, 4H), 7.31-7.26 (m, 1H), 6.26 (d, J=4.4 Hz, 1H), 5.11 (dd, J=13.2, 5.0 Hz, 1H), 4.99 (d, J=4.4 Hz, 1H), 4.45-4.26 (m, 4H), 2.91 (dd, J=21.7, 9.0 Hz, 1H), 2.61 (d, J=17.5 Hz, 1H), 2.47-2.34 (m, 1H), 2.11-1.96 (m, 1H).

### Example 2 (4-127)

### Tert butyl (2-((2-(2,6-dioxopiperidin-3-yl)-1-oxoisoindolin-5-yl)methyl)amino)-2-oxo-1-phenylethyl) carbamate

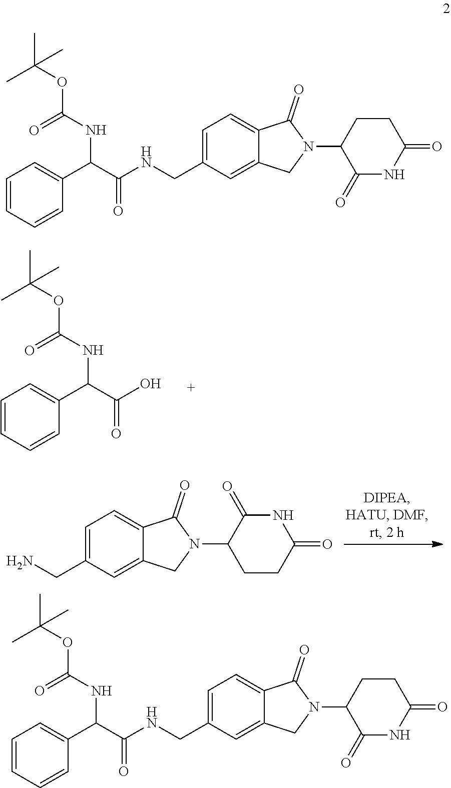

Compound 2 was prepared according to Example 1, Step 5. MS(m/z): 406.9[M−100]; ¹H NMR (400 MHz, DMSO-d₆) δ10.98 (s, 1H), 8.75 (s, 1H), 7.61 (d, J=7.7 Hz, 1H), 7.45 (d, J=7.2 Hz, 2H), 7.32 (ddd, J=19.1, 13.4, 5.6 Hz, 6H), 5.27-5.01 (m, 2H), 4.42-4.20 (m, 4H), 2.99-2.86 (m, 1H), 2.61 (d, J=17.5 Hz, 1H), 2.47-2.33 (m, 1H), 2.05-1.96 (m, 1H), 1.40 (s, 9H).

### Example 3 (4-87)

### Tert butyl ((1S)-2-((2-(2,6-dioxopiperidin-3-yl)-1-oxoisoindolin-5-yl)methyl)amino)-2-oxo-1-phenylethyl) carbamate

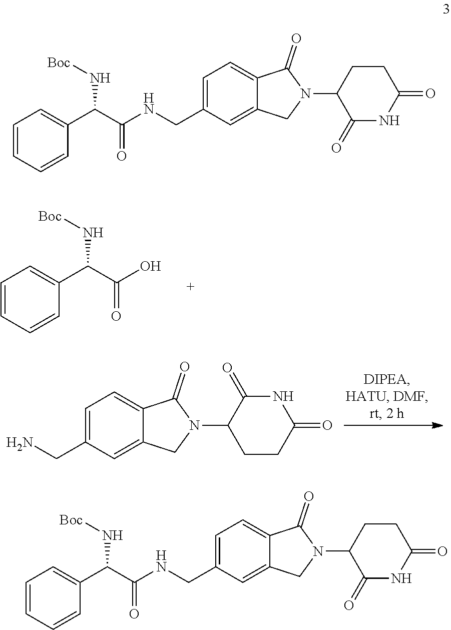 [SMILES: CC(C)(C)OC(=O)N[C@H](C(=O)NCc1ccc2c(c1)CN(C1CCC(=O)NC1=O)C2=O)c1ccccc1 ; CC(C)(C)OC(=O)N[C@H](C(=O)O)c1ccccc1 ; NCc1ccc2c(c1)CN(C1CCC(=O)NC1=O)C2=O ; CC(C)(C)OC(=O)N[C@H](C(=O)NCc1ccc2c(c1)CN(C1CCC(=O)NC1=O)C2=O)c1ccccc1]

Compound 3 was prepared according to Example 1, Step 5. MS(m/z): 406.9[M−100]; ¹H NMR (400 MHz, DMSO-d₆) δ10.99 (s, 1H), 8.76 (s, 1H), 7.60 (d, J=7.7 Hz, 1H), 7.44 (d, J=7.3 Hz, 2H), 7.38-7.25 (m, 6H), 5.20 (d, J=7.9 Hz, 1H), 5.10 (dd, J=13.2, 4.9 Hz, 1H), 4.46-4.30 (m, 3H), 4.22 (dd, J=17.3, 2.8 Hz, 1H), 2.98-2.84 (m, 1H), 2.59 (d, J=17.3 Hz, 1H), 2.39 (dd, J=13.2, 4.1 Hz, 1H), 2.04-1.93 (m, 1H), 1.39 (s, 9H).

### Example 4 (4-108)

### Tert butyl ((1R)-2-((2-(2-(2,6-Dioxopiperidin-3-yl)-1-oxoisoindolin-5-yl)methyl)amino)-2-oxo-1-phenylethyl) carbamate

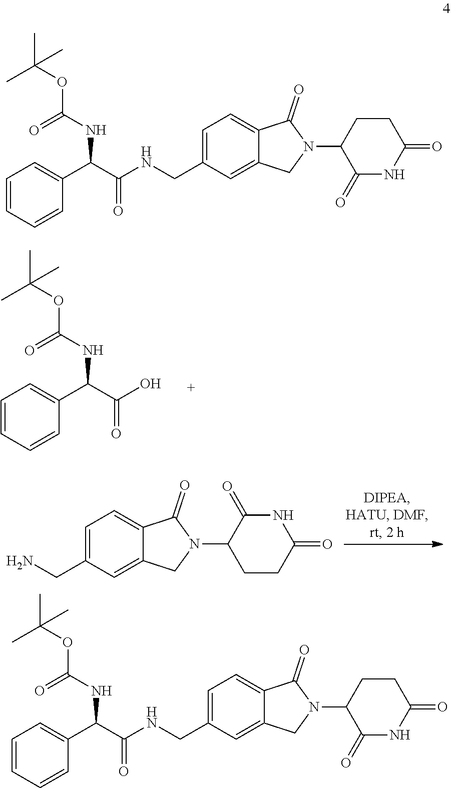

Compound 4 was prepared according to Example 1, Step 5. MS(m/z): 406.9[M−100]⁺; ¹H NMR (400 MHz, DMSO-d₆) δ¹H NMR (400 MHz, DMSO) δ 10.98 (s, 1H), 8.75 (s, 1H), 7.61 (d, J=7.8 Hz, 1H), 7.45 (d, J=7.3 Hz, 2H), 7.36 (d, J=6.9 Hz, 2H), 7.34-7.31 (m, 3H), 7.29 (d, J=4.3 Hz, 1H), 5.22 (d, J=7.6 Hz, 1H), 5.11 (dd, J=13.2, 5.0 Hz, 1H), 4.39 (s, 2H), 4.34 (s, 1H), 4.24 (dd, J=17.2, 2.6 Hz, 1H), 2.99-2.85 (m, 1H), 2.61 (d, J=17.6 Hz, 1H), 2.41 (dd, J=13.2, 4.2 Hz, 1H), 2.05-1.96 (m, 1H), 1.40 (s, 9H).

### Example 5 (4-122-2)

### (2S)—N-((2-(2,6-dioxopiperidin-3-yl)-1-oxoisoindolin-5-yl)methyl)-2-(methylamino)-2-phenylacetamide

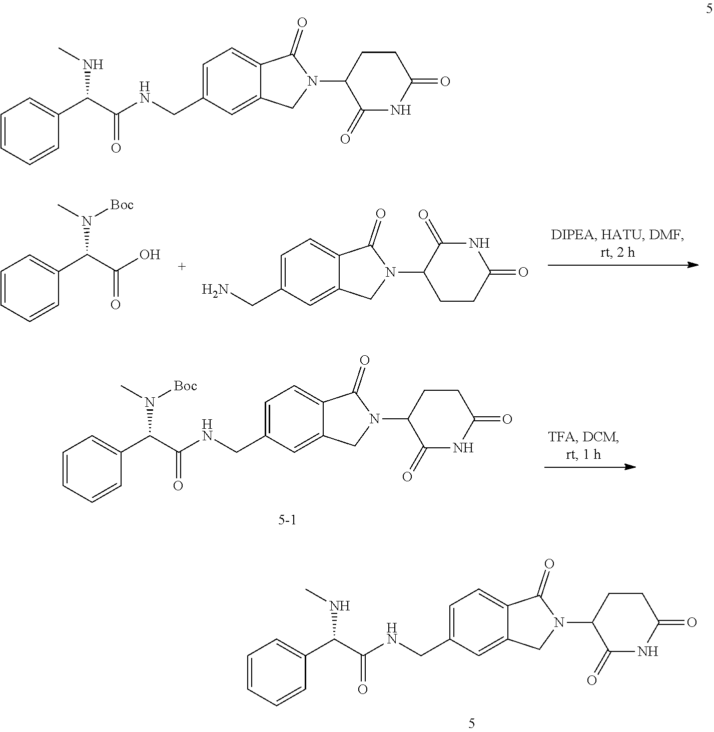 [SMILES: CN[C@H](C(=O)NCc1ccc2c(c1)CN(C1CCC(=O)NC1=O)C2=O)c1ccccc1 ; CN(C(=O)OC(C)(C)C)[C@H](C(=O)O)c1ccccc1 ; NCc1ccc2c(c1)CN(C1CCC(=O)NC1=O)C2=O ; CN(C(=O)OC(C)(C)C)[C@H](C(=O)NCc1ccc2c(c1)CN(C1CCC(=O)NC1=O)C2=O)c1ccccc1 ; CN[C@H](C(=O)NCc1ccc2c(c1)CN(C1CCC(=O)NC1=O)C2=O)c1ccccc1]

Intermediate 5-1 was prepared according to the method of Step 5 of Example 1;

Compound 5 was prepared according to Example 1, Step 4. MS(m/z): 420.9 [M+H]⁺; ¹H NMR (400 MHz, DMSO-d₆) δ10.98 (s, 1H), 8.70 (t, J=5.9 Hz, 1H), 7.63 (d, J=7.7 Hz, 1H), 7.42 (d, J=7.4 Hz, 2H), 7.35 (d, J=7.2 Hz, 2H), 7.30 (dd, J=16.0, 5.3 Hz, 3H), 5.10 (dd, J=13.3, 5.0 Hz, 1H), 4.40 (d, J=5.3 Hz, 2H), 4.37-4.21 (m, 2H), 4.10 (s, 1H), 2.91 (dd, J=21.6, 9.0 Hz, 1H), 2.66 (d, J=20.9 Hz, 1H), 2.40 (dd, J=13.1, 3.9 Hz, 1H), 2.25 (s, 3H), 2.05-1.97 (m, 1H).

### Example 6 (4-112)

### (2S)-2-Acetamido-N-((2-(2,6-dioxopiperidin-3-yl)-1-oxoisoindolin-5-yl)methyl)-2-phenyl acetamide

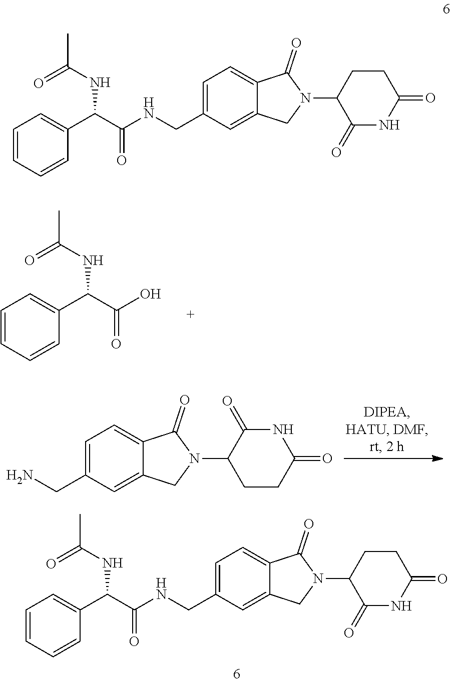

Compound 6 was prepared according to Example 1, Step 5. MS(m/z): 449.1[M+H]⁺; ¹H NMR (400 MHz, DMSO-d₆) δ10.98 (s, 1H), 8.87 (s, 1H), 8.56 (d, J=7.8 Hz, 1H), 7.63 (d, J=8.0 Hz, 1H), 7.44 (d, J=7.4 Hz, 2H), 7.37 (t, J=7.3 Hz, 2H), 7.31 (d, J=2.3 Hz, 3H), 5.51 (d, J=7.8 Hz, 1H), 5.11 (dd, J=13.2, 5.1 Hz, 1H), 4.43-4.17 (m, 4H), 2.91 (d, J=12.5 Hz, 1H), 2.70-2.59 (m, 1H), 2.41-2.30 (m, 1H), 2.07-1.98 (m, 1H), 1.92 (s, 3H).

### Example 7 (4-111)

### N-(2-((2-(2-(2,6-dioxopiperidin-3-yl)-1-oxoisoindolin-5-yl)methyl)amino)-2-oxo-1-phenyl ethyl)benzamide

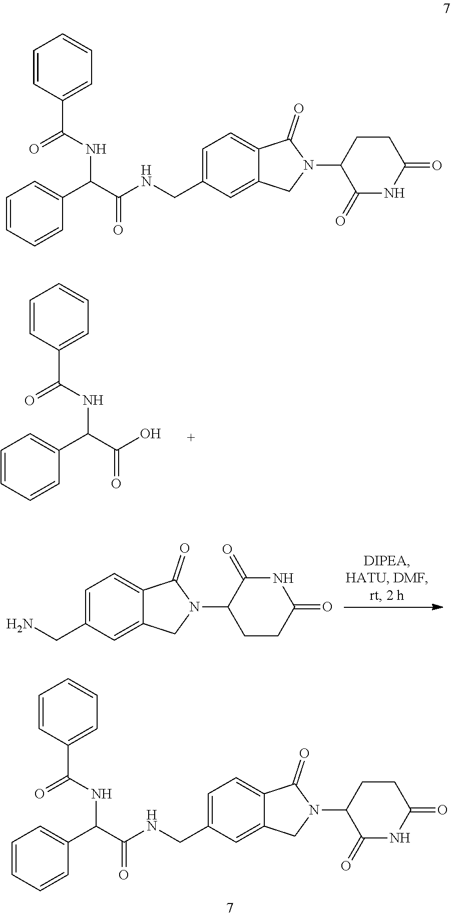

Compound 7 was prepared according to Example 1, Step 5. MS(m/z): 510.8[M+H]⁺; ¹H NMR (400 MHz, DMSO-d₆) δ¹H NMR (400 MHz, DMSO) δ 10.98 (s, 1H), 8.87 (dd, J=10.0, 6.9 Hz, 2H), 7.94 (d, J=7.9 Hz, 2H), 7.63 (d, J=8.1 Hz, 1H), 7.56 (t, J=6.8 Hz, 3H), 7.48 (t, J=7.5 Hz, 2H), 7.42-7.31 (m, 5H), 5.75 (d, J=7.6 Hz, 1H), 5.11 (dd, J=13.2, 5.0 Hz, 1H), 4.54-4.19 (m, 4H), 2.98-2.86 (m, 1H), 2.61 (d, J=17.8 Hz, 1H), 2.41 (dd, J=13.0, 4.3 Hz, 1H), 2.05-1.96 (m, 1H).

### Example 8 (4-121)

### Methyl (1S)-2-((2-(2,6-dioxypiperidin-3-yl)-1-oxoisoindoline-5-yl)methyl)amino)-2-oxo-1-phenylethyl) carbamate

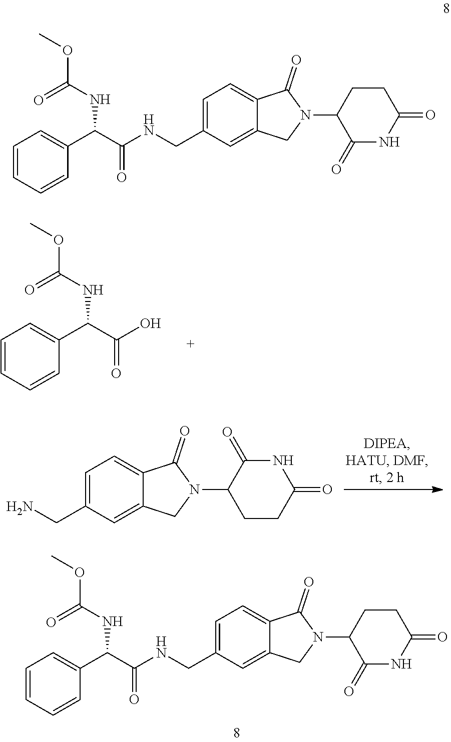

Compound 8 was prepared according to Example 1, Step 5. MS(m/z): 464.8[M+H]⁺; ¹H NMR (400 MHz, DMSO-d₆) δ10.98 (s, 1H), 8.81 (s, 1H), 7.82 (d, J=7.6 Hz, 1H), 7.62 (d, J=8.1 Hz, 1H), 7.45 (d, J=7.3 Hz, 2H), 7.40-7.26 (m, 6H), 5.27 (d, J=8.3 Hz, 1H), 5.10 (dd, J=13.3, 5.0 Hz, 1H), 4.39 (d, J=4.9 Hz, 2H), 4.36-4.21 (m, 2H), 3.57 (s, 3H), 2.98-2.87 (m, 1H), 2.66 (d, J=21.5 Hz, 1H), 2.44-2.35 (m, 1H), 2.04-1.97 (m, 1H).

### Example 9 (4-125)

### Methyl (2-(2-(2,6-dioxypiperidin-3-yl)-1-oxoisoindoline-5-yl)amino)-2-oxo-1-phenylethyl) carbamate

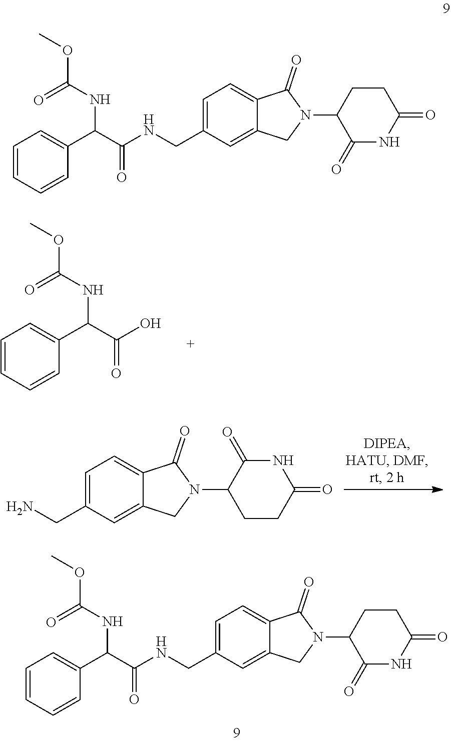

Compound 9 was prepared according to Example 1, Step 5. MS(m/z): 464.8 [M+H]⁺; ¹H NMR (400 MHz, DMSO-d₆) δ10.98 (s, 1H), 8.86 (d, J=5.7 Hz, 1H), 8.56 (d, J=7.9 Hz, 1H), 7.66-7.54 (m, 1H), 7.44 (d, J=7.4 Hz, 2H), 7.41-7.25 (m, 5H), 5.51 (d, J=7.8 Hz, 1H), 5.11 (dd, J=13.3, 5.0 Hz, 1H), 4.45-4.19 (m, 4H), 2.97-2.86 (m, 1H), 2.61 (d, J=17.6 Hz, 1H), 2.41 (dd, J=13.2, 4.3 Hz, 1H), 2.06-1.97 (m, 1H), 1.92 (s, 3H).

### Example 10 (4-138)

### Ethyl ((1S)-2-((2-(2,6-dioxypiperidin-3-yl)-1-oxoisoindoline-5-yl)methyl)amino)-2-oxo-1-phenylethyl) carbamate

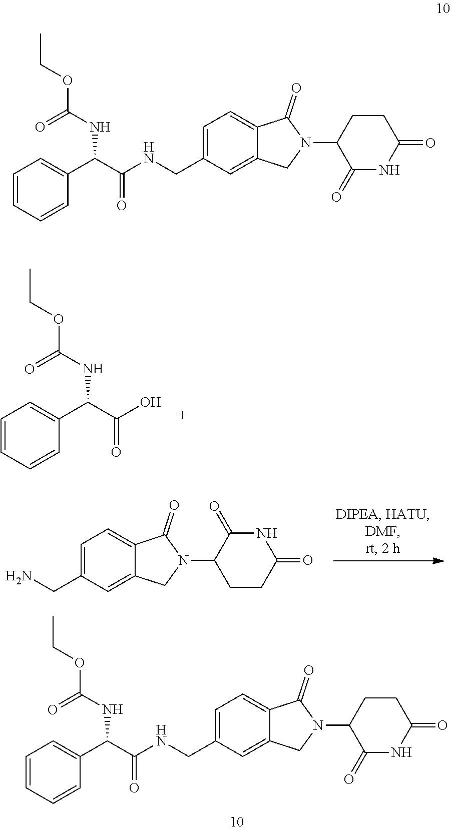

Compound 10 was prepared according to Example 1, Step 5. MS(m/z): 479.2 [M+H]⁺; ¹H NMR (400 MHz, DMSO-d₆) δ10.98 (s, 1H), 8.79 (t, J=5.3 Hz, 1H), 7.72 (d, J=7.9 Hz, 1H), 7.62 (d, J=7.7 Hz, 1H), 7.46 (d, J=7.2 Hz, 2H), 7.41-7.25 (m, 5H), 5.27 (d, J=8.1 Hz, 1H), 5.11 (dd, J=13.2, 5.0 Hz, 1H), 4.31 (dd, J=51.6, 17.6 Hz, 4H), 4.02 (dd, J=12.9, 6.0 Hz, 2H), 2.98-2.84 (m, 1H), 2.61 (d, J=17.3 Hz, 1H), 2.47-2.32 (m, 1H), 2.07-1.94 (m, 1H), 1.18 (t, J=6.8 Hz, 3H).

### Example 11 (4-160)

### Isopropyl ((1S)-2-((2-(2,6-dioxypiperidin-3-yl)-1-oxoisoindoline-5-yl)methyl)amino)-2-oxo-1-phenylethy 1) carbamate

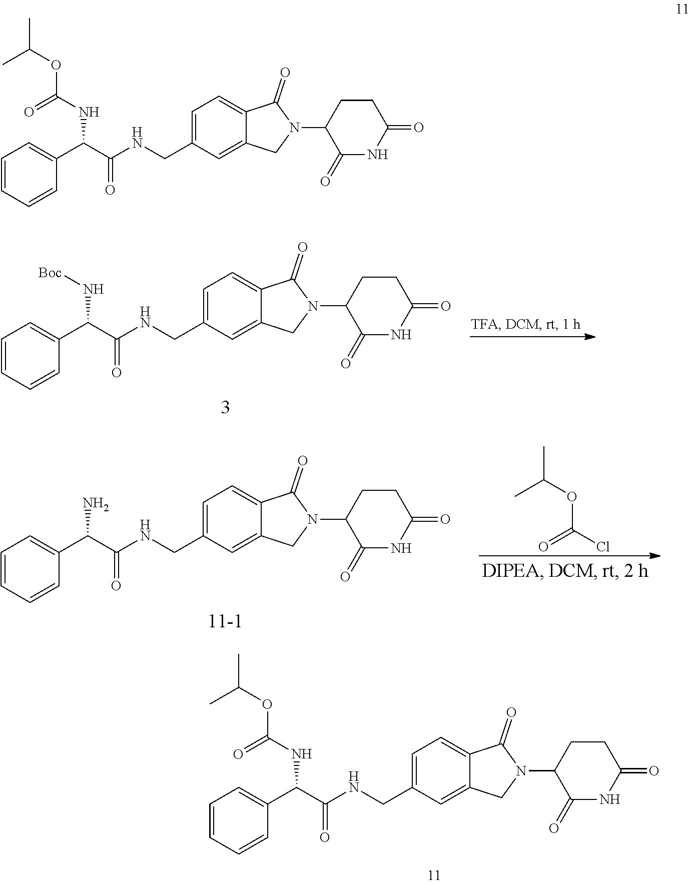

### Step 1: Intermediate 11-1 was Prepared According to the Method of Step 4 of Example 1

### Step 2: Preparation of Isopropyl ((1S)-2-((2-(2,6-dioxypiperidin-3-yl)-1-oxoisoindoline-5-yl)methyl)amino)-2-oxo-1-phenylethyl) carbamate (Example 11)

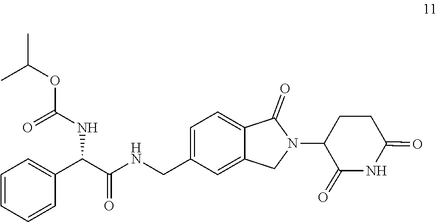 [SMILES: CC(C)OC(=O)N[C@H](C(=O)NCc1ccc2c(c1)CN(C1CCC(=O)NC1=O)C2=O)c1ccccc1]

(2S)-2-amino-N-((2-(2-(2,6-dioxopiperidin-3-yl)-1-oxoisoindolin-5-yl)methyl)-2-phenylacetamide (100.0 mg, 0.25 mmol) and N,N-diisopropylethylamine (162.0 mg, 1.3 mmol) were dissolved in dichloromethane (5 mL), isopropyl chloroformate (31.0 mg 0.25 mmol) was added and stirred at room temperature for 2 h. The reaction solution was concentrated under reduced pressure and the residue was separated by silica gel column chromatography (MeOH/DCM=1/40 elution) to afford compound 11 (77.0 mg, 62.6% yield), as a white solid. MS(m/z): 493.2 [M+H]⁺; ¹H NMR (400 MHz, DMSO-d₆) δ10.98 (s, 1H), 8.76 (s, 1H), 7.62 (d, J=7.6 Hz, 2H), 7.46 (d, J=7.3 Hz, 2H), 7.39-7.26 (m, 5H), 5.27 (d, J=8.0 Hz, 1H), 5.11 (dd, J=13.3, 5.0 Hz, 1H), 4.83-4.71 (m, 1H), 4.46-4.20 (m, 4H), 2.98-2.83 (m, 1H), 2.61 (d, J=17.1 Hz, 1H), 2.41 (dd, J=13.2, 4.3 Hz, 1H), 2.11-1.92 (m, 1H), 1.27-1.11 (m, 6H).

### Example 12 (4-159)

### Cyclopentyl (1S)-2-((2-(2,6-dioxypiperidin-3-yl)-1-oxoisoindoline-5-yl)methyl)amino)-2-oxo-1-phenylethyl) carbamate

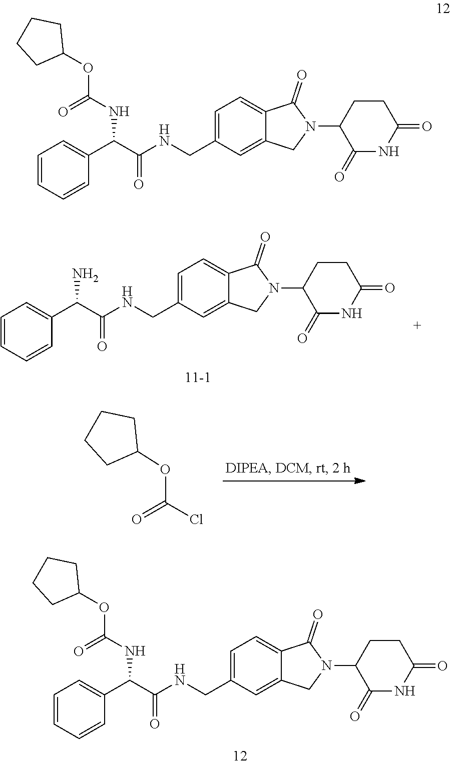 [SMILES: O=C1CCC(N2Cc3cc(CNC(=O)[C@@H](NC(=O)OC4CCCC4)c4ccccc4)ccc3C2=O)C(=O)N1 ; N[C@H](C(=O)NCc1ccc2c(c1)CN(C1CCC(=O)NC1=O)C2=O)c1ccccc1 ; O=C(Cl)OC1CCCC1 ; O=C1CCC(N2Cc3cc(CNC(=O)[C@@H](NC(=O)OC4CCCC4)c4ccccc4)ccc3C2=O)C(=O)N1]

Compound 12 was prepared according to Example 11, Step 2. MS(m/z): 519.2 [M+H]⁺; ¹H NMR (400 MHz, DMSO-d₆) δ10.98 (s, 1H), 8.76 (s, 1H), 7.60 (t, J=8.3 Hz, 2H), 7.45 (d, J=7.2 Hz, 2H), 7.38-7.26 (m, 5H), 5.26 (d, J=7.6 Hz, 1H), 5.11 (dd, J=13.2, 4.9 Hz, 1H), 4.97 (s, 1H), 4.42-4.20 (m, 4H), 2.99-2.85 (m, 1H), 2.61 (d, J=17.4 Hz, 1H), 2.47-2.33 (m, 1H), 2.06-1.95 (m, 1H), 1.79 (s, 2H), 1.68-1.50 (m, 6H).

### Example 13 (4-175)

### ((1S)-2-((2-(2,6-dioxypiperidin-3-yl)-1-oxoisoidoline-5-yl)methyl)amino)-2-oxo-1-phenylethyl) carbamate-(+)-menthol Ester

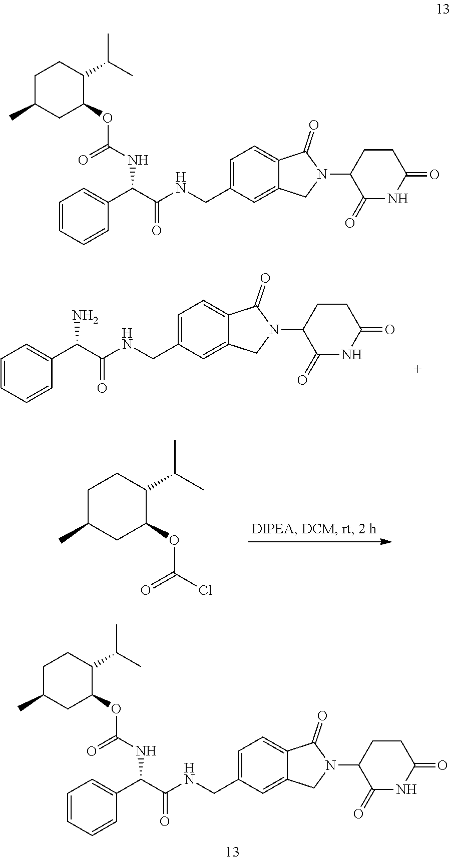

Compound 13 was prepared according to Example 11, Step 2. MS(m/z): 589.0 [M+H]⁺; ¹H NMR (400 MHz, DMSO-d₆) δ10.99 (s, 1H), 8.75 (s, 1H), 7.60 (d, J=7.8 Hz, 2H), 7.52-7.20 (m, 7H), 5.29 (d, J=8.3 Hz, 1H), 5.11 (dd, J=13.2, 5.0 Hz, 1H), 4.43 (d, J=11.2 Hz, 1H), 4.41-4.17 (m, 4H), 3.02-2.85 (m, 1H), 2.63 (t, J=18.5 Hz, 1H), 2.40 (ddd, J=26.5, 13.3, 4.4 Hz, 1H), 2.07-1.91 (m, 2H), 1.85 (d, J=11.6 Hz, 1H), 1.63 (s, 2H), 1.50-1.23 (m, 2H), 1.08-0.91 (m, 2H), 0.85 (dd, J=10.3, 6.4 Hz, 7H), 0.74 (d, J=6.4 Hz, 3H).

### Example 14 (4-195)

### Benzyl ((1S)-2-((2-(2,6-dioxypiperidin-3-yl)-1-oxoisoindoline-5-yl)methyl)amino)-2-oxo-1-phenylethy 1) aminobenzoate

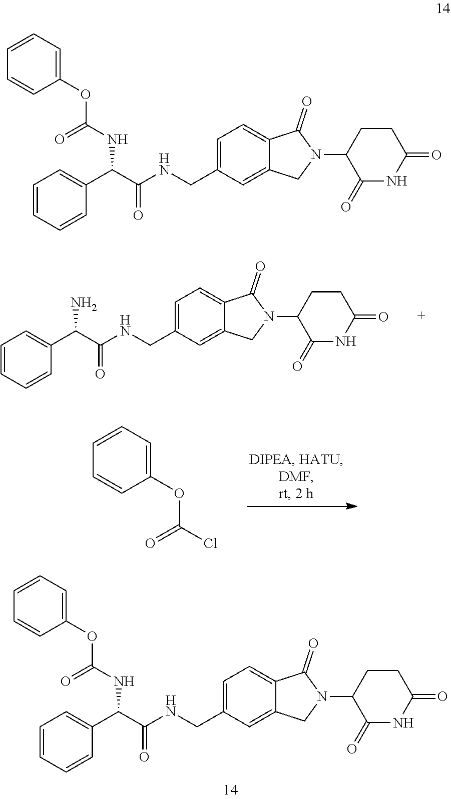

Compound 14 was prepared according to Example 11, Step 2. MS(m/z): 527.2 [M+H]⁺; H NMR (400 MHz, DMSO-d₆) δ10.96 (s, 1H), 9.00-8.76 (m, 1H), 8.46 (d, J=8.1 Hz, 1H), 7.72-7.58 (m, 1H), 7.51 (t, J=7.7 Hz, 2H), 7.43-7.28 (m, 7H), 7.20 (t, J=7.4 Hz, 1H), 7.10 (d, J=7.8 Hz, 1H), 5.33 (d, J=5.6 Hz, 1H), 5.09 (dd, J=13.0, 4.0 Hz, 1H), 4.69 (s, 1H), 4.50-4.17 (m, 4H), 2.97-2.84 (m, 1H), 2.60 (d, J=17.4 Hz, 1H), 2.45-2.31 (m, 1H), 2.05-1.94 (m, 1H).

### Example 15 (4-139)

### Benzyl ((1S)-2-((2-(2,6-dioxypiperidin-3-yl)-1-oxoisoindoline-5-yl)methyl)amino)-2-oxo-1-phenylethyl) carbamate

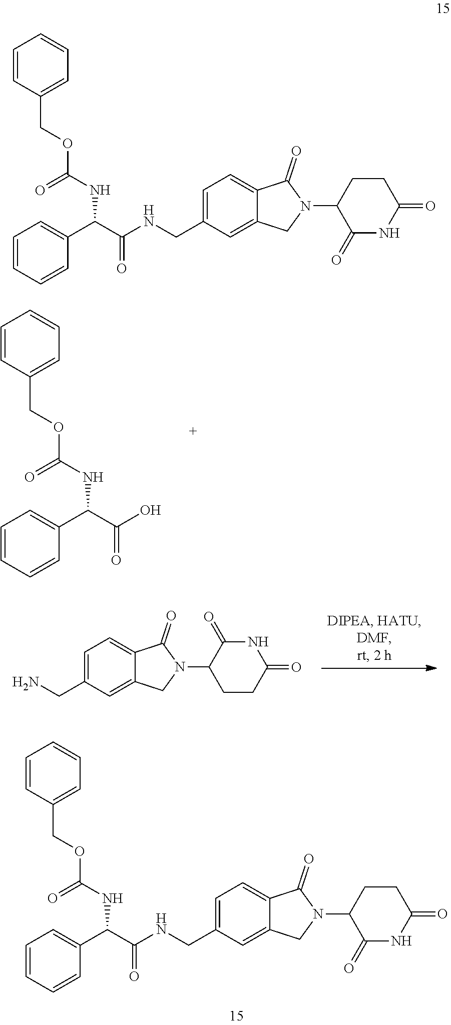

Compound 15 was prepared according to Example 11, Step 2. MS(m/z): 541.2 [M+H]⁺; ¹H NMR (400 MHz, DMSO-d₆) δ10.98 (s, 1H), 8.81 (s, 1H), 8.02-7.92 (m, 1H), 7.62 (d, J=7.8 Hz, 1H), 7.47 (d, J=7.3 Hz, 2H), 7.42-7.21 (m, 10H), 5.30 (d, J=8.2 Hz, 1H), 5.15-5.09 (m, 1H), 5.07 (s, 2H), 4.45-4.18 (m, 4H), 2.96-2.86 (m, 1H), 2.64 (t, J=19.1 Hz, 1H), 2.39 (dd, J=20.2, 11.5 Hz, 1H), 2.07-1.97 (m, 1H).

### Example 16 (4-184)

### (2S)-2-(3-benzylurea)-N-((2-(2,6-dioxopyridin-3-yl)-1-oxoisoidoline-5-yl)methyl)-2-phenylacetamide

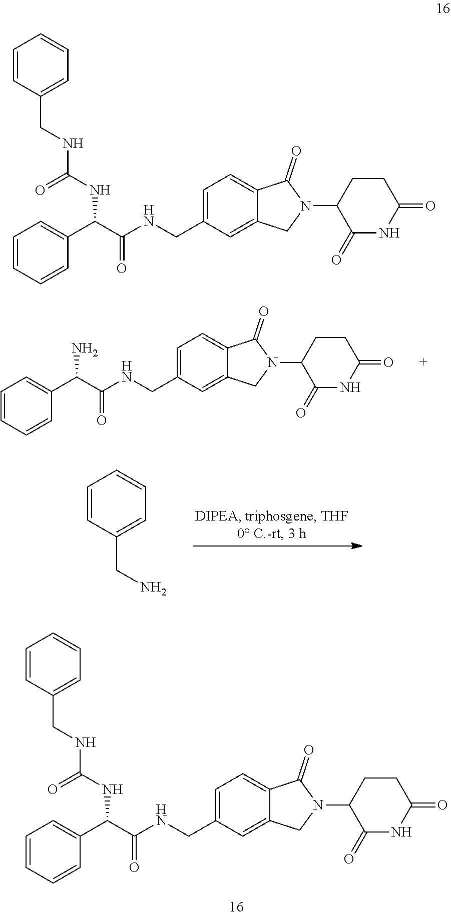 [SMILES: O=C1CCC(N2Cc3cc(CNC(=O)[C@@H](NC(=O)NCc4ccccc4)c4ccccc4)ccc3C2=O)C(=O)N1 ; N[C@H](C(=O)NCc1ccc2c(c1)CN(C1CCC(=O)NC1=O)C2=O)c1ccccc1 ; NCc1ccccc1 ; O=C1CCC(N2Cc3cc(CNC(=O)[C@@H](NC(=O)NCc4ccccc4)c4ccccc4)ccc3C2=O)C(=O)N1]

Intermediate 11-1 (204.0 mg, 0.5 mmol), N,N-diisopropylethylamine (342.0 mg, 2.7 mmol) was dissolved in tetrahydrofuran (10 mL), stirred for 10 min in ice bath, triphosgene (75.0 mg, 0.3 mmol) was added and the reaction was continued for 10 min, benzylamine (74.0 mg, 0.5 mmol) was added. The reaction was carried out under ice bath for 30 min and moved to room temperature for 1 hour. At the end of the reaction, water (20 mL) was added to the reaction system, extracted with ethyl acetate (30 mL×3), the organic phases were combined, washed with saturated sodium chloride solution (30 mL×3), dried with anhydrous sodium sulfate, filtered, concentrated under reduced pressure, and the residue was purified by column chromatography on silica gel (eluted with methanol/dichloromethane=1/40) to give compound 16 (65.0 mg, 24.1% yield), as a white solid. MS(m/z): 539.8 [M+H]⁺; ¹H NMR (400 MHz, DMSO-d₆) δ10.99 (s, 1H), 8.99 (d, J=4.3 Hz, 1H), 7.61 (d, J=7.8 Hz, 1H), 7.52-7.09 (m, 12H), 6.91 (d, J=8.4 Hz, 1H), 6.78 (t, J=5.9 Hz, 1H), 5.40 (d, J=8.0 Hz, 1H), 5.10 (dd, J=13.3, 5.1 Hz, 1H), 4.42-4.17 (m, 6H), 2.91 (dd, J=21.8, 8.8 Hz, 1H), 2.61 (d, J=18.3 Hz, 1H), 2.44-2.32 (m, 1H), 2.01 (s, 1H).

### Example 17 (4-182)

### (2S)-2-(2-(cyclopentyloxy)acetamide)-N-(2-(2,6-dioxopiridin-3-yl)-1-oxoisoidoline-5-yl) methyl)-2-phenylacetamide

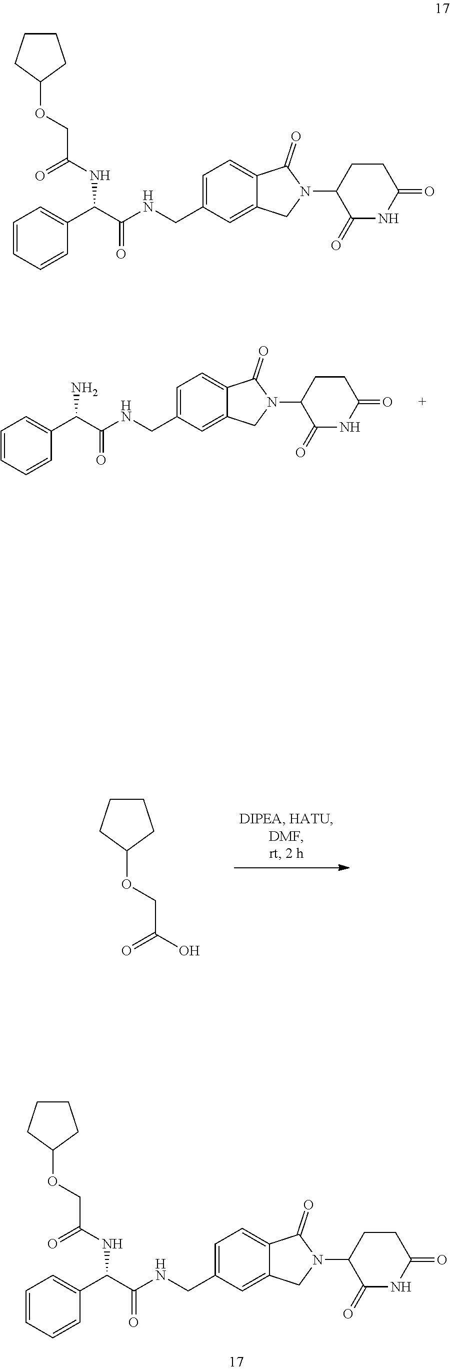 [SMILES: O=C1CCC(N2Cc3cc(CNC(=O)[C@@H](NC(=O)COC4CCCC4)c4ccccc4)ccc3C2=O)C(=O)N1 ; N[C@H](C(=O)NCc1ccc2c(c1)CN(C1CCC(=O)NC1=O)C2=O)c1ccccc1 ; O=C(O)COC1CCCC1 ; O=C1CCC(N2Cc3cc(CNC(=O)[C@@H](NC(=O)COC4CCCC4)c4ccccc4)ccc3C2=O)C(=O)N1]

Compound 17 was prepared according to Example 1, Step 5. MS(m/z): 533.2 [M+H]⁺; ¹H NMR (400 MHz, DMSO-d₆) δ10.99 (s, 1H), 8.98 (t, J=5.3 Hz, 1H), 8.01 (d, J=7.7 Hz, 1H), 7.62 (d, J=7.7 Hz, 1H), 7.47-7.20 (m, 7H), 5.52 (d, J=7.7 Hz, 1H), 5.10 (dd, J=13.3, 5.1 Hz, 1H), 4.54-4.13 (m, 4H), 3.98 (d, J=2.5 Hz, 1H), 3.90 (d, J=1.2 Hz, 2H), 2.99-2.85 (m, 1H), 2.61 (d, J=17.1 Hz, 1H), 2.40 (dd, J=13.1, 4.4 Hz, 1H), 2.00 (dd, J=8.9, 3.7 Hz, 1H), 1.68-1.62 (m, 4H), 1.51 (d, J=4.9 Hz, 2H), 1.31-1.22 (m, 2H).

### Example 18 (4-172)

### (2S)—N-(2-(2,6-dioxypiperidin-3-yl)-1-oxoisoidoline-5-yl)methyl)-2-(2-phenoxyacetamide)-2-phenylacetamide

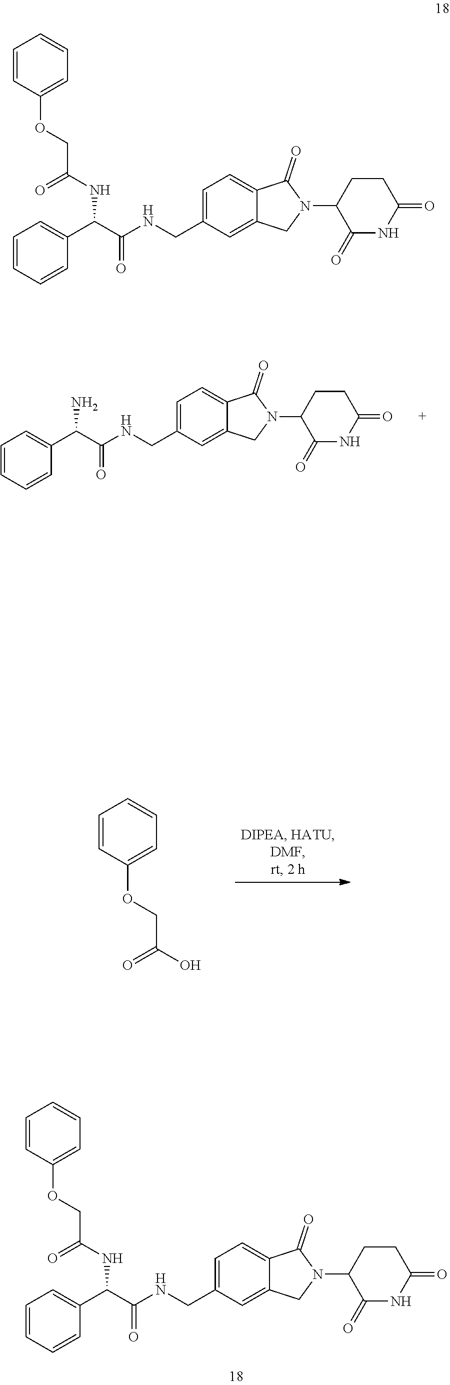 [SMILES: O=C1CCC(N2Cc3cc(CNC(=O)[C@@H](NC(=O)COc4ccccc4)c4ccccc4)ccc3C2=O)C(=O)N1 ; N[C@H](C(=O)NCc1ccc2c(c1)CN(C1CCC(=O)NC1=O)C2=O)c1ccccc1 ; O=C(O)COc1ccccc1 ; O=C1CCC(N2Cc3cc(CNC(=O)[C@@H](NC(=O)COc4ccccc4)c4ccccc4)ccc3C2=O)C(=O)N1]

Compound 18 was prepared according to Example 1, Step 5. MS(m/z): 540.8 [M+H]⁺; ¹H NMR (400 MHz, DMSO-d₆) δ10.99 (s, 1H), 8.97 (dd, J=5.7, 3.9 Hz, 1H), 8.59 (d, J=7.7 Hz, 1H), 7.62 (d, J=8.1 Hz, 1H), 7.48-7.18 (m, 9H), 6.96 (dd, J=12.4, 4.6 Hz, 3H), 5.57 (d, J=7.7 Hz, 1H), 5.11 (dd, J=13.3, 5.1 Hz, 1H), 4.80-4.49 (m, 2H), 4.47-4.18 (m, 4H), 3.04-2.82 (m, 1H), 2.61 (d, J=16.5 Hz, 1H), 2.39 (dd, J=13.1, 4.4 Hz, 1H), 2.00 (dd, J=9.0, 3.6 Hz, 1H).

### Example 19 (5-16)

### (2S)-2-(3-(tert butyl) urea)-N-(2-(2,6-dioxopyridin-3-yl)-1-oxoisoidoline-5-yl)methyl)-2-phenylacetamide

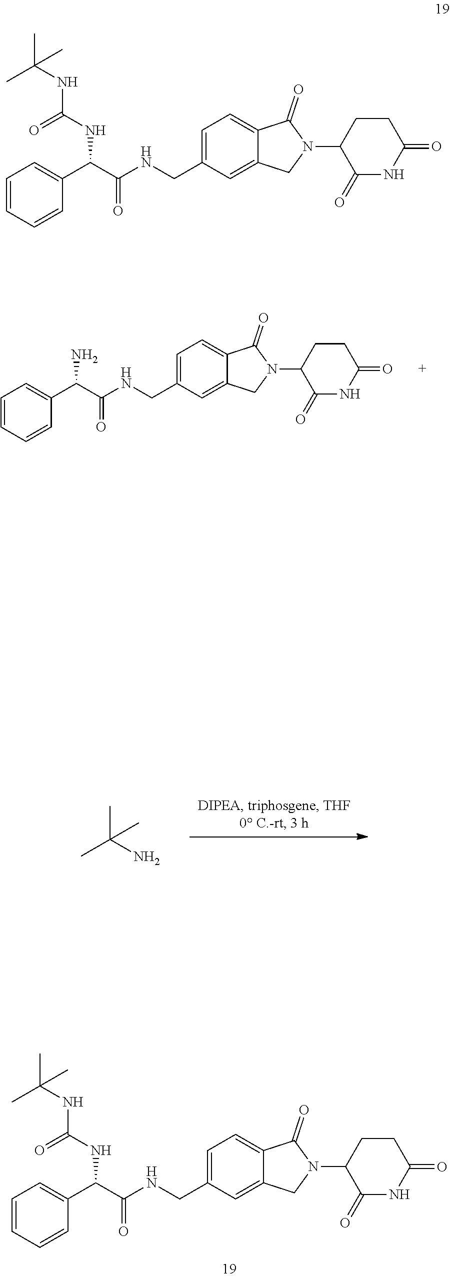

Compound 19 was prepared according to Example 16. MS(m/z): 505.9 [M+H]; ¹H NMR (400 MHz, DMSO-d₆) δ10.97 (s, 1H), 9.13-8.82 (m, 2H), 7.59 (d, J=7.7 Hz, 1H), 7.41-7.25 (m, 6H), 6.58 (d, J=8.1 Hz, 1H), 6.13 (s, 1H), 5.30 (d, J=8.0 Hz, 1H), 5.09 (dd, J=13.2, 5.0 Hz, 1H), 4.32 (dt, J=51.3, 13.5 Hz, 4H), 2.97-2.84 (m, 1H), 2.60 (d, J=16.8 Hz, 1H), 2.39 (ddd, J=26.7, 13.3, 4.4 Hz, 1H), 2.05-1.93 (m, 1H), 1.20 (s, 9H).

### Example 20 (4-126)

### 2-(3-(tert butyl) urea)-N-(2-(2,6-dioxopyridin-3-yl)-1-oxoisoidoline-5-yl)methyl)-2-phenylacetamide

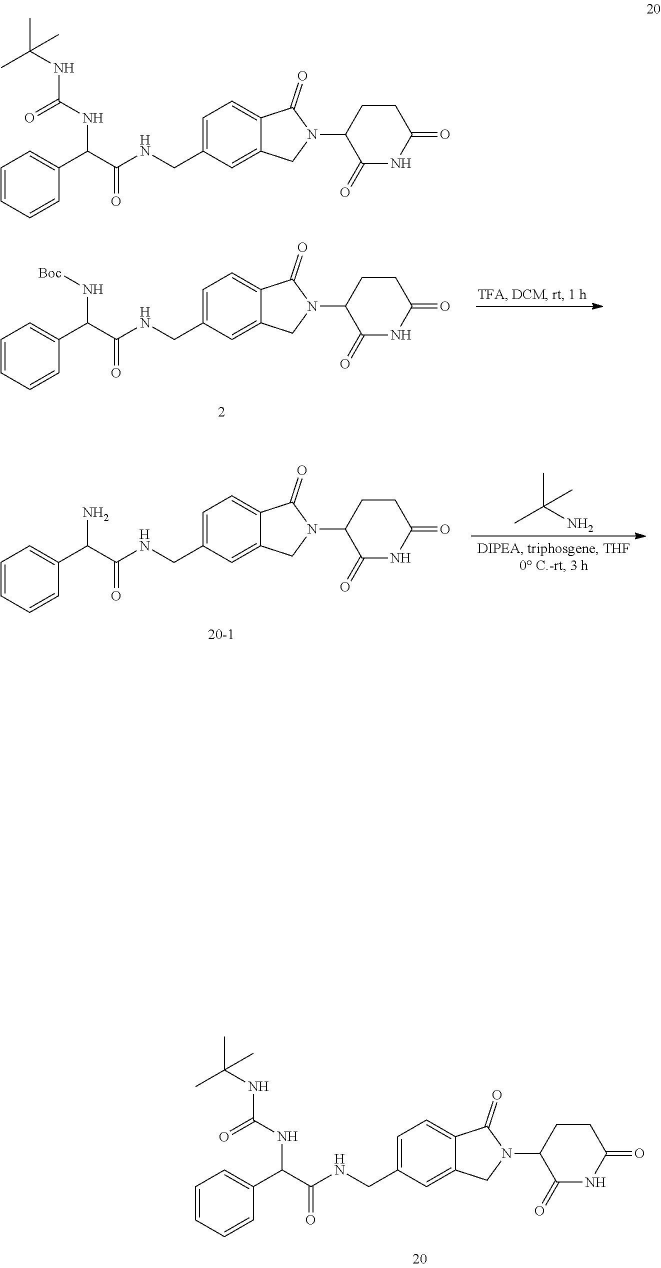

Intermediate 20-1 was prepared according to the method of Step 4 of Example 1; Compound 20 was prepared according to Example 16. MS(m/z): 505.9 [M+H]⁺

### Example 21 (4-129)

### N-(2-((2-(2,6-dioxypiperidin-3-yl)-1-oxoisoindoline-5-yl)methyl)amino)-2-oxo-1-phenylethyl) morpholine-4-formamide

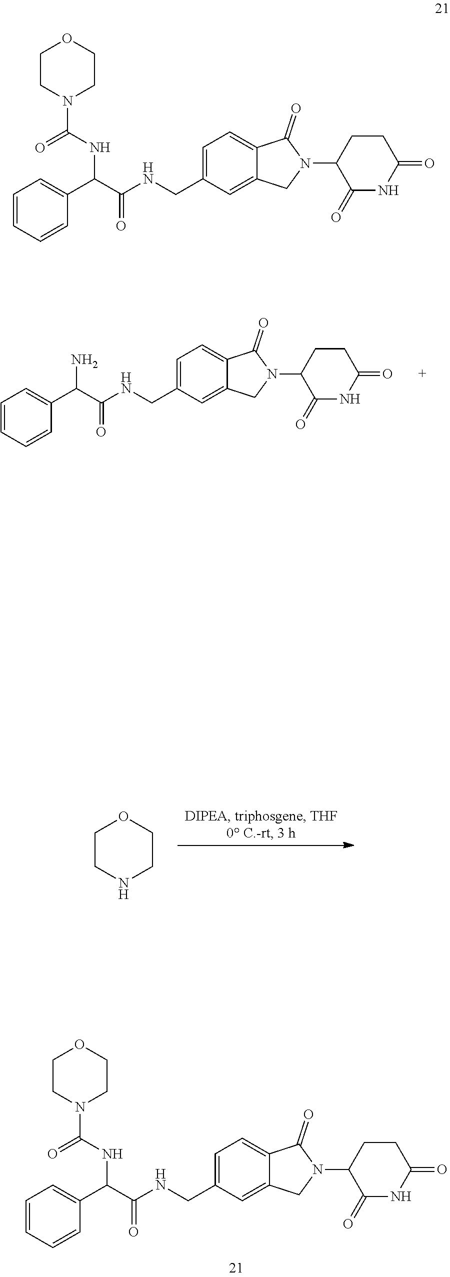 [SMILES: O=C1CCC(N2Cc3cc(CNC(=O)C(NC(=O)N4CCOCC4)c4ccccc4)ccc3C2=O)C(=O)N1 ; NC(C(=O)NCc1ccc2c(c1)CN(C1CCC(=O)NC1=O)C2=O)c1ccccc1 ; C1COCCN1 ; O=C1CCC(N2Cc3cc(CNC(=O)C(NC(=O)N4CCOCC4)c4ccccc4)ccc3C2=O)C(=O)N1]

Compound 21 was prepared according to Example 16. MS(m/z): 519.8 [M+H]⁺

### Example 22 (4-158)

### N-((1S)-2-(2-(2,6-dioxypiperidin-3-yl)-1-oxoisoidoline-5-yl)methyl)amino)-2-oxo-1-phenylethyl)-3,3-dimethylbutylamine

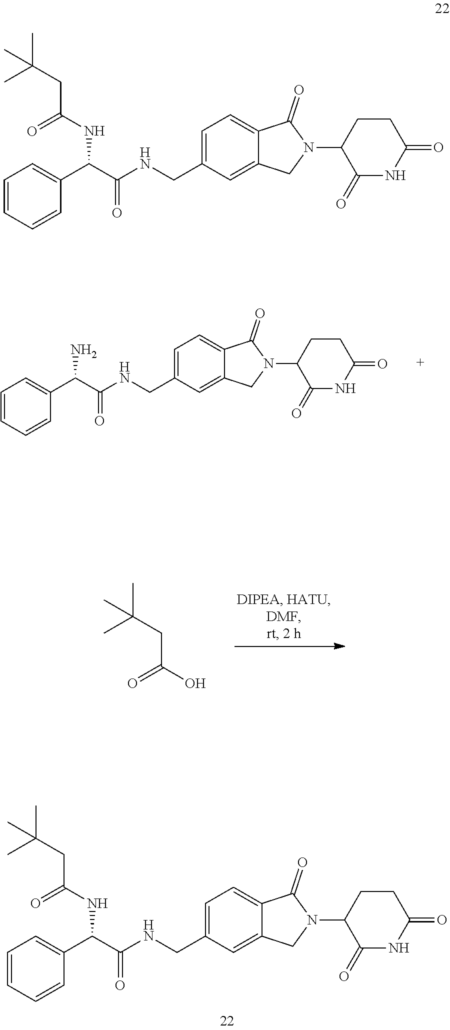

Compound 22 was prepared according to Example 1, Step 5. MS(m/z): 505.3 [M+H]⁺; ¹H NMR (400 MHz, DMSO-d₆) δ10.98 (s, 1H), 8.86 (t, J=5.2 Hz, 1H), 8.40 (d, J=7.8 Hz, 1H), 7.61 (d, J=8.0 Hz, 1H), 7.45 (d, J=7.5 Hz, 2H), 7.40-7.28 (m, 5H), 5.55 (d, J=7.8 Hz, 1H), 5.11 (dd, J=13.2, 5.0 Hz, 1H), 4.46-4.20 (m, 4H), 2.98-2.86 (m, 1H), 2.61 (d, J=17.0 Hz, 1H), 2.48-2.31 (m, 1H), 2.18-2.08 (m, 2H), 2.05-1.95 (m, 1H), 0.95 (s, 9H).

### Example 23 (4-165)

### (2S)-2-(2-cyclopropylacetamide)-N-(2-(2,6-dioxopyridin-3-yl)-1-oxoisoidoline-5-yl) methyl)-2-phenylacetamide

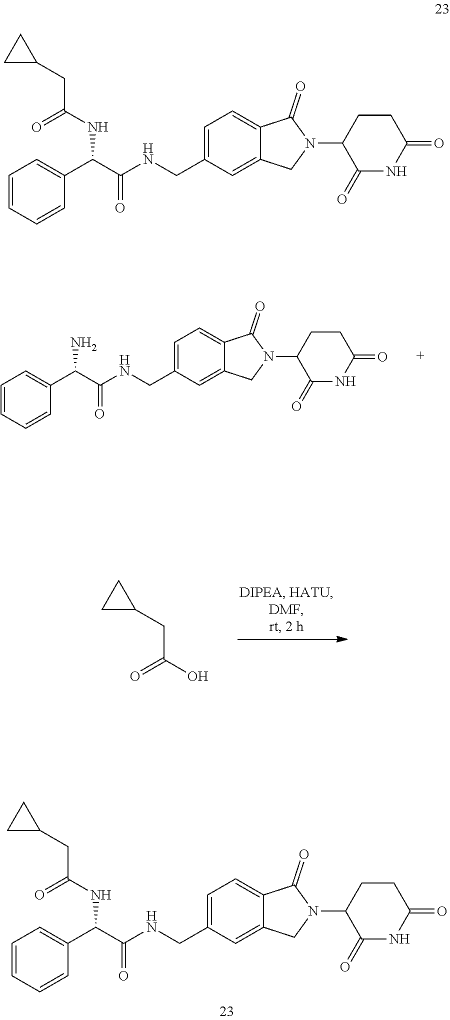

Compound 23 was prepared according to Example 1, Step 5. MS(m/z): 489.3 [M+H]⁺; ¹H NMR (400 MHz, DMSO-d₆) δ10.99 (s, 1H), 8.89 (s, 1H), 8.41 (d, J=7.9 Hz, 1H), 7.63 (d, J=8.0 Hz, 1H), 7.45 (d, J=7.4 Hz, 2H), 7.37 (t, J=7.3 Hz, 2H), 7.31 (d, J=7.2 Hz, 3H), 5.54 (d, J=7.9 Hz, 1H), 5.11 (dd, J=13.3, 5.0 Hz, 1H), 4.31 (dd, J=52.2, 17.8 Hz, 4H), 2.97-2.86 (m, 1H), 2.61 (d, J=17.5 Hz, 1H), 2.41 (qd, J=13.2, 4.3 Hz, 1H), 2.22-2.08 (m, 2H), 2.05-1.95 (m, 1H), 0.96 (dd, J=9.8, 4.9 Hz, 1H), 0.47-0.36 (m, 2H), 0.14 (d, J=4.6 Hz, 2H).

### Example 24 (4-176)

### (2S)—N-(2-(2,6-dioxypiperidin-3-yl)-1-oxoisoindoline-5-yl)methyl)-2-((4-methylphenyl) sulfonamide)-2-phenylacetamide

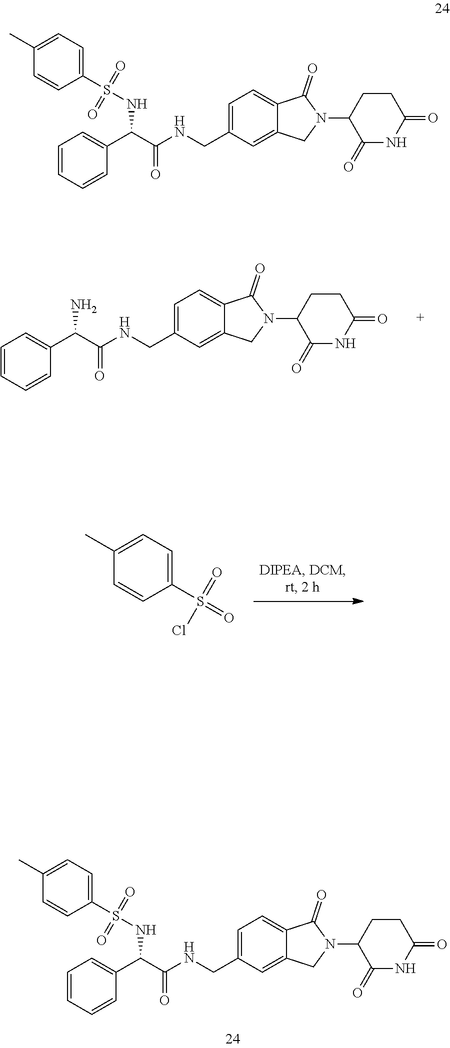

Compound 24 was prepared according to Example 11, Step 2. MS(m/z): 560.8 [M+H]⁺; ¹H NMR (400 MHz, DMSO-d₆) δ10.99 (s, 1H), 8.76 (t, J=5.8 Hz, 1H), 8.54 (d, J=8.7 Hz, 1H), 7.61 (dd, J=10.3, 8.2 Hz, 3H), 7.44 (dd, J=35.1, 7.4 Hz, 1H), 7.36-7.30 (m, 2H), 7.26 (dd, J=12.4, 7.7 Hz, 4H), 7.21 (d, J=2.9 Hz, 1H), 7.17 (d, J=7.9 Hz, 1H), 5.11 (dd, J=13.3, 5.0 Hz, 1H), 5.02 (d, J=8.4 Hz, 1H), 4.50-4.15 (m, 4H), 2.97-2.84 (m, 1H), 2.61 (d, J=16.3 Hz, 1H), 2.48-2.37 (m, 1H), 2.37-2.29 (m, 3H), 2.08-1.94 (m, 1H).

### Example 25 (4-177)

### (2S)-2-(cyclopropane sulfonamide)-N-(2-(2,6-dioxypiperidin-3-yl)-1-oxoisoidoline-5-yl) methyl)-2-phenylacetamide

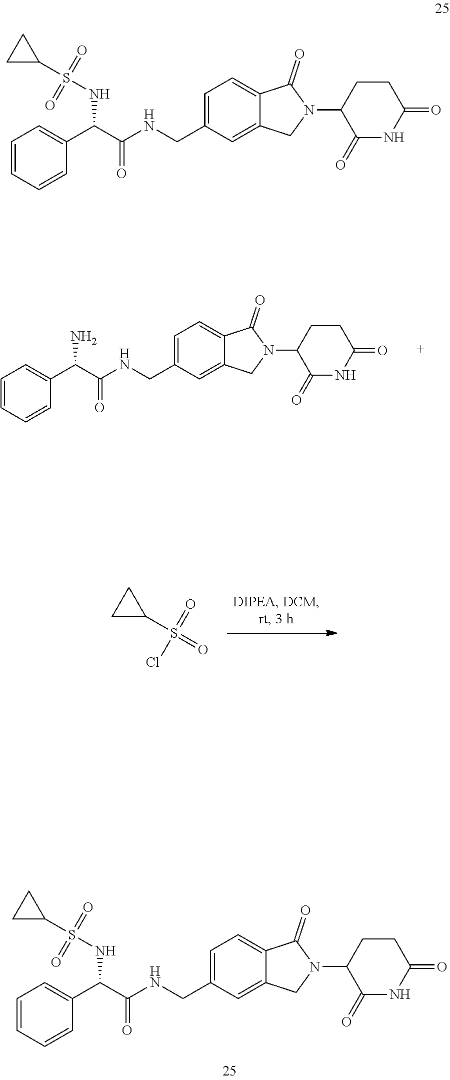

Compound 25 was prepared according to Example 11, Step 2. MS(m/z): 510.8 [M+H]⁺; ¹H NMR (400 MHz, DMSO-d₆) δ10.99 (s, 1H), 8.89 (t, J=5.4 Hz, 1H), 8.09 (d, J=9.5 Hz, 1H), 7.63 (d, J=7.6 Hz, 1H), 7.50 (dd, J=15.2, 7.1 Hz, 2H), 7.35 (ddt, J=21.9, 14.7, 7.4 Hz, 5H), 5.11 (dd, J=11.6, 5.8 Hz, 2H), 4.50-4.18 (m, 4H), 2.92 (ddd, J=13.5, 11.9, 5.3 Hz, 1H), 2.61 (d, J=17.0 Hz, 1H), 2.48-2.32 (m, 1H), 2.33-2.22 (m, 1H), 2.11-1.94 (m, 1H), 1.25 (s, 1H), 0.96-0.65 (m, 3H).

### Example 26 (4-197)

### N-((1S)-2-(2-(2,6-dioxypiperidin-3-yl)-1-oxoisoindoline-5-yl)methyl)amino)-2-oxo-1-phen ylethyl)-2-hydroxy-3-methylbutanamide

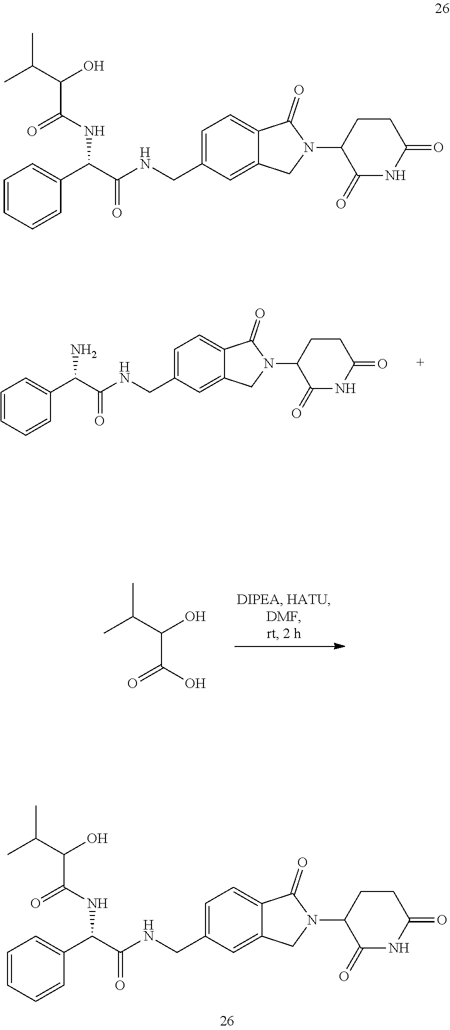

Compound 26 was prepared according to Example 1, Step 5. MS(m/z): 506.9 [M+H]⁺; ¹H NMR (400 MHz, DMSO-d₆) δ10.96 (s, 1H), 8.95 (s, 1H), 8.11 (dd, J=25.1, 7.9 Hz, 1H), 7.67-7.53 (m, 1H), 7.34 (ddd, J=29.5, 20.2, 6.4 Hz, 6H), 5.66-5.41 (m, 2H), 5.09 (dd, J=13.3, 4.8 Hz, 1H), 4.54-4.13 (m, 4H), 4.08 (d, J=5.2 Hz, 1H), 3.87-3.65 (m, 1H), 2.97-2.84 (m, 1H), 2.60 (d, J=16.6 Hz, 1H), 2.40 (d, J=13.0 Hz, 1H), 1.98 (d, J=5.9 Hz, 2H), 0.93-0.83 (m, 3H), 0.72 (dd, J=35.5, 6.7 Hz, 3H).

### Example 27 (4-192)

### (2S)—N-(2-(2,6-dioxopyridin-3-yl)-1-oxoisoidoline-5-yl)methyl)-2-(2-hydroxy-2-phenylacetamide)-2-phenylacetamide

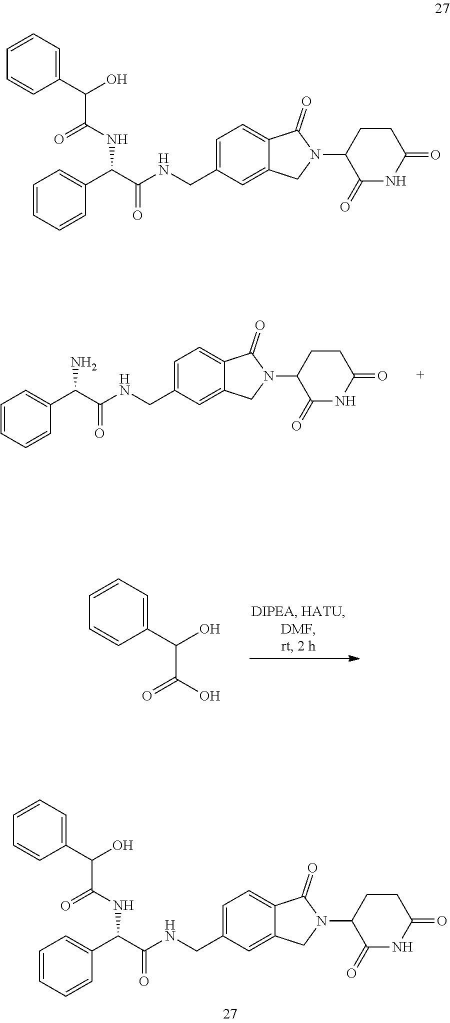

Compound 27 was prepared according to Example 1, Step 5. MS(m/z): 540.8 [M+H]⁺; ¹H NMR (400 MHz, DMSO-d₆) δ10.96 (s, 1H), 8.95 (d, J=3.6 Hz, 1H), 8.39 (dd, J=10.9, 8.1 Hz, 1H), 7.64-7.58 (m, 1H), 7.45-7.40 (m, 2H), 7.38 (d, J=7.5 Hz, 2H), 7.32 (dd, J=13.9, 6.7 Hz, 4H), 7.27 (dd, J=10.4, 5.6 Hz, 3H), 6.38 (t, J=4.5 Hz, 1H), 5.48 (dd, J=12.9, 8.0 Hz, 1H), 5.09 (dd, J=13.2, 5.1 Hz, 1H), 5.03 (d, J=4.9 Hz, 1H), 4.43-4.37 (m, 2H), 4.37-4.30 (m, 1H), 4.22 (dd, J=17.3, 7.1 Hz, 1H), 4.07 (q, J=5.3 Hz, 1H), 2.98-2.85 (m, 1H), 2.61 (d, J=17.1 Hz, 1H), 2.40 (d, J=12.9 Hz, 1H), 2.05-1.96 (m, 1H).

### Example 28 (4-199)

### (2S)—N-(2-(2,6-dioxypiperidin-3-yl)-1-oxoisoindolin-5-yl)methyl)-2-(2-methoxy-2-phenylacetamide)-2-phenylacetamide

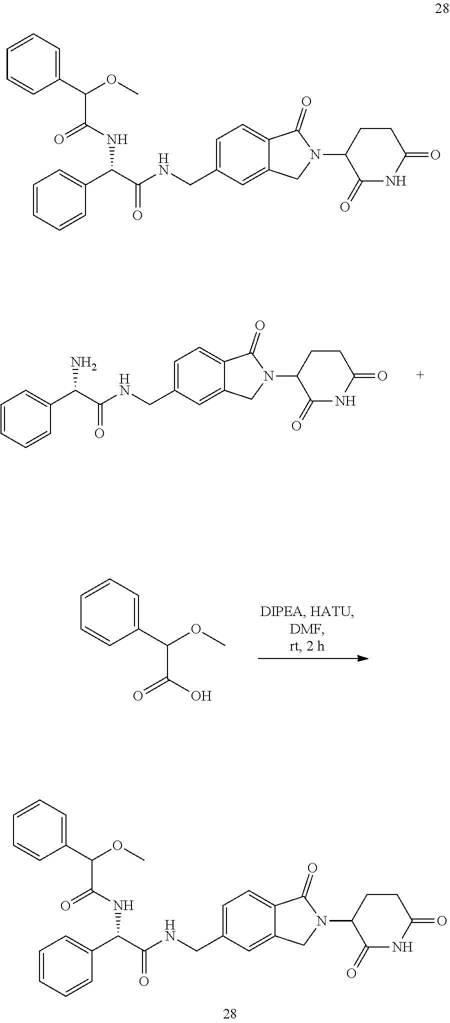

Compound 28 was prepared according to Example 1, Step 5. MS(m/z): 555.2[M+H]⁺; ¹H NMR (400 MHz, DMSO-d₆) δ10.97 (s, 1H), 8.92 (s, 1H), 8.45 (t, J=7.9 Hz, 1H), 7.60 (d, J=7.7 Hz, 1H), 7.49-7.16 (m, 12H), 5.50 (dd, J=11.1, 8.0 Hz, 1H), 5.09 (dd, J=13.3, 5.0 Hz, 1H), 4.84 (s, 1H), 4.50-4.14 (m, 4H), 2.91 (ddd, J=13.6, 11.8, 5.4 Hz, 1H), 2.60 (d, J=17.1 Hz, 1H), 2.39 (dd, J=13.1, 4.3 Hz, 1H), 2.04-1.94 (m, 1H), 1.25 (d, J=6.0 Hz, 3H).

### Example 29 (4-198)

### (2S)—N-(2-(2,6-dioxypiperidin-3-yl)-1-oxoisoindoline-5-yl)methyl)-2-(2-oxo-2-phenylacetamide)-2-phenylacetamide

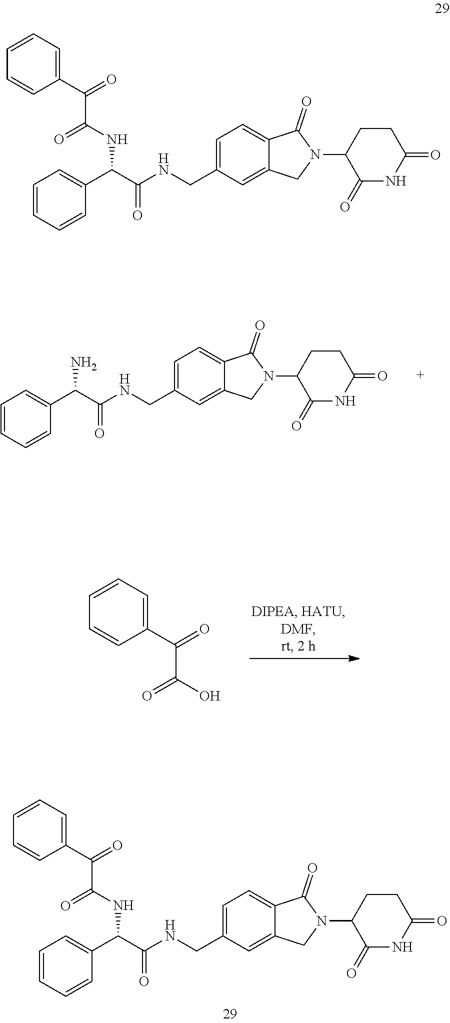

Compound 29 was prepared according to Example 1, Step 5. MS(m/z): 538.8 [M+H]⁺; ¹H NMR (400 MHz, DMSO-d₆) δ10.95 (s, 1H), 9.59 (d, J=7.6 Hz, 1H), 8.97 (t, J=4.8 Hz, 1H), 7.93 (d, J=8.2 Hz, 2H), 7.71 (t, J=7.4 Hz, 1H), 7.62 (d, J=8.0 Hz, 1H), 7.59-7.48 (m, 4H), 7.44-7.35 (m, 3H), 7.32 (d, J=7.9 Hz, 2H), 5.67 (d, J=7.6 Hz, 1H), 5.09 (dd, J=13.2, 5.0 Hz, 1H), 4.55-4.16 (m, 4H), 2.96-2.84 (m, 1H), 2.60 (d, J=17.5 Hz, 1H), 2.39 (d, J=12.9 Hz, 1H), 2.07-1.93 (m, 1H).

### Example 30 (4-196-2)

### (2S)—N-((2-(2,6-dioxypiperidin-3-yl)-1-oxoisoindolin-5-yl)methyl)-2-((S)-2-(methylamino)-2-phenylacetamide)-2-phenylacetamide

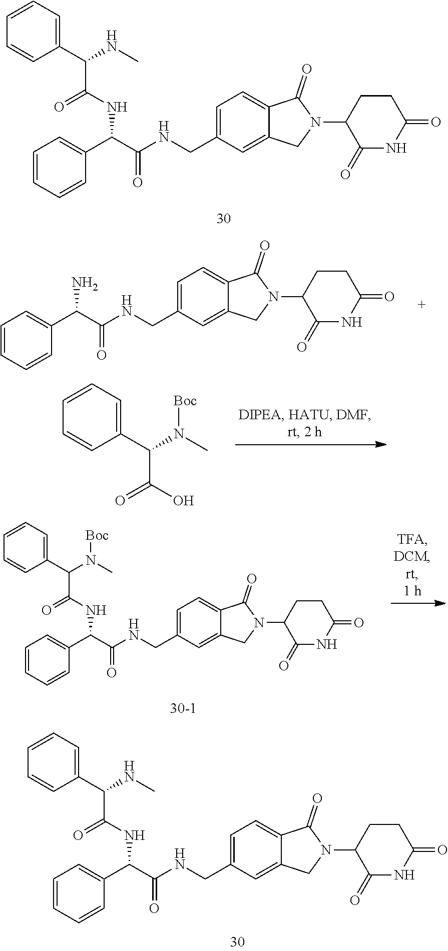

Intermediate 30-1 was prepared according to the method of Step 5 of Example 1. MS(m/z): 553.8 [M+H]⁺; ¹H NMR (400 MHz, DMSO-d₆) δ10.97 (s, 1H), 9.56-9.21 (m, 3H), 8.91 (t, J=5.6 Hz, 1H), 7.55 (dd, J=10.3, 5.1 Hz, 3H), 7.47 (dd, J=5.4, 2.8 Hz, 4H), 7.36 (dt, J=20.1, 6.9 Hz, 3H), 7.22-7.16 (m, 2H), 5.57 (d, J=7.7 Hz, 1H), 5.17-5.01 (m, 2H), 4.44-4.16 (m, 4H), 4.08 (s, 1H), 2.98-2.85 (m, 1H), 2.61 (d, J=17.0 Hz, 1H), 2.43 (dd, J=13.3, 4.5 Hz, 1H), 2.38 (s, 2H), 2.00 (dd, J=9.1, 3.6 Hz, 1H).

Compound 30 was prepared according to Example 1, Step 4.

### Example 31 (4-193)

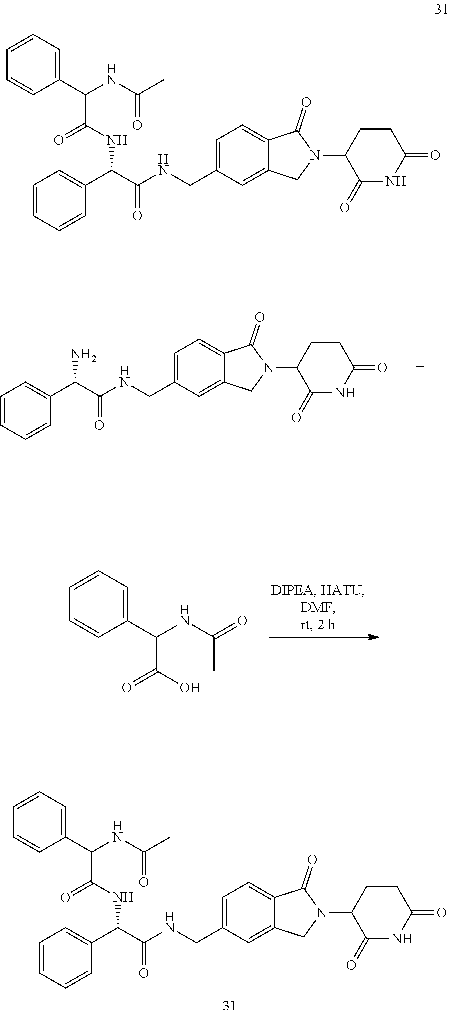

Compound 31 was prepared according to Example 1, Step 5. MS(m/z): 581.8 [M+H]⁺; ¹H NMR (400 MHz, DMSO-d₆) δ10.96 (s, 1H), 8.93 (d, J=7.8 Hz, 1H), 8.77 (d, J=3.3 Hz, 1H), 8.46 (d, J=8.2 Hz, 1H), 7.59 (dd, J=20.7, 7.9 Hz, 1H), 7.44 (dd, J=15.8, 6.9 Hz, 3H), 7.37 (t, J=7.3 Hz, 2H), 7.33-7.26 (m, 5H), 7.21 (t, J=7.2 Hz, 2H), 5.73 (dd, J=14.3, 8.1 Hz, 1H), 5.51 (d, J=7.9 Hz, 1H), 5.09 (dd, J=13.1, 4.9 Hz, 1H), 4.37 (dd, J=23.3, 5.4 Hz, 2H), 4.23 (dd, J=33.0, 14.4 Hz, 1H), 4.06 (d, J=5.2 Hz, 1H), 2.89 (dd, J=8.2, 4.5 Hz, 1H), 2.60 (d, J=17.3 Hz, 1H), 2.39 (dd, J=13.0, 4.2 Hz, 1H), 2.05-1.95 (m, 1H), 1.89 (d, J=6.8 Hz, 3H).

### Example 32 (4-109)

### Tert butyl ((1S)-2-((2-(2,6-dioxypiperidin-3-yl)-1-oxoisoindoline-4-yl) amino)-2-oxo-1-phenylethyl) carbamate

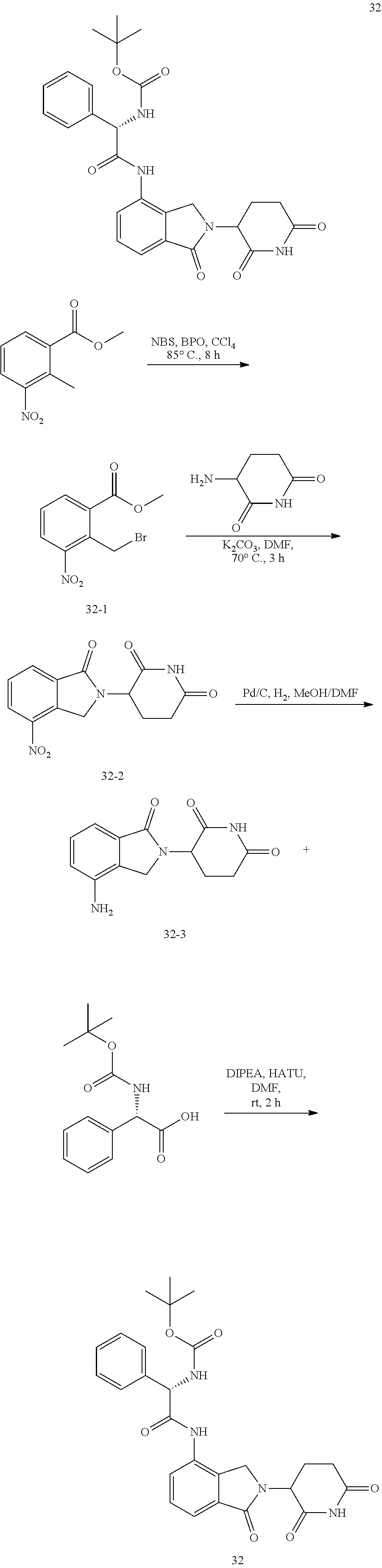

### Step 1: Preparation of methyl 2-(bromomethyl)-3-nitrobenzoate (32-1)

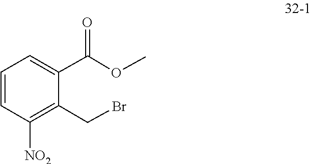

Methyl 2-methyl-3-nitrobenzoate (3.4 g, 17.4 mmol), N-bromosuccinimide (3.4 g, 19.1 mmol) and benzoyl peroxide (43.0 mg, 0.18 mmol) were dissolved in carbon tetrachloride (40 mL) under nitrogen protection and the reaction was stirred at 85° C. for 8 hours. The reaction solution was concentrated under reduced pressure. The residue was separated by silica gel column chromatography (eluting with petroleum ether) to give Intermediate 32-1 (1.9 g, 40.0% yield), as a white solid. ¹H NMR (400 MHz, DMSO-d₆) δ 8.16-8.14 (m, 1H), 8.02-8.00 (m, 1H), 7.79-7.77 (m, 1H), 4.76 (s, 2H), 3.89 (s, 3H).

### Step 2: Preparation of 3-(4-nitro-1-oxoisoindolin-2-yl)piperidine-2,6-dione (32-2)

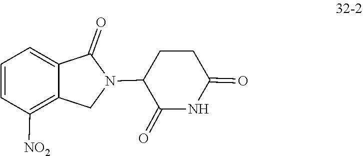 [SMILES: O=C1CCC(N2Cc3c(cccc3[N+](=O)[O-])C2=O)C(=O)N1]

Under nitrogen protection, 32-1 (1.9 g, 6.6 mmol), 3-aminopiperidine-2,6-dione hydrochloride (1.1 g, 6.6 mmol) and anhydrous potassium carbonate (2.8 g, 19.7 mmol) were dissolved in N,N-dimethylformamide (20 mL) and the reaction was stirred at 75° C. for 3 hours. The reaction solution was concentrated under reduced pressure. The residue was stirred with water (30 mL) for 0.5 h at room temperature, filtered and dried to obtain 32-2 (1.2 g, yield 63.2%), as an off-white solid. MS(m/z): 289.9 [M+H]⁺; ¹H NMR (400 MHz, DMSO-d₆) δ 11.06 (s, 1H), 8.48 (d, J=8.0 Hz, 1H), 8.20 (d, J=6.0 Hz, 1H), 7.85-7.82 (m, 1H), 5.20 (d, J=8.0 Hz, 1H), 4.86 (dd, J=16.0, 12.0 Hz, 2H), 2.91 (d, J=12.0 Hz, 1H), 2.68-2.54 (m, 2H), 2.05-2.03 (m, 1H).

### Step 3: Preparation of 3-(4-amino-1-oxoisoindolin-2-yl)piperidine-2,6-dione (32-3)

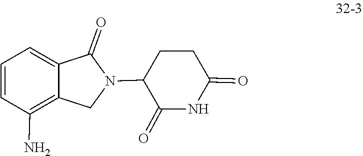 [SMILES: Nc1cccc2c1CN(C1CCC(=O)NC1=O)C2=O]

Under hydrogen conditions, 32-2 (1.2 g, 4.1 mmol), palladium carbon (0.4 g) were added to N,N-dimethylformamide (10 mL) and methanol (20 mL), and the reaction was stirred at room temperature overnight. The diatomite filtration was carried out and the reaction solution was concentrated under reduced pressure to give 32-3 (0.8 g, 72.7% yield) as an off-white solid. ¹H NMR (400 MHz, DMSO-d₆) δ 11.01 (s, 1H), 7.19-7.17 (m, 1H), 6.92-6.90 (m, 1H), 6.80-6.78 (m, 1H), 5.43-5.41 (m, 2H), 5.09-5.07 (m, 1H), 4.15 (dd, J=16.0, 8.0 Hz, 2H), 2.73-2.71 (m, 1H), 2.61 (d, J=16.0 Hz, 1H), 2.31-2.29 (m, 1H), 2.04-2.02 (m, 1H).

### Step 4: Preparation of tert butyl ((1S)-2-((2-(2,6-dioxypiperidin-3-yl)-1-oxoisoindoline-4-yl) amino)-2-oxo-1-phenylethyl) carbamate (32)

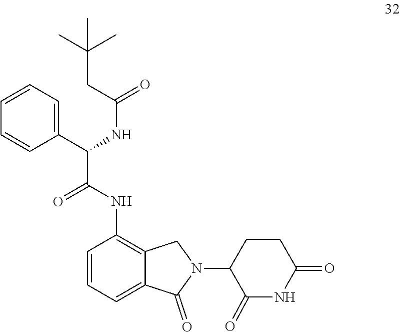 [SMILES: CC(C)(C)CC(=O)N[C@H](C(=O)Nc1cccc2c1CN(C1CCC(=O)NC1=O)C2=O)c1ccccc1]

Compound 32 was prepared according to Example 1, Step 5. MS(m/z): 392.9 [M−100]; ¹H NMR (400 MHz, DMSO-d₆) δ11.03 (s, 1H), 10.19 (s, 1H), 7.74 (d, J=7.6 Hz, 1H), 7.59-7.47 (m, 5H), 7.44-7.29 (m, 3H), 5.43 (d, J=7.3 Hz, 1H), 5.14 (dd, J=12.9, 4.6 Hz, 1H), 4.24 (s, 2H), 2.96-2.87 (m, 1H), 2.67 (d, J=12.6 Hz, 1H), 2.34-2.24 (m, 1H), 2.04 (d, J=10.9 Hz, 1H), 1.41 (s, 9H).

### Example 33 (4-118)

### Tert butyl ((1S)-2-((2-(2,6-dioxypiperidin-3-yl)-1-oxoisoindoline-5-yl) amino)-2-oxo-1-phenylethyl) carbamate

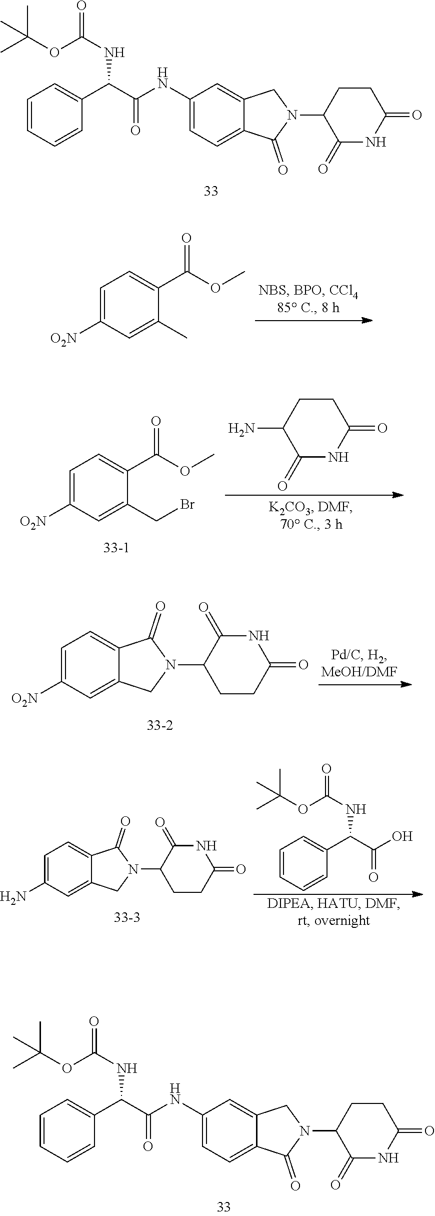

Intermediate 33-3 was prepared according to the method of Step 1 to Step 3 of Example 32.

Compound 33 was prepared according to Example 1, Step 5. MS(m/z): 392.9 [M−100]; ¹H NMR (400 MHI-z, DMSO-d₆) 310.97 (s, 1H), 10.59 (s, 1H), 7.95 (s, 1H), 7.69-7.48 (m, 5H), 7.34 (dt, J=23.1, 7.2 Hz, 3H), 5.40 (d, J=7.8 Hz, 1H), 5.08 (dd, J=13.3, 4.9 Hz, 1H), 4.36 (dd, J=53.9, 16.5 Hz, 2H), 2.90 (dd, J=21.8, 9.2 Hz, 1H), 2.62 (s, 1H), 2.42-2.33 (m, 1H), 2.01 (s, 1H), 1.41 (s, 9H).

### Example 34 (4-153)

### Tert butyl ((1S)-2-((2-(2,6-dioxopyridin-3-yl)-3-oxisoquinoline-5-yl)methyl)amino)-2-oxo-1-phenylethyl) carbamate

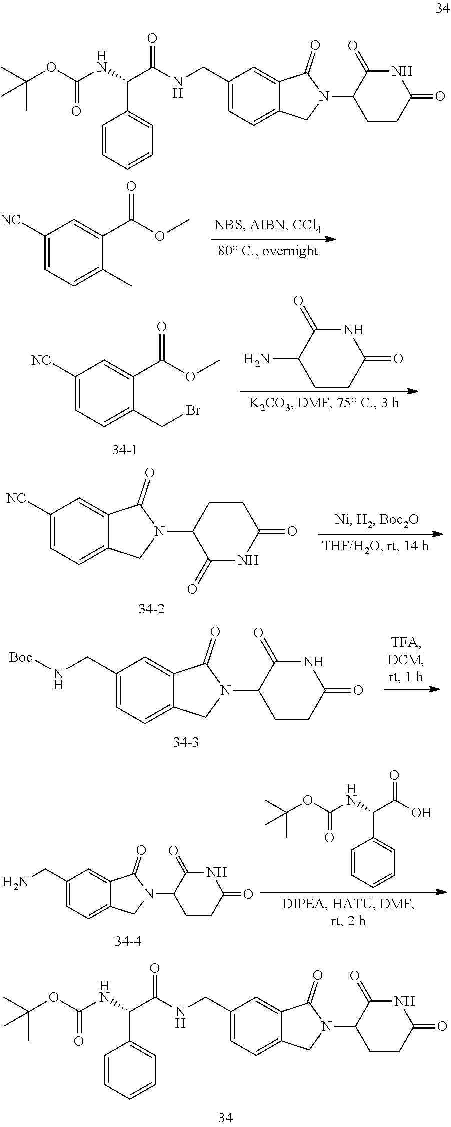

Intermediate 34-4 was prepared according to the method of Step 1 to Step 4 of Example 1.

Compound 34 was prepared according to Example 1, Step 5. MS (m/z): 406.9 [M−100]⁺; ¹H NMR (400 MHZ, DMSO-d₆) δ10.99 (s, 1H), 8.73 (t, J=5.6 Hz, 1H), 7.60 (s, 1H), 7.50 (d, J=7.8 Hz, 1H), 7.43 (d, J=6.9 Hz, 3H), 7.32 (dt, J=13.8, 7.1 Hz, 4H), 5.23 (d, J=8.0 Hz, 1H), 5.12 (dd, J=13.3, 5.0 Hz, 1H), 4.37 (dt, J=41.0, 17.3 Hz, 4H), 2.98-2.86 (m, 1H), 2.61 (d, J=17.1 Hz, 1H), 2.46-2.32 (m, 1H), 2.05-1.96 (m, 1H), 1.40 (s, 9H).

### Example 35 (4-167)

### Tert butyl ((1S)-2-((2-(2,6-dioxypiperidin-3-yl)-3-oxisoquinolin-4-yl)methyl)amino)-2-oxo-1-phenylethyl) carbamate

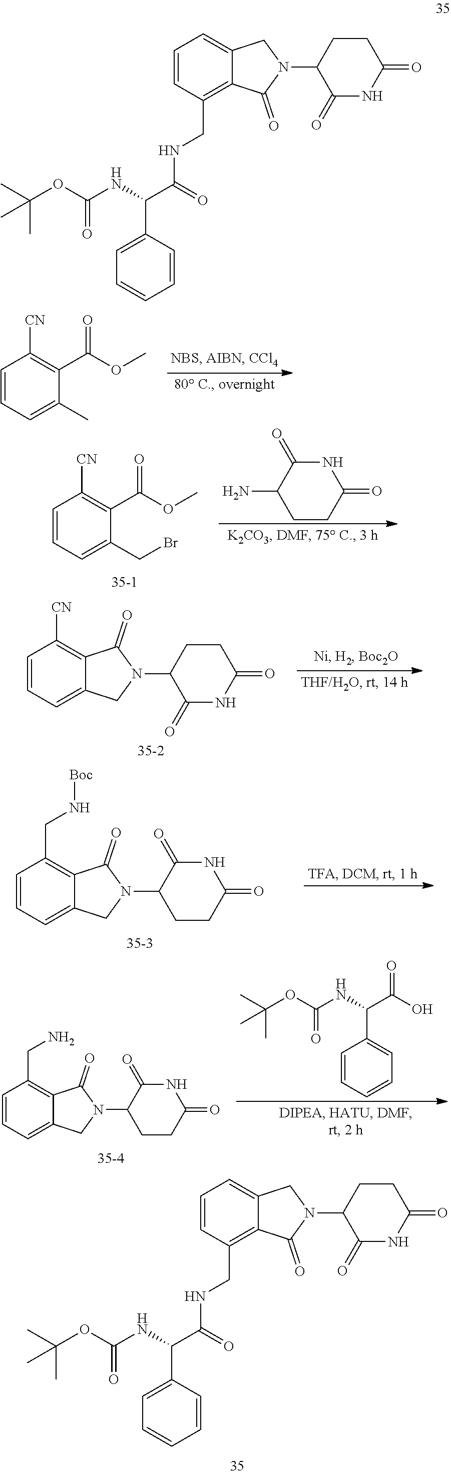

Intermediate 35-4 was prepared according to the method of Step 1 to Step 4 of Example 1.

Compound 35 was prepared according to Example 1, Step 5. MS(m/z): 406.9 [M−100]⁺; ¹H NMR (400 MHz, DMSO-d₆) δ10.96 (s, 1H), 8.61 (d, J=4.7 Hz, 1H), 7.48-7.40 (m, 4H), 7.38-7.25 (m, 4H), 7.13 (s, 1H), 5.22 (d, J=7.4 Hz, 1H), 5.08 (dd, J=13.2, 5.0 Hz, 1H), 4.86-4.71 (m, 2H), 4.36 (dd, J=48.0, 17.5 Hz, 2H), 2.89 (s, 1H), 2.60 (d, J=16.7 Hz, 1H), 2.45-2.33 (m, 1H), 2.07-1.94 (m, 1H), 1.39 (s, 9H).

### Example 36 (4-117)

### Tert butyl (2-((2-(2,6-dioxypiperidin-3-yl)-1-oxoisoindoline-5-yl)amino)-2-oxoethyl) carbamate

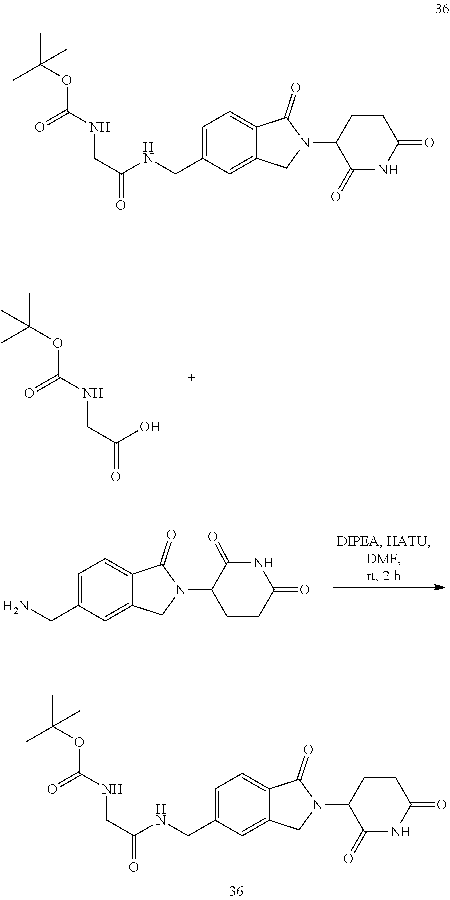 [SMILES: CC(C)(C)OC(=O)NCC(=O)NCc1ccc2c(c1)CN(C1CCC(=O)NC1=O)C2=O ; CC(C)(C)OC(=O)NCC(=O)O ; NCc1ccc2c(c1)CN(C1CCC(=O)NC1=O)C2=O ; CC(C)(C)OC(=O)NCC(=O)NCc1ccc2c(c1)CN(C1CCC(=O)NC1=O)C2=O]

Compound 36 was prepared according to Example 1, Step 5. MS(m/z): 330.9 [M−100]⁺; ¹H NMR (400 MHz, DMSO-d₆) δ10.98 (s, 1H), 8.41 (s, 1H), 7.67 (d, J=7.8 Hz, 1H), 7.57-7.33 (m, 2H), 7.02 (s, 1H), 5.12 (dd, J=13.3, 5.1 Hz, 1H), 4.54-4.21 (m, 4H), 3.60 (d, J=5.7 Hz, 2H), 3.04-2.84 (m, 1H), 2.66 (d, J=20.3 Hz, 1H), 2.41 (dd, J=13.3, 4.1 Hz, 1H), 2.09-1.94 (m, 1H), 1.37 (d, J=29.6 Hz, 9H).

### Example 37 (4-123)

### Tert butyl ((2R)-1-((2-(2,6-dioxypiperidin-3-yl)-1-oxoidoline-5-yl)methyl)amino)-4-methyl-1-oxolan-2-yl) carbamate

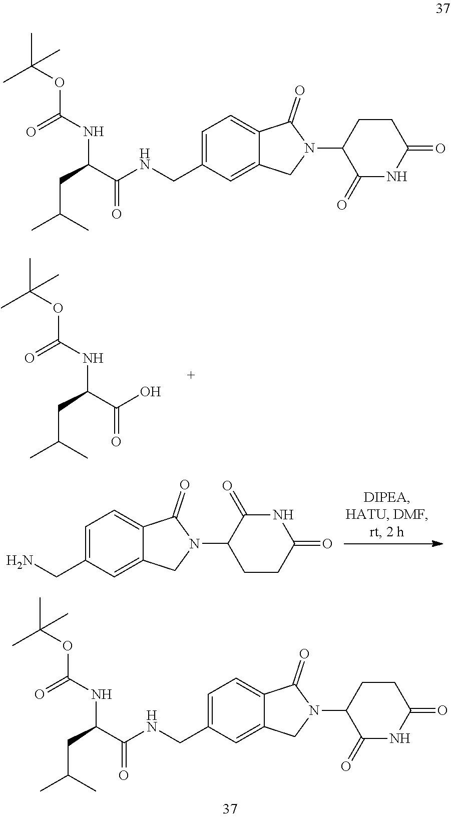

Compound 37 was prepared according to Example 1, Step 5. MS(m/z): 387.0[M−100]⁺; ¹H NMR (400 MHz, DMSO-d₆) δ10.98 (s, 1H), 8.46 (dd, J=13.0, 7.2 Hz, 1H), 7.63 (dd, J=26.2, 7.7 Hz, 1H), 7.49-7.33 (m, 2H), 6.94 (d, J=8.0 Hz, 1H), 5.12 (dd, J=13.2, 5.0 Hz, 1H), 4.47-4.31 (m, 4H), 3.18 (d, J=5.2 Hz, 2H), 3.00-2.86 (m, 1H), 2.61 (d, J=17.4 Hz, 1H), 2.41 (dd, J=13.1, 3.9 Hz, 1H), 2.06-1.96 (m, 1H), 1.66-1.55 (m, 1H), 1.40 (s, 9H), 0.88 (dd, J=11.2, 6.6 Hz, 6H).

### Example 38 (5-57)

### Tert butyl ((1S)-1-cyclopentyl-2-((2-(2,6-dioxopyridin-3-yl)-1-oxoisoidoline-5-yl) methyl) amino)-2-oxoethyl) carbamate

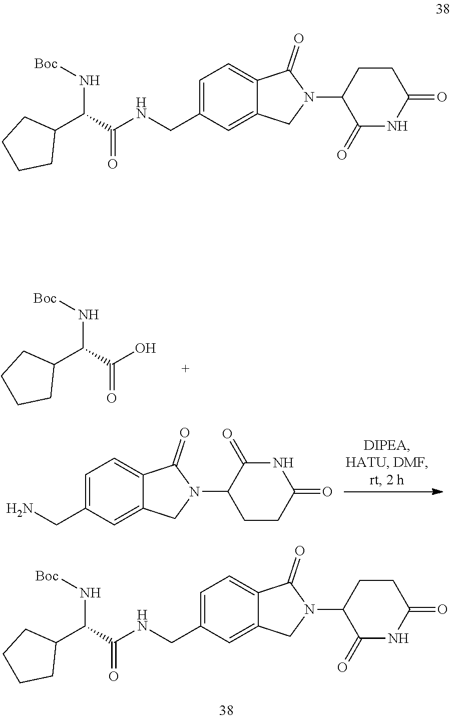

Compound 38 was prepared according to Example 1, Step 5. MS(m/z): 399.0[M−100]⁺; ¹H NMR (400 MHz, DMSO-d₆) δ10.95 (s, 1H), 8.45 (s, 1H), 7.64 (d, J=7.7 Hz, 1H), 7.49-7.28 (m, 2H), 6.83 (d, J=8.2 Hz, 1H), 5.74 (d, J=2.8 Hz, 1H), 5.10 (dd, J=13.2, 4.9 Hz, 1H), 4.50-4.22 (m, 4H), 2.98-2.78 (m, 1H), 2.60 (d, J=17.5 Hz, 1H), 2.45-2.29 (m, 1H), 2.19-1.94 (m, 2H), 1.66 (s, 1H), 1.52 (d, J=14.6 Hz, 2H), 1.48-1.43 (m, 2H), 1.39 (s, 9H), 1.33-1.16 (m, 4H).

### Example 39 (5-56)

### Tert butyl ((1S)-1-cyclohexyl-2-((2-(2,6-dioxopyridin-3-yl)-1-oxoisoidoline-5-yl) methyl) amino)-2-oxoethyl) carbamate

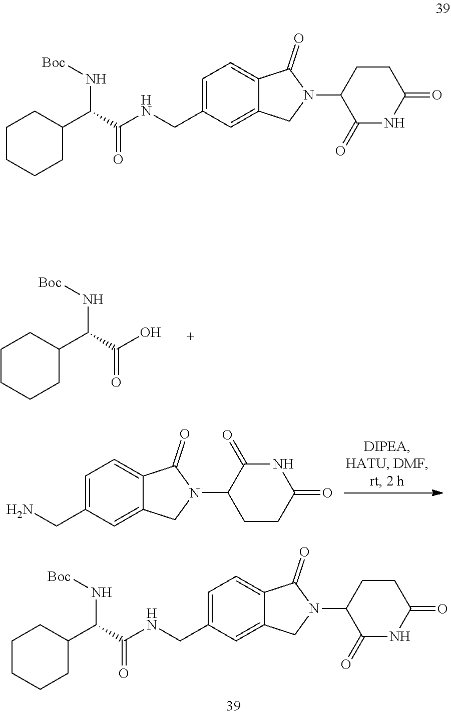

Compound 39 was prepared according to Example 1, Step 5. MS(m/z): 413.0[M−100]H NMR (400 MHz, DMSO-d₆) δ10.96 (s, 1H), 8.47 (s, 1H), 7.64 (d, J=7.8 Hz, 1H), 7.55-7.34 (m, 2H), 6.71 (d, J=8.6 Hz, 1H), 5.10 (dd, J=13.2, 5.0 Hz, 1H), 4.54-4.20 (m, 4H), 3.81 (t, J=7.8 Hz, 1H), 3.02-2.80 (m, 1H), 2.60 (d, J=17.0 Hz, 1H), 2.40 (dt, J=13.2, 8.9 Hz, 1H), 2.10-1.94 (m, 1H), 1.75-1.46 (m, 6H), 1.45-1.21 (m, 9H), 1.20-0.89 (m, 5H).

### Example 40 (4-124)

### Tert butyl (1-cyclohexyl-2-((2-(2,6-dioxypiperidin-3-yl)-1-oxoisoindoline-5-yl) methyl) amino)-2-oxoethyl) carbamate

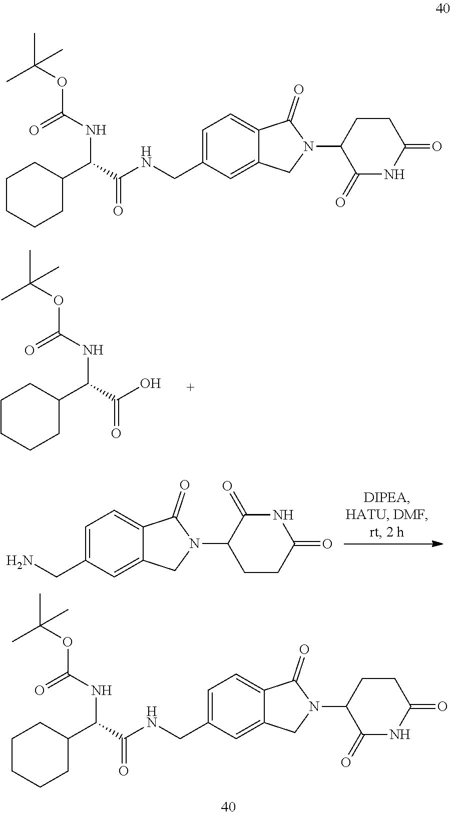

Compound 40 was prepared according to Example 1, Step 5. MS(m/z): 413.0[M−100]⁺; ¹H NMR (400 MHz, DMSO-d₆) δ10.98 (s, 1H), 8.49 (s, 1H), 7.62 (dd, J=26.3, 7.8 Hz, 1H), 7.51-7.34 (m, 2H), 6.74 (d, J=8.3 Hz, 1H), 5.11 (dd, J=13.2, 4.9 Hz, 1H), 4.36 (dt, J=22.2, 17.2 Hz, 4H), 3.82 (t, J=7.7 Hz, 1H), 2.91 (dd, J=21.9, 9.3 Hz, 1H), 2.69-2.57 (m, 1H), 2.40 (dd, J=21.9, 13.4 Hz, 1H), 2.06-1.96 (m, 1H), 1.71-1.47 (m, 6H), 1.37 (d, J=26.9 Hz, 9H), 1.18-0.93 (m, 5H).

### Example 41 (4-133)

### tert butyl ((1S)-2-((2-(2,6-dioxypiperidin-3-yl)-1-oxoisoidoline-5-yl)methyl)amino)-1-(naphthalene-2-yl)-2-oxoethyl) carbamate

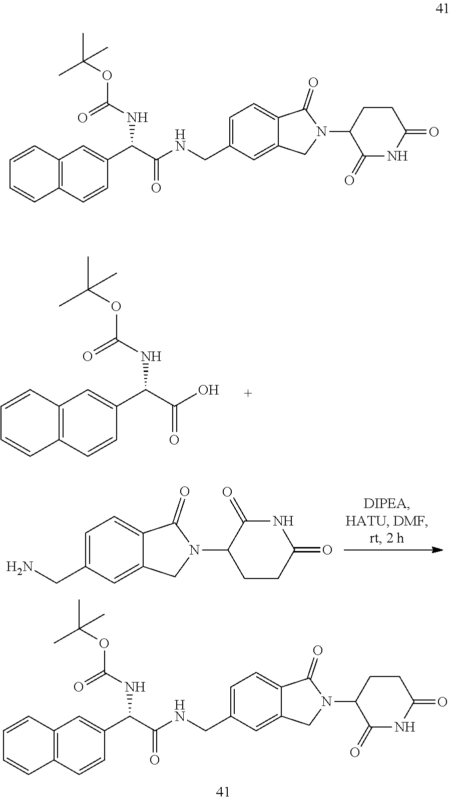

Compound 41 was prepared according to Example 1, Step 5. MS(m/z): 456.2 [M−100]⁺; ¹H NMR (400 MHz, DMSO-d₆) δ10.99 (s, 1H), 8.81 (s, 1H), 7.93 (dd, J=12.1, 6.9 Hz, 3H), 7.87 (d, J=8.5 Hz, 1H), 7.59 (dd, J=13.7, 8.3 Hz, 2H), 7.55-7.51 (m, 2H), 7.46 (d, J=7.5 Hz, 1H), 7.31 (d, J=7.8 Hz, 1H), 7.15 (s, 1H), 5.39 (d, J=7.8 Hz, 1H), 5.08 (dd, J=13.2, 5.0 Hz, 1H), 4.39 (dt, J=16.0, 10.0 Hz, 2H), 4.17-4.06 (m, 2H), 2.99-2.84 (m, 1H), 2.62 (d, J=17.1 Hz, 1H), 2.32 (dt, J=13.2, 8.9 Hz, 2H), 2.06-1.93 (m, 1H), 1.41 (s, 9H).

### Example 42 (4-134)

### Tert butyl 1-Oxdiisoindoline-1-(((2-(2,6-dioxopidol-3-yl)-1-oxdiisoindoline-5-yl) methyl) carbamoyl)-isoindoline-2-carboxylate

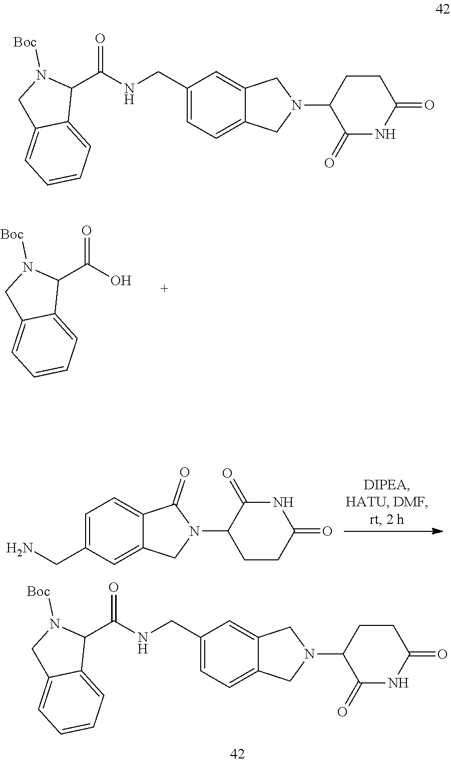

Compound 42 was prepared according to Example 1, Step 5. MS(m/z): 419.1 [M−100]⁺; ¹H NMR (400 MHz, DMSO-d₆) δ 10.98 (s, 1H), 8.85 (d, J=24.7 Hz, 1H), 7.65 (dd, J=14.6, 7.8 Hz, 1H), 7.50-7.23 (m, 6H), 5.35 (d, J=13.8 Hz, 1H), 5.12 (dd, J=13.3, 4.8 Hz, 1H), 4.81-4.61 (m, 2H), 4.56-4.35 (m, 2H), 4.30 (dd, J=20.7, 11.7 Hz, 2H), 2.91 (dd, J=21.8, 9.1 Hz, 1H), 2.61 (d, J=17.4 Hz, 1H), 2.42 (d, J=9.8 Hz, 1H), 2.06-1.95 (m, 1H), 1.48 (d, J=6.7 Hz, 3H), 1.42 (s, 1H), 1.33 (s, 5H).

### Example 43 (5-32)

### Tert butyl 1-(((2-(2,6-dioxypiperidine-3-yl)-1-oxoisoidoline-5-yl)methyl)carbamoyl)-3,4-dihydroisoquino line-2-carboxylate

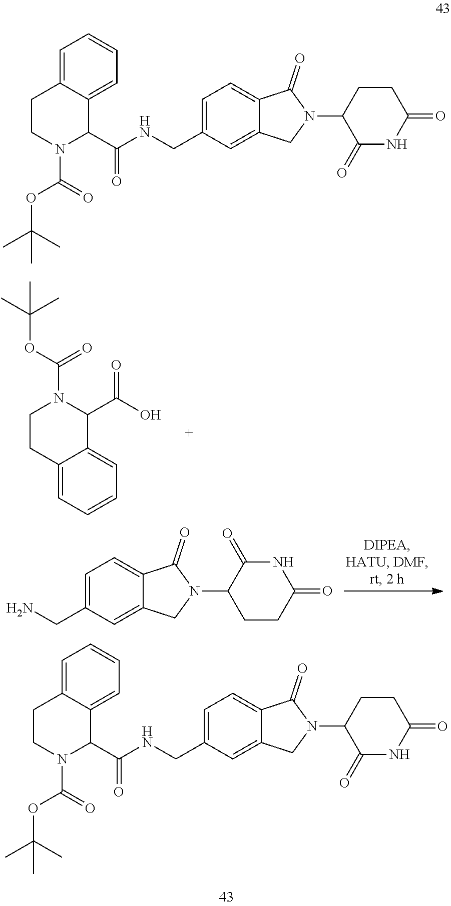

Compound 43 was prepared according to Example 1, Step 5. MS(m/z): 432.9 [M−100]⁺; ¹H NMR (400 MHz, DMSO-d₆) δ10.94 (s, 1H), 8.84 (d, J=17.4 Hz, 1H), 7.56 (d, J=35.9 Hz, 2H), 7.35 (d, J=7.3 Hz, 2H), 7.21 (dt, J=9.0, 5.1 Hz, 3H), 5.09 (dd, J=13.2, 5.0 Hz, 1H), 4.46-4.16 (m, 4H), 3.87 (dd, J=12.0, 5.3 Hz, 1H), 3.69-3.57 (m, 1H), 3.48 (s, 1H), 3.07-2.84 (m, 2H), 2.81-2.70 (m, 1H), 2.60 (d, J=16.8 Hz, 1H), 2.46-2.33 (m, 1H), 1.99 (dd, J=7.1, 5.4 Hz, 1H), 1.40 (d, J=46.8 Hz, 9H).

### Example 44 (4-136)

### Tert butyl ((1S)-2-((2-(2,6-dioxypiperidin-3-yl)-1-oxoisoidoline-5-yl)methyl)amino)-1-(4-fluorophenyl)-2-oxoethyl) carbamate

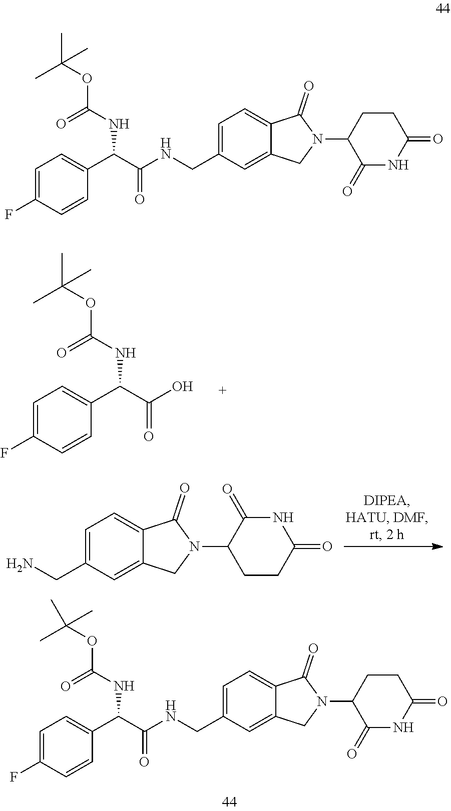

Compound 44 was prepared according to Example 1, Step 5. MS(m/z): 424.9 [M−100]⁺; ¹H NMR (400 MHz, DMSO-d₆) δ10.98 (s, 1H), 8.76 (t, J=5.6 Hz, 1H), 7.62 (d, J=7.9 Hz, 1H), 7.48 (dd, J=8.0, 5.8 Hz, 2H), 7.41 (d, J=7.8 Hz, 1H), 7.31 (d, J=7.8 Hz, 2H), 7.19 (t, J=8.8 Hz, 2H), 5.22 (d, J=7.7 Hz, 1H), 5.11 (dd, J=13.3, 5.0 Hz, 1H), 4.43-4.22 (m, 4H), 2.92 (ddd, J=13.7, 12.1, 5.3 Hz, 1H), 2.61 (d, J=17.0 Hz, 1H), 2.47-2.32 (m, 1H), 2.07-1.96 (m, 1H), 1.40 (s, 9H).

### Example 45 (5-5)

### Tert butyl (2-((2-(2,6-dioxypiperidin-3-yl)-1-oxoisoidoline-5-yl) methyl) amino)-1-(2-fluorophenyl)-2-oxoethyl) carbamate

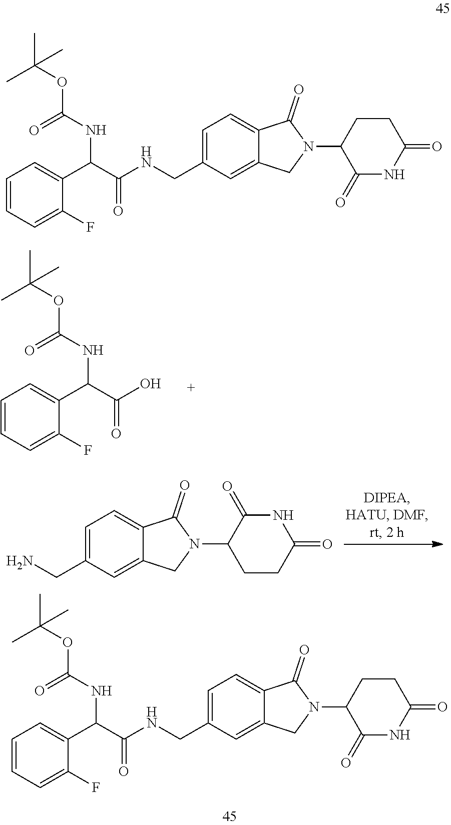

Compound 45 was prepared according to Example 1, Step 5. MS(m/z): 424.9 [M−100]⁺; ¹H NMR (400 MHz, DMSO-d₆) δ10.97 (s, 1H), 8.69 (s, 1H), 7.63 (d, J=7.8 Hz, 1H), 7.50 (d, J=7.6 Hz, 1H), 7.46-7.31 (m, 4H), 7.25-7.13 (m, 2H), 5.45 (d, J=7.7 Hz, 1H), 5.10 (dd, J=13.2, 4.9 Hz, 1H), 4.52-4.18 (m, 4H), 2.98-2.83 (m, 1H), 2.60 (d, J=17.1 Hz, 1H), 2.40 (dd, J=13.0, 4.0 Hz, 1H), 2.06-1.92 (m, 1H), 1.39 (s, 9H).

### Example 46 (4-200)

### Tert butyl (1-(2-chlorophenyl)-2-((2-(2,6-dioxypiperidin-3-yl)-1-oxoisoindoline-5-yl) methyl) amino)-2-oxoethyl) carbamate

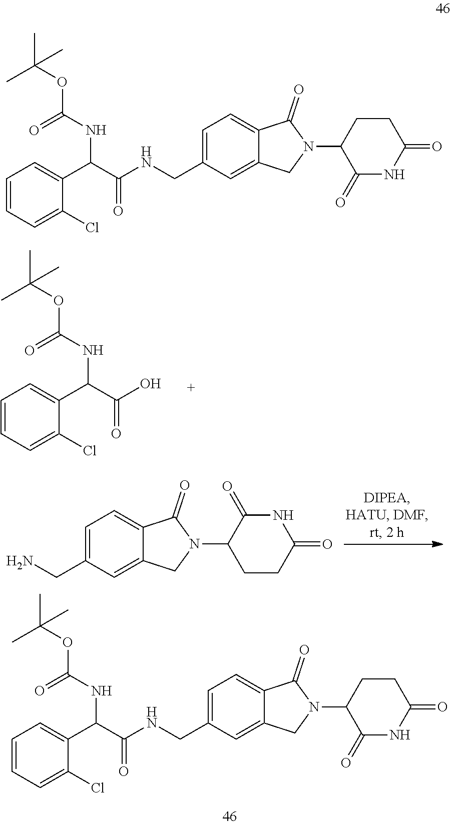

Compound 46 was prepared according to Example 1, Step 5. MS(m/z): 441.2 [M−100]⁺; ¹H NMR (400 MHz, DMSO-d₆) δ10.96 (s, 1H), 8.67 (s, 1H), 7.64 (d, J=7.8 Hz, 1H), 7.57 (d, J=7.8 Hz, 1H), 7.43 (dt, J=12.9, 6.5 Hz, 3H), 7.38 (d, J=8.0 Hz, 1H), 7.32 (dd, J=5.4, 3.6 Hz, 2H), 5.54 (d, J=7.8 Hz, 1H), 5.10 (dd, J=13.2, 4.9 Hz, 1H), 4.46-4.22 (m, 4H), 2.91 (ddd, J=13.6, 11.8, 5.2 Hz, 1H), 2.60 (d, J=17.4 Hz, 1H), 2.47-2.33 (m, 1H), 2.06-1.95 (m, 1H), 1.39 (s, 9H).

### Example 47 (5-17)

### Tert butyl ((1S)-1-(2-chlorophenyl)-2-((2-(2,6-dioxopyridin-3-yl)-1-oxoisoidoline-5-yl) methyl) amino)-2-oxoethyl) carbamate

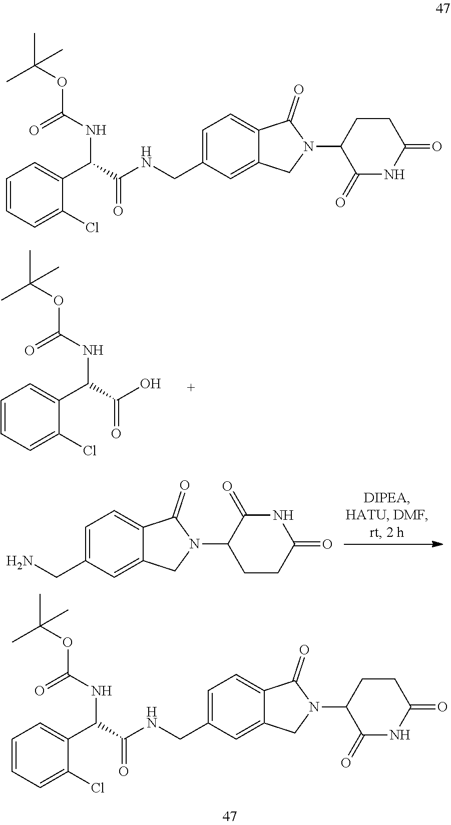

Compound 47 was prepared according to Example 1, Step 5. MS(m/z): 441.2 [M−100]⁺; ¹H NMR (400 MHz, DMSO-d₆) δ10.95 (s, 1H), 8.65 (t, J=5.5 Hz, 1H), 7.69-7.26 (m, 8H), 5.54 (d, J=7.6 Hz, 1H), 5.10 (dd, J=13.2, 5.0 Hz, 1H), 4.52-4.18 (m, 4H), 2.89 (td, J=9.4, 5.0 Hz, 1H), 2.60 (d, J=17.0 Hz, 1H), 2.40 (dd, J=13.1, 4.2 Hz, 1H), 2.09-1.90 (m, 1H), 1.39 (s, 9H).

### Example 48 (4-140)

### Tert butyl ((1S)-1-(3-chlorophenyl)-2-((2-(2,6-dioxopyridin-3-yl)-1-oxoisoidoline-5-yl) methyl) amino)-2-oxoethyl) carbamate

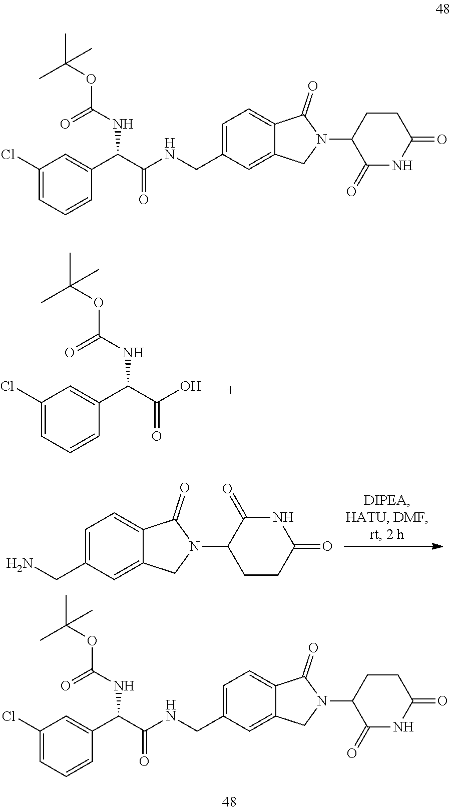

Compound 48 was prepared according to Example 1, Step 5. MS(m/z): 441.2 [M−100]⁺; ¹H NMR (400 MHz, DMSO-d₆) δ10.98 (s, 1H), 8.81 (s, 1H), 7.62 (d, J=7.8 Hz, 1H), 7.53 (d, J=12.2 Hz, 2H), 7.39 (t, J=5.1 Hz, 3H), 7.31 (d, J=6.1 Hz, 2H), 5.24 (d, J=8.0 Hz, 1H), 5.11 (dd, J=13.2, 5.0 Hz, 1H), 4.43-4.23 (m, 4H), 2.97-2.85 (m, 1H), 2.67-2.57 (m, 1H), 2.41 (ddd, J=26.4, 13.1, 4.1 Hz, 1H), 2.06-1.96 (m, 1H), 1.40 (s, 9H).

### Example 49 (4-150)

### Tert butyl ((1S)-1-(4-chlorophenyl)-2-((2-(2,6-dioxopyridin-3-yl)-1-oxoisoidoline-5-yl) methyl) amino)-2-oxoethyl) carbamate

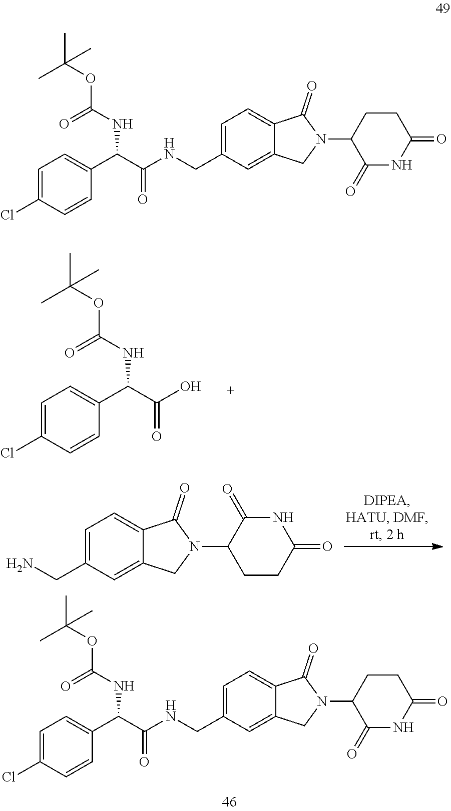

Compound 49 was prepared according to Example 1, Step 5. MS(m/z): 441.2 [M−100]⁺; ¹H NMR (400 MHz, DMSO-d₆) δ10.99 (s, 1H), 8.78 (t, J=5.6 Hz, 1H), 7.63 (d, J=7.8 Hz, 1H), 7.45 (q, J=8.4 Hz, 5H), 7.30 (dd, J=12.0, 6.1 Hz, 2H), 5.23 (d, J=7.8 Hz, 1H), 5.11 (dd, J=13.2, 5.0 Hz, 1H), 4.48-4.19 (m, 4H), 2.98-2.86 (m, 1H), 2.68-2.55 (m, 1H), 2.39 (ddd, J=26.1, 13.1, 4.1 Hz, 1H), 2.07-1.94 (m, 1H), 1.40 (s, 9H).

### Example 50 (5-7)

### Tert butyl ((1S)-1-(4-bromophenyl)-2-((2-(2,6-dioxopyridin-3-yl)-1-oxoisoidoline-5-yl) methyl) amino)-2-oxoethyl) carbamate

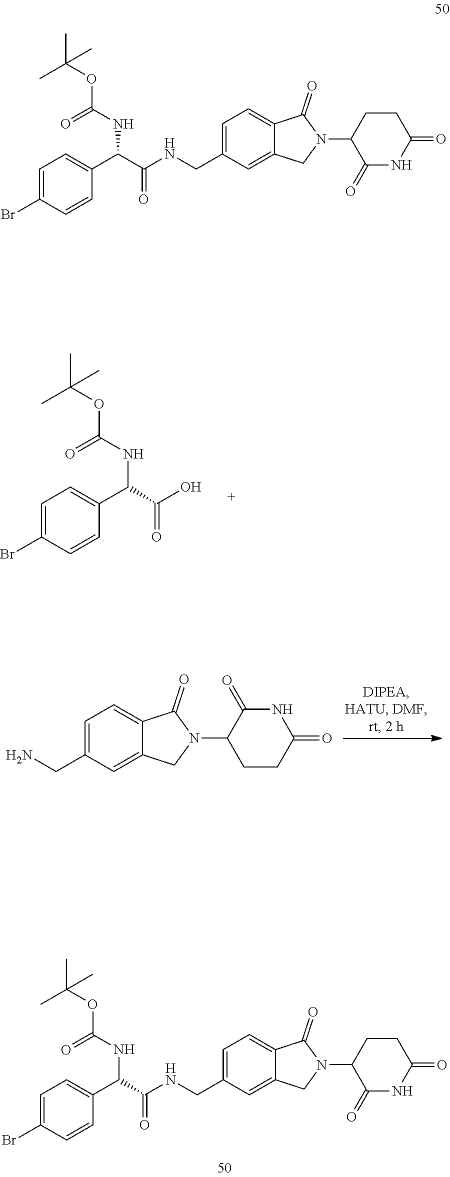

Compound 50 was prepared according to Example 1, Step 5. MS(m/z): 485.1 [M−100]⁺; ¹H NMR (400 MHz, DMSO-d₆) δ10.96 (s, 1H), 8.75 (t, J=5.7 Hz, 1H), 7.61 (d, J=7.8 Hz, 1H), 7.55 (d, J=8.3 Hz, 2H), 7.39 (d, J=8.4 Hz, 2H), 7.30 (d, J=7.8 Hz, 1H), 7.24 (d, J=3.6 Hz, 1H), 5.20 (d, J=7.1 Hz, 1H), 5.09 (dd, J=13.2, 4.9 Hz, 1H), 4.47-4.18 (m, 4H), 2.98-2.83 (m, 1H), 2.61 (d, J=16.8 Hz, 1H), 2.38 (dd, J=13.1, 4.3 Hz, 1H), 2.09-1.94 (m, 1H), 1.38 (s, 9H).

### Example 51 (4-152)

### Tert butyl (1-(3-chloro-4-fluorophenyl)-2-((2-(2,6-dioxopyridin-3-yl)-1-oxoisoindoline-5-yl) methyl) amino)-2-oxoethyl) carbamate

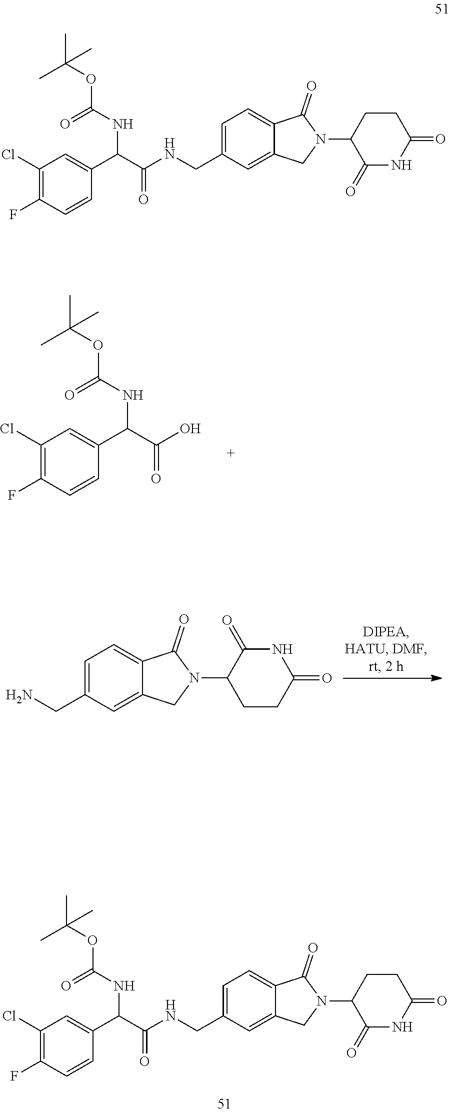 [SMILES: CC(C)(C)OC(=O)NC(C(=O)NCc1ccc2c(c1)CN(C1CCC(=O)NC1=O)C2=O)c1ccc(F)c(Cl)c1 ; CC(C)(C)OC(=O)NC(C(=O)O)c1ccc(F)c(Cl)c1 ; NCc1ccc2c(c1)CN(C1CCC(=O)NC1=O)C2=O ; CC(C)(C)OC(=O)NC(C(=O)NCc1ccc2c(c1)CN(C1CCC(=O)NC1=O)C2=O)c1ccc(F)c(Cl)c1]

Compound 51 was prepared according to Example 1, Step 5. MS(m/z): 458.2 [M−100]⁺; ¹H NMR (400 MHz, DMSO-d₆) δ10.99 (s, 1H), 8.80 (t, J=5.1 Hz, 1H), 7.64 (t, J=7.4 Hz, 2H), 7.55 (d, J=7.4 Hz, 1H), 7.42 (dd, J=16.8, 7.6 Hz, 2H), 7.33 (dd, J=11.1, 5.6 Hz, 2H), 5.24 (d, J=7.7 Hz, 1H), 5.11 (dd, J=13.3, 5.0 Hz, 1H), 4.35 (dt, J=49.5, 11.5 Hz, 4H), 3.00-2.84 (m, 1H), 2.61 (d, J=17.7 Hz, 1H), 2.45-2.33 (m, 1H), 2.06-1.95 (m, 1H), 1.40 (s, 9H).

### Example 52 (4-151)

### Tert butyl (1-(3,4-dichlorophenyl)-2-((2-(2,6-dioxopyridin-3-yl)-1-oxoisoindoline-5-yl) methyl) amino)-2-oxoethyl) carbamate

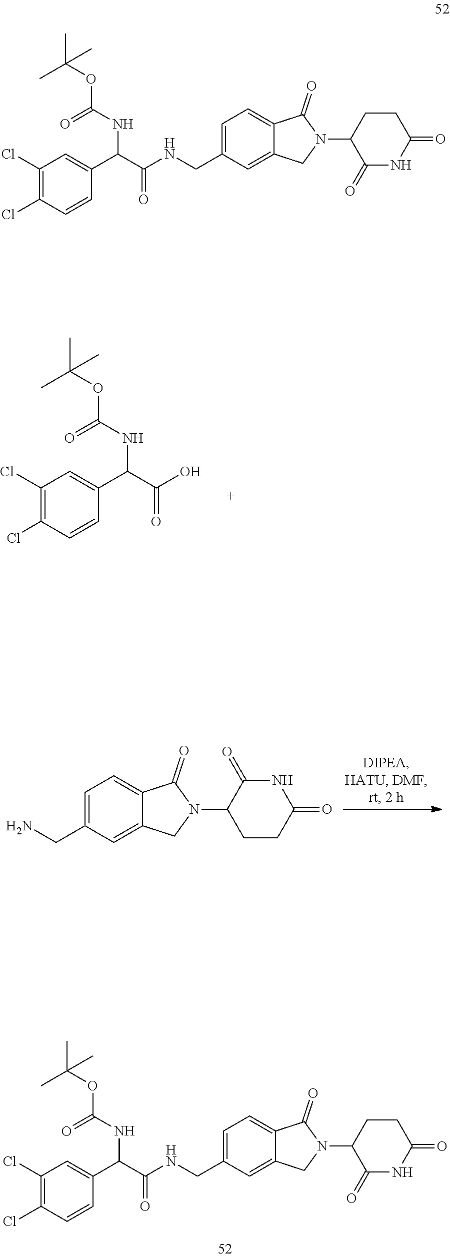 [SMILES: CC(C)(C)OC(=O)NC(C(=O)NCc1ccc2c(c1)CN(C1CCC(=O)NC1=O)C2=O)c1ccc(Cl)c(Cl)c1 ; CC(C)(C)OC(=O)NC(C(=O)O)c1ccc(Cl)c(Cl)c1 ; NCc1ccc2c(c1)CN(C1CCC(=O)NC1=O)C2=O ; CC(C)(C)OC(=O)NC(C(=O)NCc1ccc2c(c1)CN(C1CCC(=O)NC1=O)C2=O)c1ccc(Cl)c(Cl)c1]

Compound 52 was prepared according to Example 1, Step 5. MS(m/z): 474.2 [M−100]⁺; ¹H NMR (400 MHz, DMSO-d₆) δ10.99 (s, 1H), 8.83 (d, J=5.4 Hz, 1H), 7.70 (s, 1H), 7.67-7.54 (m, 3H), 7.44 (d, J=7.8 Hz, 1H), 7.31 (s, 2H), 5.25 (d, J=7.8 Hz, 1H), 5.11 (dd, J=13.2, 5.0 Hz, 1H), 4.42-4.21 (m, 4H), 2.98-2.84 (m, 1H), 2.62 (d, J=17.4 Hz, 1H), 2.40 (dd, J=13.1, 4.3 Hz, 1H), 2.06-1.96 (m, 1H), 1.40 (s, 9H).

### Example 53 (5-6)

### Tert butyl (2-((2-(2,6-dioxypiperidin-3-yl)-1-oxoisoindoline-5-yl)methyl)amino)-2-oxo-1-(4-(trifluoromethyl) phenyl) ethyl) carbamate

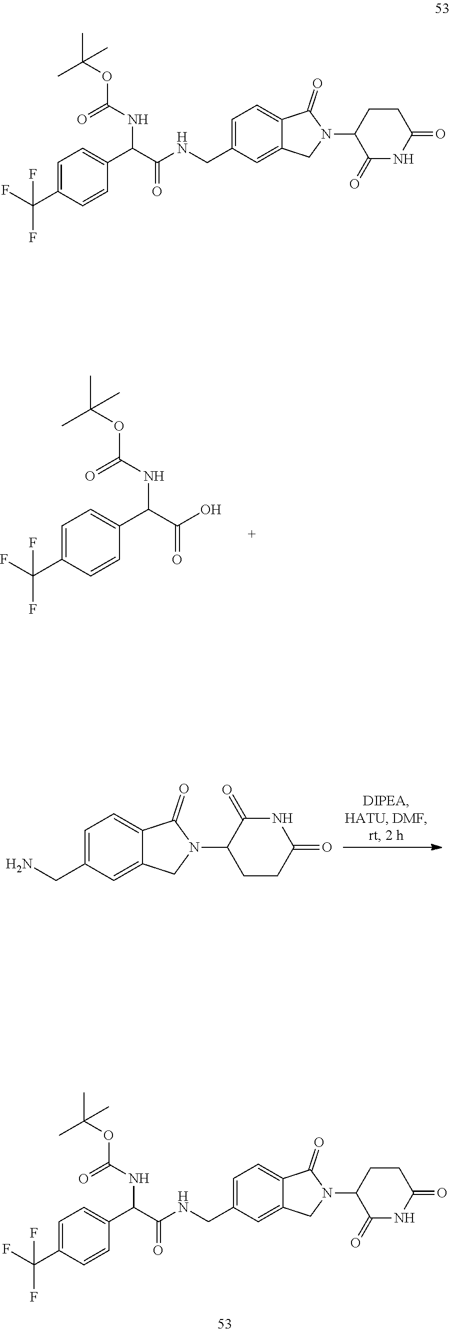 [SMILES: CC(C)(C)OC(=O)NC(C(=O)NCc1ccc2c(c1)CN(C1CCC(=O)NC1=O)C2=O)c1ccc(C(F)(F)F)cc1 ; CC(C)(C)OC(=O)NC(C(=O)O)c1ccc(C(F)(F)F)cc1 ; NCc1ccc2c(c1)CN(C1CCC(=O)NC1=O)C2=O ; CC(C)(C)OC(=O)NC(C(=O)NCc1ccc2c(c1)CN(C1CCC(=O)NC1=O)C2=O)c1ccc(C(F)(F)F)cc1]

Compound 53 was prepared according to Example 1, Step 5. MS(m/z): 475.2 [M−100]⁺; ¹H NMR (400 MHz, DMSO-d₆) δ10.97 (s, 1H), 8.85 (t, J=5.4 Hz, 1H), 7.73 (d, J=8.2 Hz, 2H), 7.66 (d, J=8.2 Hz, 2H), 7.61 (d, J=8.1 Hz, 1H), 7.54 (d, J=7.4 Hz, 1H), 7.29 (d, J=6.0 Hz, 2H), 5.33 (d, J=7.7 Hz, 1H), 5.10 (dd, J=13.2, 4.9 Hz, 1H), 4.48-4.13 (m, 4H), 2.97-2.86 (m, 1H), 2.60 (d, J=17.1 Hz, 1H), 2.36 (dd, J=13.2, 4.3 Hz, 1H), 2.05-1.94 (m, 1H), 1.39 (s, 9H).

### Example 54 (5-13)

### Tert butyl (2-((2-(2,6-dioxypiperidin-3-yl)-1-oxoisoindolin-5-yl)methyl)amino)-1-(4-isopropoxyphenyl)-2-oxoethyl) carbamate

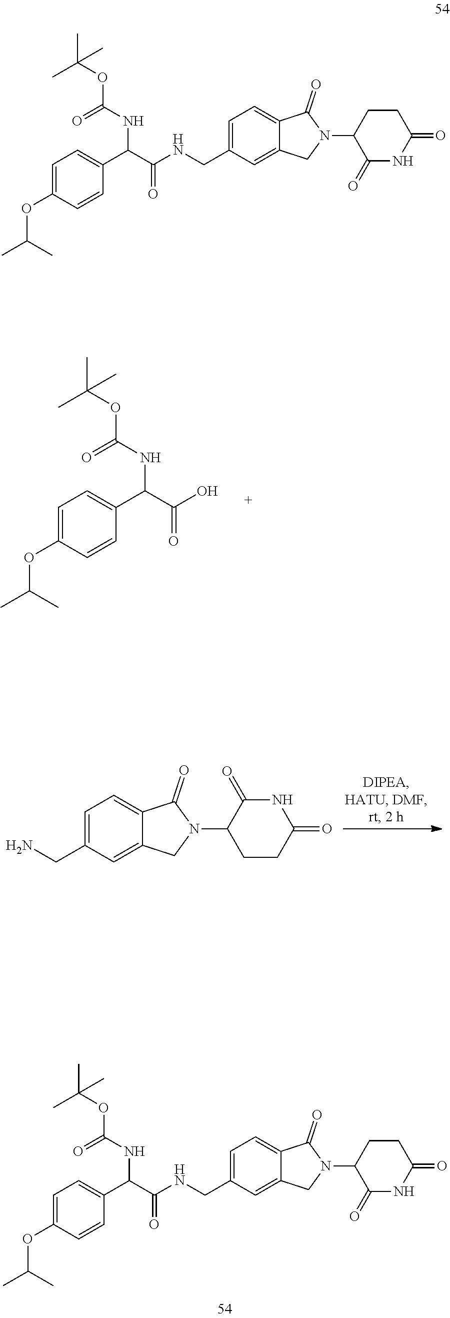 [SMILES: CC(C)Oc1ccc(C(NC(=O)OC(C)(C)C)C(=O)NCc2ccc3c(c2)CN(C2CCC(=O)NC2=O)C3=O)cc1 ; CC(C)Oc1ccc(C(NC(=O)OC(C)(C)C)C(=O)O)cc1 ; NCc1ccc2c(c1)CN(C1CCC(=O)NC1=O)C2=O ; CC(C)Oc1ccc(C(NC(=O)OC(C)(C)C)C(=O)NCc2ccc3c(c2)CN(C2CCC(=O)NC2=O)C3=O)cc1]

Compound 54 was prepared according to Example 1, Step 5. MS(m/z): 465.2 [M−100]⁺; ¹H NMR (400 MHz, DMSO-d₆) δ10.96 (s, 1H), 8.65 (d, J=5.1 Hz, 1H), 7.60 (d, J=7.8 Hz, 1H), 7.39-7.12 (m, 5H), 6.87 (d, J=8.4 Hz, 2H), 5.09 (dd, J=13.0, 4.2 Hz, 2H), 4.66-4.51 (m, 1H), 4.47-4.16 (m, 4H), 2.91 (ddd, J=13.7, 11.3, 5.4 Hz, 1H), 2.60 (d, J=16.8 Hz, 1H), 2.36 (qd, J=13.1, 4.2 Hz, 1H), 2.07-1.92 (m, 1H), 1.38 (s, 6H), 1.26 (s, 9H).

### Example 55 (4-137)

### Tert butyl ((1S)-2-((2-(2,6-dioxypiperidin-3-yl)-1-oxoisoidoline-5-yl)methyl)amino)-1-(4-hydroxyphenyl)-2-oxoethyl) carbamate

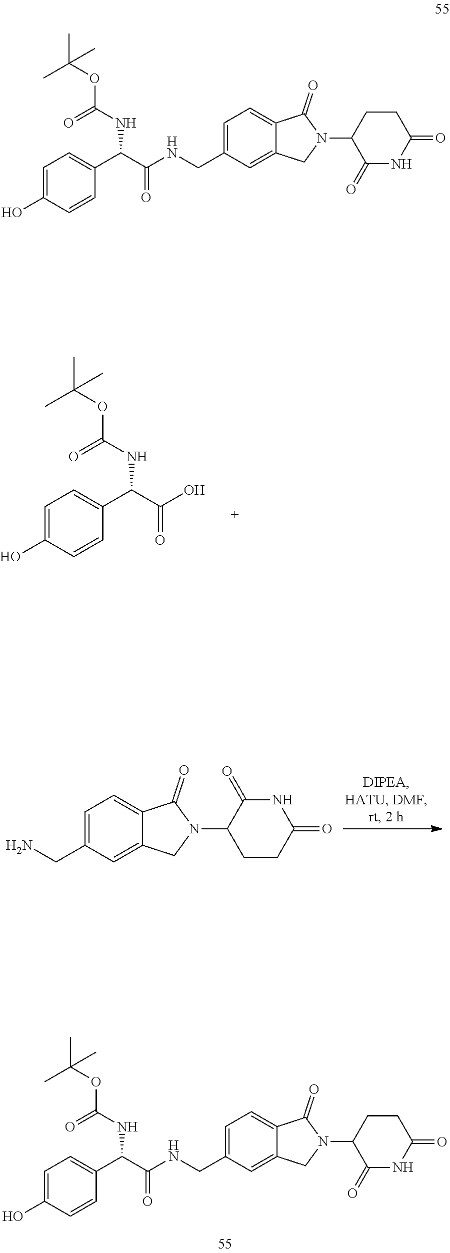 [SMILES: CC(C)(C)OC(=O)N[C@H](C(=O)NCc1ccc2c(c1)CN(C1CCC(=O)NC1=O)C2=O)c1ccc(O)cc1 ; CC(C)(C)OC(=O)N[C@H](C(=O)O)c1ccc(O)cc1 ; NCc1ccc2c(c1)CN(C1CCC(=O)NC1=O)C2=O ; CC(C)(C)OC(=O)N[C@H](C(=O)NCc1ccc2c(c1)CN(C1CCC(=O)NC1=O)C2=O)c1ccc(O)cc1]

Compound 55 was prepared according to Example 1, Step 5. MS(m/z): 422.2 [M−100]⁺; ¹H NMR (400 MHz, DMSO-d₆) δ10.99 (s, 1H), 9.41 (s, 1H), 8.62 (t, J=5.7 Hz, 1H), 7.59 (t, J=10.9 Hz, 1H), 7.38-7.09 (m, 5H), 6.72 (d, J=8.3 Hz, 2H), 5.10 (dd, J=18.8, 6.0 Hz, 2H), 4.47-4.19 (m, 4H), 2.99-2.86 (m, 1H), 2.61 (d, J=17.4 Hz, 1H), 2.44-2.32 (m, 1H), 2.06-1.95 (m, 1H), 1.41 (d, J=11.2 Hz, 9H).

### Example 56 (4-166)

### Tert butyl ((1S)-2-((2-(2,6-dioxypiperidin-3-yl)-1-oxoisoindoline-5-yl) methyl) amino)-2-oxo-1-(p-tolyl) ethyl) carbamate

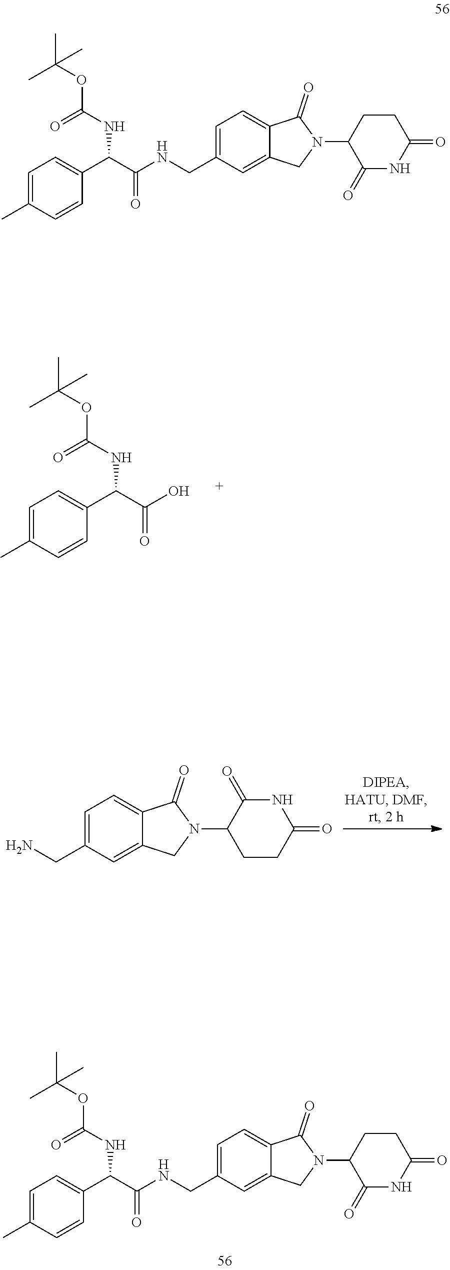 [SMILES: Cc1ccc([C@H](NC(=O)OC(C)(C)C)C(=O)NCc2ccc3c(c2)CN(C2CCC(=O)NC2=O)C3=O)cc1 ; Cc1ccc([C@H](NC(=O)OC(C)(C)C)C(=O)O)cc1 ; NCc1ccc2c(c1)CN(C1CCC(=O)NC1=O)C2=O ; Cc1ccc([C@H](NC(=O)OC(C)(C)C)C(=O)NCc2ccc3c(c2)CN(C2CCC(=O)NC2=O)C3=O)cc1]

Compound 56 was prepared according to Example 1, Step 5. MS(m/z): 421.2 [M−100]; ¹H NMR (400 MHz, DMSO-d₆) δ10.99 (s, 1H), 8.71 (s, 1H), 7.61 (d, J=7.8 Hz, 1H), 7.32 (t, J=6.8 Hz, 3H), 7.26 (s, 2H), 7.15 (d, J=7.8 Hz, 2H), 5.20-5.06 (m, 2H), 4.46-4.19 (m, 4H), 3.00-2.86 (m, 1H), 2.61 (d, J=16.8 Hz, 1H), 2.46-2.33 (m, 1H), 2.30 (s, 3H), 2.07-1.95 (m, 1H), 1.40 (s, 9H).

### Example 57 (4-130)

### Tert butyl (3-((2-(2,6-dioxypiperidin-3-yl)-1-oxoisoindolin-5-yl)methyl)amino)-3-oxo-1-phenylpropyl) carbamate

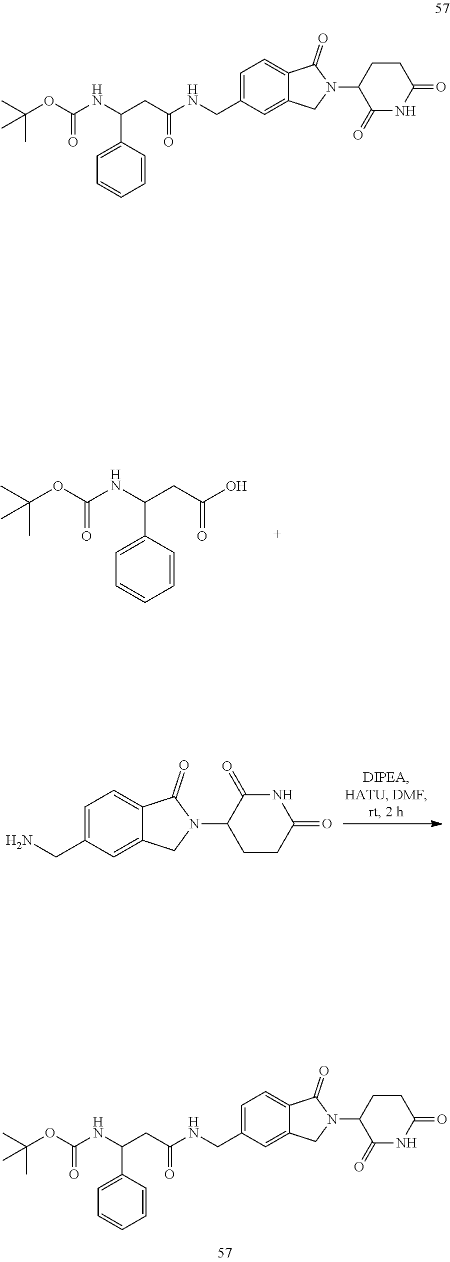 [SMILES: CC(C)(C)OC(=O)NC(CC(=O)NCc1ccc2c(c1)CN(C1CCC(=O)NC1=O)C2=O)c1ccccc1 ; CC(C)(C)OC(=O)NC(CC(=O)O)c1ccccc1 ; NCc1ccc2c(c1)CN(C1CCC(=O)NC1=O)C2=O ; CC(C)(C)OC(=O)NC(CC(=O)NCc1ccc2c(c1)CN(C1CCC(=O)NC1=O)C2=O)c1ccccc1]

Compound 57 was prepared according to Example 1, Step 5. MS(m/z): 420.9 [M−100]⁺; ¹H NMR (400 MHz, DMSO-d₆) δ10.99 (s, 1H), 8.40 (t, J=5.6 Hz, 1H), 7.58 (d, J=7.9 Hz, 1H), 7.45 (d, J=8.4 Hz, 1H), 7.37-7.23 (m, 5H), 7.17 (d, J=3.2 Hz, 2H), 5.11 (dd, J=13.2, 5.0 Hz, 1H), 4.97 (d, J=7.5 Hz, 1H), 4.32 (dd, J=49.5, 17.3 Hz, 4H), 2.99-2.86 (m, 1H), 2.61 (d, J=7.4 Hz, 3H), 2.43 (dd, J=13.4, 4.1 Hz, 1H), 2.06-1.96 (m, 1H), 1.32 (d, J=31.0 Hz, 9H).

### Example 58 (4-131)

### Tert butyl (3-((2-(2,6-dioxypiperidin-3-yl)-1-oxoisoindoline-5-yl) amino)-3-oxo-2-phenylpropyl) carbamate

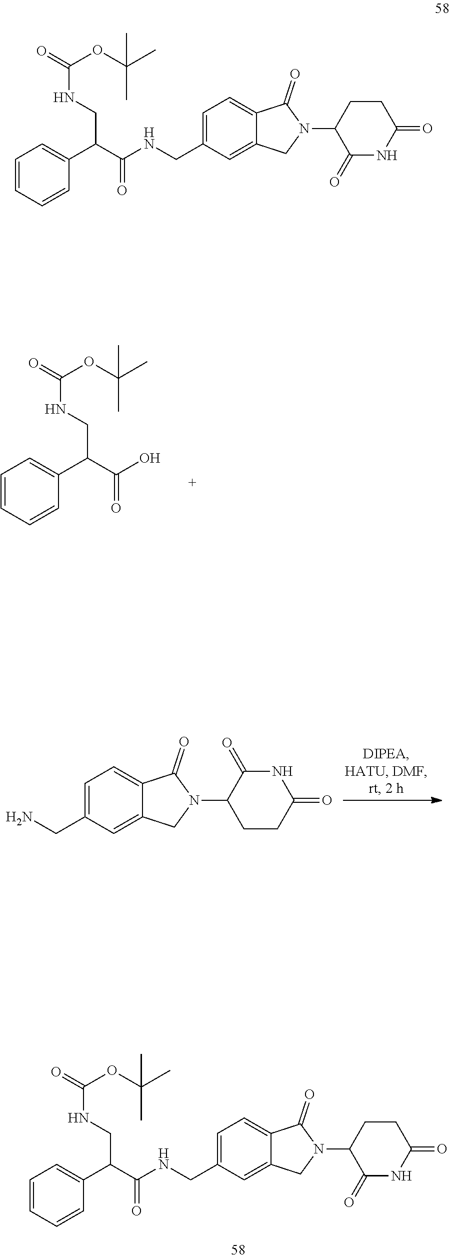 [SMILES: CC(C)(C)OC(=O)NCC(C(=O)NCc1ccc2c(c1)CN(C1CCC(=O)NC1=O)C2=O)c1ccccc1 ; CC(C)(C)OC(=O)NCC(C(=O)O)c1ccccc1 ; NCc1ccc2c(c1)CN(C1CCC(=O)NC1=O)C2=O ; CC(C)(C)OC(=O)NCC(C(=O)NCc1ccc2c(c1)CN(C1CCC(=O)NC1=O)C2=O)c1ccccc1]

Compound 58 was prepared according to Example 1, Step 5. MS(m/z): 421.2 [M−100]⁺; ¹H NMR (400 MHz, DMSO-d₆) 310.98 (s, 1H), 8.70 (s, 1H), 7.61 (d, J=8.0 Hz, 1H), 7.40-7.18 (m, 7H), 6.86 (s, 1H), 5.10 (dd, J=13.2, 5.0 Hz, 1H), 4.46-4.20 (m, 4H), 3.88 (t, J=7.4 Hz, 1H), 3.44 (dd, J=12.6, 6.0 Hz, 1H), 3.30-3.22 (m, 1H), 2.99-2.84 (m, 1H), 2.61 (d, J=17.5 Hz, 1H), 2.44-2.33 (m, 1H), 2.05-1.94 (m, 1H), 1.35 (s, 9H).

### Example 59 (4-201)

### ((1S)-2-((2-(2,6-dioxypiperidin-3-yl)-1-oxoisoindoline-5-yl)methyl)amino)-2-oxo-1-phenyl ethyl) glycine Tert Butyl Ester

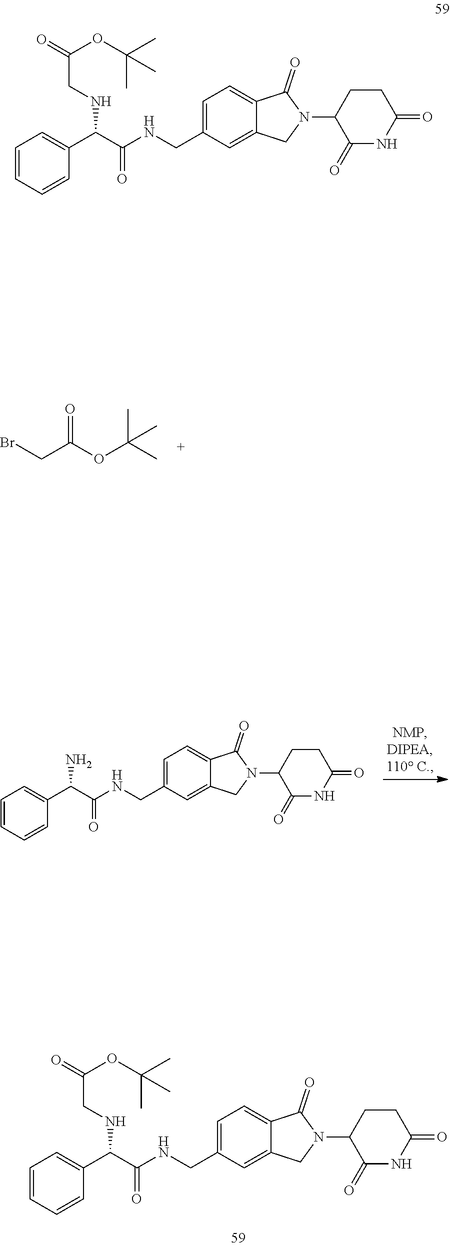

Intermediate 11-1 (250.0 mg, 0.6 mmol) and N,N-diisopropylethylamine (232.0 mg, 1.8 mmol) were dissolved in N-methylpyrrolidone (10 mL), tert-butyl bromoacetate (120.0 mg, 0.6 mmol) was added, and stirred in an oil bath at 110° C. for 3 h. To the reaction system was added water (20 mL), and the organic phase was extracted with ethyl acetate (30 mL×3), the organic phases were combined, washed with saturated sodium chloride solution (30 mL×3), dried with anhydrous sodium sulfate, filtered, concentrated under reduced pressure, and the residue was purified by silica gel column chromatography (methanol/dichloromethane=1/40 elution) to give Compound 59 (135.0 mg, 43.1% yield), as a white solid. MS(m/z): 520.8 [M+H]⁺; ¹H NMR (400 MHz, DMSO-d₆) 310.95 (s, 1H), 8.68 (s, 1H), 7.75-7.24 (m, 9H), 5.09 (dd, J=13.2, 5.0 Hz, 1H), 4.35 (dd, J=21.4, 8.2 Hz, 4H), 4.23 (dd, J=17.2, 3.9 Hz, 1H), 2.97-2.84 (m, 1H), 2.69 (s, 2H), 2.60 (d, J=16.9 Hz, 1H), 2.39 (dd, J=13.1, 4.3 Hz, 1H), 2.03-1.95 (m, 1H), 1.52-1.29 (m, 9H).

### Example 60 (5-39)

### Isopropyl ((2S)-1-(2-(2,6-dioxypiperidin-3-yl)-1-oxoisoindoline-5-yl)methyl)amino)-1-oxo-3-phenylprop-2-yl) carbamate

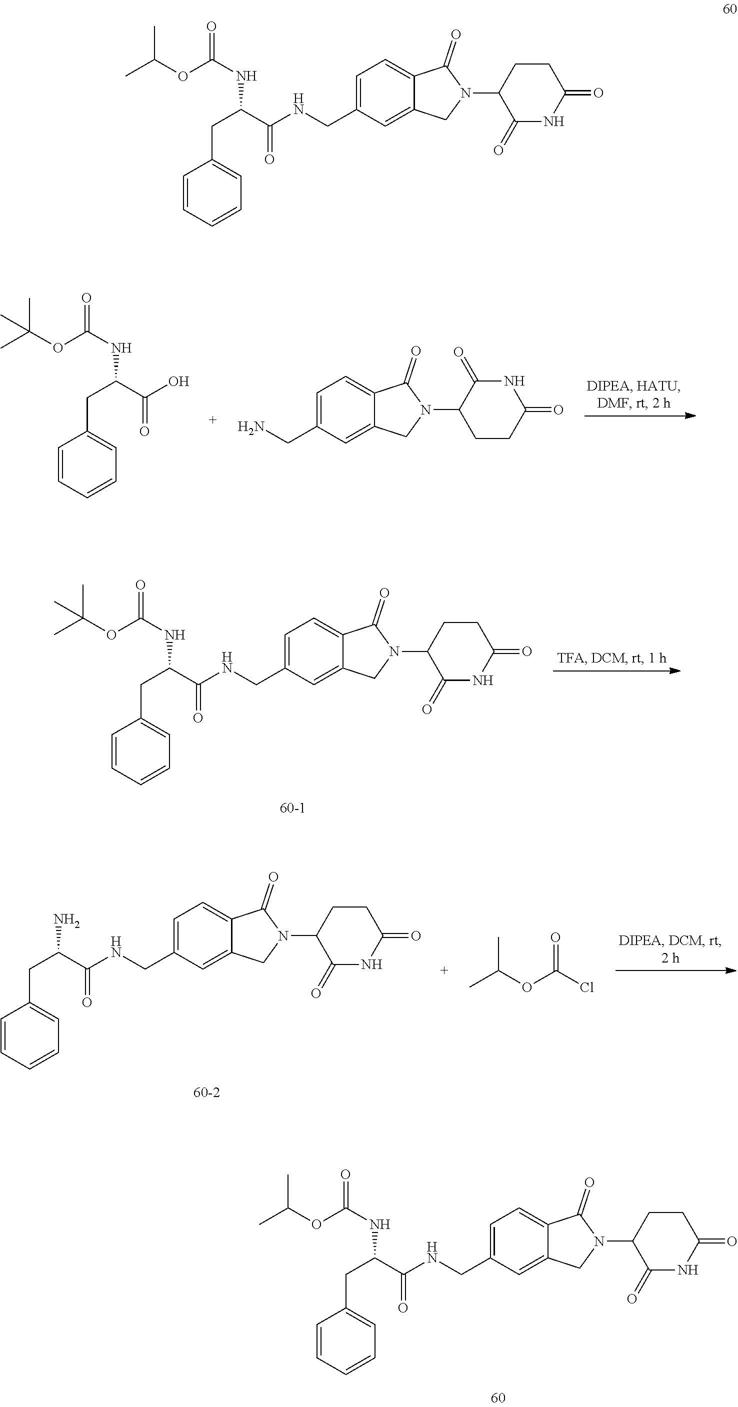

Intermediate 60-1 was prepared according to the method of Step 5, Example 1.

Intermediate 60-2 was prepared according to the method of Step 4, Example 1.

Compound 60 was prepared according to Example 11, Step 2. MS(m/z): 506.9 [M+H]⁺; ¹H NMR (400 MHz, DMSO-d₆) δ10.96 (s, 1H), 9.71 (d, J=5.0 Hz, 1H), 8.83 (t, J=5.7 Hz, 1H), 7.66 (d, J=7.8 Hz, 1H), 7.37 (d, J=7.8 Hz, 1H), 7.33 (d, J=7.9 Hz, 1H), 7.27 (d, J=4.2 Hz, 4H), 7.23-7.19 (m, 1H), 5.11 (dd, J=13.2, 5.0 Hz, 1H), 4.63 (s, 1H), 4.42 (qd, J=15.8, 5.7 Hz, 4H), 4.30 (dd, J=17.4, 2.3 Hz, 1H), 3.31 (s, 6H), 3.15 (dd, J=13.7, 4.9 Hz, 1H), 3.01-2.86 (m, 2H), 2.66-2.57 (m, 1H), 2.41 (qd, J=13.2, 4.3 Hz, 1H), 2.05-1.97 (m, 1H).

### Example 61 (5-37)

### Tert butyl ((2S)-1-(2-(2,6-dioxypiperidin-3-yl)-1-oxoisoindoline-5-yl) methyl) amino)-1-oxo-3-phenylprop-2-yl) carbamate

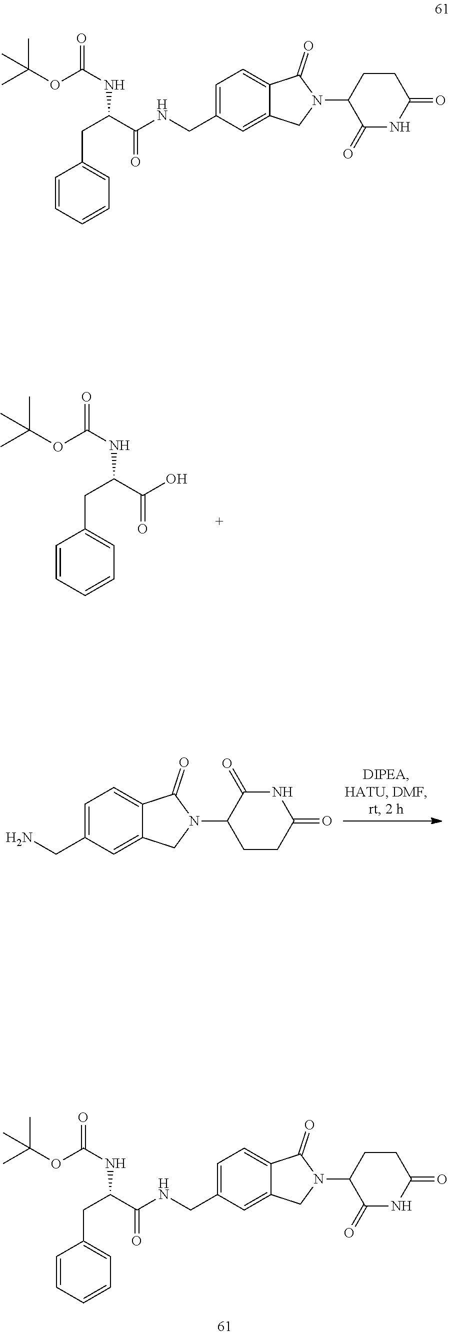 [SMILES: CC(C)(C)OC(=O)N[C@@H](Cc1ccccc1)C(=O)NCc1ccc2c(c1)CN(C1CCC(=O)NC1=O)C2=O ; CC(C)(C)OC(=O)N[C@@H](Cc1ccccc1)C(=O)O ; NCc1ccc2c(c1)CN(C1CCC(=O)NC1=O)C2=O ; CC(C)(C)OC(=O)N[C@@H](Cc1ccccc1)C(=O)NCc1ccc2c(c1)CN(C1CCC(=O)NC1=O)C2=O]

Compound 61 was prepared according to Example 1, Step 5. MS(m/z): 420.2 [M−100]⁺; ¹H NMR (400 MHz, DMSO-d₆) δ10.96 (s, 1H), 8.50 (t, J=5.5 Hz, 1H), 7.95 (s, 1H), 7.63 (d, J=7.7 Hz, 1H), 7.41-7.29 (m, 2H), 7.29-7.17 (m, 4H), 6.98 (d, J=8.0 Hz, 1H), 5.10 (dd, J=13.2, 5.0 Hz, 1H), 4.44-4.19 (m, 4H), 2.97 (dd, J=14.0, 5.2 Hz, 1H), 2.88 (d, J=9.3 Hz, 2H), 2.69 (s, 1H), 2.60 (d, J=17.1 Hz, 1H), 2.40 (dd, J=13.2, 4.2 Hz, 1H), 2.05-1.96 (m, 1H), 1.30 (d, J=19.5 Hz, 9H).

### Example 62 (5-22)

### Isopropyl ((2R)-1-((2-(2,6-dioxypiperidin-3-yl)-1-oxoisoindolin-5-yl)methyl)amino)-1-oxo-3-phenylprop-2-yl) carbamate

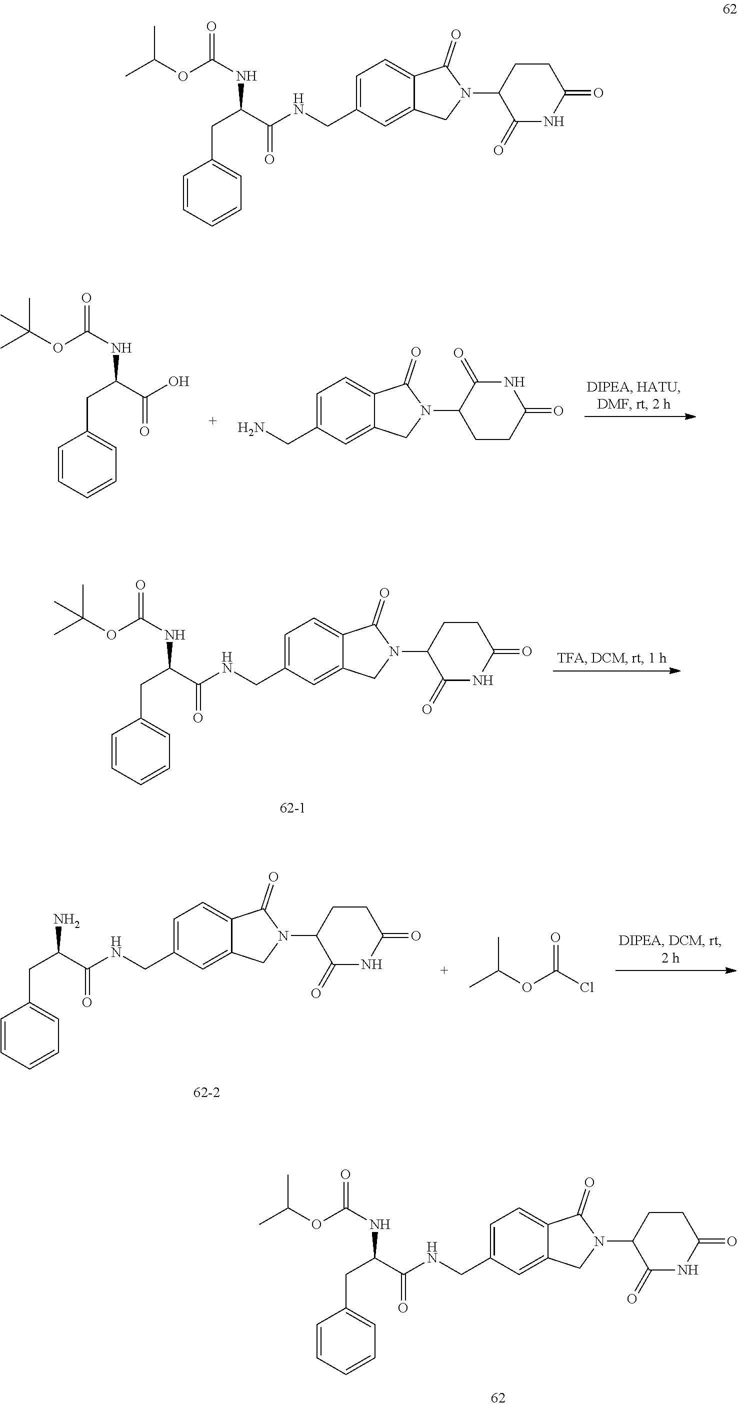

Intermediate 62-1 was prepared according to the method of Step 5, Example 1.

Intermediate 62-2 was prepared according to the method of Step 4, Example 1.

Compound 62 was prepared according to Example 11, Step 2. MS(m/z): 506.9 [M+H]⁺; ¹H NMR (400 MHz, DMSO-d₆) 10.97 (s, 1H), 9.73 (d, J=8.2 Hz, 1H), 8.84 (t, J=5.7 Hz, 1H), 7.66 (d, J=7.8 Hz, 1H), 7.38 (s, 1H), 7.33 (d, J=7.8 Hz, 1H), 7.26 (t, J=6.4 Hz, 4H), 7.22 (d, J=4.7 Hz, 1H), 5.11 (dd, J=13.3, 5.1 Hz, 1H), 4.63 (s, 1H), 4.53-4.34 (m, 4H), 4.29 (dd, J=17.3, 2.3 Hz, 1H), 3.31 (s, 6H), 3.19-3.10 (m, 1H), 3.03-2.85 (m, 2H), 2.60 (d, J=15.8 Hz, 1H), 2.41 (dd, J=13.2, 4.5 Hz, 1H), 2.05-1.95 (m, 1H).

### Example 63 (4-132)

### Tert butyl ((2R)-1-((2-(2,6-dioxypiperidin-3-yl)-1-oxoisoindoline-5-yl)methyl)amino)-1-oxo-3-phenylpro p-2-yl) carbamate

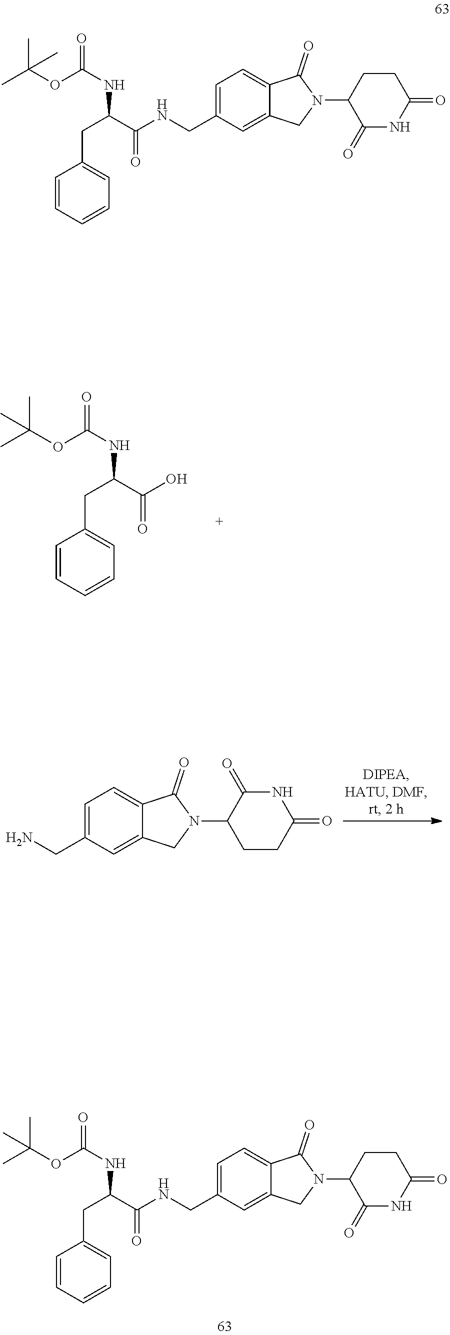 [SMILES: CC(C)(C)OC(=O)N[C@H](Cc1ccccc1)C(=O)NCc1ccc2c(c1)CN(C1CCC(=O)NC1=O)C2=O ; CC(C)(C)OC(=O)N[C@H](Cc1ccccc1)C(=O)O ; NCc1ccc2c(c1)CN(C1CCC(=O)NC1=O)C2=O ; CC(C)(C)OC(=O)N[C@H](Cc1ccccc1)C(=O)NCc1ccc2c(c1)CN(C1CCC(=O)NC1=O)C2=O]

Compound 63 was prepared according to Example 1, Step 5. MS(m/z): 421.2 [M−100]⁺; ¹H NMR (400 MHz, DMSO-d₆) δ10.98 (s, 1H), 8.53 (t, J=5.5 Hz, 1H), 7.64 (d, J=7.7 Hz, 1H), 7.44-7.14 (m, 7H), 7.02 (d, J=8.2 Hz, 1H), 5.12 (dd, J=13.3, 4.9 Hz, 1H), 4.47-4.29 (m, 4H), 4.28-4.18 (m, 1H), 3.04-2.87 (m, 2H), 2.86-2.76 (m, 1H), 2.61 (d, J=17.4 Hz, 1H), 2.42 (dd, J=13.2, 4.2 Hz, 1H), 2.06-1.96 (m, 1H), 1.39-1.26 (m, 9H).

### Example 64 (4-171)

### Tert butyl ((2R)-2-benzyl-3-((2-(2,6-dioxypiperidin-3-yl)-1-oxoisoindolin-5-yl)methyl)amino)-3-oxoprop yl) carbamate

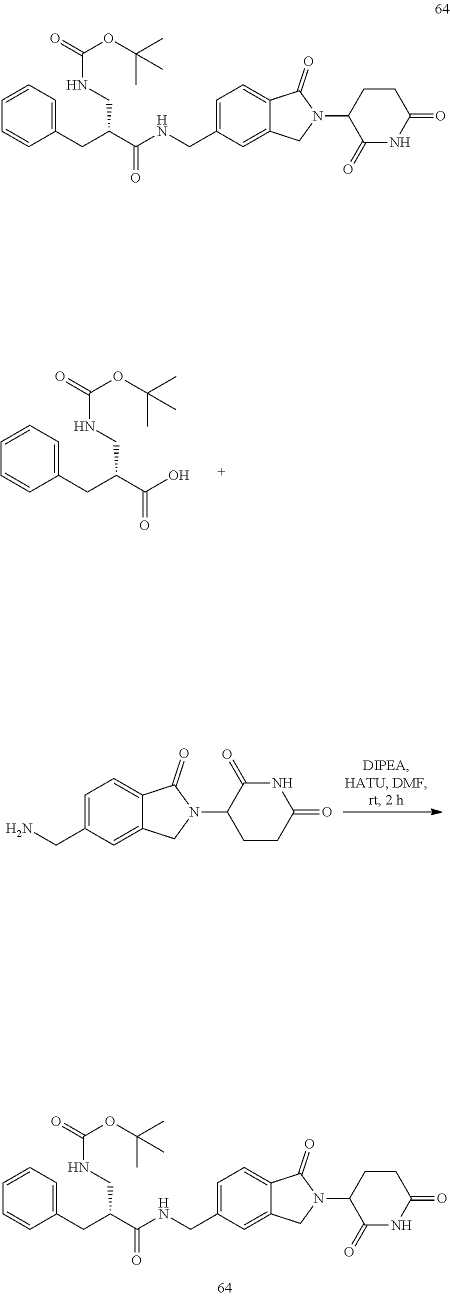 [SMILES: CC(C)(C)OC(=O)NC[C@@H](Cc1ccccc1)C(=O)NCc1ccc2c(c1)CN(C1CCC(=O)NC1=O)C2=O ; CC(C)(C)OC(=O)NC[C@@H](Cc1ccccc1)C(=O)O ; NCc1ccc2c(c1)CN(C1CCC(=O)NC1=O)C2=O ; CC(C)(C)OC(=O)NC[C@@H](Cc1ccccc1)C(=O)NCc1ccc2c(c1)CN(C1CCC(=O)NC1=O)C2=O]

Compound 64 was prepared according to Example 1, Step 5. MS(m/z): 434.9 [M−100]; ¹H NMR (400 MHz, DMSO-d₆) δ10.99 (s, 1H), 8.38 (s, 1H), 7.56 (d, J=7.9 Hz, 1H), 7.38-7.03 (m, 7H), 6.86 (t, J=5.3 Hz, 1H), 5.11 (dd, J=13.3, 5.1 Hz, 1H), 4.54-4.08 (m, 4H), 3.21-3.02 (m, 2H), 2.98-2.87 (m, 1H), 2.83-2.77 (m, 1H), 2.74 (d, J=7.6 Hz, 2H), 2.62 (d, J=16.8 Hz, 1H), 2.43 (dd, J=13.2, 4.2 Hz, 1H), 2.07-1.95 (m, 1H), 1.39 (s, 9H).

### Example 65 (4-186)

### 2-(2-(tert butylamino)-2-oxoethyl)amino)-N-(2-(2,6-dioxopyridin-3-yl)-1-oxoisoindoline-5-yl) methyl)-3-phenylpropanamide

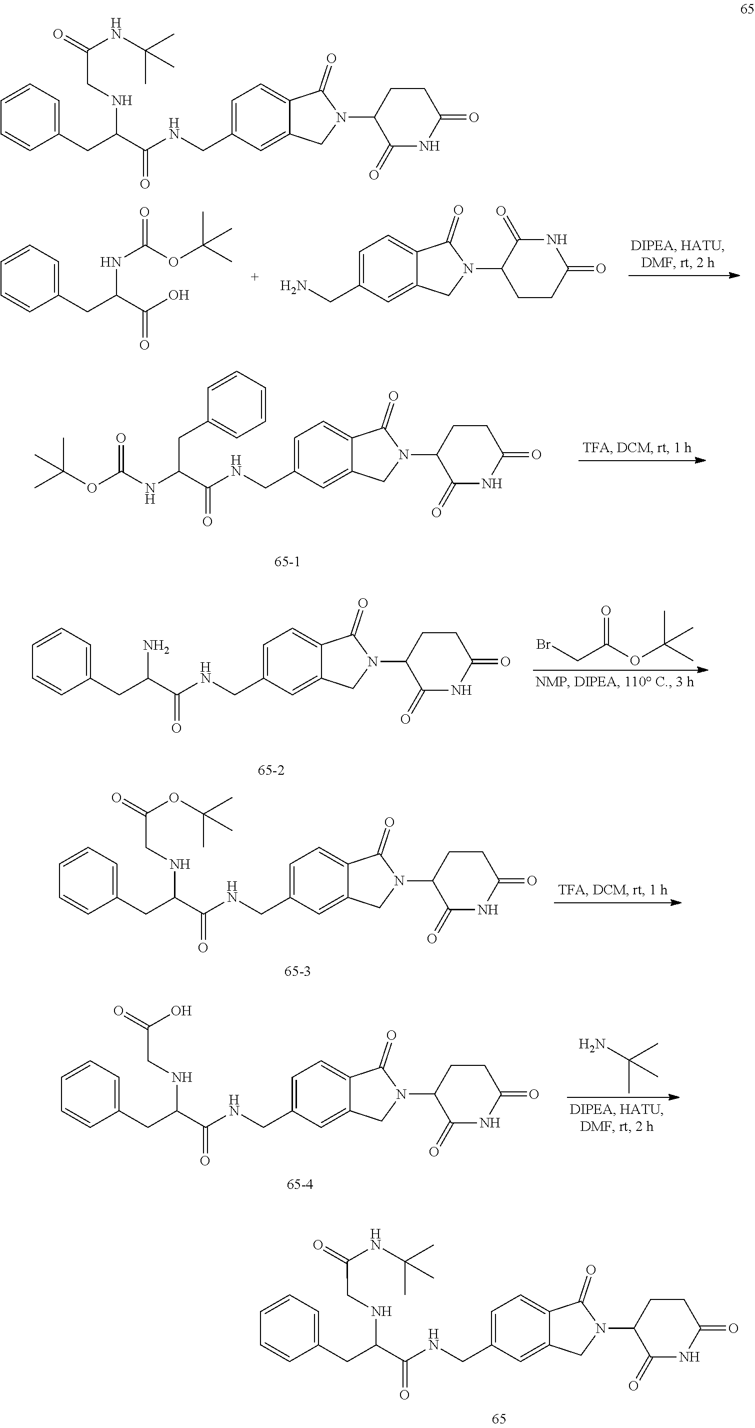

Intermediate 65-1 was prepared according to the method of Step 5, Example 1.

Intermediate 65-2 was prepared according to the method of Step 4, Example 1.

Intermediate 65-3 was prepared according to the method of Example 59.

Intermediate 65-4 was prepared according to the method of Step 4, Example 1.

Compound 65 was prepared according to Example 1, Step 5. MS(m/z): 533.8 [M+H]⁺; ¹H NMR (400 MHz, DMSO-d₆) δ10.99 (s, 1H), 8.63 (t, J=5.7 Hz, 1H), 7.65 (d, J=7.8 Hz, 1H), 7.36-7.19 (m, 7H), 7.08 (s, 1H), 5.12 (dd, J=13.2, 5.0 Hz, 1H), 4.52-4.25 (m, 4H), 3.28 (s, 1H), 3.06 (d, J=16.1 Hz, 1H), 2.99-2.84 (m, 2H), 2.80-2.67 (m, 2H), 2.61 (d, J=16.6 Hz, 1H), 2.42 (dd, J=13.2, 4.3 Hz, 1H), 2.06-1.97 (m, 1H), 1.13 (s, 9H).

### Example 66 (4-187)

### N-(2-benzyl-3-((2-(2,6-dioxypiperidin-3-yl)-1-oxoisoindoline-5-yl)methyl)amino)-3-oxopropyl)-3,3-dimethylbutanamide

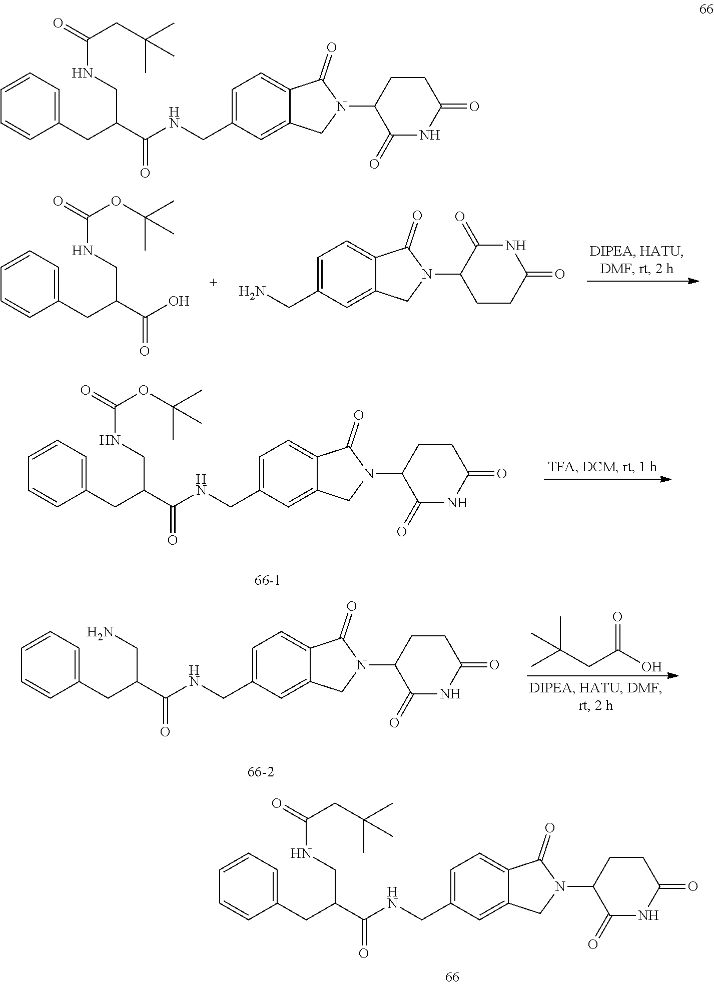

Intermediate 66-1 was prepared according to the method of Step 5, Example 1.

Intermediate 66-2 was prepared according to the method of Step 4, Example 1.

Compound 66 was prepared according to Example 1, Step 5. MS(m/z): 532.9 [M+H]⁺; ¹H NMR (400 MHz, DMSO-d₆) δ10.99 (s, 1H), 8.40 (t, J=5.1 Hz, 1H), 7.85 (d, J=1.9 Hz, 1H), 7.56 (d, J=8.0 Hz, 1H), 7.26 (ddd, J=15.5, 9.3, 4.9 Hz, 3H), 7.20-7.15 (m, 2H), 7.12 (t, J=4.8 Hz, 2H), 5.12 (dd, J=13.3, 5.0 Hz, 1H), 4.45-4.14 (m, 4H), 2.99-2.87 (m, 1H), 2.83 (dd, J=13.8, 7.0 Hz, 1H), 2.75 (d, J=6.7 Hz, 2H), 2.62 (d, J=17.5 Hz, 1H), 2.46-2.35 (m, 1H), 2.00 (s, 1H), 1.97 (s, 2H), 0.99 (s, 2H), 0.96 (s, 9H).

### Example 67 (4-188)

### 2-Benzyl-3-(3-(tert butyl) urea)-N-((2-(2,6-dioxypiperidin-3-yl)-1-oxoisoindoline-5-yl) methyl) propionamide

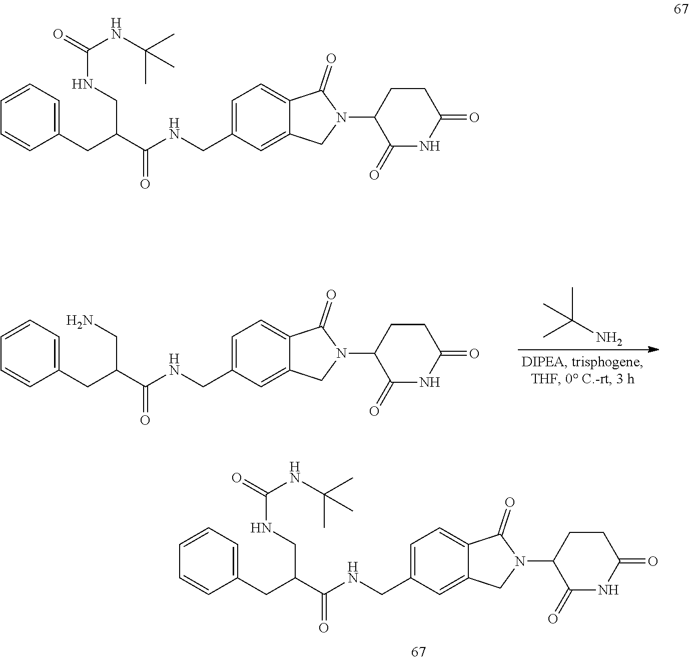

Compound 67 was prepared according to Example 16 MS(m/z): 533.8 [M+H]⁺; ¹H NMR (400 MHz, DMSO-d₆) δ10.99 (s, 1H), 8.45 (s, 1H), 7.58 (d, J=8.1 Hz, 1H), 7.30-7.22 (m, 3H), 7.17 (dd, J=10.7, 4.7 Hz, 4H), 5.81-5.71 (m, 2H), 5.12 (dd, J=13.3, 5.1 Hz, 1H), 4.50-4.35 (m, 2H), 4.30-4.20 (m, 2H), 3.12-3.03 (m, 1H), 3.00-2.85 (m, 1H), 2.82-2.72 (m, 2H), 2.68 (dd, J=18.7, 11.8 Hz, 2H), 2.60 (s, 1H), 2.42 (dt, J=13.3, 8.9 Hz, 1H), 2.06-1.96 (m, 1H), 1.22 (s, 9H).

### Example 68 (4-189)

### N-(2-benzyl-3-((2-(2,6-dioxypiperidin-3-yl)-1-oxoisoindoline-5-yl)methyl)amino)-3-oxopropyl) cyclopentanamide

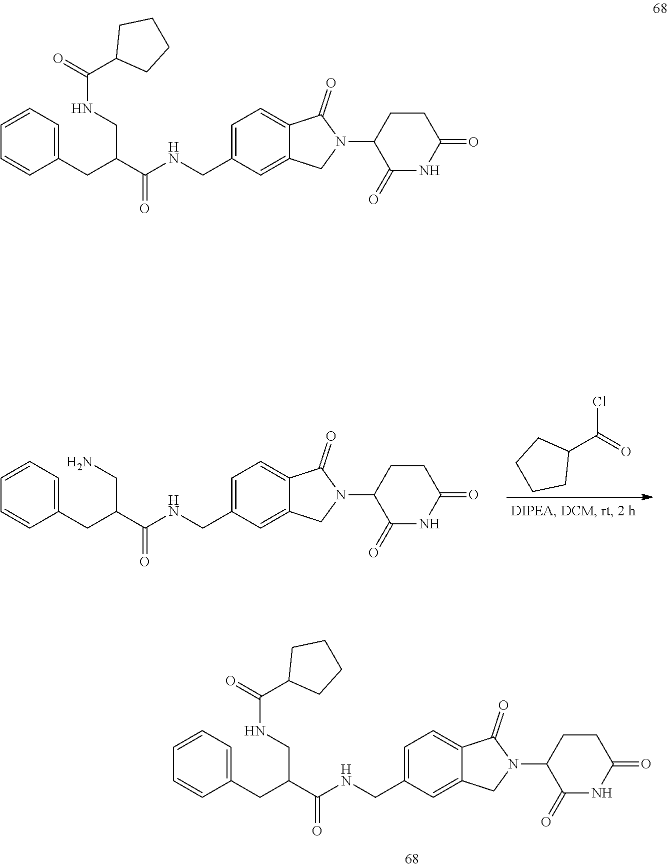 [SMILES: O=C1CCC(N2Cc3cc(CNC(=O)C(CNC(=O)C4CCCC4)Cc4ccccc4)ccc3C2=O)C(=O)N1 ; NCC(Cc1ccccc1)C(=O)NCc1ccc2c(c1)CN(C1CCC(=O)NC1=O)C2=O ; O=C(Cl)C1CCCC1 ; O=C1CCC(N2Cc3cc(CNC(=O)C(CNC(=O)C4CCCC4)Cc4ccccc4)ccc3C2=O)C(=O)N1]

Compound 68 was prepared according to Example 11, Step 2. MS(m/z): 531.3 [M+H]⁺; ¹H NMR (400 MHz, DMSO-d₆) δ10.99 (s, 1H), 8.41 (t, J=5.4 Hz, 1H), 7.86 (t, J=5.3 Hz, 1H), 7.56 (d, J=8.0 Hz, 1H), 7.31-7.05 (m, 7H), 5.12 (dd, J=13.3, 5.1 Hz, 1H), 4.45-4.11 (m, 4H), 3.20 (ddd, J=38.3, 13.6, 7.0 Hz, 2H), 2.98-2.79 (m, 2H), 2.77-2.66 (m, 2H), 2.58 (dd, J=28.6, 13.8 Hz, 2H), 2.43 (dd, J=13.1, 4.3 Hz, 1H), 2.06-1.95 (m, 1H), 1.73-1.65 (m, 2H), 1.60 (d, J=1.9 Hz, 4H), 1.51 (dd, J=15.4, 8.5 Hz, 2H).

### Example 69 (5-8-2)

### 3-amino-N-((2-(2,6-dioxopyridin-3-yl)-1-oxoisoidoline-5-yl)methyl)-2-phenylpropanamide

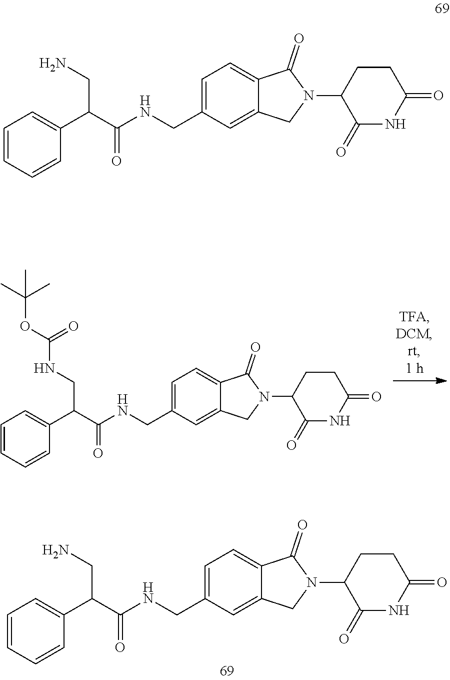 [SMILES: NCC(C(=O)NCc1ccc2c(c1)CN(C1CCC(=O)NC1=O)C2=O)c1ccccc1 ; CC(C)(C)OC(=O)NCC(C(=O)NCc1ccc2c(c1)CN(C1CCC(=O)NC1=O)C2=O)c1ccccc1 ; NCC(C(=O)NCc1ccc2c(c1)CN(C1CCC(=O)NC1=O)C2=O)c1ccccc1]

Compound 69 was prepared according to Example 1, Step 4. MS(m/z): 420.2 [M+H]⁺; ¹H NMR (400 MHz, DMSO-d₆) δ10.97 (s, 1H), 8.81 (t, J=5.7 Hz, 1H), 7.87 (s, 3H), 7.59 (d, J=7.8 Hz, 1H), 7.37 (dd, J=14.2, 6.7 Hz, 4H), 7.25 (dd, J=12.1, 5.6 Hz, 2H), 5.09 (dd, J=13.2, 4.9 Hz, 1H), 4.56-4.16 (m, 4H), 3.43 (d, J=9.7 Hz, 2H), 3.08 (s, 1H), 2.98-2.84 (m, 1H), 2.60 (d, J=16.9 Hz, 1H), 2.46-2.31 (m, 1H), 2.07-1.93 (m, 1H).

### Example 70 (5-2)

### N-(3-((2-(2,6-dioxypiperidin-3-yl)-1-oxoisoidoline-5-yl)methyl)amino)-3-oxo-2-phenylpropyl)-3,3-dimethylbutanamide

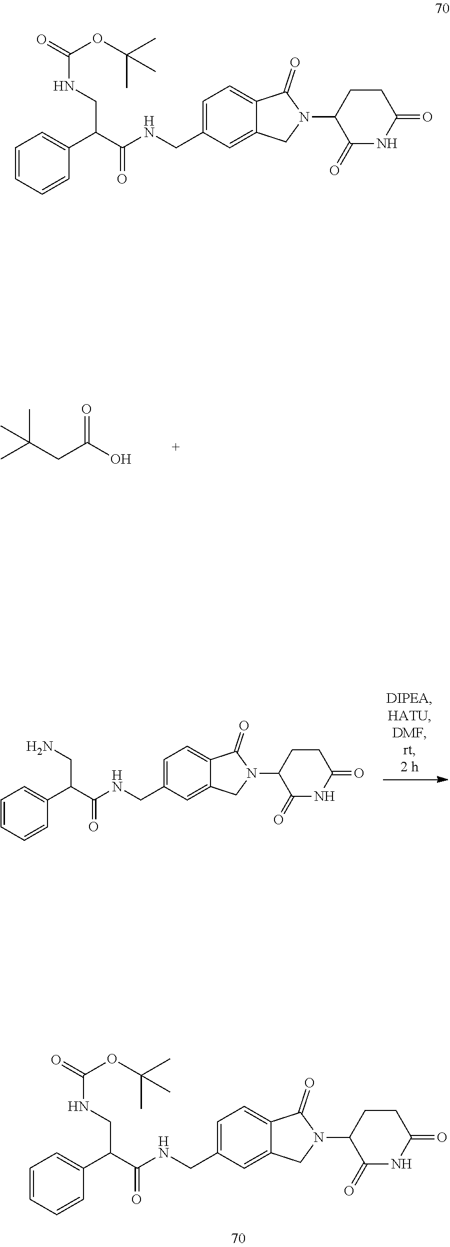

Compound 70 was prepared according to Example 1, Step 5. MS(m/z): 519.3 [M+H]⁺; ¹H NMR (400 MHz, DMSO-d₆) δ10.95 (s, 1H), 8.68 (t, J=5.9 Hz, 1H), 7.86 (d, J=5.1 Hz, 1H), 7.59 (d, J=8.1 Hz, 1H), 7.36-7.22 (m, 7H), 5.09 (dd, J=13.2, 3.3 Hz, 1H), 4.43-4.23 (m, 4H), 3.86 (t, J=7.4 Hz, 1H), 3.44 (d, J=6.4 Hz, 2H), 2.97-2.83 (m, 1H), 2.60 (d, J=17.9 Hz, 1H), 2.38 (d, J=13.3 Hz, 1H), 2.02-1.95 (m, 1H), 1.91 (d, J=4.0 Hz, 2H), 0.88 (s, 9H).

### Example 71 (5-3)

### Cyclopentyl (3-((2-(2,6-dioxypiperidin-3-yl)-1-oxoisoindolin-5-yl)methyl)amino)-3-oxo-2-phenylpropyl) carbamate

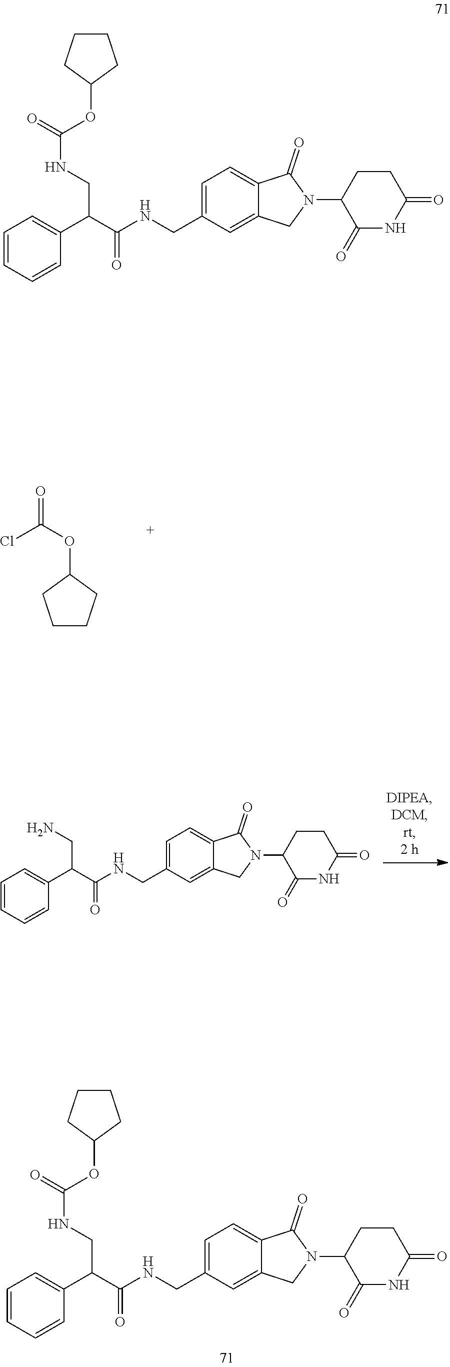 [SMILES: O=C1CCC(N2Cc3cc(CNC(=O)C(CNC(=O)OC4CCCC4)c4ccccc4)ccc3C2=O)C(=O)N1 ; O=C(Cl)OC1CCCC1 ; NCC(C(=O)NCc1ccc2c(c1)CN(C1CCC(=O)NC1=O)C2=O)c1ccccc1 ; O=C1CCC(N2Cc3cc(CNC(=O)C(CNC(=O)OC4CCCC4)c4ccccc4)ccc3C2=O)C(=O)N1]

Compound 71 was prepared according to Example 11, Step 2. MS(m/z): 532.8 [M+H]⁺; ¹H NMR (400 MHz, DMSO-d₆) δ10.97 (s, 1H), 8.70 (s, 1H), 7.59 (d, J=8.2 Hz, 1H), 7.38-7.23 (m, 7H), 7.08 (s, 1H), 5.09 (dd, J=13.2, 5.0 Hz, 1H), 4.91 (s, 1H), 4.48-4.19 (m, 4H), 3.86 (t, J=7.4 Hz, 1H), 3.71 (s, 1H), 3.59-3.42 (m, 1H), 3.30-3.22 (m, 1H), 2.98-2.84 (m, 1H), 2.60 (d, J=16.8 Hz, 1H), 2.44-2.31 (m, 1H), 2.06-1.94 (m, 1H), 1.74 (d, J=5.6 Hz, 2H), 1.51 (s, 5H).

### Example 72 (5-9)

### N-(3-((2-(2,6-dioxypiperidin-3-yl)-1-oxoisoindolin-5-yl)methyl)amino)-3-oxo-2-phenylpropyl) cyclopentyl Formamide

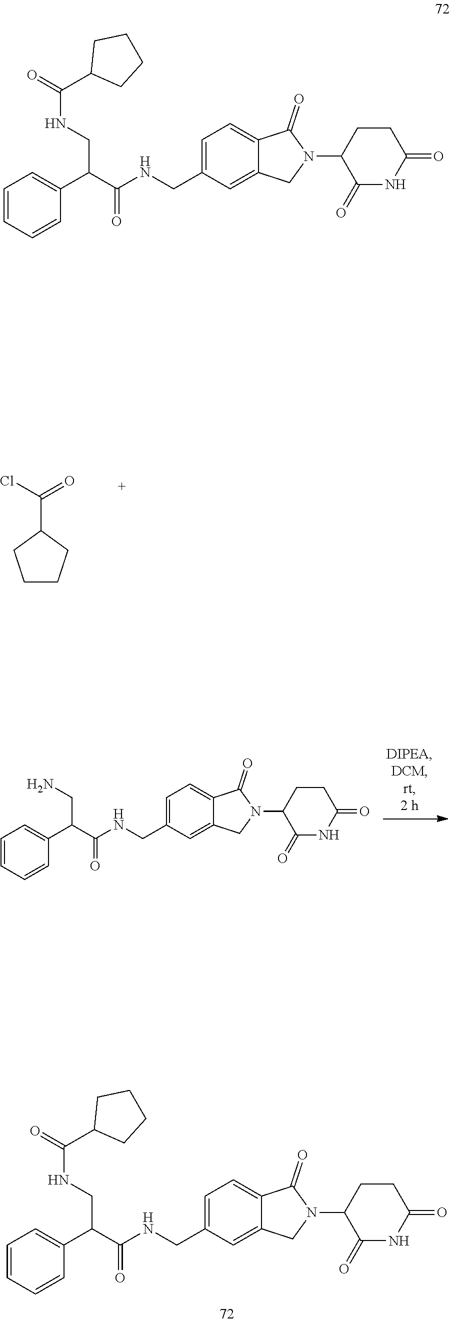 [SMILES: O=C1CCC(N2Cc3cc(CNC(=O)C(CNC(=O)C4CCCC4)c4ccccc4)ccc3C2=O)C(=O)N1 ; O=C(Cl)C1CCCC1 ; NCC(C(=O)NCc1ccc2c(c1)CN(C1CCC(=O)NC1=O)C2=O)c1ccccc1 ; O=C1CCC(N2Cc3cc(CNC(=O)C(CNC(=O)C4CCCC4)c4ccccc4)ccc3C2=O)C(=O)N1]

Compound 72 was prepared according to Example 11, Step 2. MS(m/z): 517.2 [M+H]⁺; ¹H NMR (400 MHz, DMSO-d₆) δ10.97 (s, 1H), 8.69 (s, 1H), 7.59 (d, J=8.0 Hz, 1H), 7.36-7.22 (m, 7H), 7.07 (s, 1H), 5.09 (dd, J=13.1, 4.8 Hz, 1H), 4.91 (s, 1H), 4.47-4.18 (m, 5H), 3.86 (t, J=7.2 Hz, 1H), 3.48 (d, J=6.3 Hz, 1H), 2.98-2.83 (m, 1H), 2.60 (d, J=17.3 Hz, 1H), 2.38 (dt, J=23.0, 11.5 Hz, 1H), 2.06-1.91 (m, 1H), 1.74 (d, J=5.3 Hz, 2H), 1.51 (s, 6H).

### Example 73 (5-10)

### Phenyl (3-((2-(2,6-dioxypiperidin-3-yl)-1-oxoisoindolin-5-yl)methyl)amino)-3-oxo-2-phenylpropyl) carbamate

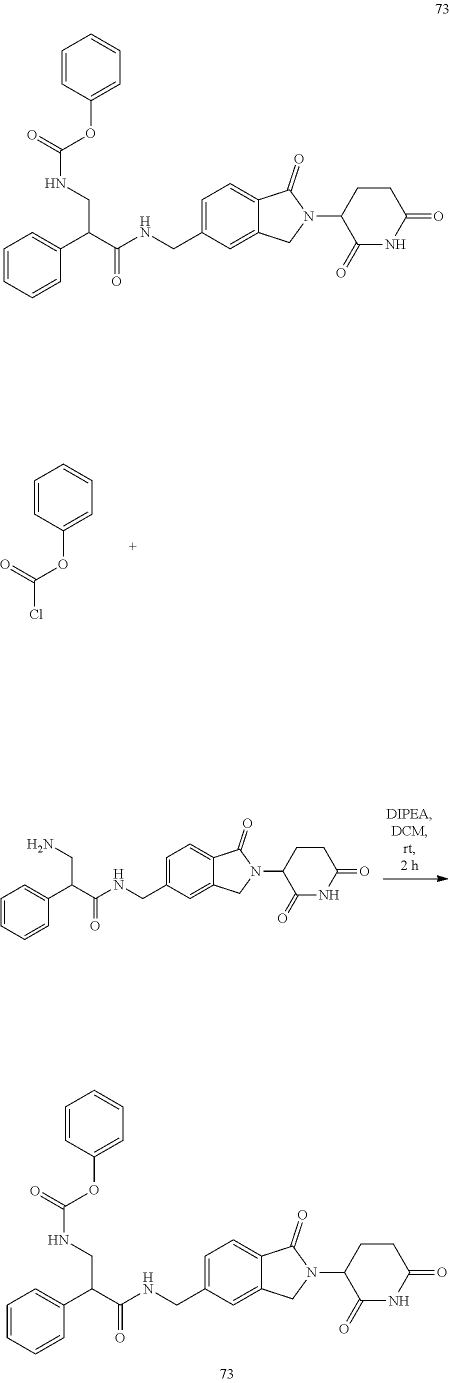 [SMILES: O=C1CCC(N2Cc3cc(CNC(=O)C(CNC(=O)Oc4ccccc4)c4ccccc4)ccc3C2=O)C(=O)N1 ; O=C(Cl)Oc1ccccc1 ; NCC(C(=O)NCc1ccc2c(c1)CN(C1CCC(=O)NC1=O)C2=O)c1ccccc1 ; O=C1CCC(N2Cc3cc(CNC(=O)C(CNC(=O)Oc4ccccc4)c4ccccc4)ccc3C2=O)C(=O)N1]

Compound 73 was prepared according to Example 11, Step 2. MS(m/z): 540.8 [M+H]⁺; ¹H NMR (400 MHz, DMSO-d₆) δ10.95 (s, 1H), 8.78 (t, J=5.3 Hz, 1H), 7.93 (dt, J=11.0, 5.3 Hz, 1H), 7.59 (d, J=7.6 Hz, 1H), 7.43-7.25 (m, 9H), 7.18 (t, J=7.3 Hz, 1H), 7.00 (dd, J=7.4, 3.5 Hz, 2H), 5.07 (dd, J=13.2, 4.7 Hz, 1H), 4.54-4.12 (m, 4H), 3.96 (t, J=5.7 Hz, 1H), 3.62 (dd, J=8.3, 4.8 Hz, 1H), 3.46-3.35 (m, 1H), 2.96-2.81 (m, 1H), 2.58 (d, J=17.2 Hz, 1H), 2.39-2.19 (m, 1H), 1.95 (dd, J=14.2, 9.0 Hz, 1H).

### Example 74 (5-11)

### N-((2-(2,6-dioxypiperidin-3-yl)-1-oxoisoindolin-5-yl) methyl)-2-phenyl-3-(3-phenylurea) propionamide

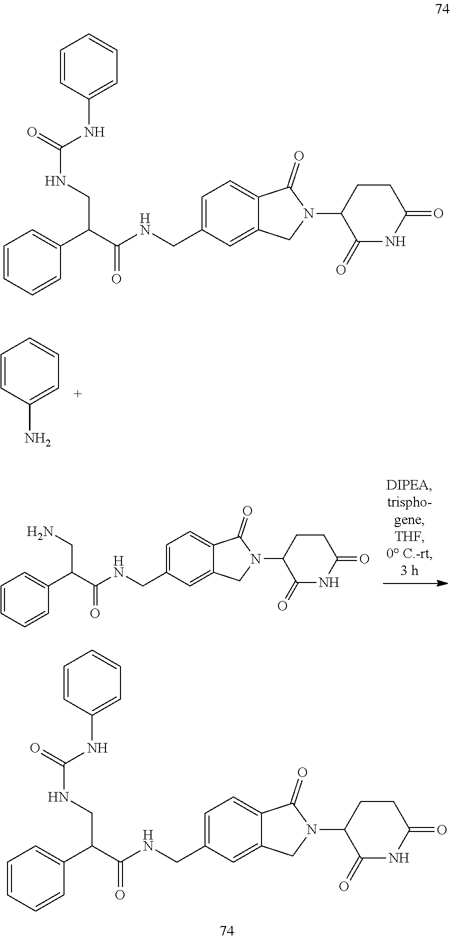

Compound 74 was prepared according to Example 16. MS(m/z): 539.8 [M+H]⁺; ¹H NMR (400 MHz, DMSO-d₆) δ10.97 (d, J=16.8 Hz, 1H), 8.73 (d, J=4.7 Hz, 1H), 8.56 (d, J=2.1 Hz, 1H), 7.58 (dd, J=7.8, 2.5 Hz, 1H), 7.37 (dd, J=9.1, 7.7 Hz, 6H), 7.30 (dd, J=15.1, 4.8 Hz, 3H), 7.21 (t, J=7.6 Hz, 2H), 6.87 (t, J=7.3 Hz, 1H), 6.30 (dt, J=15.5, 5.9 Hz, 1H), 5.07 (dt, J=13.2, 4.7 Hz, 1H), 4.52 (td, J=16.1, 6.3 Hz, 1H), 4.35-4.03 (m, 4H), 3.58-3.43 (m, 2H), 2.97-2.82 (m, 1H), 2.58 (d, J=18.4 Hz, 1H), 2.29-2.06 (m, 1H), 2.04-1.86 (m, 1H).

### Example 75 (5-12)

### 3-(3-(tert butyl)urea)-N-(2-(2,6-dioxopyridin-3-yl)-1-oxoisoindolin-5-yl)methyl)-2-phenylpropanamide

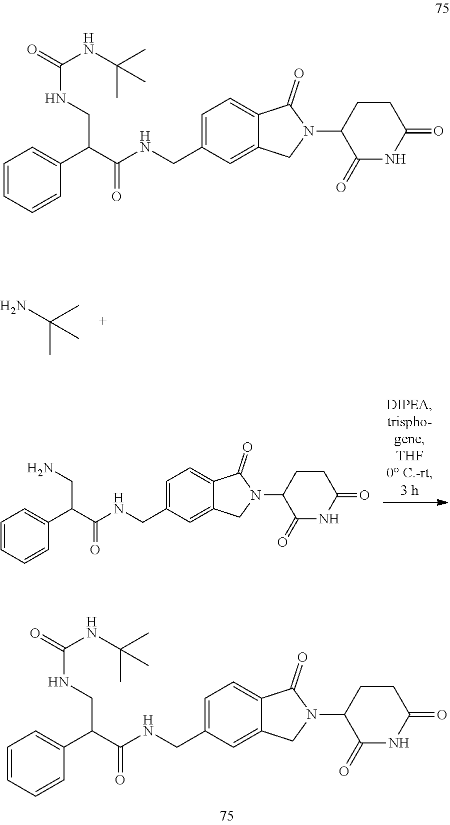

Compound 75 was prepared according to Example 16. MS(m/z): 519.9 [M+H]⁺; ¹H NMR (400 MHz, DMSO-d₆) δ10.96 (s, 1H), 8.67 (d, J=1.6 Hz, 1H), 7.60 (d, J=8.0 Hz, 1H), 7.46-7.20 (m, 7H), 5.77 (d, J=10.4 Hz, 2H), 5.09 (dd, J=13.2, 5.0 Hz, 1H), 4.52-4.20 (m, 4H), 3.77 (dd, J=8.6, 6.2 Hz, 1H), 3.43-3.32 (m, 2H), 3.02-2.80 (m, 1H), 2.60 (d, J=17.2 Hz, 1H), 2.37 (dd, J=13.2, 4.3 Hz, 1H), 2.11-1.94 (m, 1H), 1.20 (s, 9H).

### Example 76 (5-24)

### N-((2-(2,6-dioxopyridin-3-yl)-1-oxoisoidoline-5-yl)methyl)-3-isobutyrylamino-2-phenylpropanamide

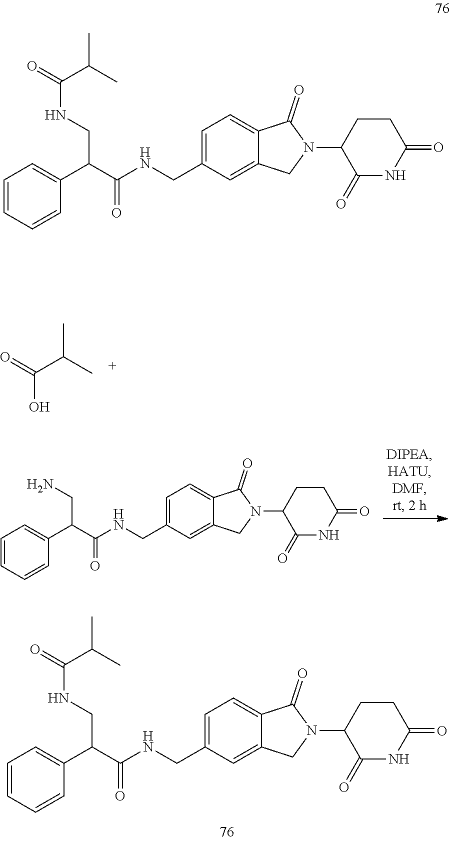

Compound 76 was prepared according to Example 1, Step 5. MS(m/z): 490.9 [M+H]⁺; ¹H NMR (400 MHz, DMSO-d₆) δ10.97 (s, 1H), 8.72 (t, J=5.4 Hz, 1H), 7.92 (t, J=5.5 Hz, 1H), 7.60 (d, J=8.0 Hz, 1H), 7.42-7.20 (m, 7H), 5.09 (dd, J=13.2, 5.0 Hz, 1H), 4.51-4.18 (m, 4H), 3.95-3.81 (m, 1H), 3.51-3.36 (m, 2H), 2.90 (ddd, J=13.6, 12.4, 5.4 Hz, 1H), 2.60 (d, J=17.4 Hz, 1H), 2.40 (dd, J=13.1, 4.3 Hz, 1H), 2.32 (dt, J=13.6, 6.9 Hz, 1H), 2.04-1.94 (m, 1H), 0.96-0.85 (m, 6H).

### Example 77 (5-23)

### N-((2-(2,6-dioxypiperidin-3-yl)-1-oxoisoidoline-5-yl)methyl)-2-phenyl-3-terpentylaminopropionamide

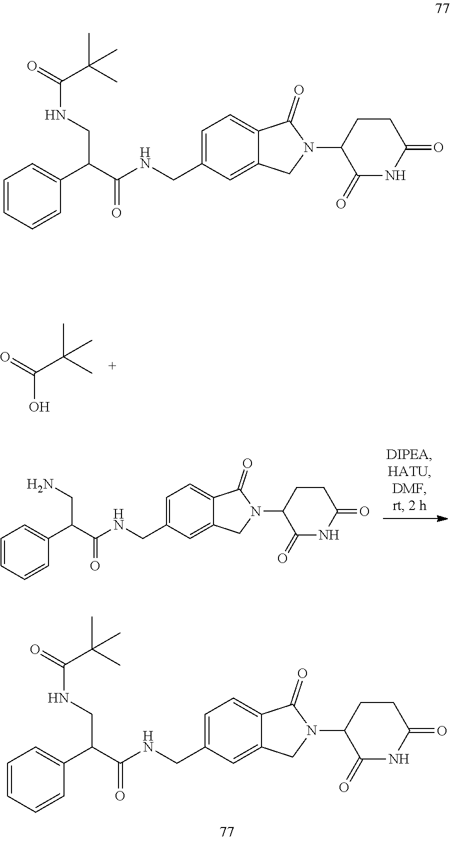

Compound 77 was prepared according to Example 1, Step 5. MS(m/z): 504.9 [M+H]⁺; ¹H NMR (400 MHz, DMSO-d₆) δ10.96 (s, 1H), 8.70 (t, J=5.9 Hz, 1H), 7.65-7.46 (m, 2H), 7.42-7.18 (m, 7H), 5.09 (dd, J=13.2, 4.9 Hz, 1H), 4.49-4.19 (m, 4H), 3.91 (dd, J=8.2, 6.5 Hz, 1H), 3.50 (td, J=8.1, 4.3 Hz, 1H), 3.44-3.35 (m, 1H), 2.97-2.84 (m, 1H), 2.60 (d, J=16.5 Hz, 1H), 2.40 (dd, J=13.0, 4.2 Hz, 1H), 2.05-1.94 (m, 1H), 0.99 (s, 9H).

### Example 78 (5-25)

### N-(3-((2-(2,6-dioxypiperidin-3-yl)-1-oxoisoindolin-5-yl)methyl)amino)-3-oxo-2-phenylpr opyl) cyclohexylformamide

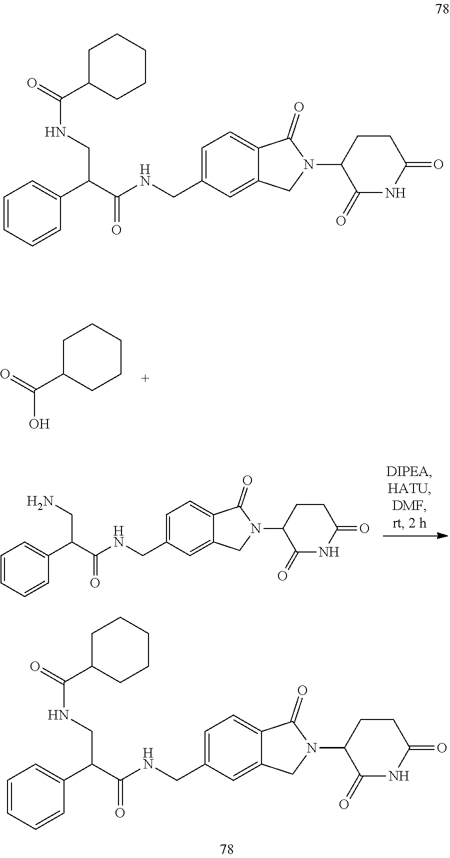

Compound 78 was prepared according to Example 1, Step 5. MS(m/z): 531.0 [M+H]⁺; ¹H NMR (400 MHz, DMSO-d₆) δ10.97 (s, 1H), 8.71 (t, J=5.7 Hz, 1H), 7.85 (dd, J=10.4, 5.3 Hz, 1H), 7.60 (d, J=7.7 Hz, 1H), 7.48-7.12 (m, 7H), 5.10 (dd, J=13.2, 4.8 Hz, 1H), 4.51-4.19 (m, 4H), 3.86 (dd, J=8.4, 6.5 Hz, 1H), 3.41 (ddd, J=19.3, 10.7, 5.8 Hz, 2H), 2.89 (dd, J=13.3, 4.6 Hz, 1H), 2.60 (d, J=17.4 Hz, 1H), 2.38 (dd, J=13.0, 4.4 Hz, 1H), 2.01 (dt, J=10.7, 9.4 Hz, 2H), 1.69-1.52 (m, 4H), 1.47 (d, J=12.8 Hz, 1H), 1.23 (t, J=11.6 Hz, 2H), 1.17-1.04 (m, 3H).

### Example 79 (5-26)

### N-(3-((2-(2,6-dioxypiperidin-3-yl)-1-oxoisoindolin-5-yl)methyl)amino)-3-oxo-2-phenylpr opyl) benzamide

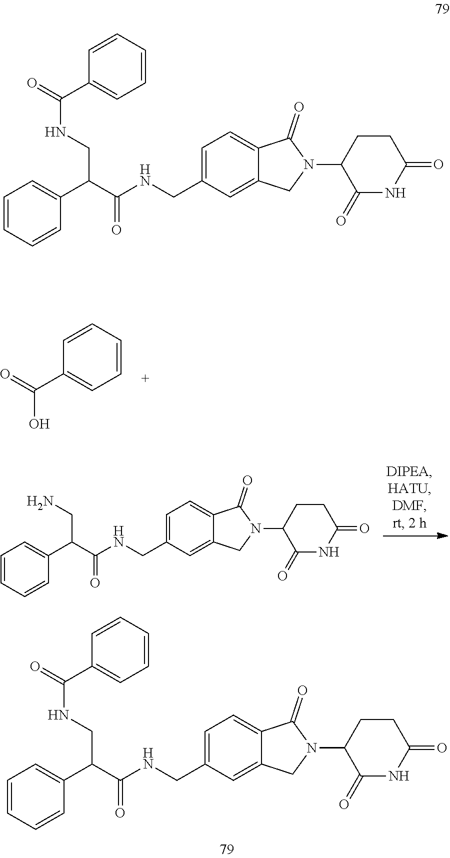

Compound 79 was prepared according to Example 1, Step 5. MS(m/z): 524.9 [M+H]⁺; ¹H NMR (400 MHz, DMSO-d₆) δ10.97 (d, J=2.0 Hz, 1H), 8.75 (t, J=5.8 Hz, 1H), 8.67 (s, 1H), 7.83-7.76 (m, 2H), 7.50 (t, J=7.2 Hz, 2H), 7.42 (dd, J=14.3, 7.3 Hz, 4H), 7.34 (t, J=7.4 Hz, 2H), 7.26 (dd, J=14.9, 7.2 Hz, 3H), 5.07 (dd, J=13.3, 5.0 Hz, 1H), 4.51 (dd, J=15.5, 6.3 Hz, 1H), 4.26-3.99 (m, 4H), 3.79-3.56 (m, 2H), 2.98-2.82 (m, 1H), 2.62 (d, J=16.7 Hz, 1H), 2.40-2.26 (m, 1H), 1.98 (d, J=5.7 Hz, 1H).

### Example 80 (5-29)

### 2-(2-cyclopentylacetamido)-N-((2-(2,6-dioxopyridin-3-yl)-1-oxoisoidoline-5-yl) methyl)-2-phenylacetamide

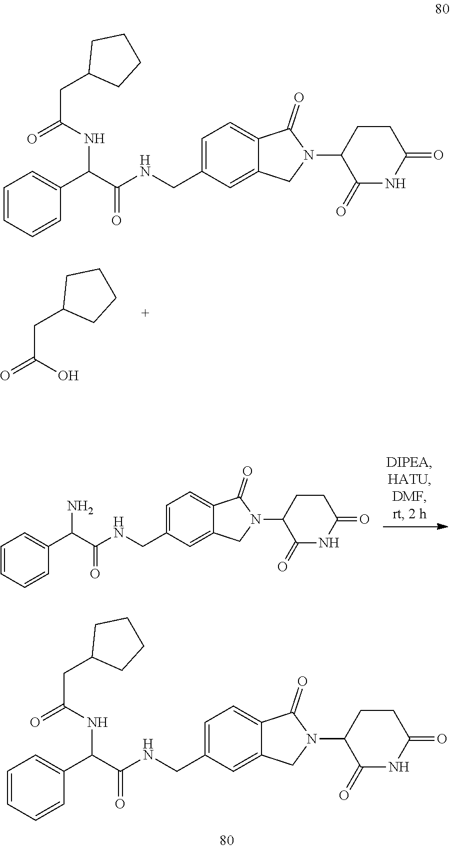 [SMILES: O=C1CCC(N2Cc3cc(CNC(=O)C(NC(=O)CC4CCCC4)c4ccccc4)ccc3C2=O)C(=O)N1 ; O=C(O)CC1CCCC1 ; NC(C(=O)NCc1ccc2c(c1)CN(C1CCC(=O)NC1=O)C2=O)c1ccccc1 ; O=C1CCC(N2Cc3cc(CNC(=O)C(NC(=O)CC4CCCC4)c4ccccc4)ccc3C2=O)C(=O)N1]

Compound 80 was prepared according to Example 1, Step 5. MS(m/z): 517.3 [M+H]⁺; ¹H NMR (400 MHz, DMSO-d₆) δ10.97 (s, 1H), 8.83 (s, 1H), 8.44 (d, J=7.9 Hz, 1H), 7.61 (d, J=8.1 Hz, 1H), 7.43 (d, J=7.3 Hz, 2H), 7.35 (t, J=7.3 Hz, 2H), 7.30 (d, J=7.0 Hz, 3H), 5.53 (d, J=7.9 Hz, 1H), 5.09 (dd, J=13.3, 4.9 Hz, 1H), 4.50-4.14 (m, 4H), 3.02-2.82 (m, 1H), 2.60 (d, J=17.6 Hz, 1H), 2.45-2.31 (m, 1H), 2.22 (d, J=7.3 Hz, 2H), 2.12 (dt, J=15.0, 7.5 Hz, 1H), 2.05-1.93 (m, 1H), 1.74-1.38 (m, 6H), 1.19-1.06 (m, 2H).

### Example 81 (5-30)

### N-(2-(2-(2,6-dioxopyridin-3-yl)-1-oxoisoidoline-5-yl)methyl) amino)-2-oxo-1-phenylethyl) cyclopentanamide

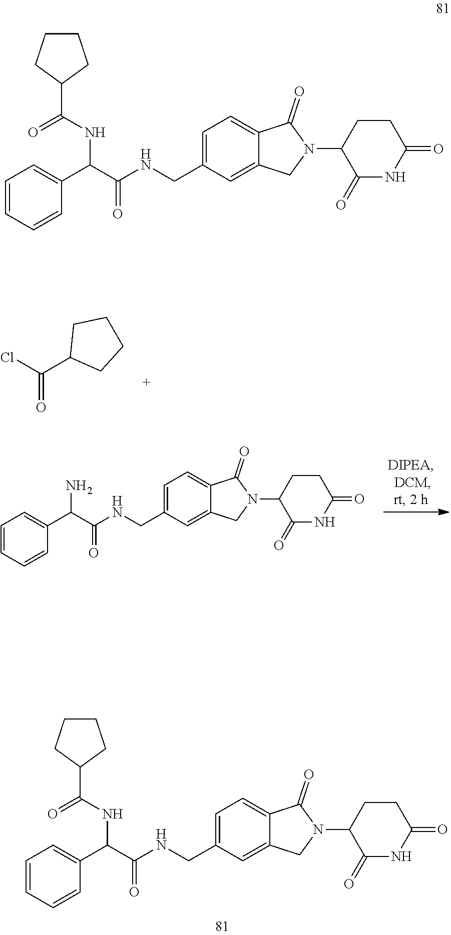 [SMILES: O=C1CCC(N2Cc3cc(CNC(=O)C(NC(=O)C4CCCC4)c4ccccc4)ccc3C2=O)C(=O)N1 ; O=C(Cl)C1CCCC1 ; NC(C(=O)NCc1ccc2c(c1)CN(C1CCC(=O)NC1=O)C2=O)c1ccccc1 ; O=C1CCC(N2Cc3cc(CNC(=O)C(NC(=O)C4CCCC4)c4ccccc4)ccc3C2=O)C(=O)N1]

Compound 81 was prepared according to Example 11, Step 2. MS(m/z): 503.3 [M+H]⁺; ¹H NMR (400 MHz, DMSO-d₆) δ10.97 (s, 1H), 8.83 (d, J=2.4 Hz, 1H), 8.40 (d, J=7.9 Hz, 1H), 7.61 (d, J=8.0 Hz, 1H), 7.43 (d, J=7.3 Hz, 2H), 7.35 (t, J=7.3 Hz, 2H), 7.30 (dd, J=7.1, 5.1 Hz, 3H), 5.51 (d, J=7.9 Hz, 1H), 5.09 (dd, J=13.3, 5.0 Hz, 1H), 4.46-4.19 (m, 4H), 2.96-2.75 (m, 2H), 2.60 (d, J=17.1 Hz, 1H), 2.47-2.31 (m, 1H), 2.06-1.95 (m, 1H), 1.79-1.68 (m, 2H), 1.67-1.52 (m, 4H), 1.48 (dd, J=12.8, 5.0 Hz, 2H).

### Example 82 (5-33)

### Tert butyl (2-((2-(2,6-dioxypiperidine-3-yl)-1-oxoisoidoline-5-yl)carbamoyl)-4-methylpentyl) carbamate

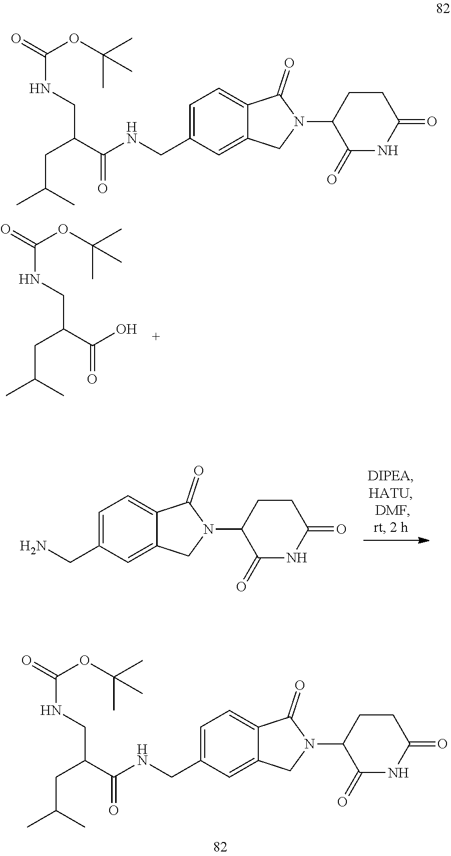

Compound 82 was prepared according to Example 1, Step 5. MS(m/z): 401.0 [M−100]; ¹H NMR (400 MHz, DMSO-d₆) δ10.94 (s, 1H), 8.48 (s, 1H), 7.65 (d, J=7.8 Hz, 1H), 7.55-7.32 (m, 2H), 6.70 (s, 1H), 5.10 (dd, J=13.2, 5.1 Hz, 1H), 4.54-4.24 (m, 4H), 3.11-3.00 (m, 1H), 2.92 (ddd, J=17.6, 12.6, 5.5 Hz, 2H), 2.60 (d, J=18.1 Hz, 1H), 2.45-2.31 (m, 1H), 2.06-1.94 (m, 1H), 1.45 (dd, J=17.0, 9.7 Hz, 2H), 1.36 (s, 9H), 1.26-1.10 (m, 2H), 0.85 (dd, J=12.0, 6.3 Hz, 6H).

### Example 83 (5-38)

### N-(2-((2-(2,6-dioxypiperidin-3-yl)-1-oxoisoindolin-5-yl)methyl)carbamoyl)-4-methylpent yl) cyclopentyl Formamide

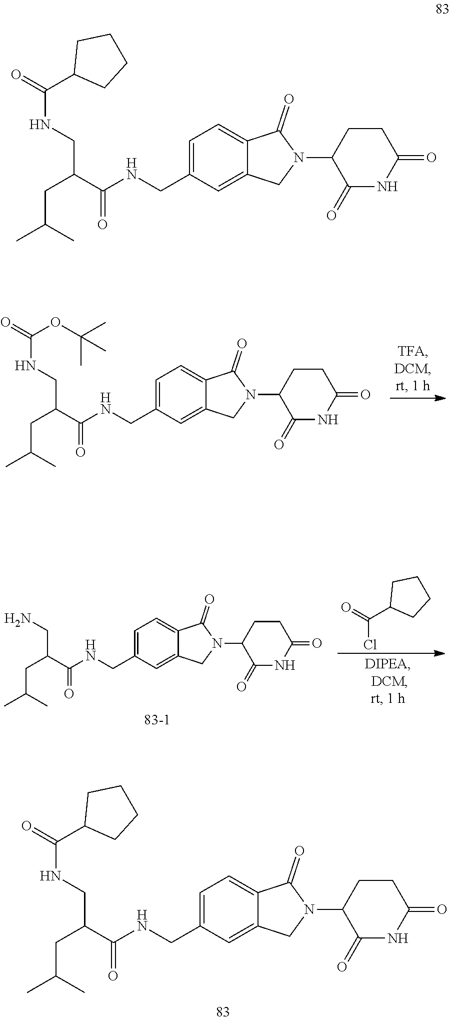

Intermediate 83-1 was prepared according to the method of Step 4, Example 1.

Compound 83 was prepared according to Example 11, Step 2. MS(m/z): 497.0 [M+H]⁺; ¹H NMR (400 MHz, DMSO-d₆) δ10.97 (s, 1H), 8.53 (t, J=5.5 Hz, 1H), 7.79 (s, 1H), 7.65 (d, J=7.8 Hz, 1H), 7.50-7.34 (m, 2H), 5.11 (dd, J=13.2, 4.9 Hz, 1H), 4.51-4.24 (m, 4H), 3.20-3.09 (m, 2H), 3.07-2.85 (m, 2H), 2.60 (d, J=16.8 Hz, 2H), 2.40 (dd, J=13.1, 3.9 Hz, 1H), 2.06-1.93 (m, 1H), 1.57 (s, 4H), 1.41 (d, J=22.2 Hz, 4H), 1.30-1.21 (m, 2H), 1.13 (dd, J=12.6, 4.6 Hz, 1H), 0.86 (dd, J=11.1, 6.4 Hz, 6H).

### Example 84 (5-41)

### Tert butyl (2-(4-chlorophenyl)-3-((2-(2,6-dioxypiperidin-3-yl)-1-oxoisoindoline-5-yl) methyl) amino)-3-oxopropyl) carbamate

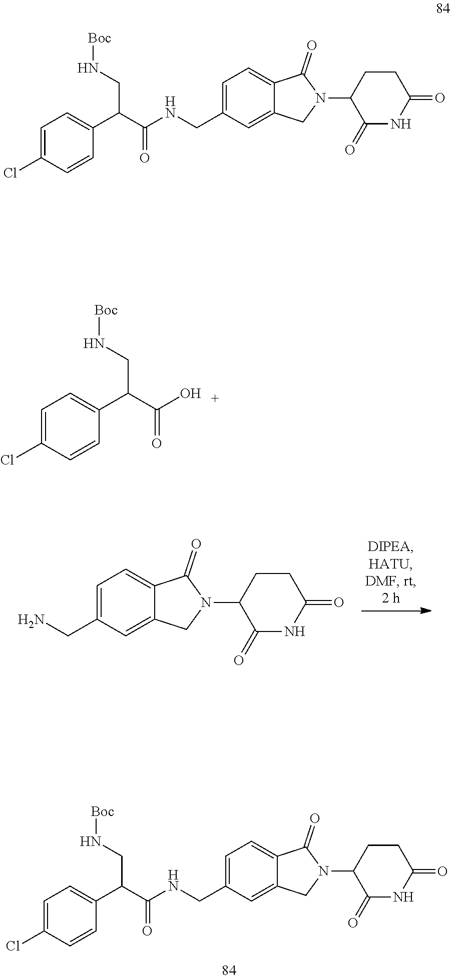 [SMILES: CC(C)(C)OC(=O)NCC(C(=O)NCc1ccc2c(c1)CN(C1CCC(=O)NC1=O)C2=O)c1ccc(Cl)cc1 ; CC(C)(C)OC(=O)NCC(C(=O)O)c1ccc(Cl)cc1 ; NCc1ccc2c(c1)CN(C1CCC(=O)NC1=O)C2=O ; CC(C)(C)OC(=O)NCC(C(=O)NCc1ccc2c(c1)CN(C1CCC(=O)NC1=O)C2=O)c1ccc(Cl)cc1]

Compound 84 was prepared according to Example 1, Step 5. MS(m/z): 454.8 [M−100]⁺; ¹H NMR (400 MHz, DMSO-d₆) δ10.95 (s, 1H), 8.70 (t, J=5.6 Hz, 1H), 7.61 (d, J=8.0 Hz, 1H), 7.38 (d, J=8.3 Hz, 2H), 7.31 (dd, J=14.3, 7.9 Hz, 4H), 6.84 (s, 1H), 5.09 (dd, J=13.2, 4.9 Hz, 1H), 4.34 (ddt, J=36.0, 25.6, 12.4 Hz, 5H), 3.87 (t, J=7.3 Hz, 1H), 3.40 (dd, J=12.5, 6.3 Hz, 1H), 2.98-2.80 (m, 1H), 2.60 (d, J=17.0 Hz, 1H), 2.37 (dd, J=13.0, 4.2 Hz, 1H), 2.08-1.84 (m, 1H), 1.33 (s, 9H).

### Example 85 (5-43)

### Tert butyl ((2S)-4-amino-1-((2-(2,6-dioxypiperidin-3-yl)-1-oxoisoindolin-5-yl) methyl) amino)-1,4-dioxobutane-2-yl) carbamate

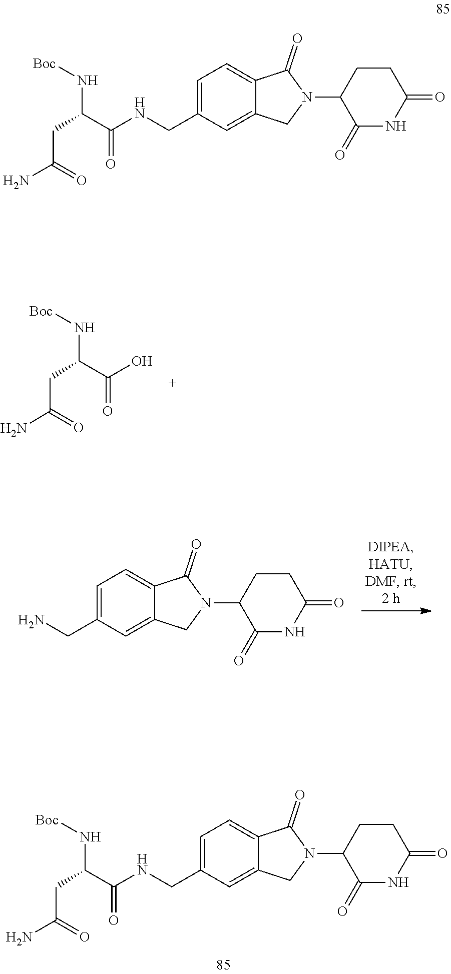 [SMILES: CC(C)(C)OC(=O)N[C@@H](CC(N)=O)C(=O)NCc1ccc2c(c1)CN(C1CCC(=O)NC1=O)C2=O ; CC(C)(C)OC(=O)N[C@@H](CC(N)=O)C(=O)O ; NCc1ccc2c(c1)CN(C1CCC(=O)NC1=O)C2=O ; CC(C)(C)OC(=O)N[C@@H](CC(N)=O)C(=O)NCc1ccc2c(c1)CN(C1CCC(=O)NC1=O)C2=O]

Compound 85 was prepared according to Example 1, Step 5. MS(m/z): 387.9 [M−100]⁺

### Example 86 (5-46)

### Tert butyl ((2S)-3-cyclohexyl-1-(2-((2,6-dioxopyridin-3-yl)-1-oxoisoidoline-5-yl) methyl) amino)-1-oxopropan-2-yl) carbamate

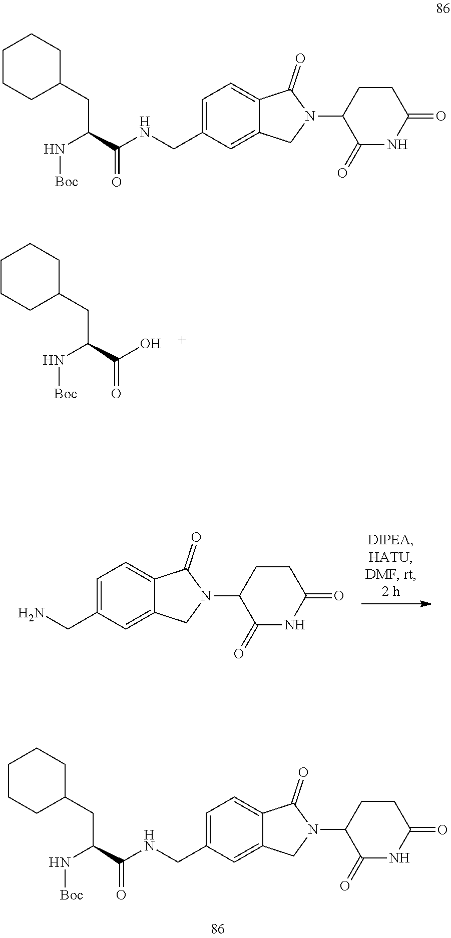 [SMILES: CC(C)(C)OC(=O)N[C@@H](CC1CCCCC1)C(=O)NCc1ccc2c(c1)CN(C1CCC(=O)NC1=O)C2=O ; CC(C)(C)OC(=O)N[C@@H](CC1CCCCC1)C(=O)O ; NCc1ccc2c(c1)CN(C1CCC(=O)NC1=O)C2=O ; CC(C)(C)OC(=O)N[C@@H](CC1CCCCC1)C(=O)NCc1ccc2c(c1)CN(C1CCC(=O)NC1=O)C2=O]

Compound 86 was prepared according to Example 1, Step 5. MS(m/z): 427.0 [M−100]⁺; ¹H NMR (400 MHz, DMSO-d₆) δ10.95 (s, 1H), 8.41 (t, J=5.7 Hz, 1H), 7.64 (d, J=7.8 Hz, 1H), 7.51-7.30 (m, 2H), 6.87 (d, J=8.0 Hz, 1H), 5.10 (dd, J=13.3, 5.1 Hz, 1H), 4.46-4.24 (m, 4H), 3.02-2.86 (m, 2H), 2.60 (d, J=16.7 Hz, 1H), 2.39 (dd, J=13.2, 4.3 Hz, 1H), 2.04-1.94 (m, 1H), 1.66 (dd, J=27.7, 15.8 Hz, 5H), 1.46-1.41 (m, 2H), 1.35 (d, J=25.0 Hz, 9H), 1.32 (s, 1H), 1.12 (s, 3H), 0.85 (dd, J=19.2, 10.4 Hz, 2H).

### Example 87 (5-47)

### Tert butyl ((2S)-1-((2-(2,6-dioxypiperidin-3-yl)-1-oxoisoindoline-5-yl) methyl) amino)-4-methyl-1-oxapent-2-yl) (methyl) carbamate Tert Butyl Ester

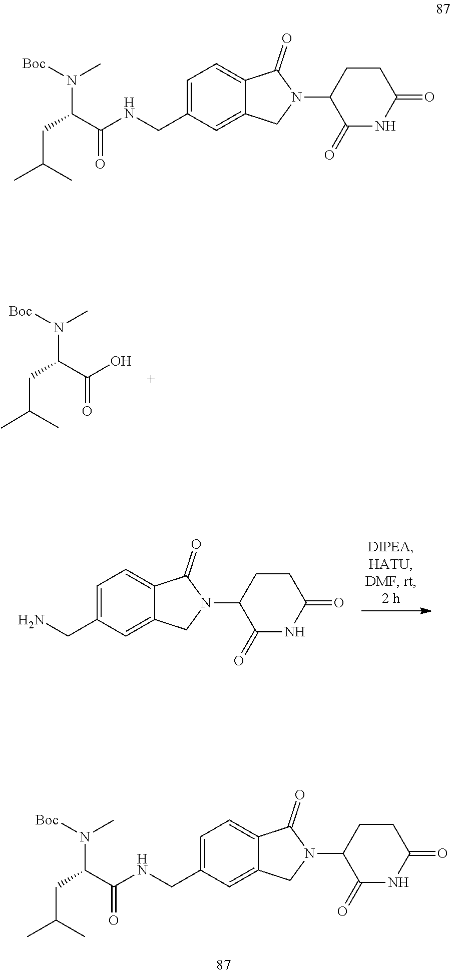 [SMILES: CC(C)C[C@@H](C(=O)NCc1ccc2c(c1)CN(C1CCC(=O)NC1=O)C2=O)N(C)C(=O)OC(C)(C)C ; CC(C)C[C@@H](C(=O)O)N(C)C(=O)OC(C)(C)C ; NCc1ccc2c(c1)CN(C1CCC(=O)NC1=O)C2=O ; CC(C)C[C@@H](C(=O)NCc1ccc2c(c1)CN(C1CCC(=O)NC1=O)C2=O)N(C)C(=O)OC(C)(C)C]

Compound 87 was prepared according to Example 1, Step 5. MS(m/z): 401.0 [M−100]; ¹H NMR (400 MHz, DMSO-d₆) δ10.96 (s, 1H), 8.48 (s, 1H), 7.66 (d, J=7.7 Hz, 1H), 7.53-7.28 (m, 2H), 5.10 (dd, J=13.3, 5.0 Hz, 1H), 4.71-4.22 (m, 5H), 2.98-2.84 (m, 1H), 2.73 (s, 3H), 2.60 (d, J=17.5 Hz, 1H), 2.39 (dd, J=13.0, 4.0 Hz, 1H), 2.06-1.95 (m, 1H), 1.59 (s, 2H), 1.39 (d, J=12.1 Hz, 9H), 1.27-1.23 (m, 1H), 0.91 (d, J=6.6 Hz, 6H).

### Example 88 (5-52)

### N-((2R)-2-(4-chlorophenyl)-3-((2-(2,6-dioxypiperidin-3-yl)-1-oxisooctyl-5-yl)methyl)amino)-3-oxopropyl) cyclopentyl Formamide

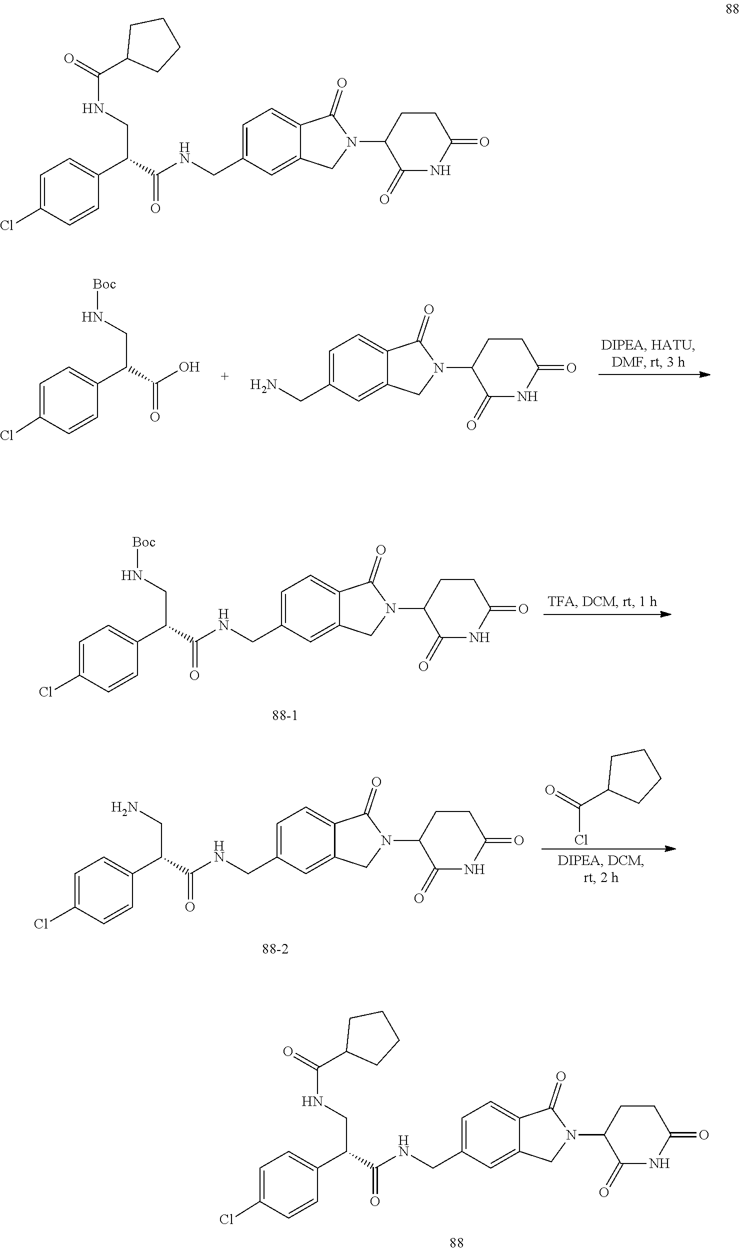

Intermediate 88-1 was prepared according to the method of Step 5, Example 1.

Intermediate 88-2 was prepared according to the method of Step 4, Example 1.

Compound 88 was prepared according to Example 11, Step 2. MS(m/z): 550.9 [M+H]⁺; ¹H NMR (400 MHz, DMSO-d₆) δ10.96 (s, 1H), 8.75 (t, J=5.9 Hz, 1H), 7.92 (s, 1H), 7.61 (d, J=8.0 Hz, 1H), 7.39 (d, J=8.5 Hz, 2H), 7.35 (s, 1H), 7.32 (d, J=9.8 Hz, 3H), 5.10 (dd, J=13.2, 5.0 Hz, 1H), 4.54-4.16 (m, 4H), 3.89 (t, J=7.5 Hz, 1H), 3.51-3.36 (m, 2H), 3.00-2.84 (m, 1H), 2.68-2.56 (m, 1H), 2.38 (dd, J=13.1, 4.3 Hz, 1H), 2.06-1.95 (m, 1H), 1.86-1.70 (m, 1H), 1.60-1.42 (m, 8H).

### Example 89 (5-53)

### N-((2R)-3-((2-(2,6-dioxypiperidin-3-yl)-1-oxoisoindolin-5-yl)methyl)amino)-3-oxo-2-phen ylpropyl) cyclopentyl Formamide

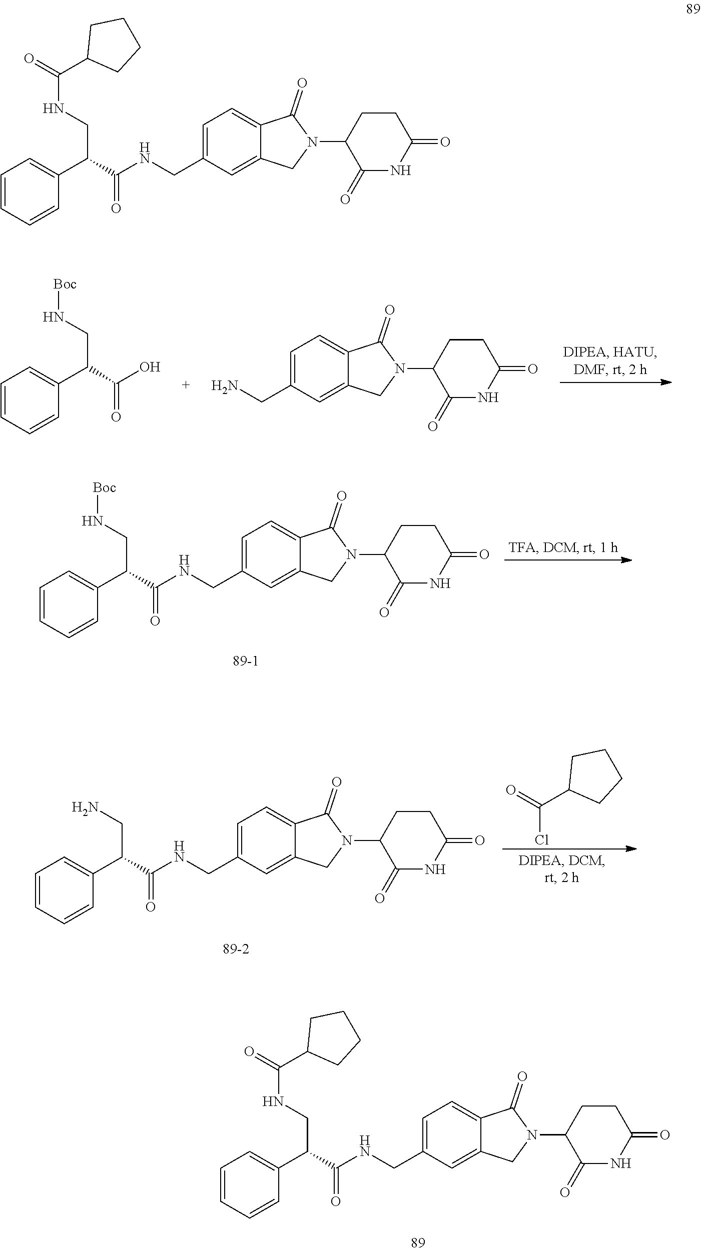

Intermediate 89-1 was prepared according to the method of Step 5, Example 1.

Intermediate 89-2 was prepared according to the method of Step 4, Example 1.

Compound 89 was prepared according to Example 11, Step 2. MS(m/z): 516.9 [M+H]⁺; ¹H NMR (400 MHz, DMSO-d₆) δ10.96 (s, 1H), 8.69 (dd, J=22.2, 16.5 Hz, 2H), 7.92 (d, J=3.1 Hz, 1H), 7.60 (d, J=7.7 Hz, 1H), 7.33 (s, 2H), 7.32 (s, 2H), 7.30 (s, 1H), 7.26 (dd, J=5.8, 2.7 Hz, 1H), 5.75 (s, 1H), 5.09 (dd, J=13.3, 5.0 Hz, 1H), 4.50-4.18 (m, 4H), 3.64-3.58 (m, 2H), 2.96-2.84 (m, 1H), 2.60 (d, J=15.2 Hz, 1H), 2.44-2.32 (m, 1H), 2.03-1.94 (m, 1H), 1.78 (s, 1H), 1.60-1.42 (m, 8H).

### Example 90 (5-54)

### N-((2S)-3-cyclohexyl-1-((2-(2,6-dioxypiperidin-3-yl)-1-oxoisoindolin-5-yl) methyl) amino)-1-oxoprop-2-yl) cyclopentyl Formamide

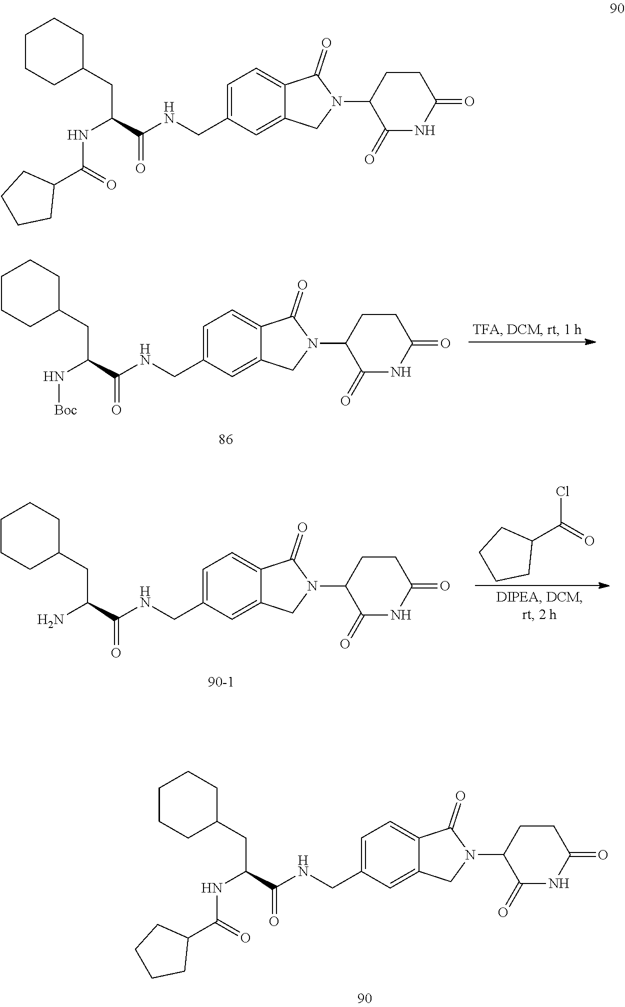

Intermediate 90-1 was prepared according to the method of Step 4, Example 1.

Compound 90 was prepared according to Example 11, Step 2. MS(m/z): 522.9 [M+H]⁺; ¹H NMR (400 MHz, DMSO-d₆) δ10.95 (s, 1H), 8.47 (t, J=5.7 Hz, 1H), 7.86 (d, J=8.2 Hz, 1H), 7.65 (d, J=7.8 Hz, 1H), 7.53-7.30 (m, 2H), 5.10 (dd, J=13.2, 5.0 Hz, 1H), 4.36 (dt, J=40.3, 17.3 Hz, 5H), 3.69-3.55 (m, 1H), 2.99-2.84 (m, 1H), 2.63 (dd, J=21.5, 13.5 Hz, 2H), 2.47-2.32 (m, 1H), 2.08-1.93 (m, 1H), 1.82-1.39 (m, 15H), 1.10 (d, J=7.4 Hz, 3H), 0.95-0.75 (m, 2H).

### Example 91 (5-55)

### Tert butyl (2-cyclohexyl-3-((2-(2,6-dioxypiperidin-3-yl)-1-oxoisoindoline-5-yl) methyl) amino)-3-oxopropyl) carbamate

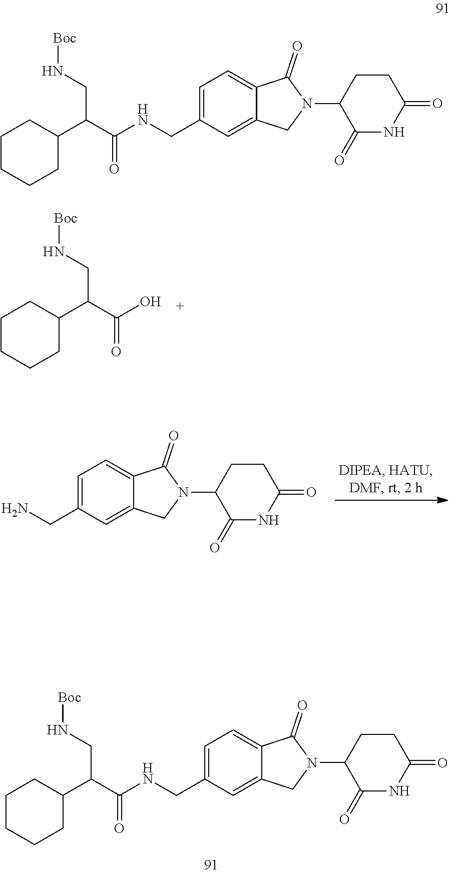

Compound 91 was prepared according to Example 1, Step 5. MS(m/z): 427.0 [M−100]⁺; ¹H NMR (400 MHz, DMSO-d₆) δ10.96 (s, 1H), 8.39 (s, 1H), 7.65 (d, J=7.8 Hz, 1H), 7.53-7.32 (m, 2H), 6.54 (s, 1H), 5.10 (dd, J=13.2, 4.9 Hz, 1H), 4.52-4.24 (m, 4H), 3.15-3.00 (m, 2H), 2.97-2.84 (m, 1H), 2.60 (d, J=17.5 Hz, 1H), 2.36 (ddd, J=26.2, 12.9, 7.0 Hz, 2H), 2.06-1.95 (m, 1H), 1.75 (d, J=12.3 Hz, 1H), 1.65 (s, 2H), 1.61-1.49 (m, 2H), 1.35 (s, 9H), 1.29-0.88 (m, 6H).

### Example 92 (5-58)

### Tert butyl ((2S)-1-((2-(2,6-dioxypiperidin-3-yl)-1-oxoisoidoline-5-yl)methyl)amino)-3-methyl-1-oxobut-2-yl) (methyl) carbamate

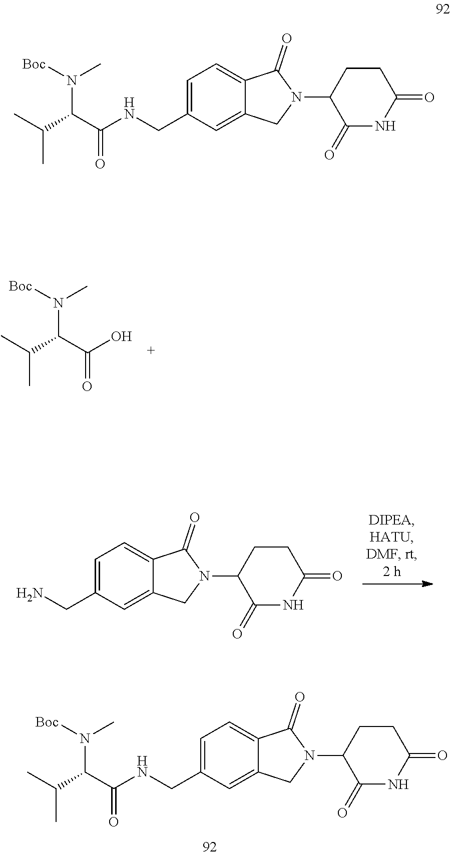 [SMILES: CC(C)[C@@H](C(=O)NCc1ccc2c(c1)CN(C1CCC(=O)NC1=O)C2=O)N(C)C(=O)OC(C)(C)C ; CC(C)[C@@H](C(=O)O)N(C)C(=O)OC(C)(C)C ; NCc1ccc2c(c1)CN(C1CCC(=O)NC1=O)C2=O ; CC(C)[C@@H](C(=O)NCc1ccc2c(c1)CN(C1CCC(=O)NC1=O)C2=O)N(C)C(=O)OC(C)(C)C]

Compound 92 was prepared according to Example 1, Step 5. MS(m/z): 387.0 [M−100]⁺; ¹H NMR (400 MHz, DMSO-d₆) δ10.95 (s, 1H), 8.54 (d, J=66.2 Hz, 1H), 7.64 (d, J=7.2 Hz, 1H), 7.51-7.28 (m, 2H), 5.10 (dd, J=13.3, 5.1 Hz, 1H), 4.48-4.13 (m, 5H), 2.98-2.82 (m, 1H), 2.78 (s, 3H), 2.60 (d, J=17.3 Hz, 1H), 2.45-2.31 (m, 1H), 2.11 (s, 1H), 1.41 (s, 9H), 1.25 (d, J=6.1 Hz, 1H), 0.82 (dd, J=18.4, 5.7 Hz, 6H).

### Example 93 (5-59)

### N-(2-cyclohexyl-3-((2-(2,6-dioxypiperidin-3-yl)-1-oxoisoindoline-5-yl) methyl) amino)-3-oxopropyl) cyclopentyl Formamide

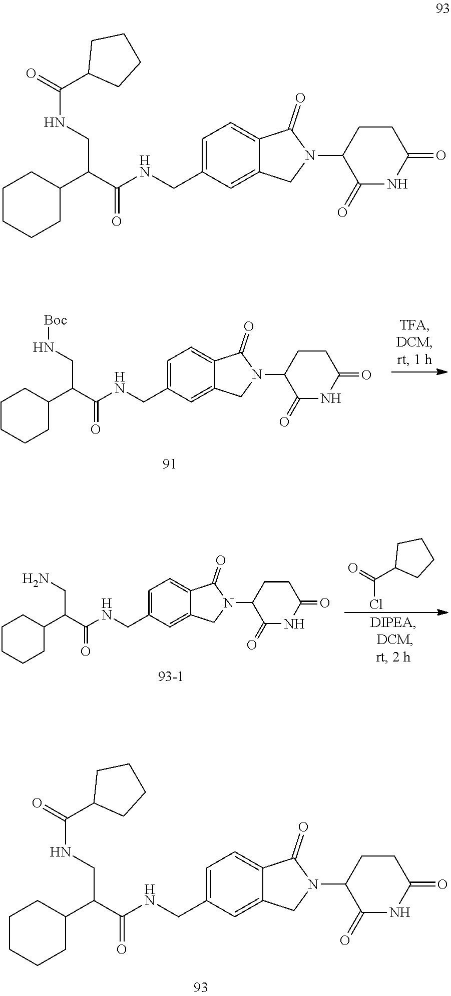

Intermediate 93-1 was prepared according to the method of Step 4, Example 1.

Compound 93 was prepared according to Example 11, Step 2. MS(m/z): 523.0 [M+H]⁺; ¹H NMR (400 MHz, DMSO-d₆) δ10.96 (s, 1H), 8.43 (t, J=5.8 Hz, 1H), 7.66 (dd, J=16.0, 6.0 Hz, 2H), 7.50-7.37 (m, 2H), 5.10 (dd, J=13.2, 5.0 Hz, 1H), 4.55-4.21 (m, 4H), 3.61 (d, J=4.1 Hz, 1H), 3.13 (dd, J=7.3, 4.3 Hz, 1H), 3.06-2.84 (m, 2H), 2.60 (d, J=14.8 Hz, 1H), 2.37 (dd, J=15.1, 10.4 Hz, 1H), 2.04-1.96 (m, 1H), 1.77 (d, J=10.2 Hz, 2H), 1.65 (d, J=8.2 Hz, 3H), 1.56 (t, J=15.5 Hz, 7H), 1.43 (s, 3H), 1.14 (dd, J=25.5, 11.2 Hz, 3H), 1.07-0.92 (m, 2H).

### Example 94 (5-69)

### (2S)-2-(4-chlorophenyl)-2-(2-cyclopentylacetamido)-N-((2-(2,6-dioxypiperidine-3-yl)-1-oxoisoidoline-5-yl) methyl) acetamide

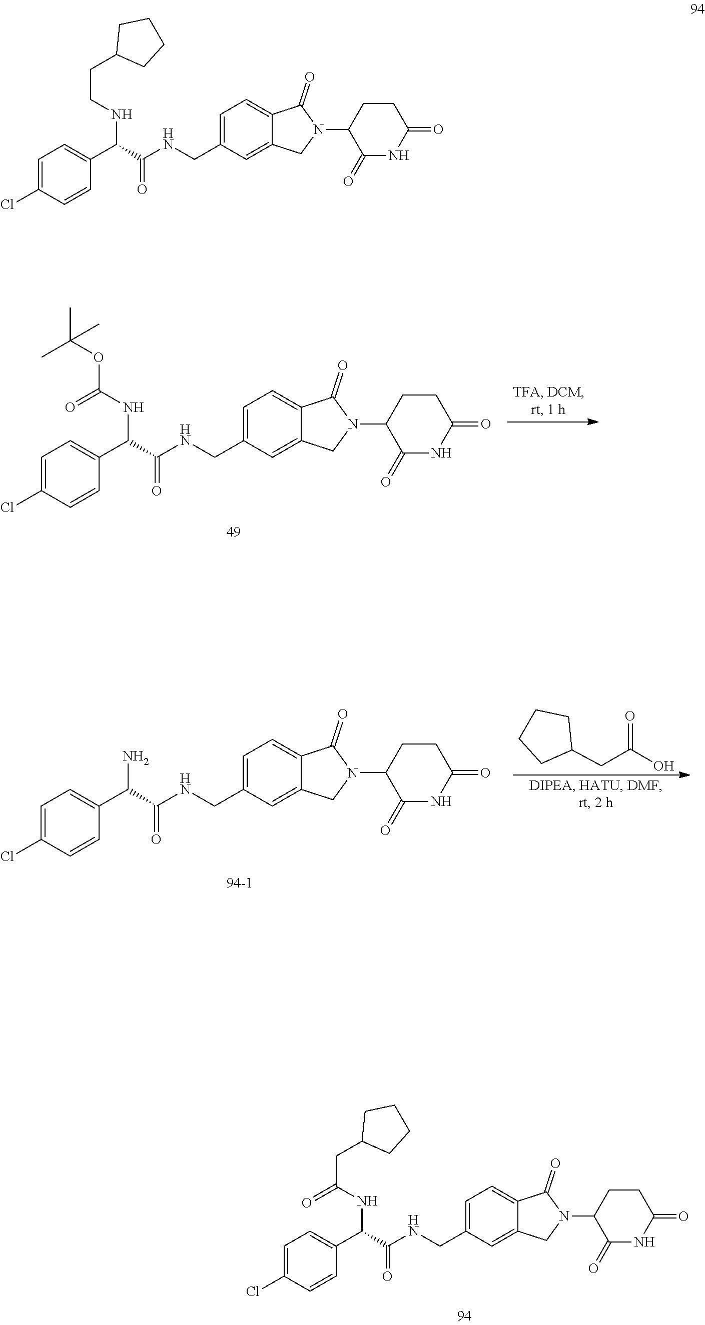

Intermediate 94-1 was prepared according to the method of Step 4, Example 1.

Compound 94 was prepared according to Example 1, Step 5. MS(m/z): 550.8 [M+H]⁺; ¹H NMR (400 MHz, DMSO-d₆) δ10.96 (s, 1H), 8.86 (t, J=5.5 Hz, 1H), 8.48 (d, J=7.9 Hz, 1H), 7.62 (d, J=7.7 Hz, 1H), 7.47-7.40 (m, 4H), 7.30 (d, J=8.4 Hz, 2H), 5.54 (d, J=7.9 Hz, 1H), 5.10 (dd, J=13.2, 4.9 Hz, 1H), 4.42-4.15 (m, 4H), 2.89 (td, J=9.1, 5.3 Hz, 1H), 2.61 (d, J=16.1 Hz, 1H), 2.38 (ddd, J=26.6, 13.3, 4.2 Hz, 1H), 2.21 (d, J=7.3 Hz, 2H), 2.11 (dt, J=15.0, 7.4 Hz, 1H), 2.01 (dd, J=8.9, 3.5 Hz, 1H), 1.71-1.40 (m, 6H), 1.11 (dt, J=11.7, 7.6 Hz, 2H).

### Example 95 (5-70)

### (2S)-2-(2-cyclopentylacetamido)-N-((2-(2,6-dioxopyridin-3-yl)-1-oxoisoidoline-5-yl) methyl)-2-(4-fluorophenyl) acetamide

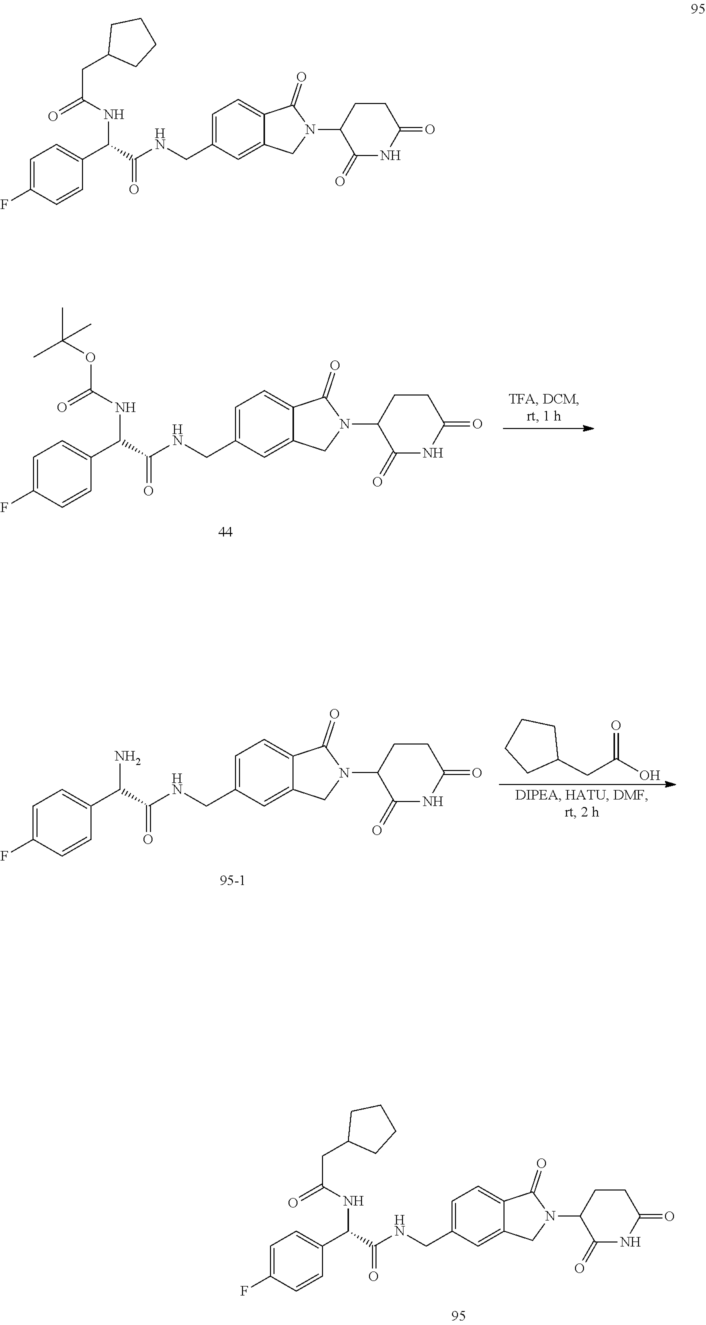

Intermediate 95-1 was prepared according to the method of Step 4, Example 1.

Compound 95 was prepared according to Example 1, Step 5. MS(m/z): 534.9 [M+H]⁺; ¹H NMR (400 MHz, DMSO-d₆) δ10.96 (s, 1H), 8.84 (t, J=5.3 Hz, 1H), 8.46 (d, J=7.9 Hz, 1H), 7.62 (d, J=7.8 Hz, 1H), 7.46 (dd, J=8.6, 5.6 Hz, 2H), 7.35-7.28 (m, 2H), 7.19 (t, J=8.8 Hz, 2H), 5.53 (d, J=7.9 Hz, 1H), 5.09 (dd, J=13.2, 5.0 Hz, 1H), 4.42-4.20 (m, 4H), 2.96-2.82 (m, 1H), 2.60 (d, J=17.5 Hz, 1H), 2.39 (dd, J=13.2, 4.3 Hz, 1H), 2.21 (d, J=6.9 Hz, 2H), 2.12 (dt, J=14.9, 7.3 Hz, 1H), 2.04-1.96 (m, 1H), 1.70-1.41 (m, 6H), 1.11 (dt, J=11.7, 7.8 Hz, 2H).

### Example 96 (4-135)

### Tert butyl ((1S)-2-((2-(2,6-dioxypiperidin-3-yl)-1-oxoisoindoline-5-yl)methyl)amino)-2-oxo-1-phenylethy 1) (methyl) carbamate

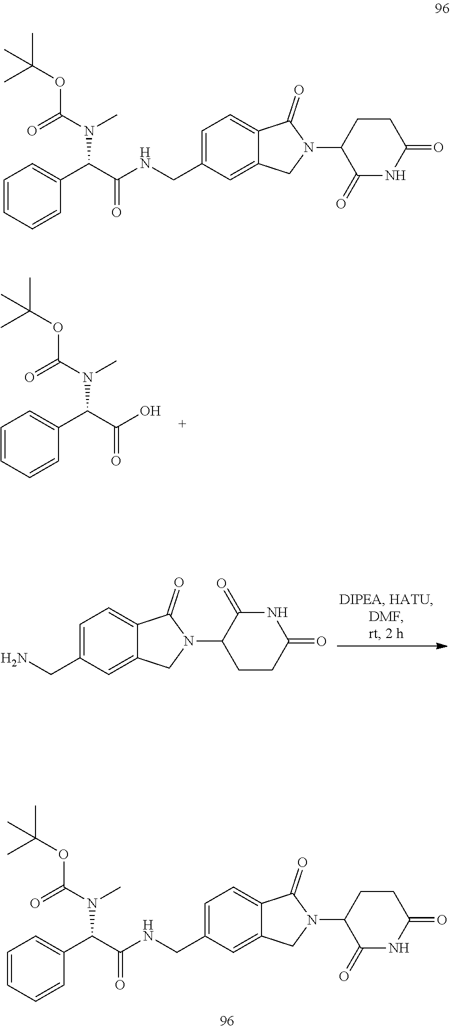 [SMILES: CN(C(=O)OC(C)(C)C)[C@H](C(=O)NCc1ccc2c(c1)CN(C1CCC(=O)NC1=O)C2=O)c1ccccc1 ; CN(C(=O)OC(C)(C)C)[C@H](C(=O)O)c1ccccc1 ; NCc1ccc2c(c1)CN(C1CCC(=O)NC1=O)C2=O ; CN(C(=O)OC(C)(C)C)[C@H](C(=O)NCc1ccc2c(c1)CN(C1CCC(=O)NC1=O)C2=O)c1ccccc1]

Compound 96 was prepared according to Example 1, Step 5. MS(m/z): 421.2 [M−100]⁺; ¹H NMR (400 MHz, DMSO-d₆) δ10.99 (s, 1H), 8.79 (d, J=5.6 Hz, 1H), 7.68 (d, J=7.8 Hz, 1H), 7.48 (s, 1H), 7.38 (dt, J=14.1, 8.3 Hz, 4H), 7.24 (d, J=7.0 Hz, 2H), 5.77 (s, 1H), 5.12 (dd, J=13.2, 5.0 Hz, 1H), 4.37 (ddd, J=21.9, 17.3, 3.9 Hz, 4H), 2.98-2.85 (m, 1H), 2.62 (d, J=15.4 Hz, 4H), 2.47-2.33 (m, 1H), 2.08-1.95 (m, 1H), 1.42 (s, 9H).

### Example 97 (5-31)

### Tert butyl ((1R)-2-((2-(2,6-dioxypiperidin-3-yl)-1-oxoisoindoline-5-yl)methyl)amino)-2-oxo-1-phenylethy 1) (methyl) carbamate

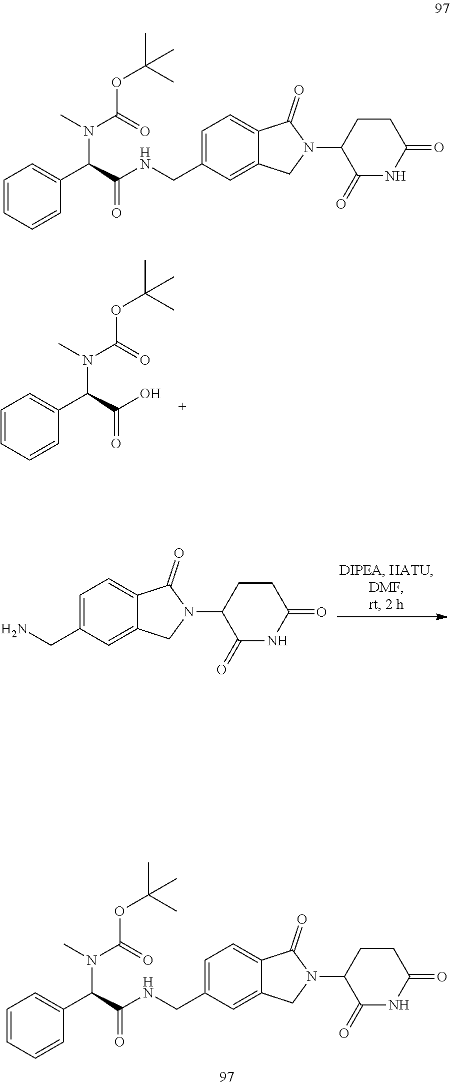 [SMILES: CN(C(=O)OC(C)(C)C)[C@@H](C(=O)NCc1ccc2c(c1)CN(C1CCC(=O)NC1=O)C2=O)c1ccccc1 ; CN(C(=O)OC(C)(C)C)[C@@H](C(=O)O)c1ccccc1 ; NCc1ccc2c(c1)CN(C1CCC(=O)NC1=O)C2=O ; CN(C(=O)OC(C)(C)C)[C@@H](C(=O)NCc1ccc2c(c1)CN(C1CCC(=O)NC1=O)C2=O)c1ccccc1]

Compound 97 was prepared according to Example 1, Step 5. MS(m/z): 421.0 [M−100]⁺; ¹H NMR (400 MHz, DMSO-d₆) δ10.96 (s, 1H), 8.77 (t, J=5.6 Hz, 1H), 7.66 (d, J=7.8 Hz, 1H), 7.46 (s, 1H), 7.37 (dt, J=14.2, 8.9 Hz, 4H), 7.23 (d, J=7.1 Hz, 2H), 5.75 (s, 1H), 5.10 (dd, J=13.3, 5.0 Hz, 1H), 4.36 (ddd, J=21.4, 17.2, 3.7 Hz, 4H), 2.91 (ddd, J=13.7, 11.3, 5.3 Hz, 1H), 2.62 (s, 1H), 2.59 (s, 3H), 2.46-2.35 (m, 1H), 2.06-1.94 (m, 1H), 1.41 (s, 9H).

### Example 98 (5-34)

### N-((1S)-2-(2-(2,6-dioxypiperidin-3-yl)-1-oxoisoidoline-5-yl)methyl)amino)-2-oxo-1-phenylethyl)-N, 3,3-trimethylbutylamide

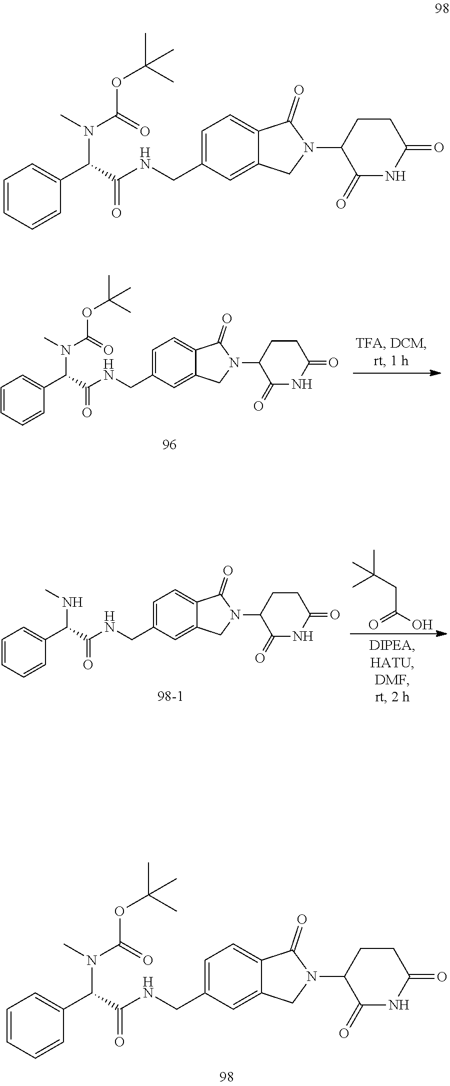

Intermediate 98-1 was prepared according to the method of Step 4, Example 1.

Compound 98 was prepared according to Example 1, Step 5. MS(m/z): 541.3 [M+H]⁺; ¹H NMR (400 MHz, DMSO-d₆) δ10.96 (s, 1H), 8.78 (s, 1H), 7.66 (d, J=7.8 Hz, 1H), 7.47 (s, 1H), 7.44-7.29 (m, 4H), 7.20 (d, J=6.9 Hz, 2H), 6.28 (s, 1H), 5.10 (dd, J=13.2, 5.0 Hz, 1H), 4.56-4.24 (m, 4H), 2.91 (ddd, J=13.6, 11.5, 5.4 Hz, 1H), 2.75 (d, J=14.5 Hz, 3H), 2.60 (d, J=16.4 Hz, 1H), 2.40 (dd, J=13.0, 4.3 Hz, 1H), 2.30 (s, 2H), 2.05-1.95 (m, 1H), 1.02 (s, 9H).

### Example 99 (5-35)

### Isopropyl ((1S)-2-((2-(2,6-dioxypiperidin-3-yl)-1-oxoisoindoline-5-yl)methyl)amino)-2-oxo-1-phenylethy 1) (methyl) carbamate

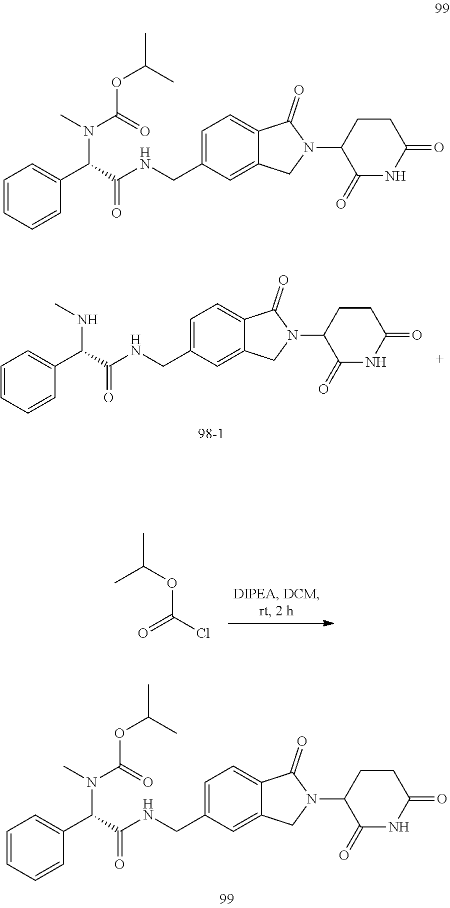

Compound 99 was prepared according to Example 11, Step 2. MS(m/z): 506.9 [M+H]⁺; ¹H NMR (400 MHz, DMSO-d₆) δ10.96 (s, 1H), 8.80 (s, 1H), 7.67 (d, J=7.8 Hz, 1H), 7.55-7.32 (m, 5H), 7.23 (d, J=6.9 Hz, 2H), 5.81 (s, 1H), 5.11 (dd, J=13.2, 4.9 Hz, 1H), 4.83 (dt, J=12.3, 6.2 Hz, 1H), 4.36 (ddd, J=52.8, 17.1, 4.5 Hz, 4H), 3.00-2.82 (m, 1H), 2.62 (s, 3H), 2.58 (s, 1H), 2.41 (dt, J=13.1, 9.0 Hz, 1H), 2.05-1.94 (m, 1H), 1.29-1.08 (m, 6H).

### Example 100 (5-36)

### Cyclopentyl ((1S)-2-((2-(2,6-dioxypiperidin-3-yl)-1-oxoisoindoline-5-yl)methyl)amino)-2-oxo-1-phenylethyl) (methyl) carbamate

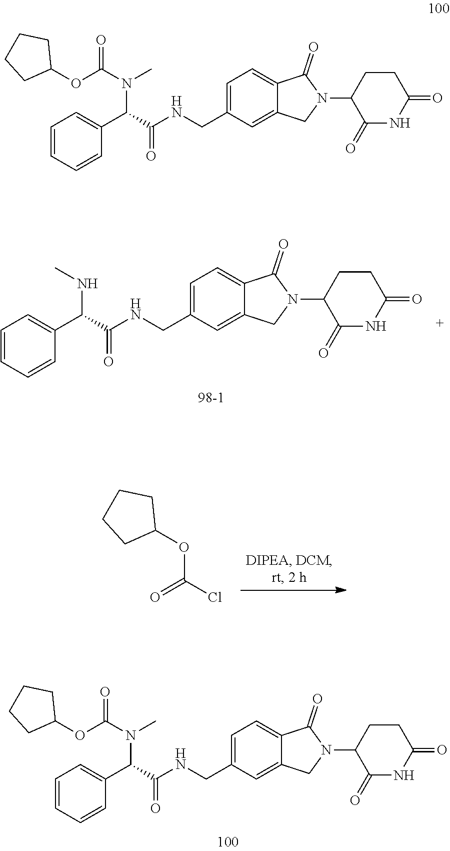 [SMILES: CN(C(=O)OC1CCCC1)[C@H](C(=O)NCc1ccc2c(c1)CN(C1CCC(=O)NC1=O)C2=O)c1ccccc1 ; CN[C@H](C(=O)NCc1ccc2c(c1)CN(C1CCC(=O)NC1=O)C2=O)c1ccccc1 ; O=C(Cl)OC1CCCC1 ; CN(C(=O)OC1CCCC1)[C@H](C(=O)NCc1ccc2c(c1)CN(C1CCC(=O)NC1=O)C2=O)c1ccccc1]

Compound 100 was prepared according to Example 11, Step 2. MS(m/z): 533.3 [M+H]⁺; ¹H NMR (400 MHz, DMSO-d₆) δ10.96 (s, 1H), 8.79 (t, J=5.4 Hz, 1H), 7.66 (d, J=7.8 Hz, 1H), 7.45 (s, 1H), 7.37 (dt, J=20.3, 7.1 Hz, 4H), 7.23 (d, J=7.1 Hz, 2H), 5.75 (s, 1H), 5.21-4.95 (m, 2H), 4.36 (ddd, J=52.5, 16.9, 5.1 Hz, 4H), 3.02-2.85 (m, 1H), 2.61 (s, 3H), 2.59-2.56 (m, 1H), 2.40 (ddd, J=26.3, 13.1, 4.3 Hz, 1H), 2.05-1.97 (m, 1H), 1.79 (s, 2H), 1.69-1.48 (m, 6H).

### Example 101 (5-40)

### (2S)-2-(3-(tert butyl)-1-methylurea)-N-((2-(2,6-dioxopyridin-3-yl)-1-oxoisoidoline-5-yl) methyl)-2-phenylacetamide

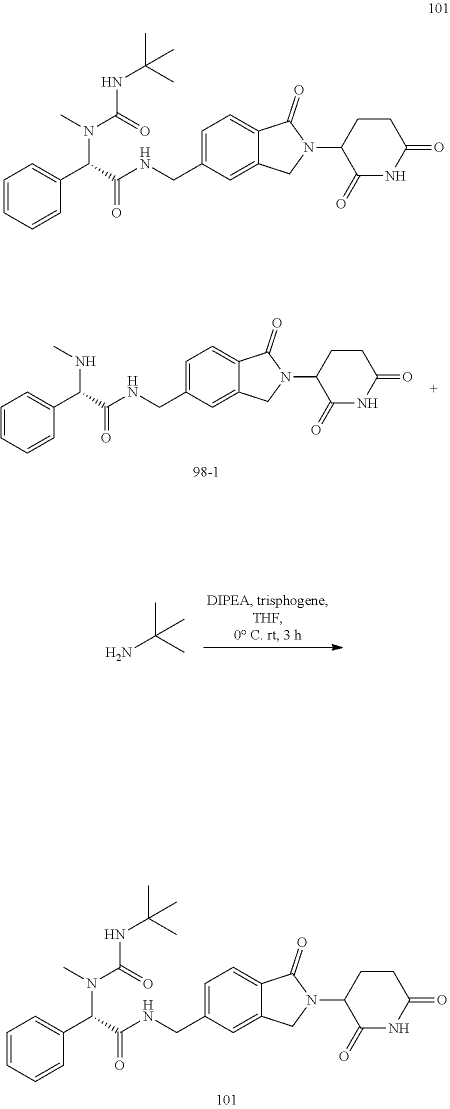

Compound 101 was prepared according to Example 19. MS(m/z): 519.9 [M+H]⁺

### Example 102 (5-75)

### Tert butyl ((1S)-2-((2-(2,6-dioxypiperidin-3-yl)-1-oxoisoidoline-5-yl)methyl)amino)-1-(4-fluorophenyl)-2-oxoethyl) (methyl) carbamate

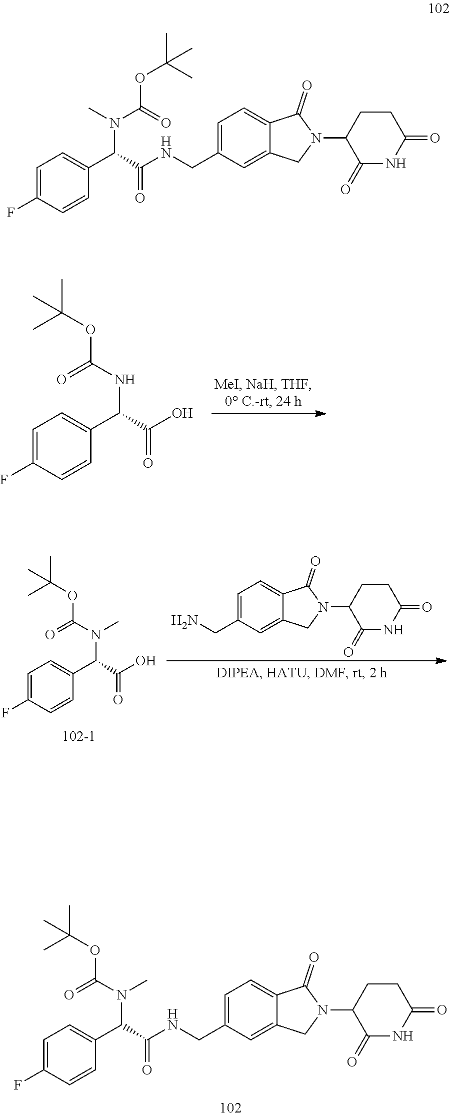

### Step 1: Preparation of Intermediate (S)-2-((tert-butoxycarbonyl)(methyl)amino)-2-(4-fluorophenyl)acetic Acid (102-1)

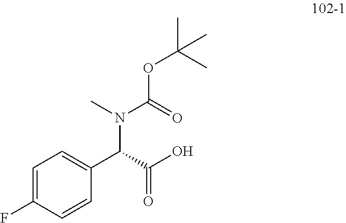 [SMILES: CN(C(=O)OC(C)(C)C)[C@H](C(=O)O)c1ccc(F)cc1]

(S)-2-((tert-butoxycarbonyl)amino)-2-(4-fluorophenyl)acetic acid (100.0 mg, 0.4 mmol) was dissolved in anhydrous tetrahydrofuran (5 mL), stirred in ice bath, and sodium hydride (65.0 mg, 1.9 mmol) was added slowly, the reaction was carried out for 1 h. Methyl iodide (264.0 mg, 1.9 mmol) was added, and the reaction was carried out for 24 h at room temperature. At the end of the reaction, water (10 mL) was added to the system and pH=3 was adjusted with citric acid solution, extracted with ethyl acetate (30 mL×3), the organic phases were combined, washed with saturated sodium chloride solution (30 mL×3), dried with anhydrous sodium sulfate, filtered and concentrated under reduced pressure to obtain the intermediate 102-1, which was directly used without purification for the next step of the reaction.

### Step 2: Preparation of tert butyl ((1S)-2-((2-(2,6-dioxypiperidin-3-yl)-1-oxoisoidoline-5-yl)methyl)amino)-1-(4-fluorophenyl)-2-oxoethyl) (methyl) carbamate (102)

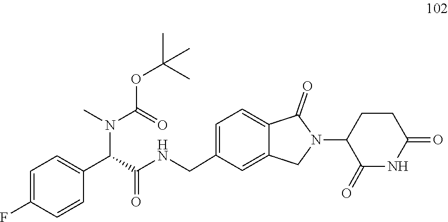 [SMILES: CN(C(=O)OC(C)(C)C)[C@H](C(=O)NCc1ccc2c(c1)CN(C1CCC(=O)NC1=O)C2=O)c1ccc(F)cc1]

Compound 102 was prepared according to Example 1, Step 5. MS(m/z): 438.9 [M−100]⁺

### Example 103 (5-76)

### Tert butyl (1-(3,4-dichlorophenyl)-2-((2-(2,6-dioxopyridin-3-yl)-1-oxoisoidoline-5-yl)methyl) amino)-2-oxoethyl (methyl) carbamate

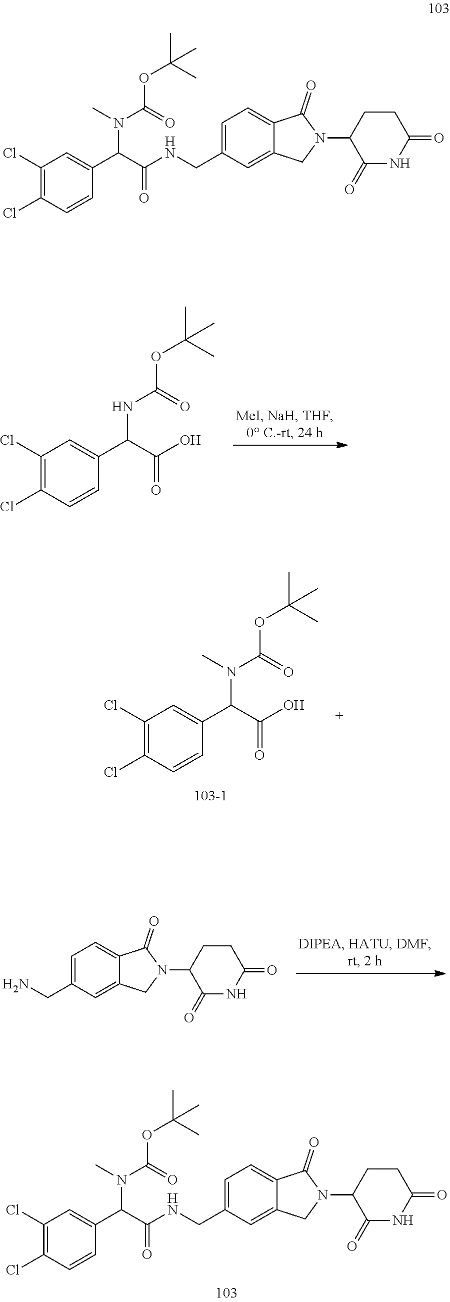 [SMILES: CN(C(=O)OC(C)(C)C)C(C(=O)NCc1ccc2c(c1)CN(C1CCC(=O)NC1=O)C2=O)c1ccc(Cl)c(Cl)c1 ; CC(C)(C)OC(=O)NC(C(=O)O)c1ccc(Cl)c(Cl)c1 ; CN(C(=O)OC(C)(C)C)C(C(=O)O)c1ccc(Cl)c(Cl)c1 ; NCc1ccc2c(c1)CN(C1CCC(=O)NC1=O)C2=O ; CN(C(=O)OC(C)(C)C)C(C(=O)NCc1ccc2c(c1)CN(C1CCC(=O)NC1=O)C2=O)c1ccc(Cl)c(Cl)c1]

Intermediate 103-1 was prepared according to the method of Step 1, Example 102.

Compound 103 was prepared according to Example 1, Step 5. MS(m/z): 488.8 [M−100]⁺

### Example 104 (5-77)

### Tert butyl (2-((2-(2,6-dioxypiperidin-3-yl)-1-oxoisoindoline-5-yl) amino)-2-oxo-1-(p-tolyl) ethyl) (methyl) carbamate

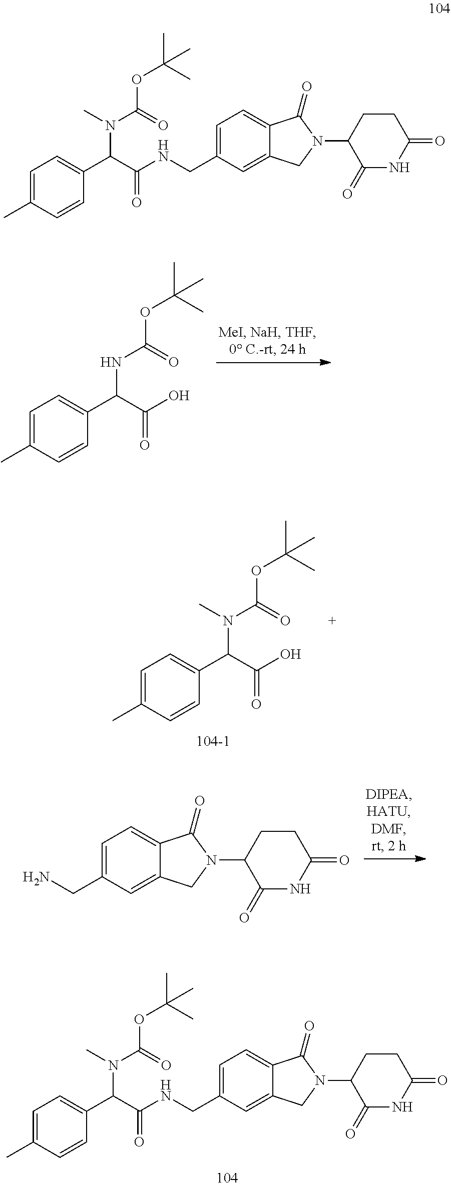 [SMILES: Cc1ccc(C(C(=O)NCc2ccc3c(c2)CN(C2CCC(=O)NC2=O)C3=O)N(C)C(=O)OC(C)(C)C)cc1 ; Cc1ccc(C(NC(=O)OC(C)(C)C)C(=O)O)cc1 ; Cc1ccc(C(C(=O)O)N(C)C(=O)OC(C)(C)C)cc1 ; NCc1ccc2c(c1)CN(C1CCC(=O)NC1=O)C2=O ; Cc1ccc(C(C(=O)NCc2ccc3c(c2)CN(C2CCC(=O)NC2=O)C3=O)N(C)C(=O)OC(C)(C)C)cc1]

Intermediate 104-1 was prepared according to the method of Step 1, Example 102.

Compound 104 was prepared according to Example 1, Step 5. MS(m/z): 435.0 [M−100]⁺; ¹H NMR (400 MHz, DMSO-d₆) δ10.95 (s, 1H), 8.71 (t, J=5.4 Hz, 1H), 7.66 (d, J=7.8 Hz, 1H), 7.50-7.34 (m, 2H), 7.15 (dd, J=33.1, 7.1 Hz, 4H), 5.72 (d, J=21.9 Hz, 1H), 5.10 (dd, J=13.3, 5.1 Hz, 1H), 4.50-4.18 (m, 4H), 2.98-2.84 (m, 1H), 2.62 (s, 1H), 2.57 (s, 3H), 2.46-2.34 (m, 1H), 2.30 (s, 3H), 2.05-1.95 (m, 1H), 1.40 (s, 9H).

### Example 105 (5-78)

### Tert butyl ((1S)-1-(4-chlorophenyl)-2-((2-(2,6-dioxopyridin-3-yl)-1-oxoisoidoline-5-yl) methyl) amino)-2-oxoethyl (methyl) carbamate

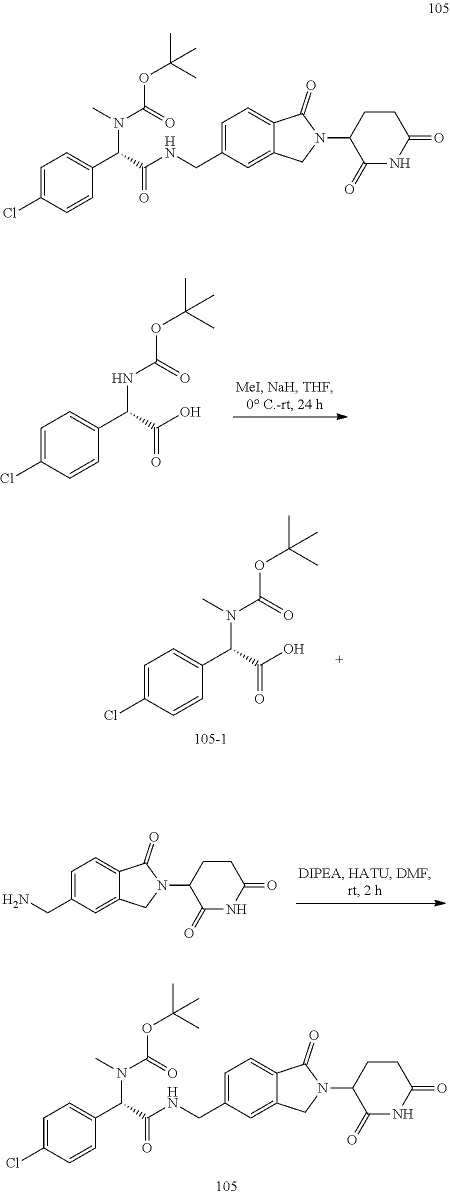 [SMILES: CN(C(=O)OC(C)(C)C)[C@H](C(=O)NCc1ccc2c(c1)CN(C1CCC(=O)NC1=O)C2=O)c1ccc(Cl)cc1 ; CC(C)(C)OC(=O)N[C@H](C(=O)O)c1ccc(Cl)cc1 ; CN(C(=O)OC(C)(C)C)[C@H](C(=O)O)c1ccc(Cl)cc1 ; NCc1ccc2c(c1)CN(C1CCC(=O)NC1=O)C2=O ; CN(C(=O)OC(C)(C)C)[C@H](C(=O)NCc1ccc2c(c1)CN(C1CCC(=O)NC1=O)C2=O)c1ccc(Cl)cc1]

Intermediate 105-1 was prepared according to the method of Step 1, Example 102.

Compound 105 was prepared according to Example 1, Step 5. MS(m/z): 454.9 [M−100]⁺; ¹H NMR (400 MHz, DMSO-d₆) δ10.96 (s, 1H), 8.79 (t, J=5.5 Hz, 1H), 7.67 (d, J=7.8 Hz, 1H), 7.43 (dd, J=20.2, 8.1 Hz, 4H), 7.25 (d, J=8.3 Hz, 2H), 5.75 (s, 1H), 5.10 (dd, J=13.2, 5.0 Hz, 1H), 4.52-4.20 (m, 4H), 2.98-2.80 (m, 1H), 2.61 (s, 4H), 2.40 (dd, J=13.1, 4.4 Hz, 1H), 2.13-1.90 (m, 1H), 1.40 (s, 9H).

### Example 106 (5-79)

### Tert butyl (2-((2-(2,6-dioxypiperidin-3-yl)-1-oxoisoidoline-5-yl)amino)-1-(4-methoxyphenyl)-2-oxoethyl) (methyl) carbamate

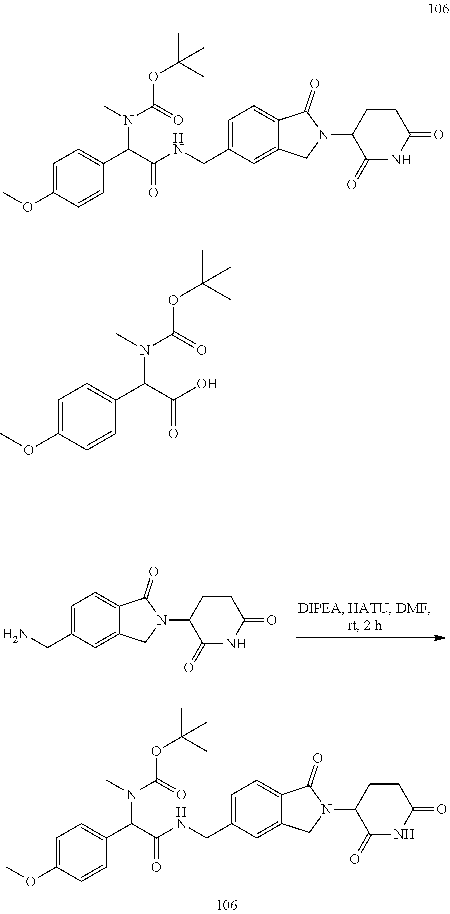 [SMILES: COc1ccc(C(C(=O)NCc2ccc3c(c2)CN(C2CCC(=O)NC2=O)C3=O)N(C)C(=O)OC(C)(C)C)cc1 ; COc1ccc(C(C(=O)O)N(C)C(=O)OC(C)(C)C)cc1 ; NCc1ccc2c(c1)CN(C1CCC(=O)NC1=O)C2=O ; COc1ccc(C(C(=O)NCc2ccc3c(c2)CN(C2CCC(=O)NC2=O)C3=O)N(C)C(=O)OC(C)(C)C)cc1]

Compound 106 was prepared according to Example 1, Step 5. MS(m/z): 450.9 [M−100]⁺; ¹H NMR (400 MHz, DMSO-d₆) δ10.97 (s, 1H), 8.70 (t, J=5.7 Hz, 1H), 7.66 (d, J=7.8 Hz, 1H), 7.53-7.30 (m, 2H), 7.16 (d, J=8.4 Hz, 2H), 6.94 (d, J=8.3 Hz, 2H), 5.75 (s, 1H), 5.11 (dd, J=13.2, 5.0 Hz, 1H), 4.54-4.16 (m, 4H), 3.76 (s, 3H), 3.00-2.80 (m, 1H), 2.62 (s, 1H), 2.57 (s, 3H), 2.46-2.32 (m, 1H), 2.05-1.94 (m, 1H), 1.40 (s, 9H).

### Example 107 (1-86)

### Tert butyl ((1S)-1-cyclohexyl-2-(2-(2,6-dioxopyridin-3-yl)-1-oxoisoidoline-5-yl) methyl) amino)-2-oxoethyl (methyl) carbamate

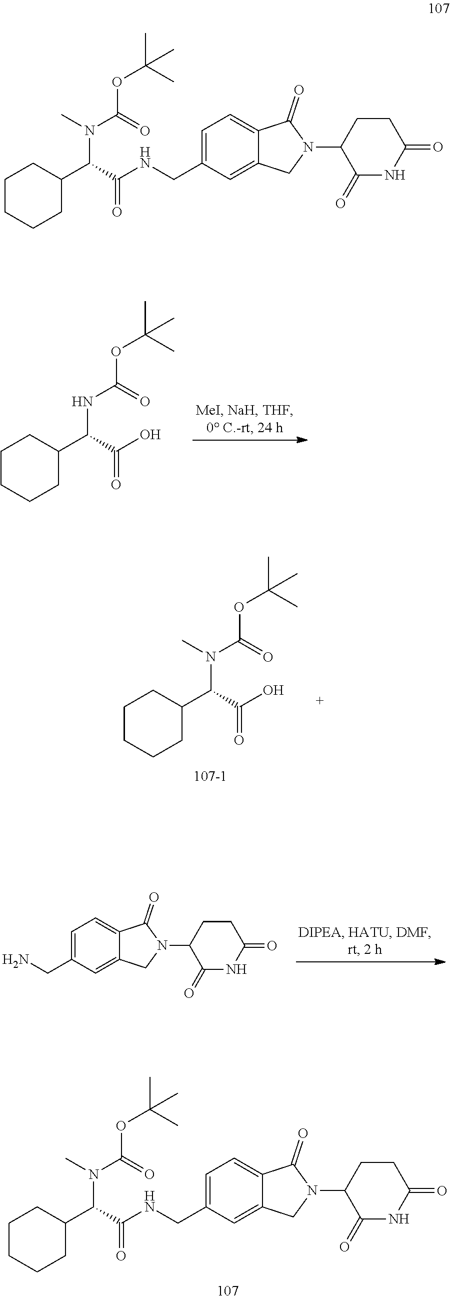 [SMILES: CN(C(=O)OC(C)(C)C)[C@H](C(=O)NCc1ccc2c(c1)CN(C1CCC(=O)NC1=O)C2=O)C1CCCCC1 ; CC(C)(C)OC(=O)N[C@H](C(=O)O)C1CCCCC1 ; CN(C(=O)OC(C)(C)C)[C@H](C(=O)O)C1CCCCC1 ; NCc1ccc2c(c1)CN(C1CCC(=O)NC1=O)C2=O ; CN(C(=O)OC(C)(C)C)[C@H](C(=O)NCc1ccc2c(c1)CN(C1CCC(=O)NC1=O)C2=O)C1CCCCC1]

Intermediate 107-1 was prepared according to the method of Step 1, Example 102.

Compound 107 was prepared according to Example 1, Step 5. MS(m/z): 426.2 [M−100]⁺; ¹H NMR (400 MHz, DMSO-d₆) δ10.96 (s, 1H), 8.66 (s, 1H), 8.16 (s, 1H), 7.64 (d, J=7.4 Hz, 1H), 7.43 (s, 1H), 7.37 (d, J=7.8 Hz, 1H), 5.75 (s, 1H), 5.10 (dd, J=13.3, 5.1 Hz, 1H), 4.42-4.22 (m, 4H), 2.91 (ddd, J=17.6, 12.3, 5.2 Hz, 1H), 2.77 (s, 3H), 2.60 (d, J=17.1 Hz, 1H), 2.45-2.31 (m, 1H), 2.04-1.94 (m, 1H), 1.82 (s, 1H), 1.63 (d, J=26.1 Hz, 4H), 1.49 (d, J=11.5 Hz, 3H), 1.41 (s, 9H), 0.90 (dd, J=28.8, 10.4 Hz, 2H).

### Example 108 (1-89)

### Tert butyl ((1S)-2-((2-(2,6-dioxypiperidin-3-yl)-1-oxoisoindoline-5-yl)methyl)amino)-2-oxo-1-phenylethy 1) (ethyl) carbamate

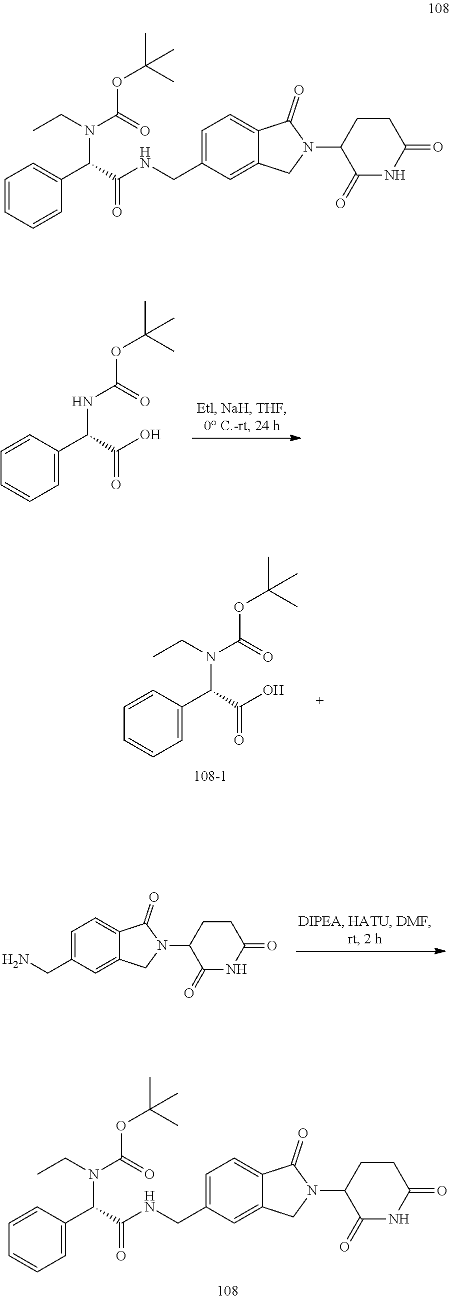 [SMILES: CCN(C(=O)OC(C)(C)C)[C@H](C(=O)NCc1ccc2c(c1)CN(C1CCC(=O)NC1=O)C2=O)c1ccccc1 ; CC(C)(C)OC(=O)N[C@H](C(=O)O)c1ccccc1 ; CCN(C(=O)OC(C)(C)C)[C@H](C(=O)O)c1ccccc1 ; NCc1ccc2c(c1)CN(C1CCC(=O)NC1=O)C2=O ; CCN(C(=O)OC(C)(C)C)[C@H](C(=O)NCc1ccc2c(c1)CN(C1CCC(=O)NC1=O)C2=O)c1ccccc1]

Intermediate 108-1 was prepared according to the method of Step 1, Example 102.

Compound 108 was prepared according to Example 1, Step 5. MS(m/z): 434.2 [M−100]

### Example 109 (5-50)

### 2-cyclopentyl-N-((1S)-2-((2-(2,6-dioxopyridin-3-yl)-1-oxoisoindoline-5-yl) methyl) amino)-2-oxo-1-phenylethyl)-N-methylacetamide

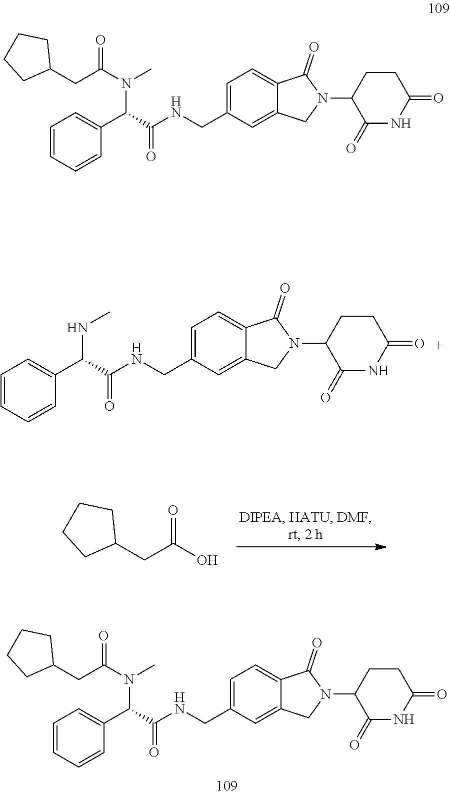 [SMILES: CN(C(=O)CC1CCCC1)[C@H](C(=O)NCc1ccc2c(c1)CN(C1CCC(=O)NC1=O)C2=O)c1ccccc1 ; CN[C@H](C(=O)NCc1ccc2c(c1)CN(C1CCC(=O)NC1=O)C2=O)c1ccccc1 ; O=C(O)CC1CCCC1 ; CN(C(=O)CC1CCCC1)[C@H](C(=O)NCc1ccc2c(c1)CN(C1CCC(=O)NC1=O)C2=O)c1ccccc1]

Compound 109 was prepared according to Example 1, Step 5. MS(m/z): 530.9 [M+H]⁺; ¹H NMR (400 MHz, DMSO-d₆) δ10.96 (s, 1H), 8.78 (t, J=5.4 Hz, 1H), 7.67 (d, J=7.7 Hz, 1H), 7.37 (ddd, J=22.1, 20.9, 13.7 Hz, 5H), 7.20 (t, J=9.1 Hz, 2H), 6.26 (s, 1H), 5.11 (dd, J=13.2, 5.0 Hz, 1H), 4.36 (ddd, J=22.3, 16.5, 5.3 Hz, 4H), 2.98-2.85 (m, 1H), 2.76 (s, 3H), 2.60 (d, J=16.2 Hz, 1H), 2.47-2.31 (m, 3H), 2.25-2.14 (m, 1H), 2.06-1.96 (m, 1H), 1.77 (d, J=4.7 Hz, 2H), 1.61-1.41 (m, 4H), 1.27-1.08 (m, 2H).

### Example 110 (5-49)

### N-((1S)-2-((2-(2,6-dioxypiperidin-3-yl)-1-oxoisoindoline-5-yl)methyl)amino)-2-oxo-1-phenylethyl)-N-methylcyclopentyl Formamide

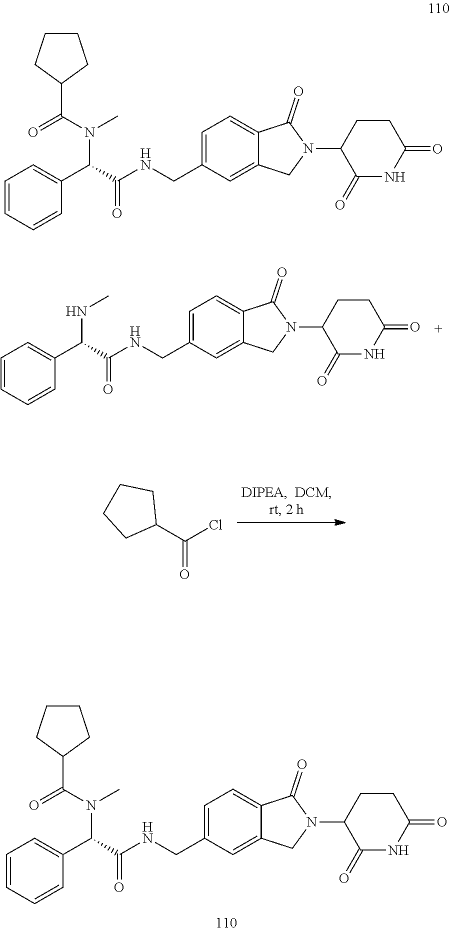 [SMILES: CN(C(=O)C1CCCC1)[C@H](C(=O)NCc1ccc2c(c1)CN(C1CCC(=O)NC1=O)C2=O)c1ccccc1 ; CN[C@H](C(=O)NCc1ccc2c(c1)CN(C1CCC(=O)NC1=O)C2=O)c1ccccc1 ; O=C(Cl)C1CCCC1 ; CN(C(=O)C1CCCC1)[C@H](C(=O)NCc1ccc2c(c1)CN(C1CCC(=O)NC1=O)C2=O)c1ccccc1]

Compound 110 was prepared according to Example 11, Step 2. MS(m/z): 516.8 [M+H]⁺; ¹H NMR (400 MHz, DMSO-d₆) δ10.96 (s, 1H), 8.77 (t, J=5.6 Hz, 1H), 7.66 (d, J=7.8 Hz, 1H), 7.51-7.28 (m, 5H), 7.21 (dd, J=19.9, 6.6 Hz, 2H), 6.24 (s, 1H), 5.75 (s, 1H), 5.11 (dd, J=13.2, 5.0 Hz, 1H), 4.55-4.22 (m, 4H), 3.10-2.99 (m, 1H), 2.79 (s, 3H), 2.60 (d, J=18.2 Hz, 1H), 2.40 (dd, J=13.1, 4.2 Hz, 1H), 2.06-1.96 (m, 1H), 1.88-1.45 (m, 8H).

### Example 111 (5-88)

### Isopropyl (1S)-2-((2-(2,6-dioxypiperidin-3-yl)-1-oxoisoindoline-5-yl)methyl)amino)-2-oxo-1-phenylethyl carbonate

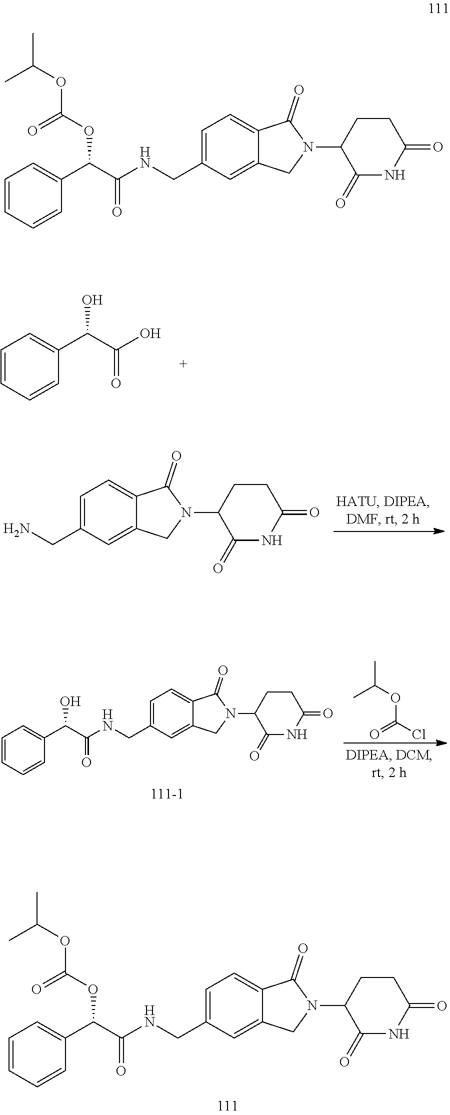

Intermediate 111-1 was prepared according to the method of Step 5, Example 1.

Compound 111 was prepared according to Example 11, Step 2. MS(m/z): 494.2 [M+H]⁺; ¹H NMR (400 MHz, DMSO-d₆) δ10.96 (s, 1H), 8.94 (t, J=5.9 Hz, 1H), 8.17 (s, 1H), 7.61 (d, J=8.1 Hz, 1H), 7.49 (dd, J=6.4, 2.7 Hz, 2H), 7.40 (dd, J=5.0, 1.8 Hz, 2H), 7.29 (d, J=5.7 Hz, 2H), 5.83 (s, 1H), 5.09 (dd, J=13.2, 5.0 Hz, 1H), 4.30 (ddd, J=21.5, 20.0, 4.8 Hz, 4H), 3.63 (dd, J=6.5, 3.9 Hz, 1H), 2.90 (ddd, J=13.7, 12.4, 5.4 Hz, 1H), 2.60 (d, J=17.0 Hz, 1H), 2.45-2.35 (m, 1H), 2.06-1.95 (m, 1H), 1.27 (s, 6H).

### Example 112 (5-82)

### Tert butyl ((1S)-2-((2-(2,6-dioxypiperidin-3-yl)-1-oxoisoindoline-5-yl)methyl)amino)-2-oxo-1-phenylethy 1) carbonate

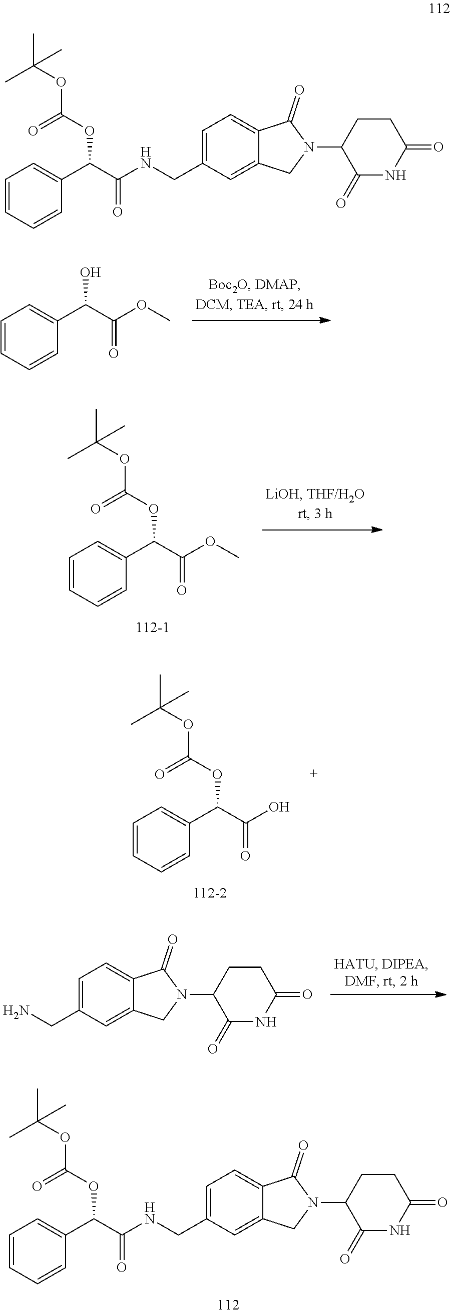 [SMILES: CC(C)(C)OC(=O)O[C@H](C(=O)NCc1ccc2c(c1)CN(C1CCC(=O)NC1=O)C2=O)c1ccccc1 ; COC(=O)[C@@H](O)c1ccccc1 ; COC(=O)[C@@H](OC(=O)OC(C)(C)C)c1ccccc1 ; CC(C)(C)OC(=O)O[C@H](C(=O)O)c1ccccc1 ; NCc1ccc2c(c1)CN(C1CCC(=O)NC1=O)C2=O ; CC(C)(C)OC(=O)O[C@H](C(=O)NCc1ccc2c(c1)CN(C1CCC(=O)NC1=O)C2=O)c1ccccc1]

### Step 1: Preparation of the Intermediate methyl (S)-2-((tert-butoxycarbonyl)oxy)-2-phenylacetate(112-1)

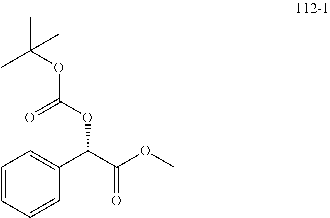 [SMILES: COC(=O)[C@@H](OC(=O)OC(C)(C)C)c1ccccc1]

Methyl (S)-2-hydroxy-2-phenylacetate (200.0 mg, 1.2 mmol), di-tert-butyl dicarbonate (394.0 mg, 1.8 mmol), triethylamine (364.0 mg, 3.6 mmol), and 4-dimethylaminopyridine (146.0 mg, 1.2 mmol) were dissolved in dichloromethane (10 mL) and the reaction was carried out at room temperature for 24 hours. At the end of the reaction, water (20 mL) was added to the system, extracted with dichloromethane (30 mL×3), the organic phases were combined, washed with saturated sodium chloride solution (30 mL×3), dried with anhydrous sodium sulfate, filtered and concentrated under reduced pressure to give Intermediate 112-1, which was directly used in the next step of the reaction without purification.

### Step 2: Preparation of Intermediate (S)-2-((tert-butoxycarbonyl)oxy)-2-phenylacetic Acid (112-2)

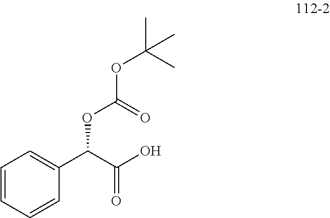 [SMILES: CC(C)(C)OC(=O)O[C@H](C(=O)O)c1ccccc1]

Intermediate 112-1 was dissolved in a mixture of tetrahydrofuran and water (2:1), lithium hydroxide monohydrate (151.0 mg, 3.6 mmol) was added and hydrolysed for 3 hours at room temperature. At the end of the reaction, the pH of the system was adjusted to 3 with 1N hydrochloric acid solution, extracted with ethyl acetate (30 mL×3), the organic phases were combined, washed with saturated sodium chloride solution (30 mL×3), dried with anhydrous sodium sulfate, filtered and concentrated under reduced pressure to give intermediate 112-2, which was directly used in the next step of the reaction without purification.

### Step 3: Preparation of tert butyl ((1S)-2-((2-(2,6-dioxypiperidin-3-yl)-1-oxoisoindoline-5-yl)methyl)amino)-2-oxo-1-phenylethyl) carbonate(112)

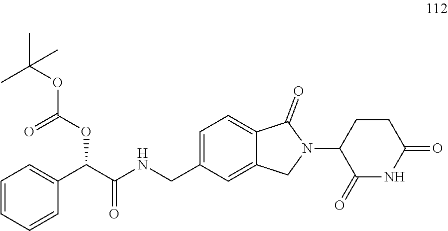 [SMILES: CC(C)(C)OC(=O)O[C@H](C(=O)NCc1ccc2c(c1)CN(C1CCC(=O)NC1=O)C2=O)c1ccccc1]

Compound 112 was prepared according to Example 1, Step 5. MS(m/z): 408.2 [M+H]⁺; ¹H NMR (400 MHz, DMSO-d₆) δ10.95 (s, 1H), 8.91 (t, J=5.9 Hz, 1H), 7.60 (d, J=8.1 Hz, 1H), 7.48 (dd, J=6.4, 2.8 Hz, 2H), 7.40 (dd, J=5.0, 1.9 Hz, 3H), 7.29 (d, J=6.8 Hz, 2H), 5.76 (d, J=7.8 Hz, 1H), 5.09 (dd, J=13.2, 4.8 Hz, 1H), 4.48-4.15 (m, 4H), 2.89 (s, 1H), 2.60 (d, J=17.1 Hz, 1H), 2.45-2.33 (m, 1H), 2.06-1.93 (m, 1H), 1.43 (s, 9H).

### Example 113 (5-87)

### Cyclopentyl ((1S)-2-((2-(2,6-dioxypiperidin-3-yl)-1-oxoisoindoline-5-yl)methyl)amino)-2-oxo-1-phenylethy 1) carbonate

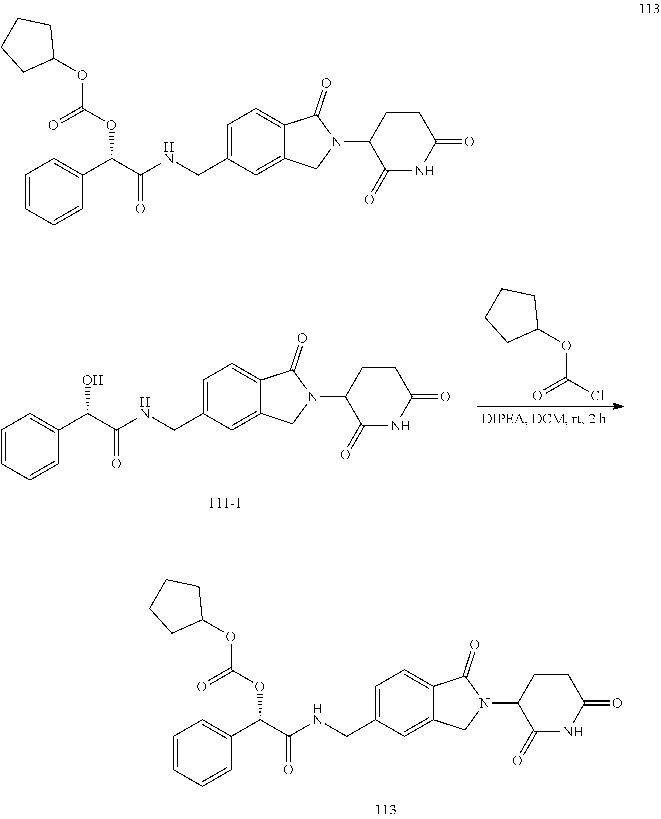 [SMILES: O=C1CCC(N2Cc3cc(CNC(=O)[C@@H](OC(=O)OC4CCCC4)c4ccccc4)ccc3C2=O)C(=O)N1 ; O=C1CCC(N2Cc3cc(CNC(=O)[C@@H](O)c4ccccc4)ccc3C2=O)C(=O)N1 ; O=C(Cl)OC1CCCC1 ; O=C1CCC(N2Cc3cc(CNC(=O)[C@@H](OC(=O)OC4CCCC4)c4ccccc4)ccc3C2=O)C(=O)N1]

Compound 113 was prepared according to Example 11, Step 2. MS(m/z): 520.2 [M+H]⁺; ¹H NMR (400 MHz, DMSO-d₆) δ10.97 (s, 1H), 8.94 (t, J=5.7 Hz, 1H), 8.17 (s, 2H), 7.61 (d, J=8.1 Hz, 1H), 7.48 (d, J=4.1 Hz, 2H), 7.41 (d, J=4.7 Hz, 2H), 7.29 (d, J=6.0 Hz, 1H), 5.75 (s, 1H), 5.09 (dd, J=13.5, 5.1 Hz, 1H), 4.31 (dd, J=43.5, 12.0 Hz, 4H), 3.62 (dd, J=10.3, 6.5 Hz, 2H), 2.97-2.84 (m, 1H), 2.60 (d, J=17.7 Hz, 1H), 2.44-2.35 (m, 1H), 2.06-1.93 (m, 1H), 1.85 (s, 2H), 1.73-1.52 (m, 5H).

### Example 114 (5-83)

### Tert butyl ((2R)-3-(2-chlorophenyl)-1-((2-(2,6-dioxopyridin-3-yl)-1-oxoisoidoline-5-yl) methyl) amino)-1-oxopropan-2-yl) carbamate

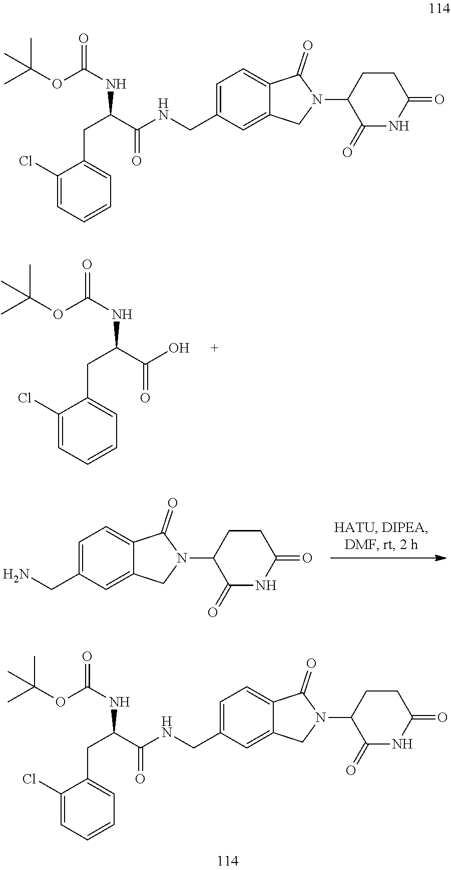

Compound 114 was prepared according to Example 1, Step 5. MS(m/z): 454.2 [M−100]⁺; ¹H NMR (400 MHz, DMSO-d₆) δ10.95 (s, 1H), 8.44 (s, 1H), 7.63 (d, J=7.7 Hz, 1H), 7.50-7.20 (m, 6H), 7.04 (d, J=8.3 Hz, 1H), 5.10 (dd, J=13.3, 5.0 Hz, 1H), 4.48-4.22 (m, 5H), 3.62 (qd, J=10.5, 6.4 Hz, 2H), 2.93-2.86 (m, 1H), 2.60 (d, J=17.0 Hz, 1H), 2.41 (dd, J=13.2, 4.0 Hz, 1H), 2.08-1.93 (m, 1H), 1.27 (d, J=5.9 Hz, 9H).

### Example 115 (5-85)

### Isopropyl ((2R)-1-((2-(2,6-dioxypiperidin-3-yl)-1-oxoisoindoline-5-yl)methyl)amino)-4-methyl-1-oxapent-2-yl) carbamate

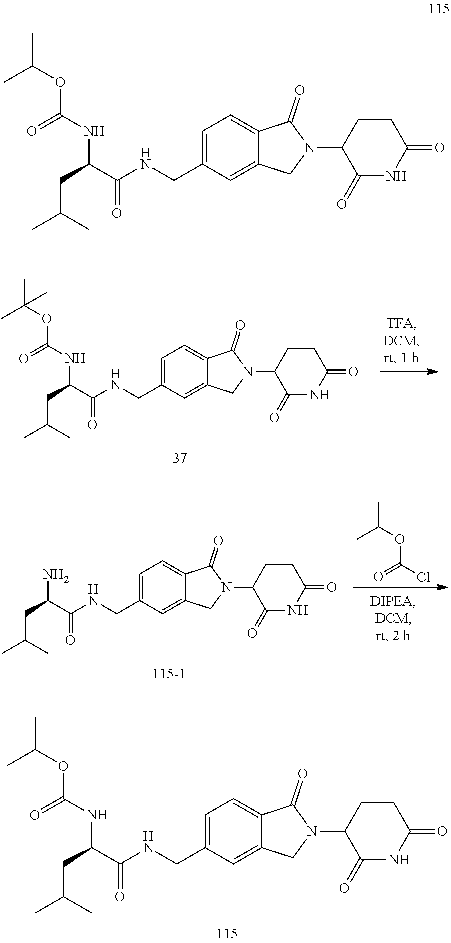

Intermediate 115-1 was prepared according to the method of Step 4, Example 1.

Compound 115 was prepared according to Example 11, Step 2. MS(m/z): 473.3 [M+H]⁺; ¹H NMR (400 MHz, DMSO-d₆) δ10.95 (s, 1H), 8.45 (t, J=5.8 Hz, 1H), 7.66 (d, J=7.9 Hz, 1H), 7.46-7.32 (m, 2H), 7.11 (d, J=8.0 Hz, 1H), 5.10 (dd, J=13.2, 5.0 Hz, 1H), 4.74 (dt, J=12.6, 6.3 Hz, 1H), 4.50-4.20 (m, 4H), 4.09-3.98 (m, 1H), 3.00-2.82 (m, 1H), 2.59 (d, J=17.3 Hz, 1H), 2.42-2.32 (m, 1H), 2.04-1.96 (m, 1H), 1.71-1.35 (m, 3H), 1.31-1.04 (m, 6H), 0.87 (dd, J=10.8, 6.6 Hz, 6H).

### Example 116 (5-84)

### Tert butyl ((2S)-1-((2-(2,6-dioxypiperidin-3-yl)-1-oxoisoidoline-5-yl)methyl)amino)-4-methyl-1-oxapent-2-yl) carbamate

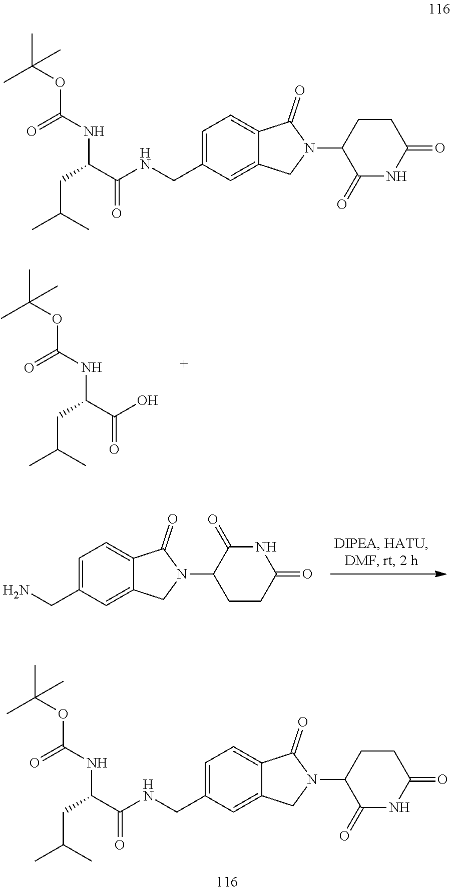

Compound 116 was prepared according to Example 1, Step 5. MS(m/z): 387.0[M−100]⁺; ¹H NMR (400 MHz, DMSO-d₆) δ10.96 (s, 1H), 8.43 (t, J=5.7 Hz, 1H), 7.64 (d, J=7.8 Hz, 1H), 7.55-7.30 (m, 2H), 6.91 (d, J=8.0 Hz, 1H), 5.10 (dd, J=13.3, 5.0 Hz, 1H), 4.50-4.20 (m, 4H), 2.97-2.85 (m, 1H), 2.60 (d, J=16.7 Hz, 1H), 2.39 (dd, J=13.0, 4.2 Hz, 1H), 2.08-1.93 (m, 1H), 1.66-1.53 (m, 1H), 1.52-1.29 (m, 11H), 1.28-1.19 (m, 1H), 0.87 (dd, J=11.1, 6.6 Hz, 6H).

### Example 117 (5-92)

### (2S)-3-cyclohexyl-2-(2-cyclopentylacetamido)-N-((2-(2,6-dioxypiperidin-3-yl)-1-oxoisoindoline-5-yl) methyl) propionamide

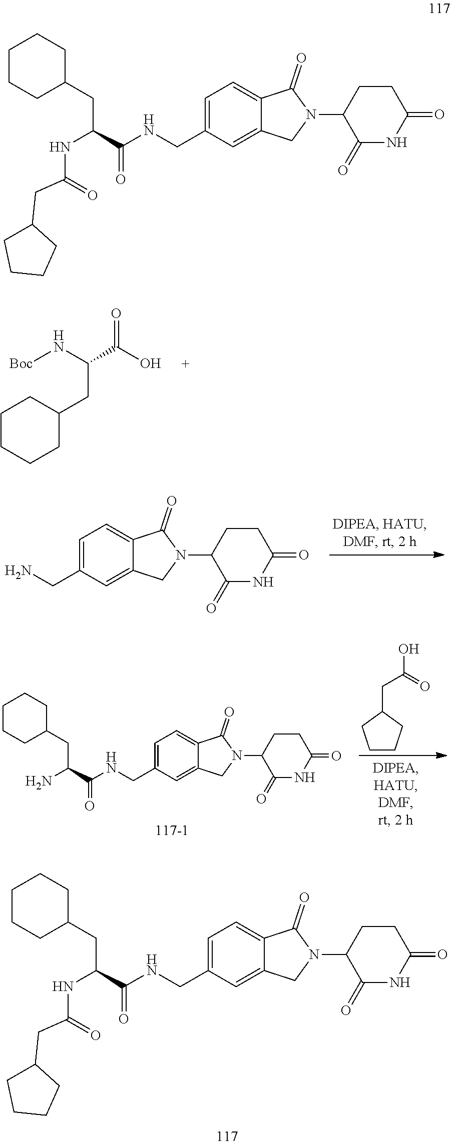

Intermediate 117-1 was prepared according to the method of Step 5, Example 1.

Compound 117 was prepared according to Example 1, Step 5. MS(m/z): 536.3 [M+H]⁺; ¹H NMR (400 MHz, DMSO-d₆) δ10.96 (s, 1H), 8.48 (t, J=5.9 Hz, 1H), 7.92 (d, J=8.2 Hz, 1H), 7.63 (t, J=16.7 Hz, 1H), 7.50-7.28 (m, 2H), 5.10 (dd, J=13.2, 5.0 Hz, 1H), 4.54-4.20 (m, 5H), 3.00-2.82 (m, 1H), 2.60 (d, J=16.8 Hz, 1H), 2.40 (dd, J=13.1, 4.3 Hz, 1H), 2.17-2.08 (m, 3H), 2.00 (dd, J=9.0, 3.5 Hz, 1H), 1.69 (d, J=34.2 Hz, 6H), 1.59-1.53 (m, 2H), 1.51-1.40 (m, 4H), 1.24 (d, J=5.4 Hz, 2H), 1.11 (s, 5H), 0.98-0.75 (m, 2H).

### Example 118 (5-93)

### Cyclopentyl ((2S)-3-cyclohexyl-1-((2-(2,6-dioxopyridin-3-yl)-1-oxoisoidoline-5-yl) methyl) amino)-1-oxopropan-2-yl) carbamate

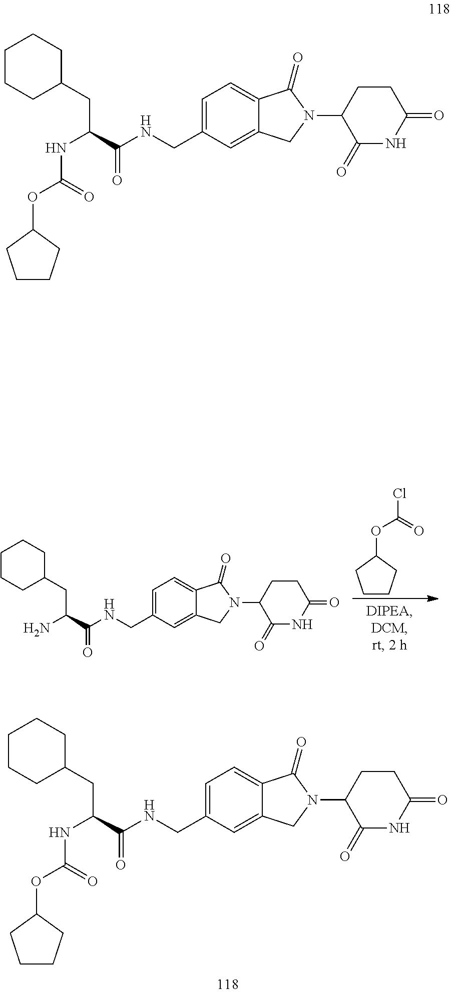

Compound 118 was prepared according to Example 11, Step 2. MS(m/z): 539.3 [M+H]⁺; ¹H NMR (400 MHz, DMSO-d₆) δ10.95 (s, 1H), 8.44 (t, J=5.9 Hz, 1H), 7.65 (d, J=7.8 Hz, 1H), 7.45 (s, 1H), 7.38 (d, J=7.9 Hz, 1H), 7.09 (d, J=7.9 Hz, 1H), 5.10 (dd, J=13.2, 5.0 Hz, 1H), 4.94 (s, 1H), 4.36 (dt, J=39.7, 17.2 Hz, 4H), 2.97-2.85 (m, 1H), 2.60 (d, J=17.6 Hz, 1H), 2.40 (ddd, J=26.2, 13.2, 4.2 Hz, 1H), 2.05-1.94 (m, 1H), 1.78 (s, 2H), 1.69-1.53 (m, 10H), 1.45 (s, 3H), 1.26 (s, 1H), 1.11 (s, 3H), 0.87 (dd, J=25.6, 14.3 Hz, 2H).

### Example 119 (5-94)

### Isopropyl ((2S)-3-cyclohexyl-1-((2-(2,6-dioxypiperidin-3-yl)-1-oxoisoindolin-5-yl) methyl) amino)-1-oxopropan-2-yl) carbamate

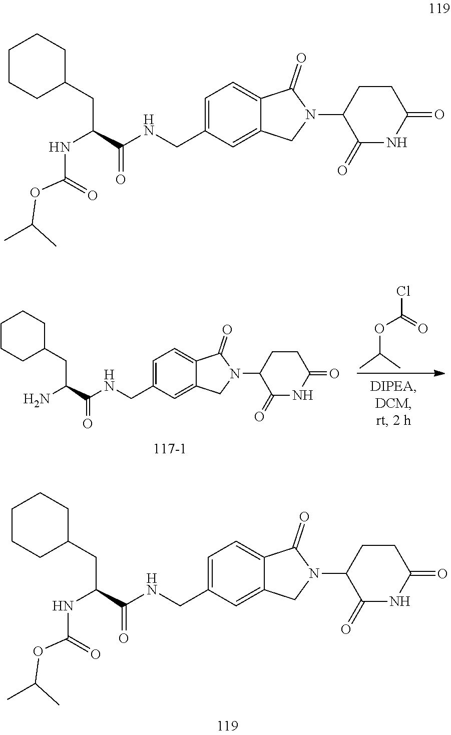

Compound 119 was prepared according to Example 11, Step 2. MS(m/z): 513.3[M+H]⁺; ¹H NMR (400 MHz, DMSO-d₆) δ10.95 (s, 1H), 8.45 (t, J=5.7 Hz, 1H), 7.65 (d, J=7.8 Hz, 1H), 7.58-7.26 (m, 2H), 7.09 (d, J=8.0 Hz, 1H), 5.75 (s, 1H), 5.10 (dd, J=13.2, 5.1 Hz, 1H), 4.87-4.66 (m, 1H), 4.48-4.22 (m, 4H), 3.00-2.82 (m, 1H), 2.60 (d, J=17.5 Hz, 1H), 2.40 (dd, J=13.1, 4.3 Hz, 1H), 1.99 (dd, J=6.7, 3.9 Hz, 1H), 1.64 (t, J=13.7 Hz, 5H), 1.45 (s, 2H), 1.34-0.97 (m, 10H), 0.96-0.77 (m, 2H).

### Example 120 (5-95)

### N-((2S)-3-cyclohexyl-1-((2-(2,6-dioxypiperidin-3-yl)-1-oxoisoindolin-5-yl) methyl) amino)-1-oxopropan-2-yl) cyclopentyl Formamide

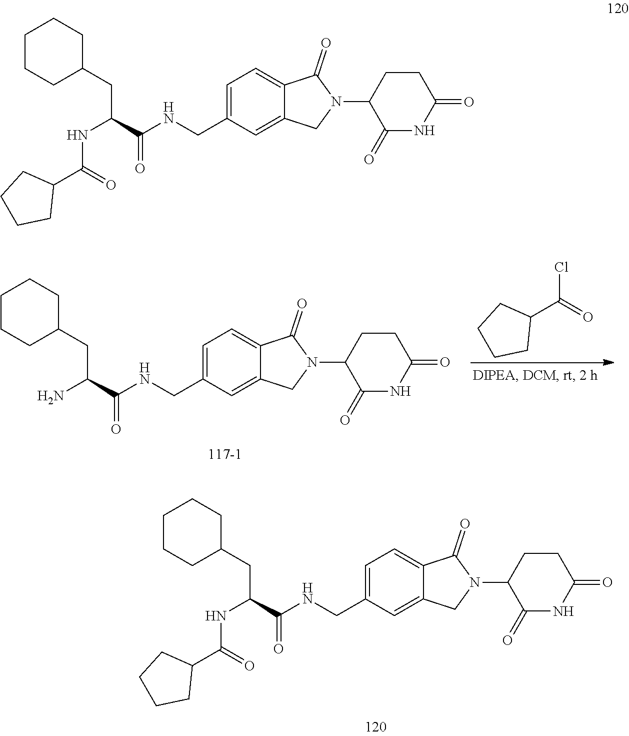 [SMILES: O=C1CCC(N2Cc3cc(CNC(=O)[C@H](CC4CCCCC4)NC(=O)C4CCCC4)ccc3C2=O)C(=O)N1 ; N[C@@H](CC1CCCCC1)C(=O)NCc1ccc2c(c1)CN(C1CCC(=O)NC1=O)C2=O ; O=C(Cl)C1CCCC1 ; O=C1CCC(N2Cc3cc(CNC(=O)[C@H](CC4CCCCC4)NC(=O)C4CCCC4)ccc3C2=O)C(=O)N1]

Compound 120 was prepared according to Example 11, Step 2. MS(m/z): 522.3 [M+H]⁺; ¹H NMR (400 MHz, DMSO-d₆) δ10.96 (s, 1H), 8.71 (t, J=5.8 Hz, 1H), 8.32 (s, 1H), 7.93 (d, J=3.3 Hz, 1H), 7.60 (d, J=7.7 Hz, 1H), 7.38-7.20 (m, 6H), 5.09 (dd, J=13.3, 5.0 Hz, 1H), 4.56-4.18 (m, 4H), 3.91-3.84 (m, 1H), 3.62 (dq, J=10.6, 6.6 Hz, 2H), 2.99-2.84 (m, 1H), 2.60 (d, J=16.4 Hz, 1H), 2.39 (dd, J=13.2, 4.3 Hz, 1H), 2.06-1.93 (m, 1H), 1.69-1.40 (m, 9H).

### Example 121 (5-97)

### Tert butyl ((3R,4S)-1-((2-(2,6-dioxypiperidin-3-yl)-1-oxoisoidoline-5-yl)methyl)amino)-4-methyl-1-oxohexane-3-yl) carbamate

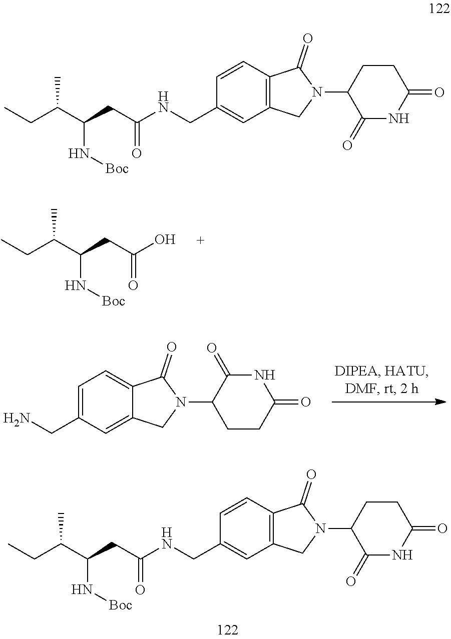 [SMILES: CC[C@H](C)[C@@H](CC(=O)NCc1ccc2c(c1)CN(C1CCC(=O)NC1=O)C2=O)NC(=O)OC(C)(C)C ; CC[C@H](C)[C@@H](CC(=O)O)NC(=O)OC(C)(C)C ; NCc1ccc2c(c1)CN(C1CCC(=O)NC1=O)C2=O ; CC[C@H](C)[C@@H](CC(=O)NCc1ccc2c(c1)CN(C1CCC(=O)NC1=O)C2=O)NC(=O)OC(C)(C)C]

Compound 122 was prepared according to Example 1, Step 5. MS(m/z): 401.3 [M−100]⁺; ¹H NMR (400 MHz, DMSO-d₆) δ10.95 (s, 1H), 8.30 (s, 1H), 7.65 (d, J=7.8 Hz, 1H), 7.51-7.32 (m, 2H), 6.58 (d, J=8.5 Hz, 1H), 5.10 (dd, J=13.2, 5.0 Hz, 1H), 4.36 (dt, J=31.4, 17.2 Hz, 4H), 3.76 (s, 1H), 2.97-2.84 (m, 1H), 2.60 (d, J=17.3 Hz, 1H), 2.44-2.32 (m, 1H), 2.32-2.15 (m, 2H), 2.04-1.95 (m, 1H), 1.53-1.18 (m, 12H), 1.04 (d, J=6.9 Hz, 1H), 0.83 (t, J=7.1 Hz, 3H), 0.78 (d, J=6.6 Hz, 2H).

### Example 122 (5-98)

### Tert butyl ((1S,2S)-2-((2-(2,6-dioxopyridin-3-yl)-1-oxoidoline-5-yl) methyl) carbamoyl) cyclohexyl) carbamate

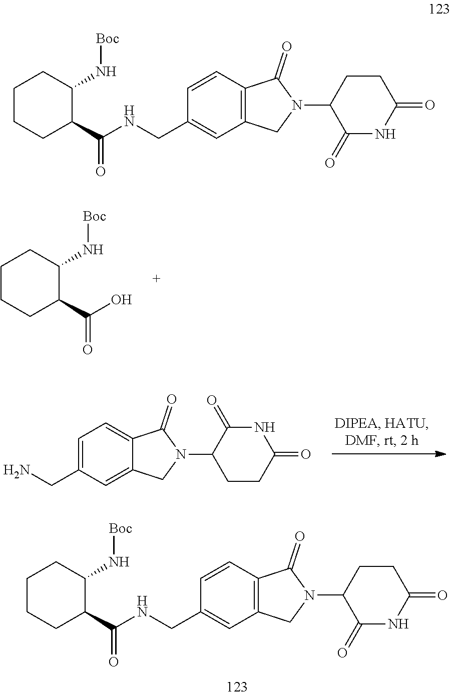

Compound 123 was prepared according to Example 1, Step 5. MS(m/z): 399.2 [M−100]; ¹H NMR (400 MHz, DMSO-d₆) δ10.95 (s, 1H), 8.05 (s, 1H), 7.63 (d, J=7.8 Hz, 1H), 7.51-7.32 (m, 2H), 6.53 (d, J=8.3 Hz, 1H), 5.10 (dd, J=13.2, 5.0 Hz, 1H), 4.54-4.24 (m, 4H), 3.42 (d, J=7.8 Hz, 1H), 3.04-2.82 (m, 1H), 2.60 (d, J=16.9 Hz, 1H), 2.41-2.29 (m, 1H), 2.22 (t, J=9.7 Hz, 1H), 2.03-1.96 (m, 1H), 1.79 (dd, J=28.9, 11.2 Hz, 2H), 1.64 (s, 2H), 1.51-1.40 (m, 1H), 1.34 (s, 9H), 1.29-1.07 (m, 4H).

### Example 123 (5-99)

### Tert butyl (1-cyclopentyl-3-((2-(2,6-dioxypiperidin-3-yl)-1-oxoisoindoline-5-yl) methyl) amino)-3-oxopropyl) carbamate

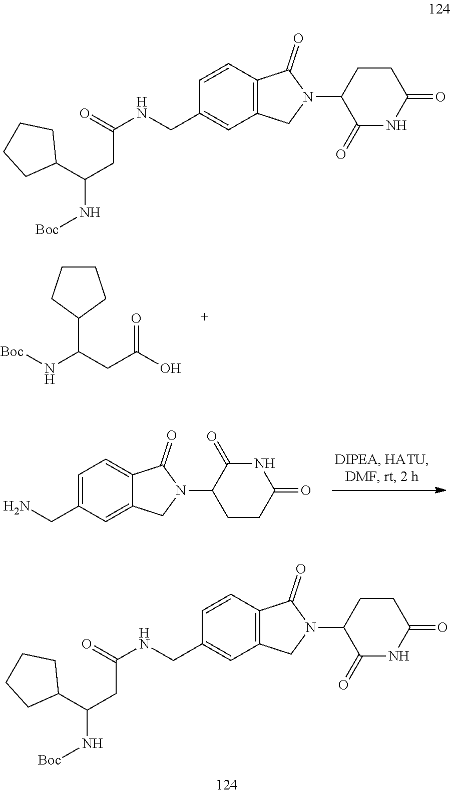

Compound 124 was prepared according to Example 1, Step 5. MS(m/z): 413.2[M−100]⁺; ¹H NMR (400 MHz, DMSO-d₆) δ10.96 (s, 1H), 8.36 (t, J=5.6 Hz, 1H), 7.64 (d, J=7.8 Hz, 1H), 7.53-7.32 (m, 2H), 6.63 (d, J=9.3 Hz, 1H), 5.10 (dd, J=13.3, 5.1 Hz, 1H), 4.48-4.28 (m, 4H), 3.74 (dd, J=14.4, 7.5 Hz, 1H), 2.97-2.85 (m, 1H), 2.60 (d, J=17.2 Hz, 1H), 2.39 (ddd, J=27.3, 13.7, 4.8 Hz, 1H), 2.32-2.21 (m, 2H), 2.08-1.95 (m, 1H), 1.55 (d, J=4.7 Hz, 5H), 1.47-1.38 (m, 3H), 1.35 (s, 9H), 1.21-1.11 (m, 1H).

### Example 124 (6-01)

### ((1S)-2-(((2-(2,6-dioxopyridin-3-yl)-1-oxoisoindoline-5-yl)methyl)amino)-2-oxo-1-phenylethyl) carbamate-β-Oxocyclopentanol Ester

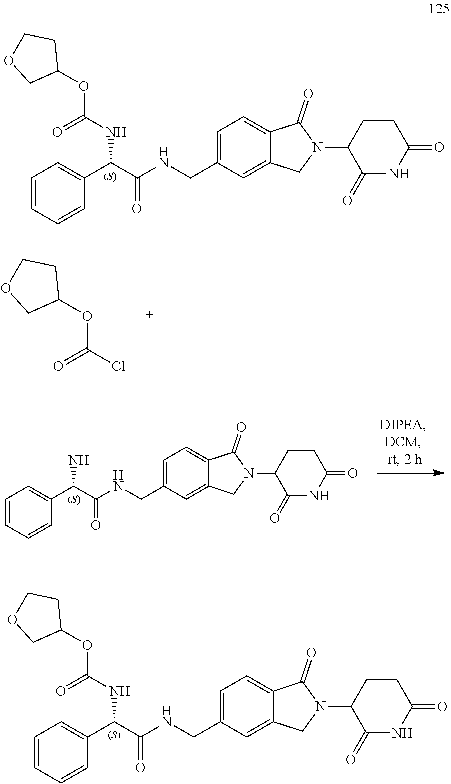

Compound 125 was prepared according to Example 11, Step 2. MS(m/z): 521.3 [M+H]; ¹H NMR (400 MHz, DMSO-d₆) δ10.99 (s, 1H), 8.79 (s, 1H), 7.85 (s, 1H), 7.60 (d, J=7.7 Hz, 1H), 7.44 (d, J=7.5 Hz, 2H), 7.37-7.25 (m, 5H), 5.25 (d, J=7.8 Hz, 1H), 5.17-5.05 (m, 2H), 4.42-4.18 (m, 3H), 3.82-3.60 (m, 5H), 2.98-2.85 (m, 1H), 2.59 (d, J=18.3 Hz, 1H), 2.47-2.34 (m, 1H), 2.10 (s, 1H), 2.03-1.93 (m, 1H), 1.86 (s, 1H)

### Example 125 (6-02)

### ((1S)-2-(((2-(2,6-dioxypiperidin-3-yl)-1-oxoisoindoline-5-yl)methyl)amino)-2-oxo-1-phen ylethyl) carbamate Cyclohexanol Ester

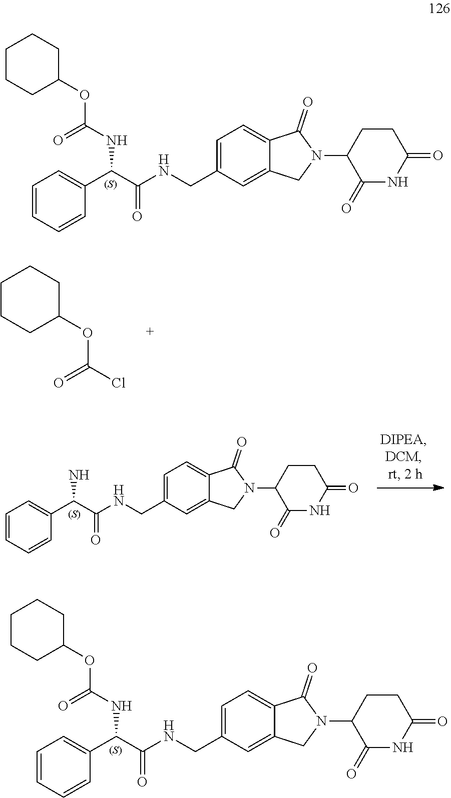

Compound 126 was prepared according to Example 11, Step 2. MS(m/z): 533.3 [M+H]⁺; ¹H NMR (400 MHz, DMSO-d₆) δ10.98 (s, 1H), 8.76 (s, 1H), 7.60 (d, J=7.9 Hz, 1H), 7.44 (d, J=7.0 Hz, 2H), 7.39-7.22 (m, 5H), 5.25 (s, 1H), 5.09 (dd, J=13.1, 4.8 Hz, 1H), 4.50 (s, 1H), 4.41-4.19 (m, 4H), 2.96-2.85 (m, 1H), 2.59 (d, J=17.5 Hz, 1H), 2.40 (d, J=12.8 Hz, 1H), 2.00 (s, 1H), 1.80 (s, 2H), 1.67 (s, 2H), 1.47 (s, 1H), 1.40-1.12 (m, 6H)

### Example 126 (6-03)

### ((1S)-2-(((2-(2,6-dioxopyridin-3-yl)-1-oxoisoindoline-5-yl)methyl)amino)-2-oxo-1-phenylethyl) carbamate-γ-Oxyhexanol Ester

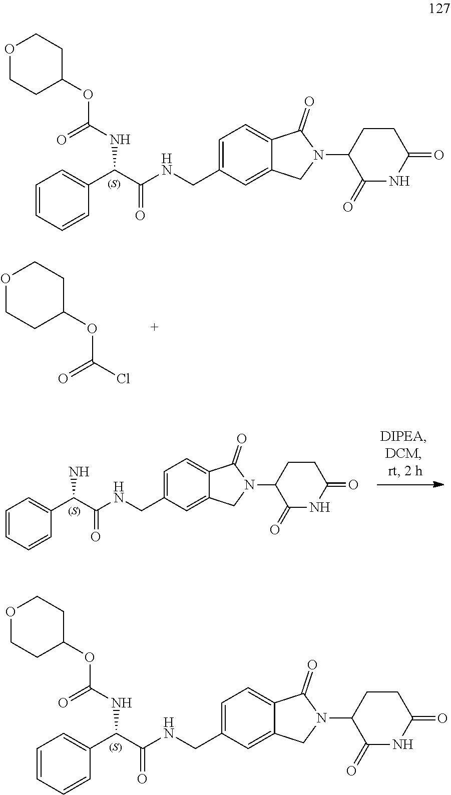

Compound 127 was prepared according to Example 11, Step 2. MS(m/z): 535.3 [M+H]⁺; ¹H NMR (400 MHz, DMSO-d₆) δ10.99 (s, 1H), 8.79 (t, J=6.1 Hz, 1H), 7.60 (d, J=7.8 Hz, 1H), 7.45 (d, J=7.1 Hz, 2H), 7.38-7.23 (m, 6H), 5.26 (d, J=5.7 Hz, 1H), 5.09 (dd, J=13.4, 5.1 Hz, 1H), 4.70 (dq, J=8.7, 4.2 Hz, 1H), 4.45-4.14 (m, 4H), 3.86-3.75 (m, 2H), 3.48-3.39 (m, 2H), 2.98-2.84 (m, 1H), 2.59 (d, J=17.6 Hz, 1H), 2.41 (td, J=13.2, 4.5 Hz, 1H), 2.04-1.93 (m, 1H), 1.89-1.75 (m, 2H), 1.60-1.41 (m, 2H).

### Example 127 (4-175b)

### ((1S)-2-((2-(2,6-dioxypiperidin-3-yl)-1-oxoisoidoline-5-yl)methyl)amino)-2-oxo-1-phenylethyl) carbamate-(−)-menthol Ester

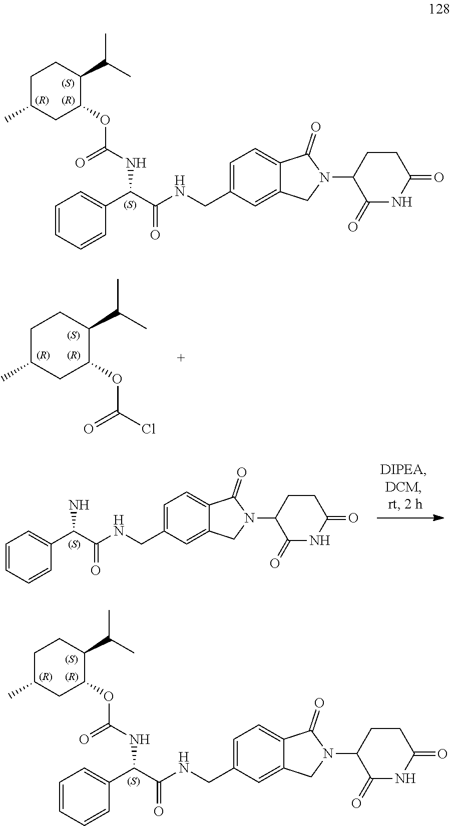

Compound 128 was prepared according to Example 11, Step 2. MS(m/z): 589.0 [M+H]⁺; ¹H NMR (400 MHz, DMSO-d₆) δ10.99 (s, 1H), 8.75 (s, 1H), 7.70-7.55 (m, 2H), 7.45 (d, J=7.1 Hz, 2H), 7.31 (dq, J=22.5, 8.6, 7.7 Hz, 5H), 5.28 (d, J=5.4 Hz, 1H), 5.10 (dd, J=13.4, 5.1 Hz, 1H), 4.43 (s, 1H), 4.35 (d, J=17.9 Hz, 3H), 4.22 (d, J=17.0 Hz, 1H), 2.91 (ddd, J=17.8, 13.6, 5.2 Hz, 1H), 2.59 (d, J=17.8 Hz, 1H), 2.39 (qd, J=14.4, 13.8, 5.0 Hz, 1H), 2.04-1.88 (m, 2H), 1.83 (d, J=12.1 Hz, 1H), 1.62 (s, 2H), 1.42 (d, J=6.8 Hz, 1H), 1.34-1.27 (m, 1H), 1.07-0.58 (m, 12H).

### Example 128 (5-17b)

### Tert butyl ((1S)-1-(2-chlorophenyl)-2-((2-((S)-2,6-dioxopyridin-3-yl)-1-oxoisoidoline-5-yl) methyl) amino)-2-oxoethyl) carbamate

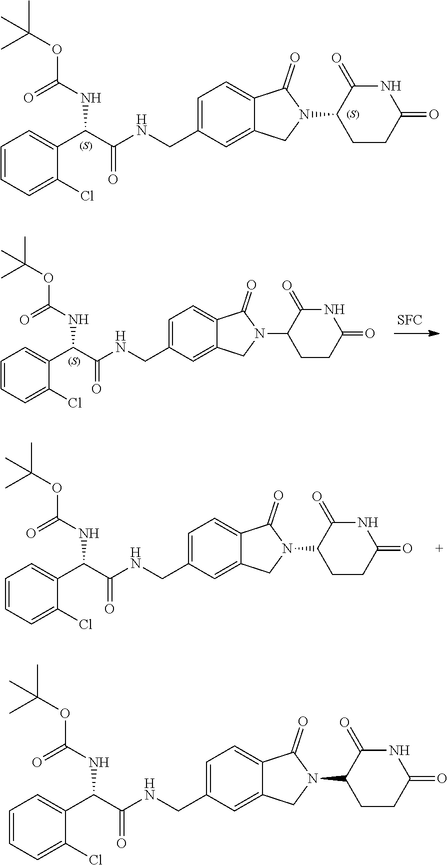

Compound 129 was obtained by SFC splitting from Example 47, ee=99%. MS(m/z): 586.3 [M+H]⁺; ¹H NMR (400 MHz, Chloroform-d) δ 7.92 (s, 1H), 7.76 (d, J=7.7 Hz, 1H), 7.42 (ddd, J=15.2, 7.6, 2.3 Hz, 2H), 7.30 (ddd, J=6.9, 4.5, 2.0 Hz, 2H), 7.24 (s, 2H), 6.43 (s, 1H), 5.95 (s, 1H), 5.70 (s, 1H), 5.20 (dd, J=13.3, 5.1 Hz, 1H), 4.53 (d, J=5.9 Hz, 2H), 4.42 (d, J=15.9 Hz, 1H), 4.26 (d, J=16.1 Hz, 1H), 2.97-2.78 (m, 2H), 2.42-2.29 (m, 1H), 2.25-2.16 (m, 1H), 1.42 (s, 9H).

### Test Example 1: Western Blot Detection of Degradation Activity of GSPT1 Protein by the Compounds

Cell line: Jeko-1 cell line was cultured with an RPMI1640 media containing 20% fetal bovine serum in an incubator at 37° C., 5% CO₂, and saturated humidity.

DMSO control group and test compound (concentration shown in the table below) were set up, after processing for 24 hours, the cells were collected, pre-cooled cell lysate was added and left on ice for 10 minutes. The total protein of the cells was extracted, and the protein concentration was measured and quantified using the BCA method. Routine gel preparation, sample loading, electrophoresis were conducted, then the membrane was transferred, and sealed, wherein the primary antibody was diluted with a blocking solution at 1:500-5000. The membrane was immersed in the diluted primary antibody and incubated overnight at 4° C. After rinsing, the secondary antibody was diluted with a sealing solution at 1:10000-20000, the membrane was immersed in the diluted secondary antibody, and incubated at room temperature for 45 minutes. After rinsing, the results were tested on ODYSSEY (Li-COR). GAPDH was used as an internal reference control.

Image J software was used to perform gray level analysis on each band and the degradation rate of GSPT1 protein by the compounds was calculated.

The results show that the compounds of the present invention have significant degradation activity on GSPT1 protein in Jeko-1 cells, as shown in the table below:

TABLE 1
Degradation activity of compounds of the present
invention on GSPT1 protein in Jeko-1 cells

| | GSPT1 | | | GSPT1 | |
|---|---|---|---|---|---|
| | degradation rate | | | degradation rate | |
| Compound | (%) | | Compound | (%) | |
| No. | 100 nM | 10 μM | No. | 100 nM | 10 μM |

Group I

| | | | | | |
|---|---|---|---|---|---|
| 4-176 | 35 | 53 | 4-186 | 26 | 56 |
| 5-82 | 29.8 | 74.8 | 4-87-2 | 21 | 15 |

Group II

| | | | | | |
|---|---|---|---|---|---|
| 4-138 | 31 | 88 | 4-160 | 96 | 89 |
| 4-87 | 89 | 93 | 4-159 | 93 | 99 |
| 4-175 | 93 | 90 | 4-139 | 98 | 97 |
| 4-184 | 21 | 85 | 4-199 | 14 | 81 |
| 4-123 | 79.9 | 92.7 | 5-57 | 75 | 98.1 |
| 5-56 | 18.8 | 84.1 | 4-133 | 79 | 86 |
| 4-136 | 82 | 97 | 5-5 | 100 | 100 |
| 4-200 | 99 | 100 | 5-17 | 94.2 | 95.6 |
| 4-150 | 90 | 99 | 5-7 | 94 | 100 |
| 4-152 | 93 | 82 | 4-151 | 77 | 95 |
| 4-166 | 82 | 93 | 5-29 | 70 | 97 |
| 5-30 | 59 | 93 | 5-31 | 84 | 99 |
| 4-135 | 80 | 93 | 5-36 | 97 | 97 |
| 1-89 | 97.8 | 98.7 | 5-76 | 65.2 | 84.6 |
| 5-50 | 94.9 | 96.9 | | | |

Group III

| | | | | | |
|---|---|---|---|---|---|
| 4-131 | 96 | 97 | 4-171 | 29 | 88 |
| 4-188 | 32 | 63 | 5-10 | 73 | 90 |
| 5-11 | 17 | 77 | 5-12 | 18 | 71 |
| 5-23 | 28 | 93 | 5-25 | 58 | 96 |
| 5-26 | 33 | 98 | 5-33 | 52 | 80 |
| 5-41 | 96.9 | 96.7 | 5-52 | 38.1 | 92.7 |
| 5-53 | 26.1 | 91.2 | 5-55 | 85.1 | 93.9 |
| 5-59 | 36 | 93.7 | 5-95 | 16 | 70 |
| 5-3 | 76 | 49 | 5-9 | 45 | 93 |

Group IV

| | | | | | |
|---|---|---|---|---|---|
| 4-130 | 51 | 94 | 5-97 | 97 | 98 |
| 5-99 | 98 | 99 | | | |

Group V

| | | | | | |
|---|---|---|---|---|---|
| 5-39 | 65.2 | 85.8 | | | |
| 5-37 | 97.8 | 99.5 | 5-22 | 77.7 | 95.7 |
| 4-132 | 93 | 96 | 5-43 | 11.6 | 87.9 |
| 5-46 | 90.6 | 93.6 | 5-54 | 41.3 | 87.1 |
| 5-83 | 96.2 | 95.5 | 5-84 | 52.7 | 82.7 |
| 5-92 | 79 | 97 | 5-93 | 99 | 100 |
| 5-94 | 85 | 95 | | | |

TABLE 2
Degradation activity of compounds of the present
invention on GSPT1 protein in Jeko-1 cells

| | GSPT1 | | | GSPT1 | |
|---|---|---|---|---|---|
| | degradation rate | | | degradation rate | |
| Compound | (%) | | Compound | (%) | |
| No. | 10 nM | 1 μM | No. | 10 nM | 1 μM |
| 6-01 | 10 | 68 | 6-02 | 95 | 96 |
| 6-03 | 28 | 90 | 4-175b | 98 | 99 |
| 5-17b | 57 | 100 | 4-108 | 10 | 95 |

### Test Example 2: CTG Method for Detecting the Inhibitory Effect of Compounds of Formula on the Proliferation of HNT 34 or HL-60 Cells

### Procedure:

HNT-34 or HL-60 cells were taken and cultured in RPMI1640 medium containing 20% fetal bovine serum. The cells were inoculated on a 96 well plate, with 1×10⁴ cells per well, and incubated at 37° C. in a 500 CO₂ incubator. After adding the test compound, it was incubated for 72 hours. Then an appropriate amount of CTG reagent was added, the luminescence value was measured, and the inhibition rate was calculated.

The experimental results indicate that the compounds of the present invention have significant anti-HL-60 or HNT-34 cell proliferation activity.

TABLE 3
The inhibitory activity of the compounds of the present
invention on the proliferation of HL-60 or HNT-34 cells

| | Cellular | | | Cellular | |
|---|---|---|---|---|---|
| | proliferation | | | proliferation | |
| | inhibition | | | inhibition | |
| Compound | IC₅₀(nM) | | Compound | IC₅₀(nM) | |
| No. | HL-60 | HNT-34 | No. | HL-60 | HNT-34 |

Group II

| | | | | | |
|---|---|---|---|---|---|
| 4-151 | 11.6 | 7.8 | 4-136 | / | 3.6 |
| 5-17 | 4.2 | 3.2 | 1-89 | 5.0 | 3.8 |
| 4-123 | 28.9 | 26.9 | 5-50 | 6.9 | 5.8 |
| 5-31 | 12.5 | 6.6 | 4-152 | 7.9 | 6.6 |
| 4-200 | 8.3 | 3.2 | 5-56 | 25.8 | 18.0 |
| 4-150 | 9.2 | 6.5 | 5-57 | 15.0 | 7.5 |
| 5-7 | 12.4 | 8.7 | 4-135 | 14.3 | 6.2 |
| 4-166 | 12.8 | 11.3 | 4-133 | 14.3 | 3.2 |
| 5-17b | 4.6 | / | | | |

Group III

| | | | | | |
|---|---|---|---|---|---|
| 5-33 | 35.4 | 32.7 | 5-10 | 37.7 | 36.9 |

Group IV

| | | |
|---|---|---|
| 5-97 | 11.0 | 6.9 |

Group V

| | | | | | |
|---|---|---|---|---|---|
| 5-22 | 18.6 | 21.3 | 5-92 | 25.2 | 25.2 |
| 5-94 | 12.8 | 7.4 | 5-46 | 8.3 | 3.7 |

### Test Example 3: CCK8 Method for Detecting Inhibition of Jeko-1 Cell Proliferation by Compound of Formula

### Procedure:

Cell culture: The Jeko-1 cell line (human lymphoma cells) was cultured in RPMI-1640 complete medium containing 1000 fetal bovine serum and 1% penicillin streptomycin, and incubated in an incubator at 37° C., 5% CO₂, and saturated humidity.

Cell laying: Jeko-1 cells in logarithmic growth phase was taken, centrifuged, and an appropriate amount of complete culture medium was added to obtain a single-cell suspension, a hemocytometer was used for cell counting, and a cell suspension of 1.5×105 cells/mL was prepared, the cells were inoculated into a 96 well culture plate at a rate of 100 μL cell suspension per well and incubated in a CO₂ cell incubator for 24 hours.

Cell administration: The compounds to be tested in the example were taken and prepared into 2.5 μM mother liquor, as shown in FIG. 1, 25 μL of the compounds in Example was added to each well, shaken, placed in a CO₂ cell culture incubator, and continued to culture for 72 hours.

CCK-8 detection: 72 hours after administration, 10% CCK-8 solution was added to each well, placed in a CO₂ cell culture incubator, and incubated for 1-4 hours. The absorbance of each well was measured using a microplate reader at 450 nm.

Calculation of cellular proliferation inhibition rate:

Cellular proliferation inhibition rate (%)=[(Ac−As)/(Ac−Ab)]×100%
As: absorbance in experimental wells (including cells, culture medium, CCK-8 solution, and drug solution)
Ac: absorbance in control wells (including cells, culture medium, CCK-8 solution, without drugs)
Ab: absorbance in blank wells (including culture medium, CCK-8 solution, without cells or drugs)

The experimental results indicate that the compounds of the present invention have significant anti-Jeko-1 cell proliferation activity.

TABLE 4
Inhibition activity of compounds of the present
invention on Jeko-1 cell proliferation

| | Cellular | | Cellular |
|---|---|---|---|
| | proliferation | | proliferation |
| Compound | inhibition | Compound | inhibition |
| No. | IC₅₀(nM) | No. | IC₅₀(nM) |
| 4-151 | 1.3 | 4-152 | 0.7 |
| 5-17 | 0.1 | 5-41 | 0.04 |
| 4-87 | 0.2 | 5-37 | 0.6 |
| 5-22 | 0.6 | 5-46 | 0.2 |
| 5-5 | 0.2 | 4-159 | 0.02 |
| 5-36 | 1.0 | 4-175 | 0.03 |
| 4-150 | 3.9 | | |

### Test Example 4: Compound's In Vivo Efficacy in MV-4-11 Subcutaneous Transplant Tumors

Experimental method: Human acute monocytic leukemia cell line MV-4-11 was used to construct a subcutaneous transplant tumor model in SCID mice.

Grouping: solvent group, reference compound (IV: 2 mg/kg, Day 0-12; IP: 20 mg/kg, Day 13-26) group, 5-17 and 5-22 (IV: 0.5 mg/kg, Day 0-12; IP: 10 mg/kg, Day 13-26), with 5 animals in each group, once a day. The tumor volume on the 27th day was measured and the tumor inhibition rate was calculated. Tumor inhibition rate (%)=[(tumor volume in solvent group−tumor volume in drug group)/tumor volume in solvent group]×100%

The experimental results show that the compounds of the present invention have significant in vivo anti-tumor activity.

| Compound No. | Inhibition rate (%) |
|---|---|
| 5-17 | 99.6 |
| 5-22 | 99.8 |
| Reference | 28.3 |
| compound | |

Formula of the reference compound:

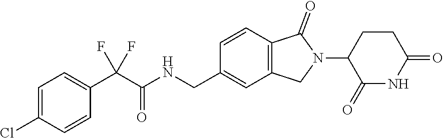 [SMILES: O=C1CCC(N2Cc3cc(CNC(=O)C(F)(F)c4ccc(Cl)cc4)ccc3C2=O)C(=O)N1]

Those skilled in the art can understand that the above embodiments are specific examples of the present invention, and in practical applications, various changes can be made in form and details without deviating from the spirit and scope of the present invention.

1. A compound of Formula I, or its pharmaceutically acceptable salts, solvates, or stereoisomers: 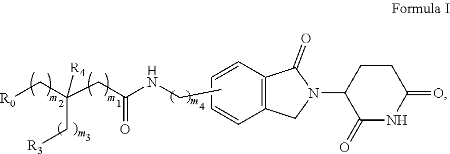
wherein:
R₀ is NR₁R₂ or OR₂;
R₁ is selected from: H, C₁₋₈ alkyl, C₂₋₈ alkenyl, C₂₋₈ alkynyl, C₃₋₁₀ cycloalkyl, 3- to 8-membered heterocyclic groups, aryl and heteroaryl;
R₂ is selected from: H, C₁₋₈ alkyl, C₂₋₈ alkenyl, C₂₋₈ alkynyl, C₃₋₁₀ cycloalkyl, 3- to 8-membered heterocyclic groups, aryl, heteroaryl, (CH₂)ₙC(O)R₅, C(O)(CHR₆)ₙR₅, C(O)(CHR₆)ₙOR₅, S(O)₂R₅, C(O)C(O)R₅; wherein R₅ is independently selected from: H, C₁₋₈ alkyl, C₂₋₈ alkenyl, C₂₋₈ alkynyl, C₃₋₁₀ cycloalkyl, 3- to 8-membered heterocyclic groups, aryl, heteroaryl, NR₇R₈; R₇ is selected from: H, C₁₋₈ alkyl, R₈ is selected from: H, C₁₋₈ alkyl, C₂₋₈ alkenyl, C₂₋₈ alkynyl, C₃₋₁₀ cycloalkyl, 3- to 8-membered heterocyclic groups, aryl, heteroaryl, alkyl carbonyl, alkoxycarbonyl, or R₇ and R₈ are connected into a ring; R₆ is independently selected from: H, C₁₋₈ alkyl, C₂₋₈ alkenyl, C₂₋₈ alkynyl, C₃₋₁₀ cycloalkyl, 3- to 8-membered heterocyclic groups, aryl, heteroaryl, n is independently 0 or 1;
R₃ is selected from: C₁₋₈ alkyl, C₂₋₈ alkenyl, C₂₋₈ alkynyl, C₃₋₁₀ cycloalkyl, 3- to 8-membered heterocyclic groups, aryl, heteroaryl, C(O)R₉; wherein, R₉ is selected from: C₁₋₈ alkyl, C₂₋₈ alkenyl, C₂₋₈ alkynyl, C₃₋₁₀ cycloalkyl, 3- to 8-membered heterocyclic groups, aryl, heteroaryl, NH₂; or R₂ and R₃ is connected into a ring;
R₄ is selected from: H, C₁₋₈ alkyl, C₂₋₈ alkenyl, C₂₋₈ alkynyl, C₃₋₁₀ cycloalkyl, 3- to 8-membered heterocyclic groups, aryl, heteroaryl; or R₃ and R₄ is connected into a ring (Preferably, a 3- to 20-membered ring, either non-substituted or substituted by 1-3 substituents, which can be saturated, unsaturated carbon ring, or a heterocyclic ring);
Wherein, each of the above-mentioned alkyl, alkenyl, alkynyl, cycloalkyl, heterocyclic, aryl, and heteroaryl groups can be optionally and independently substituted by 1-3 substituents, the substituents are independently halogens, C₁₋₄ alkyl, C₂₋₄ alkenyl, C₂₋₄ alkynyl, C₃₋₈ cycloalkyl, 3- to 8-membered heterocyclic groups, aryl, heteroaryl, single or multi-halogenated C₁₋₄ alkyl (like —CF₃), alkoxy, alkyl carbonyl, alkoxycarbonyl, CN, OH, NH₂, or NO₂;
m₁ is 0, 1 or 2;
m₂ is 0, 1 or 2;
m₃ is 0, 1 or 2;
m₄ is 0, 1 or 2.

2. The compound of claim 1, or its pharmaceutically acceptable salts, solvates, or stereoisomers, wherein,
m₁ is 0, m₂ is 0, and m₃ is 0; or
m₁ is 1 or 2, m₂ is 0, and m₃ is 0; or
m₁ is 0, m₂ is 1 or 2, and m₃ is 0; or
m₁ is 0, m₂ is 0, and m₃ is 1 or 2.

3. The compound of claim 1, or its pharmaceutically acceptable salts, solvates, or stereoisomers, wherein, the structure of the compound is shown in Formula I′:

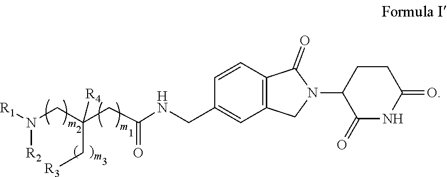

4. The compound of claim 1, or its pharmaceutically acceptable salts, solvates, or stereoisomers, wherein, the structure of the compound is shown in Formula I″: 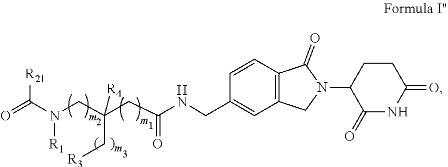
Wherein, R₂₁ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl, OR₂₂; wherein, R₂₂ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl;
Wherein, each of the above-mentioned alkyl, alkenyl, alkynyl, cycloalkyl, heterocyclic groups, aryl, and heteroaryl groups can be optionally and independently substituted by 1-3 substituents, the substituents are independently halogens, C₁₋₄ alkyl, C₂₋₄ alkenyl, C₂₋₄ alkynyl, C₃₋₈ cycloalkyl, 3- to 8-membered heterocyclic groups, aryl, heteroaryl, CN, OH, NH₂, or NO₂.

5. The compound of claim 1, or its pharmaceutically acceptable salts, solvates, or stereoisomers, wherein, the structure of the compound is shown in Formula II: 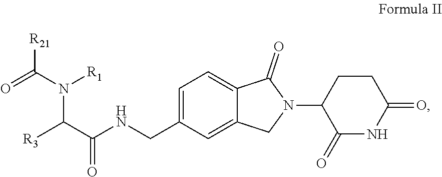
Wherein,
R₁ is selected from: H, C₁₋₈ alkyl, C₂₋₈ alkenyl, C₂₋₈ alkynyl, C₃₋₈ cycloalkyl, 3- to 8-membered heterocyclic groups, aryl, and heteroaryl;
R₃ is selected from: C₁₋₈ alkyl, C₂₋₈ alkenyl, C₂₋₈ alkynyl, C₃₋₈ cycloalkyl, 3- to 8-membered heterocyclic groups, aryl, and heteroaryl;
R₂₁ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl, OR₂₂; wherein, R₂₂ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl;
Wherein, each of the above-mentioned alkyl, alkenyl, alkynyl, cycloalkyl, heterocyclic groups, aryl, and heteroaryl groups can be optionally and independently substituted by 1-3 substituents, the substituents are independently halogens, C₁₋₄ alkyl, C₂₋₄ alkenyl, C₂₋₄ alkynyl, C₃₋₈ cycloalkyl, 3- to 8-membered heterocyclic groups, aryl, heteroaryl, CN, OH, NH₂, or NO₂.

6. The compound of claim 1, or its pharmaceutically acceptable salts, solvates, or stereoisomers, wherein, the structure of the compound is shown in Formula III: 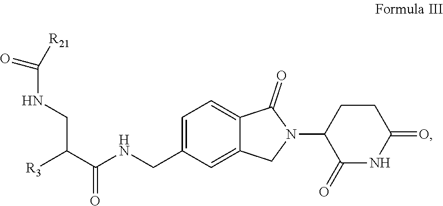
Wherein,
R₃ is selected from: C₁₋₈ alkyl, C₂₋₈ alkenyl, C₂₋₈ alkynyl, C₃₋₈ cycloalkyl, 3- to 8-membered heterocyclic groups, aryl, and heteroaryl;
R₂₁ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl, OR₂₂; wherein, R₂₂ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl;
Wherein, each of the above-mentioned alkyl, alkenyl, alkynyl, cycloalkyl, heterocyclic groups, aryl, and heteroaryl groups can be optionally and independently substituted by 1-3 substituents, the substituents are independently halogens, C₁₋₄ alkyl, C₂₋₄ alkenyl, C₂₋₄ alkynyl, C₃₋₈ cycloalkyl, 3- to 8-membered heterocyclic groups, aryl, heteroaryl, CN, OH, NH₂, or NO₂.

7. The compound of claim 1, or its pharmaceutically acceptable salts, solvates, or stereoisomers, wherein, the structure of the compound is shown in Formula IV: 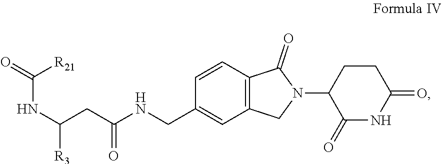
Wherein,
R₃ is selected from: C₁₋₈ alkyl, C₂₋₈ alkenyl, C₂₋₈ alkynyl, C₃₋₈ cycloalkyl, 3- to 8-membered heterocyclic groups, aryl, and heteroaryl;
R₂₁ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl, OR₂₂; wherein, R₂₂ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl;
Wherein, each of the above-mentioned alkyl, alkenyl, alkynyl, cycloalkyl, heterocyclic groups, aryl, and heteroaryl groups can be optionally and independently substituted by 1-3 substituents, the substituents are independently halogens, C₁₋₄ alkyl, C₂₋₄ alkenyl, C₂₋₄ alkynyl, C₃₋₈ cycloalkyl, 3- to 8-membered heterocyclic groups, aryl, heteroaryl, CN, OH, NH₂, or NO₂.

8. The compound of claim 1, or its pharmaceutically acceptable salts, solvates, or stereoisomers, wherein, the structure of the compound is shown in Formula V: 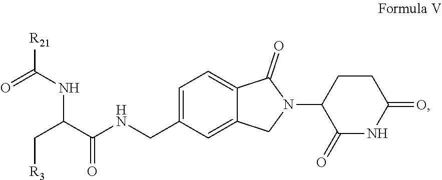
Wherein,
R₃ is selected from: C₁₋₈ alkyl, C₂₋₈ alkenyl, C₂₋₈ alkynyl, C₃₋₈ cycloalkyl, 3- to 8-membered heterocyclic groups, aryl, and heteroaryl;
R₂₁ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl, OR₂₂; wherein, R₂₂ is selected from: C₁₋₈ alkyl, C₂₋₈ alkenyl, C₂₋₈ alkynyl, C₃₋₈ cycloalkyl, 3- to 8-membered heterocyclic groups, aryl, heteroaryl;
Wherein, each of the above-mentioned alkyl, alkenyl, alkynyl, cycloalkyl, heterocyclic groups, aryl, and heteroaryl can be optionally and independently substituted by 1-3 substituents, the substituents are independently halogens, C₁₋₄ alkyl, C₂₋₄ alkenyl, C₂₋₄ alkynyl, C₃₋₈ cycloalkyl, 3- to 8-membered heterocyclic groups, aryl, heteroaryl, CN, OH, NH₂, or NO₂.

9. The compound of claim 1, or its pharmaceutically acceptable salts, solvates, or stereoisomers, wherein, the compound is not:

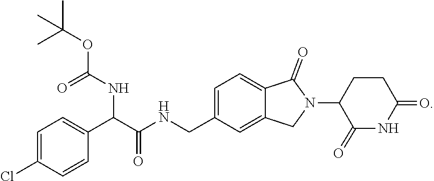 [SMILES: CC(C)(C)OC(=O)NC(C(=O)NCc1ccc2c(c1)CN(C1CCC(=O)NC1=O)C2=O)c1ccc(Cl)cc1]

10. The compound of claim 1, or its pharmaceutically acceptable salts, solvates, or stereoisomers, wherein, the compound is selected from the group consisting of:

| No. | Structure |
|---|---|
| 4-85 | 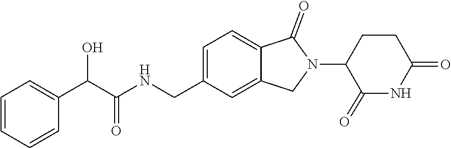 [SMILES: O=C1CCC(N2Cc3cc(CNC(=O)C(O)c4ccccc4)ccc3C2=O)C(=O)N1] |
| 5-15-2 | 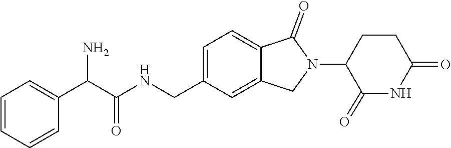 [SMILES: NC(C(=O)NCc1ccc2c(c1)CN(C1CCC(=O)NC1=O)C2=O)c1ccccc1] |
| 4-87-2 | 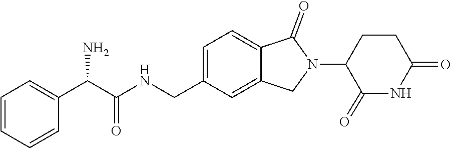 [SMILES: N[C@H](C(=O)NCc1ccc2c(c1)CN(C1CCC(=O)NC1=O)C2=O)c1ccccc1] |
| 4-122-2 | 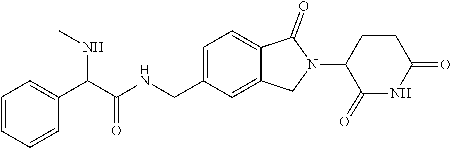 [SMILES: CNC(C(=O)NCc1ccc2c(c1)CN(C1CCC(=O)NC1=O)C2=O)c1ccccc1] |
| 4-112 | 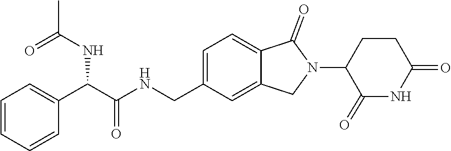 [SMILES: CC(=O)N[C@H](C(=O)NCc1ccc2c(c1)CN(C1CCC(=O)NC1=O)C2=O)c1ccccc1] |
| 4-111 | 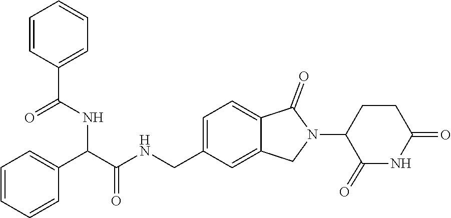 [SMILES: O=C1CCC(N2Cc3cc(CNC(=O)C(NC(=O)c4ccccc4)c4ccccc4)ccc3C2=O)C(=O)N1] |
| 4-121 | 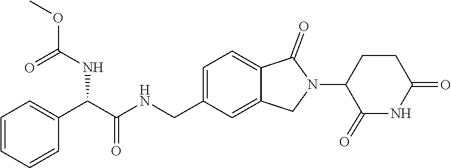 [SMILES: COC(=O)N[C@H](C(=O)NCc1ccc2c(c1)CN(C1CCC(=O)NC1=O)C2=O)c1ccccc1] |
| 4-125 | 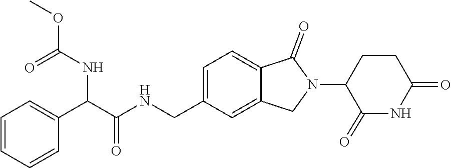 [SMILES: COC(=O)NC(C(=O)NCc1ccc2c(c1)CN(C1CCC(=O)NC1=O)C2=O)c1ccccc1] |
| 4-138 | 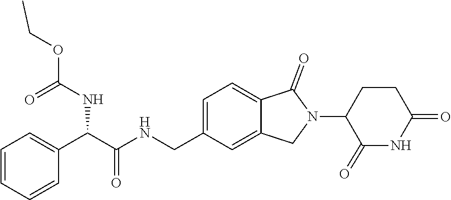 [SMILES: CCOC(=O)N[C@H](C(=O)NCc1ccc2c(c1)CN(C1CCC(=O)NC1=O)C2=O)c1ccccc1] |
| 4-160 | 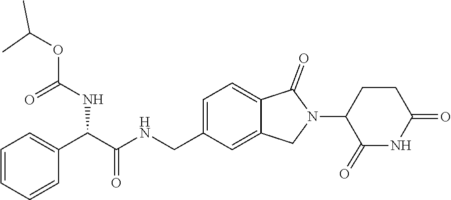 [SMILES: CC(C)OC(=O)N[C@H](C(=O)NCc1ccc2c(c1)CN(C1CCC(=O)NC1=O)C2=O)c1ccccc1] |
| 4-87 | 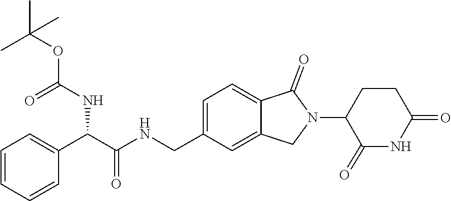 [SMILES: CC(C)(C)OC(=O)N[C@H](C(=O)NCc1ccc2c(c1)CN(C1CCC(=O)NC1=O)C2=O)c1ccccc1] |
| 4-108 | 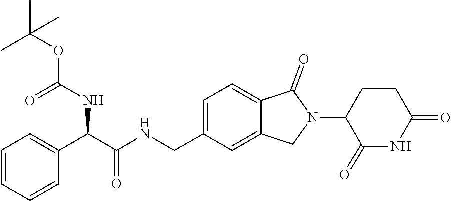 [SMILES: CC(C)(C)OC(=O)N[C@@H](C(=O)NCc1ccc2c(c1)CN(C1CCC(=O)NC1=O)C2=O)c1ccccc1] |
| 4-127 | 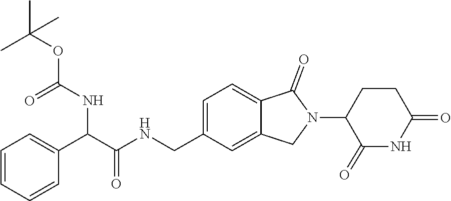 [SMILES: CC(C)(C)OC(=O)NC(C(=O)NCc1ccc2c(c1)CN(C1CCC(=O)NC1=O)C2=O)c1ccccc1] |
| 4-159 | 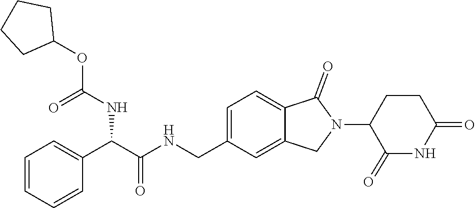 [SMILES: O=C1CCC(N2Cc3cc(CNC(=O)[C@@H](NC(=O)OC4CCCC4)c4ccccc4)ccc3C2=O)C(=O)N1] |
| 4-175 | 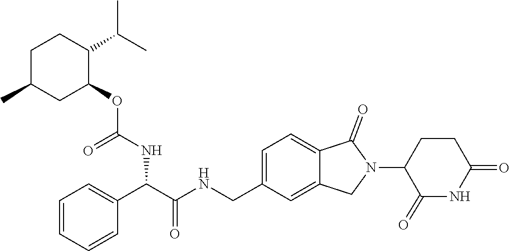 |
| 4-195 | 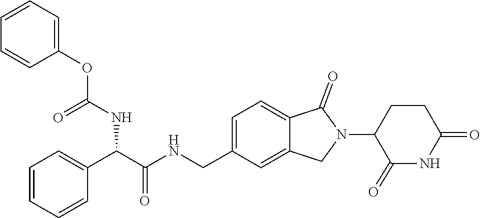 [SMILES: O=C1CCC(N2Cc3cc(CNC(=O)[C@@H](NC(=O)Oc4ccccc4)c4ccccc4)ccc3C2=O)C(=O)N1] |
| 4-139 | 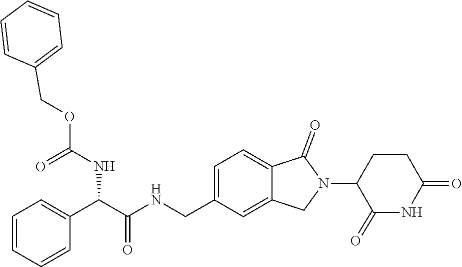 [SMILES: O=C1CCC(N2Cc3cc(CNC(=O)[C@@H](NC(=O)OCc4ccccc4)c4ccccc4)ccc3C2=O)C(=O)N1] |
| 4-184 | 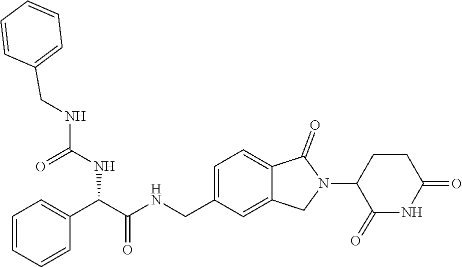 [SMILES: O=C1CCC(N2Cc3cc(CNC(=O)[C@@H](NC(=O)NCc4ccccc4)c4ccccc4)ccc3C2=O)C(=O)N1] |
| 4-182 | 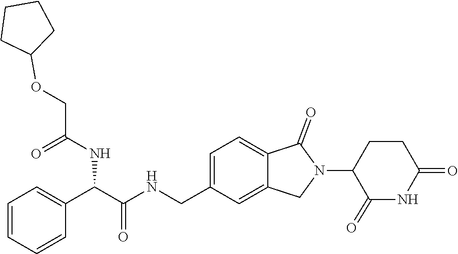 [SMILES: O=C1CCC(N2Cc3cc(CNC(=O)[C@@H](NC(=O)COC4CCCC4)c4ccccc4)ccc3C2=O)C(=O)N1] |
| 4-172 | 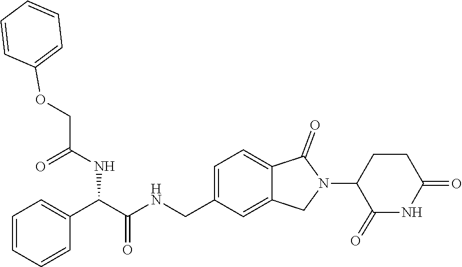 [SMILES: O=C1CCC(N2Cc3cc(CNC(=O)[C@@H](NC(=O)COc4ccccc4)c4ccccc4)ccc3C2=O)C(=O)N1] |
| 5-16 | 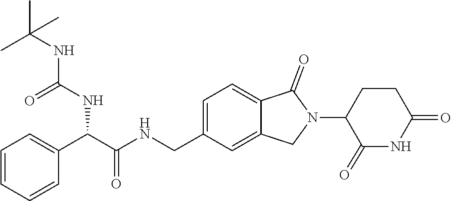 [SMILES: CC(C)(C)NC(=O)N[C@H](C(=O)NCc1ccc2c(c1)CN(C1CCC(=O)NC1=O)C2=O)c1ccccc1] |
| 4-126 | 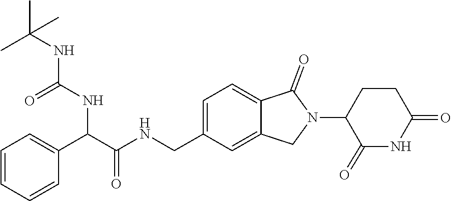 [SMILES: CC(C)(C)NC(=O)NC(C(=O)NCc1ccc2c(c1)CN(C1CCC(=O)NC1=O)C2=O)c1ccccc1] |
| 4-129 | 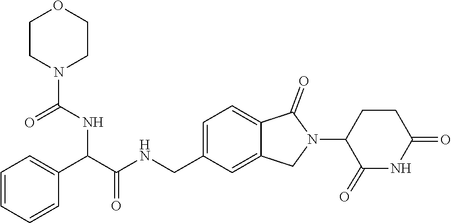 [SMILES: O=C1CCC(N2Cc3cc(CNC(=O)C(NC(=O)N4CCOCC4)c4ccccc4)ccc3C2=O)C(=O)N1] |
| 4-158 | 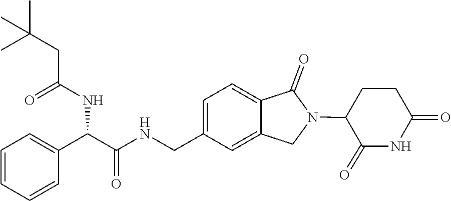 [SMILES: CC(C)(C)CC(=O)N[C@H](C(=O)NCc1ccc2c(c1)CN(C1CCC(=O)NC1=O)C2=O)c1ccccc1] |
| 4-165 | 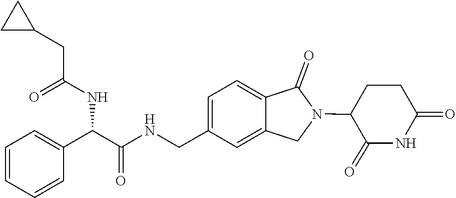 [SMILES: O=C1CCC(N2Cc3cc(CNC(=O)[C@@H](NC(=O)CC4CC4)c4ccccc4)ccc3C2=O)C(=O)N1] |
| 4-176 | 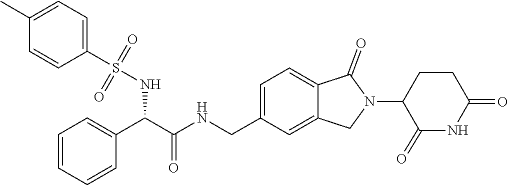 [SMILES: Cc1ccc(S(=O)(=O)N[C@H](C(=O)NCc2ccc3c(c2)CN(C2CCC(=O)NC2=O)C3=O)c2ccccc2)cc1] |
| 4-177 | 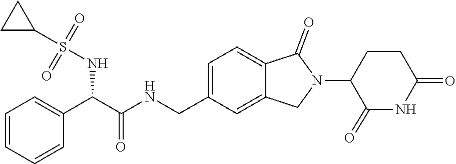 [SMILES: O=C1CCC(N2Cc3cc(CNC(=O)[C@@H](NS(=O)(=O)C4CC4)c4ccccc4)ccc3C2=O)C(=O)N1] |
| 4-197 | 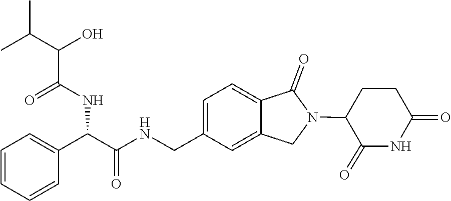 [SMILES: CC(C)C(O)C(=O)N[C@H](C(=O)NCc1ccc2c(c1)CN(C1CCC(=O)NC1=O)C2=O)c1ccccc1] |
| 4-192 | 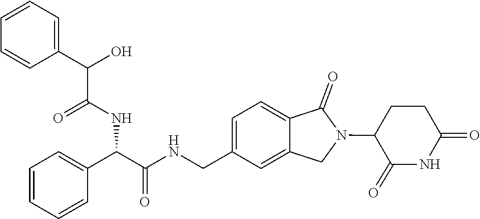 [SMILES: O=C1CCC(N2Cc3cc(CNC(=O)[C@@H](NC(=O)C(O)c4ccccc4)c4ccccc4)ccc3C2=O)C(=O)N1] |
| 4-199 | 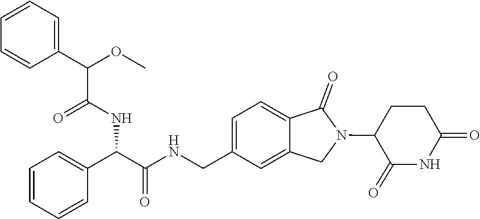 [SMILES: COC(C(=O)N[C@H](C(=O)NCc1ccc2c(c1)CN(C1CCC(=O)NC1=O)C2=O)c1ccccc1)c1ccccc1] |
| 4-198 | 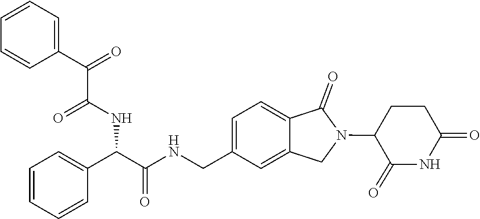 [SMILES: O=C1CCC(N2Cc3cc(CNC(=O)[C@@H](NC(=O)C(=O)c4ccccc4)c4ccccc4)ccc3C2=O)C(=O)N1] |
| 4-196-2 | 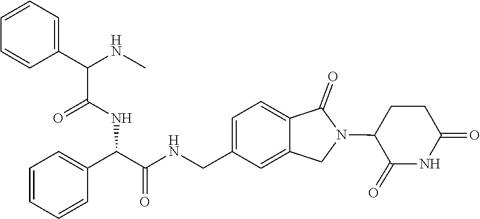 [SMILES: CNC(C(=O)N[C@H](C(=O)NCc1ccc2c(c1)CN(C1CCC(=O)NC1=O)C2=O)c1ccccc1)c1ccccc1] |
| 4-193 | 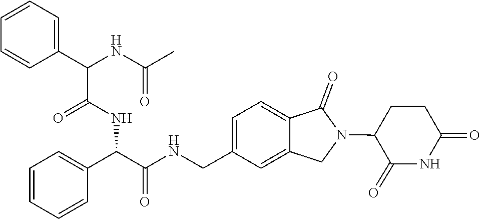 [SMILES: CC(=O)NC(C(=O)N[C@H](C(=O)NCc1ccc2c(c1)CN(C1CCC(=O)NC1=O)C2=O)c1ccccc1)c1ccccc1] |
| 4-109 | 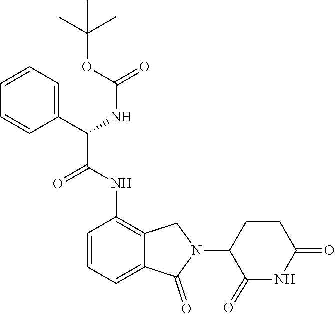 [SMILES: CC(C)(C)OC(=O)N[C@H](C(=O)Nc1cccc2c1CN(C1CCC(=O)NC1=O)C2=O)c1ccccc1] |
| 4-118 | 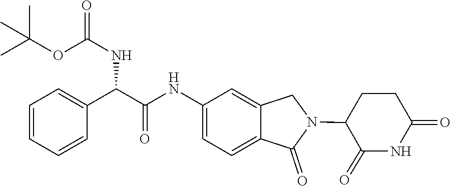 [SMILES: CC(C)(C)OC(=O)N[C@H](C(=O)Nc1ccc2c(c1)CN(C1CCC(=O)NC1=O)C2=O)c1ccccc1] |
| 4-153 | 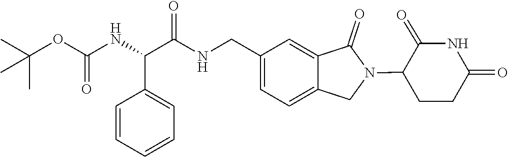 [SMILES: CC(C)(C)OC(=O)N[C@H](C(=O)NCc1ccc2c(c1)C(=O)N(C1CCC(=O)NC1=O)C2)c1ccccc1] |
| 4-167 | 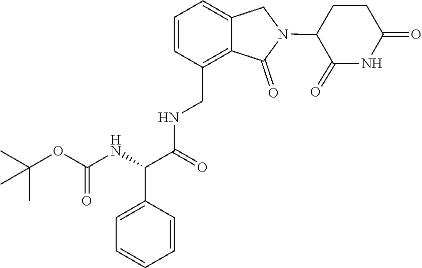 [SMILES: CC(C)(C)OC(=O)N[C@H](C(=O)NCc1cccc2c1C(=O)N(C1CCC(=O)NC1=O)C2)c1ccccc1] |
| 4-117 | 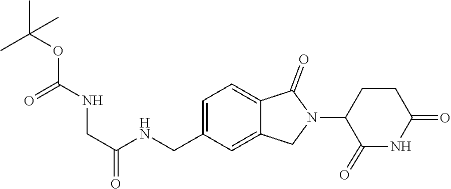 [SMILES: CC(C)(C)OC(=O)NCC(=O)NCc1ccc2c(c1)CN(C1CCC(=O)NC1=O)C2=O] |
| 4-123 | 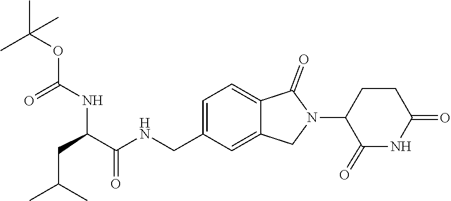 [SMILES: CC(C)C[C@@H](NC(=O)OC(C)(C)C)C(=O)NCc1ccc2c(c1)CN(C1CCC(=O)NC1=O)C2=O] |
| 5-57 | 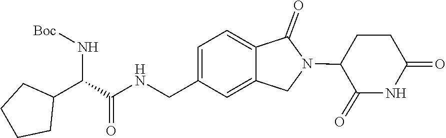 [SMILES: CC(C)(C)OC(=O)N[C@H](C(=O)NCc1ccc2c(c1)CN(C1CCC(=O)NC1=O)C2=O)C1CCCC1] |
| 5-56 | 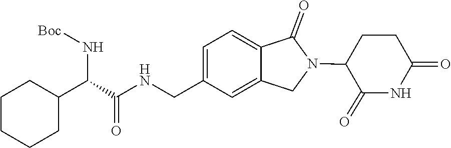 [SMILES: CC(C)(C)OC(=O)N[C@H](C(=O)NCc1ccc2c(c1)CN(C1CCC(=O)NC1=O)C2=O)C1CCCCC1] |
| 4-124 | 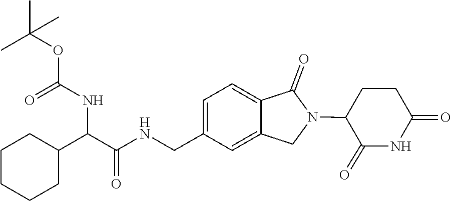 [SMILES: CC(C)(C)OC(=O)NC(C(=O)NCc1ccc2c(c1)CN(C1CCC(=O)NC1=O)C2=O)C1CCCCC1] |
| 4-133 | 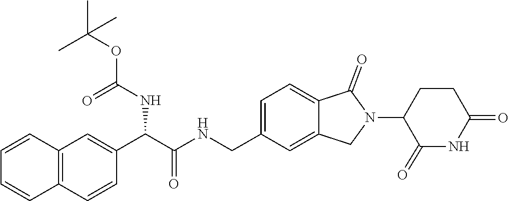 [SMILES: CC(C)(C)OC(=O)N[C@H](C(=O)NCc1ccc2c(c1)CN(C1CCC(=O)NC1=O)C2=O)c1ccc2ccccc2c1] |
| 4-134 | 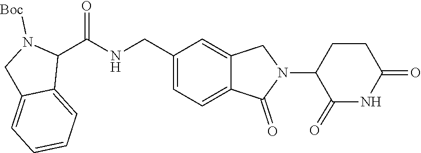 [SMILES: CC(C)(C)OC(=O)N1Cc2ccccc2C1C(=O)NCc1ccc2c(c1)CN(C1CCC(=O)NC1=O)C2=O] |
| 5-32 | 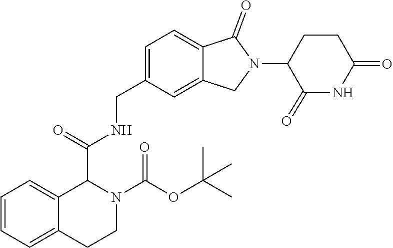 [SMILES: CC(C)(C)OC(=O)N1CCc2ccccc2C1C(=O)NCc1ccc2c(c1)CN(C1CCC(=O)NC1=O)C2=O] |
| 4-136 | 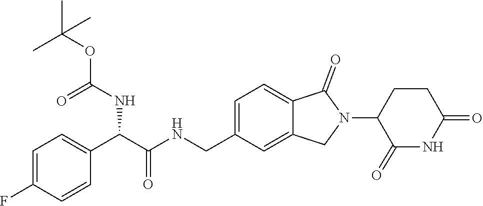 [SMILES: CC(C)(C)OC(=O)N[C@H](C(=O)NCc1ccc2c(c1)CN(C1CCC(=O)NC1=O)C2=O)c1ccc(F)cc1] |
| 5-5 | 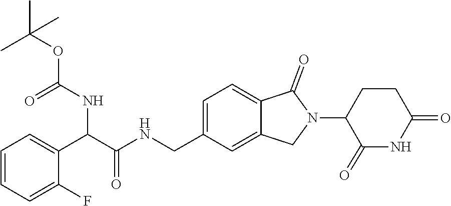 [SMILES: CC(C)(C)OC(=O)NC(C(=O)NCc1ccc2c(c1)CN(C1CCC(=O)NC1=O)C2=O)c1ccccc1F] |
| 4-200 | 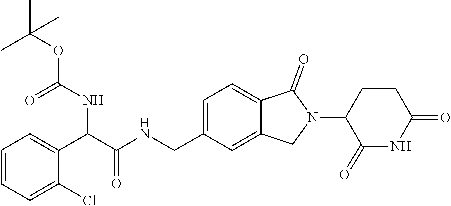 [SMILES: CC(C)(C)OC(=O)NC(C(=O)NCc1ccc2c(c1)CN(C1CCC(=O)NC1=O)C2=O)c1ccccc1Cl] |
| 5-17 | 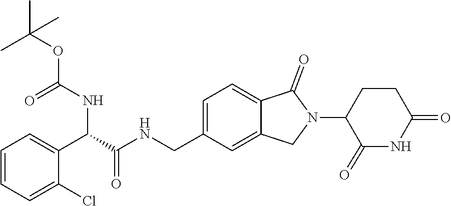 [SMILES: CC(C)(C)OC(=O)N[C@H](C(=O)NCc1ccc2c(c1)CN(C1CCC(=O)NC1=O)C2=O)c1ccccc1Cl] |
| 4-140 | 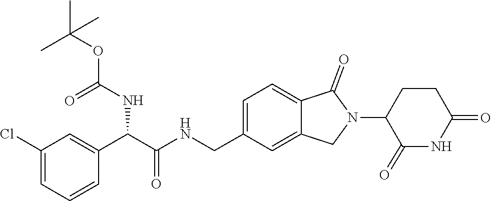 [SMILES: CC(C)(C)OC(=O)N[C@H](C(=O)NCc1ccc2c(c1)CN(C1CCC(=O)NC1=O)C2=O)c1cccc(Cl)c1] |
| 4-150 | 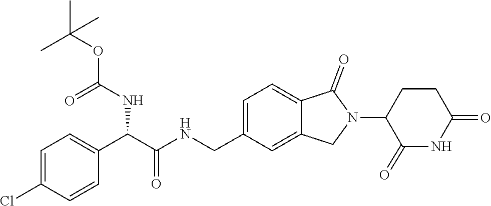 [SMILES: CC(C)(C)OC(=O)N[C@H](C(=O)NCc1ccc2c(c1)CN(C1CCC(=O)NC1=O)C2=O)c1ccc(Cl)cc1] |
| 5-7 | 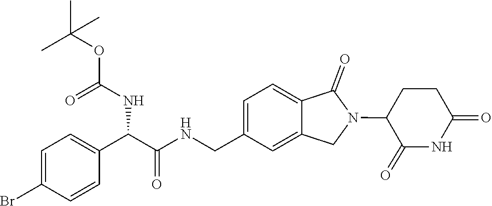 [SMILES: CC(C)(C)OC(=O)N[C@H](C(=O)NCc1ccc2c(c1)CN(C1CCC(=O)NC1=O)C2=O)c1ccc(Br)cc1] |
| 4-152 | 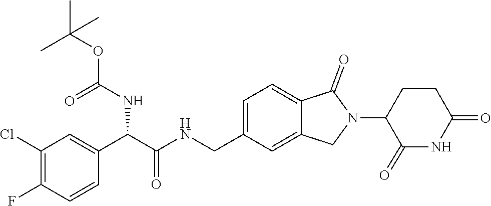 [SMILES: CC(C)(C)OC(=O)N[C@H](C(=O)NCc1ccc2c(c1)CN(C1CCC(=O)NC1=O)C2=O)c1ccc(F)c(Cl)c1] |
| 4-151 | 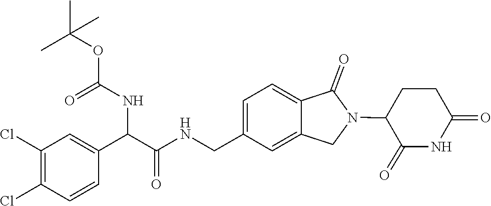 [SMILES: CC(C)(C)OC(=O)NC(C(=O)NCc1ccc2c(c1)CN(C1CCC(=O)NC1=O)C2=O)c1ccc(Cl)c(Cl)c1] |
| 5-6 | 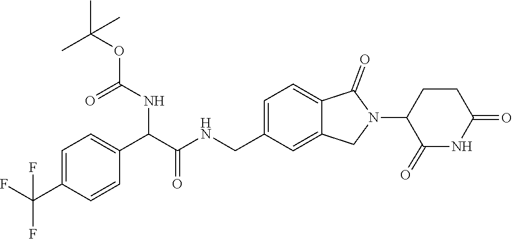 [SMILES: CC(C)(C)OC(=O)NC(C(=O)NCc1ccc2c(c1)CN(C1CCC(=O)NC1=O)C2=O)c1ccc(C(F)(F)F)cc1] |
| 5-13 | 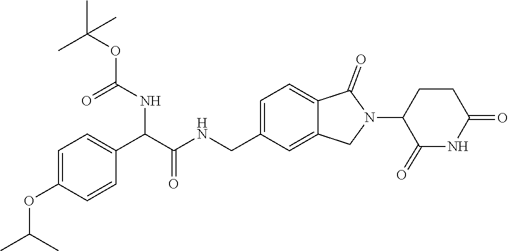 [SMILES: CC(C)Oc1ccc(C(NC(=O)OC(C)(C)C)C(=O)NCc2ccc3c(c2)CN(C2CCC(=O)NC2=O)C3=O)cc1] |
| 4-137 | 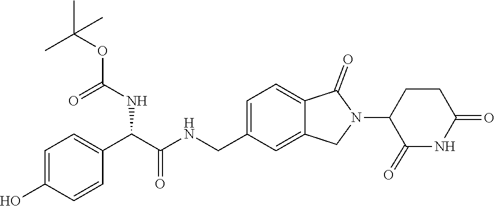 [SMILES: CC(C)(C)OC(=O)N[C@H](C(=O)NCc1ccc2c(c1)CN(C1CCC(=O)NC1=O)C2=O)c1ccc(O)cc1] |
| 4-166 | 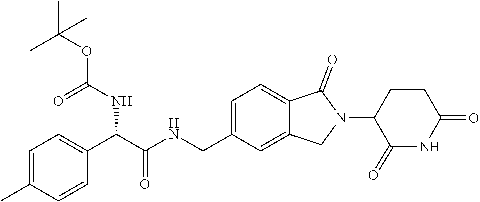 [SMILES: Cc1ccc([C@H](NC(=O)OC(C)(C)C)C(=O)NCc2ccc3c(c2)CN(C2CCC(=O)NC2=O)C3=O)cc1] |
| 4-130 | 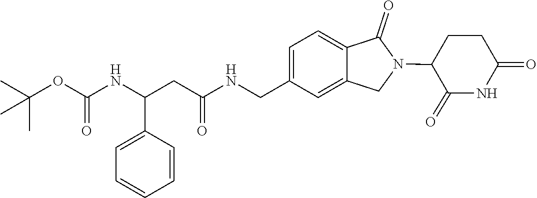 [SMILES: CC(C)(C)OC(=O)NC(CC(=O)NCc1ccc2c(c1)CN(C1CCC(=O)NC1=O)C2=O)c1ccccc1] |
| 4-131 | 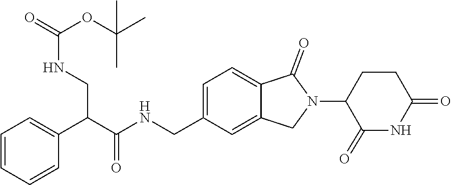 [SMILES: CC(C)(C)OC(=O)NCC(C(=O)NCc1ccc2c(c1)CN(C1CCC(=O)NC1=O)C2=O)c1ccccc1] |
| 4-201 | 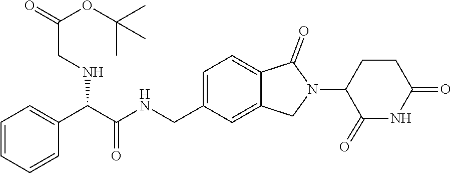 [SMILES: CC(C)(C)OC(=O)CN[C@H](C(=O)NCc1ccc2c(c1)CN(C1CCC(=O)NC1=O)C2=O)c1ccccc1] |
| 5-39 | 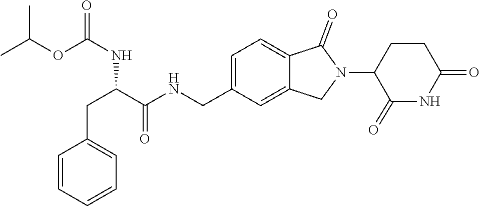 [SMILES: CC(C)OC(=O)N[C@@H](Cc1ccccc1)C(=O)NCc1ccc2c(c1)CN(C1CCC(=O)NC1=O)C2=O] |
| 5-37 | 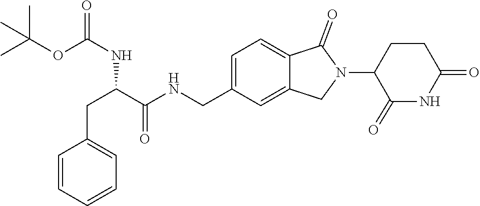 [SMILES: CC(C)(C)OC(=O)N[C@@H](Cc1ccccc1)C(=O)NCc1ccc2c(c1)CN(C1CCC(=O)NC1=O)C2=O] |
| 5-22 | 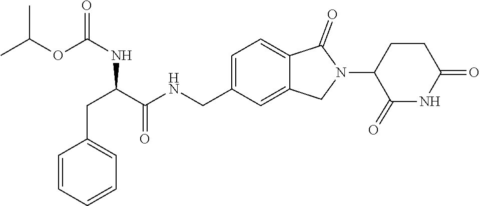 [SMILES: CC(C)OC(=O)N[C@H](Cc1ccccc1)C(=O)NCc1ccc2c(c1)CN(C1CCC(=O)NC1=O)C2=O] |
| 4-132 | 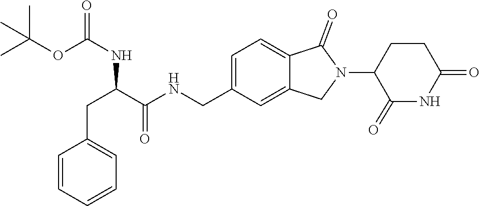 [SMILES: CC(C)(C)OC(=O)N[C@H](Cc1ccccc1)C(=O)NCc1ccc2c(c1)CN(C1CCC(=O)NC1=O)C2=O] |
| 4-171 | 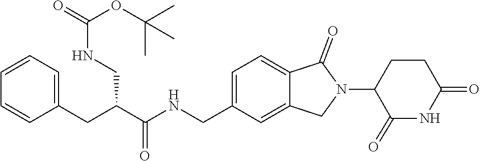 [SMILES: CC(C)(C)OC(=O)NC[C@@H](Cc1ccccc1)C(=O)NCc1ccc2c(c1)CN(C1CCC(=O)NC1=O)C2=O] |
| 4-186 | 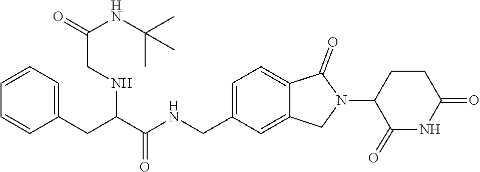 [SMILES: CC(C)(C)NC(=O)CNC(Cc1ccccc1)C(=O)NCc1ccc2c(c1)CN(C1CCC(=O)NC1=O)C2=O] |
| 4-187 | 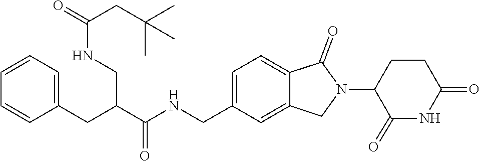 [SMILES: CC(C)(C)CC(=O)NCC(Cc1ccccc1)C(=O)NCc1ccc2c(c1)CN(C1CCC(=O)NC1=O)C2=O] |
| 4-188 | 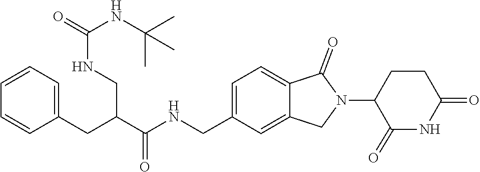 [SMILES: CC(C)(C)NC(=O)NCC(Cc1ccccc1)C(=O)NCc1ccc2c(c1)CN(C1CCC(=O)NC1=O)C2=O] |
| 4-189 | 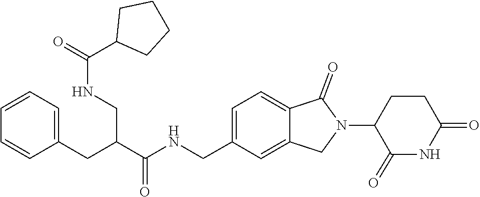 [SMILES: O=C1CCC(N2Cc3cc(CNC(=O)C(CNC(=O)C4CCCC4)Cc4ccccc4)ccc3C2=O)C(=O)N1] |
| 5-8-2 | 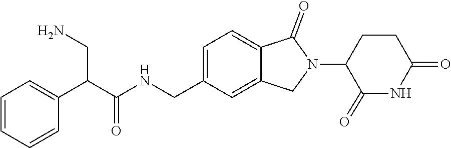 [SMILES: NCC(C(=O)NCc1ccc2c(c1)CN(C1CCC(=O)NC1=O)C2=O)c1ccccc1] |
| 5-2 | 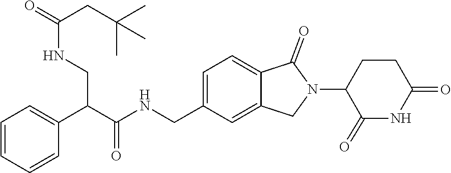 [SMILES: CC(C)(C)CC(=O)NCC(C(=O)NCc1ccc2c(c1)CN(C1CCC(=O)NC1=O)C2=O)c1ccccc1] |
| 5-3 | 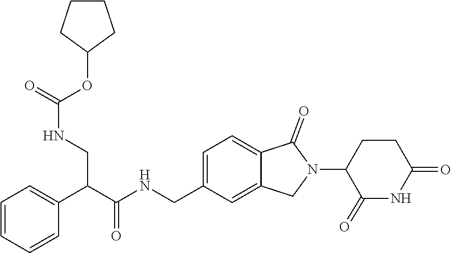 [SMILES: O=C1CCC(N2Cc3cc(CNC(=O)C(CNC(=O)OC4CCCC4)c4ccccc4)ccc3C2=O)C(=O)N1] |
| 5-9 | 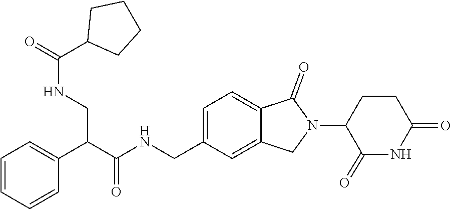 [SMILES: O=C1CCC(N2Cc3cc(CNC(=O)C(CNC(=O)C4CCCC4)c4ccccc4)ccc3C2=O)C(=O)N1] |
| 5-10 | 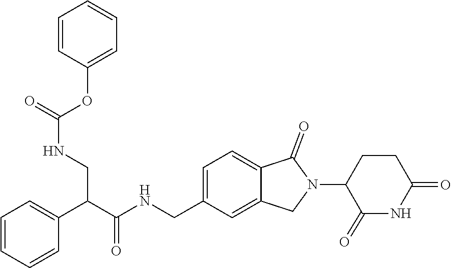 [SMILES: O=C1CCC(N2Cc3cc(CNC(=O)C(CNC(=O)Oc4ccccc4)c4ccccc4)ccc3C2=O)C(=O)N1] |
| 5-11 | 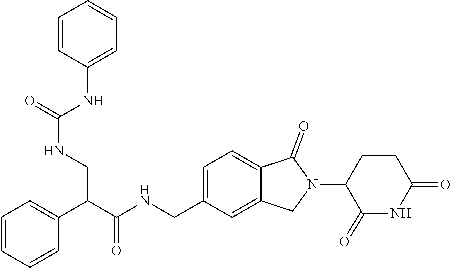 [SMILES: O=C1CCC(N2Cc3cc(CNC(=O)C(CNC(=O)Nc4ccccc4)c4ccccc4)ccc3C2=O)C(=O)N1] |
| 5-12 | 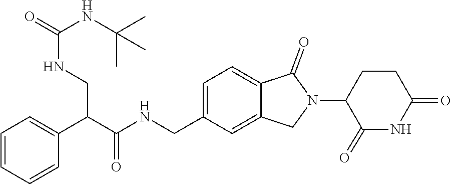 [SMILES: CC(C)(C)NC(=O)NCC(C(=O)NCc1ccc2c(c1)CN(C1CCC(=O)NC1=O)C2=O)c1ccccc1] |
| 5-24 | 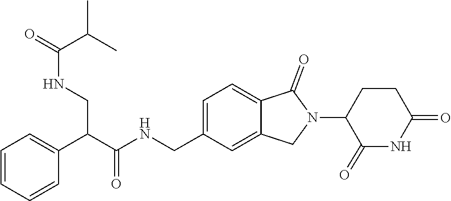 [SMILES: CC(C)C(=O)NCC(C(=O)NCc1ccc2c(c1)CN(C1CCC(=O)NC1=O)C2=O)c1ccccc1] |
| 5-23 | 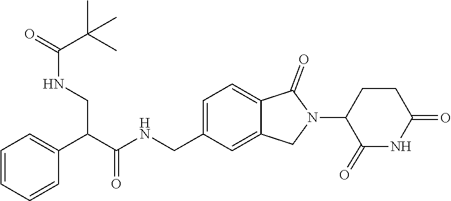 [SMILES: CC(C)(C)C(=O)NCC(C(=O)NCc1ccc2c(c1)CN(C1CCC(=O)NC1=O)C2=O)c1ccccc1] |
| 5-25 | 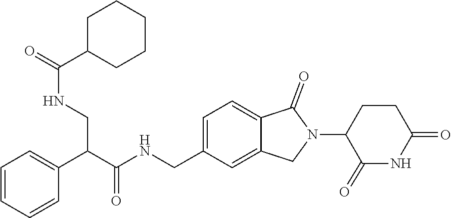 [SMILES: O=C1CCC(N2Cc3cc(CNC(=O)C(CNC(=O)C4CCCCC4)c4ccccc4)ccc3C2=O)C(=O)N1] |
| 5-26 | 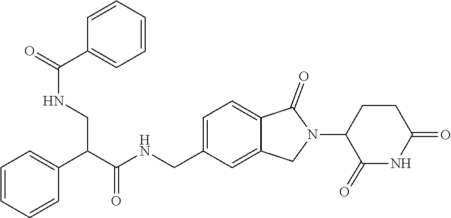 [SMILES: O=C1CCC(N2Cc3cc(CNC(=O)C(CNC(=O)c4ccccc4)c4ccccc4)ccc3C2=O)C(=O)N1] |
| 5-29 | 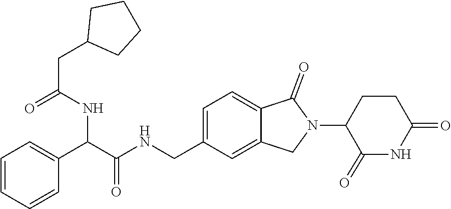 [SMILES: O=C1CCC(N2Cc3cc(CNC(=O)C(NC(=O)CC4CCCC4)c4ccccc4)ccc3C2=O)C(=O)N1] |
| 5-30 | 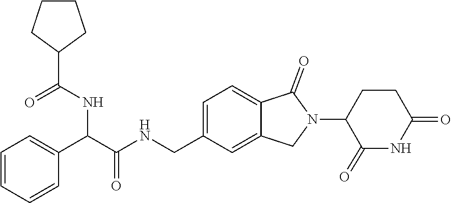 [SMILES: O=C1CCC(N2Cc3cc(CNC(=O)C(NC(=O)C4CCCC4)c4ccccc4)ccc3C2=O)C(=O)N1] |
| 5-33 | 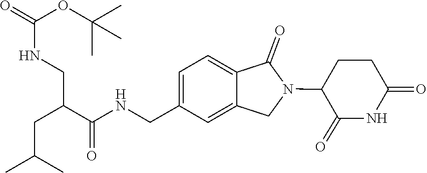 [SMILES: CC(C)CC(CNC(=O)OC(C)(C)C)C(=O)NCc1ccc2c(c1)CN(C1CCC(=O)NC1=O)C2=O] |
| 5-38 | 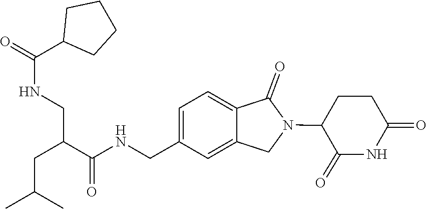 [SMILES: CC(C)CC(CNC(=O)C1CCCC1)C(=O)NCc1ccc2c(c1)CN(C1CCC(=O)NC1=O)C2=O] |
| 5-41 | 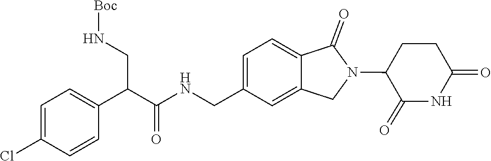 [SMILES: CC(C)(C)OC(=O)NCC(C(=O)NCc1ccc2c(c1)CN(C1CCC(=O)NC1=O)C2=O)c1ccc(Cl)cc1] |
| 5-43 | 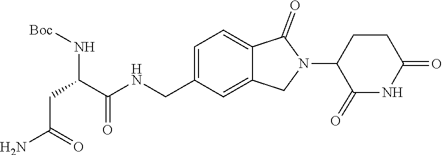 [SMILES: CC(C)(C)OC(=O)N[C@@H](CC(N)=O)C(=O)NCc1ccc2c(c1)CN(C1CCC(=O)NC1=O)C2=O] |
| 5-46 | 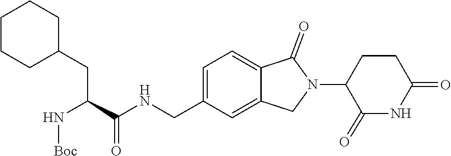 [SMILES: CC(C)(C)OC(=O)N[C@@H](CC1CCCCC1)C(=O)NCc1ccc2c(c1)CN(C1CCC(=O)NC1=O)C2=O] |
| 5-47 | 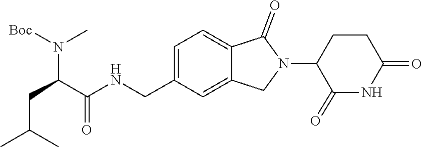 [SMILES: CC(C)C[C@H](C(=O)NCc1ccc2c(c1)CN(C1CCC(=O)NC1=O)C2=O)N(C)C(=O)OC(C)(C)C] |
| 5-52 | 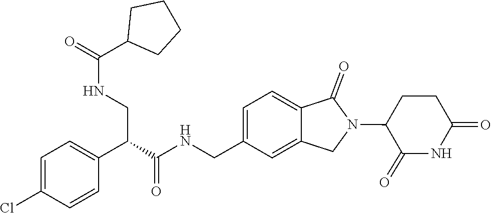 [SMILES: O=C1CCC(N2Cc3cc(CNC(=O)[C@@H](CNC(=O)C4CCCC4)c4ccc(Cl)cc4)ccc3C2=O)C(=O)N1] |
| 5-53 | 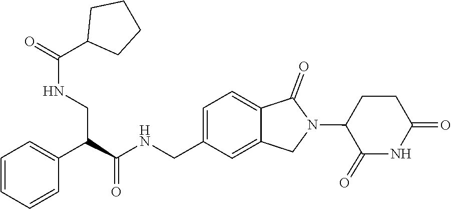 |
| 5-54 | 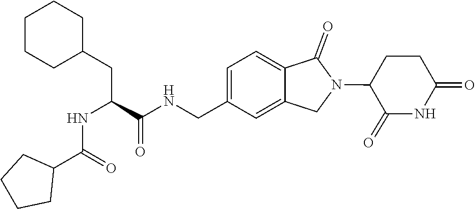 [SMILES: O=C1CCC(N2Cc3cc(CNC(=O)[C@H](CC4CCCCC4)NC(=O)C4CCCC4)ccc3C2=O)C(=O)N1] |
| 5-55 | 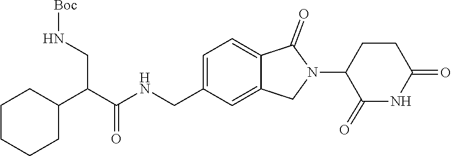 [SMILES: CC(C)(C)OC(=O)NCC(C(=O)NCc1ccc2c(c1)CN(C1CCC(=O)NC1=O)C2=O)C1CCCCC1] |
| 5-58 | 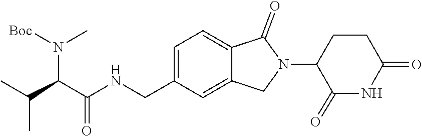 [SMILES: CC(C)[C@H](C(=O)NCc1ccc2c(c1)CN(C1CCC(=O)NC1=O)C2=O)N(C)C(=O)OC(C)(C)C] |
| 5-59 | 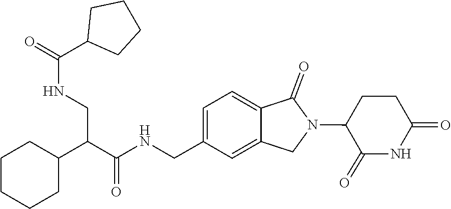 [SMILES: O=C1CCC(N2Cc3cc(CNC(=O)C(CNC(=O)C4CCCC4)C4CCCCC4)ccc3C2=O)C(=O)N1] |
| 5-69 | 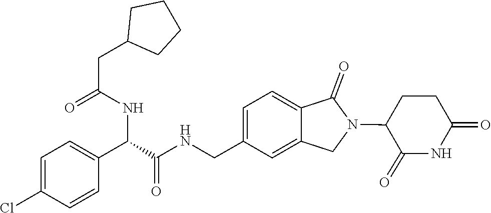 [SMILES: O=C1CCC(N2Cc3cc(CNC(=O)[C@@H](NC(=O)CC4CCCC4)c4ccc(Cl)cc4)ccc3C2=O)C(=O)N1] |
| 5-70 | 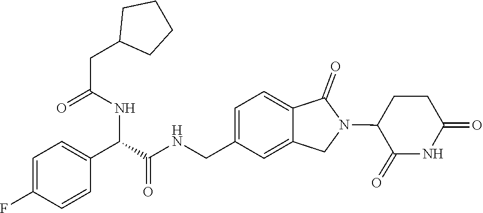 [SMILES: O=C1CCC(N2Cc3cc(CNC(=O)[C@@H](NC(=O)CC4CCCC4)c4ccc(F)cc4)ccc3C2=O)C(=O)N1] |
| 4-135 | 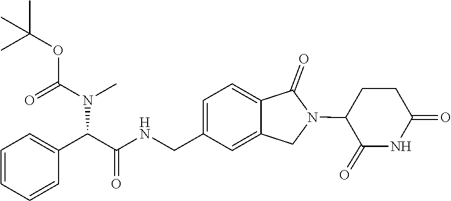 [SMILES: CN(C(=O)OC(C)(C)C)[C@H](C(=O)NCc1ccc2c(c1)CN(C1CCC(=O)NC1=O)C2=O)c1ccccc1] |
| 5-31 | 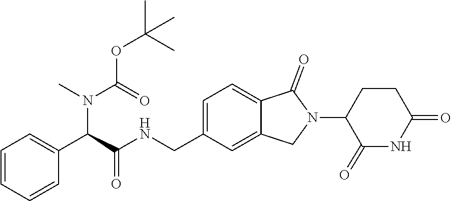 [SMILES: CN(C(=O)OC(C)(C)C)[C@@H](C(=O)NCc1ccc2c(c1)CN(C1CCC(=O)NC1=O)C2=O)c1ccccc1] |
| 5-34 | 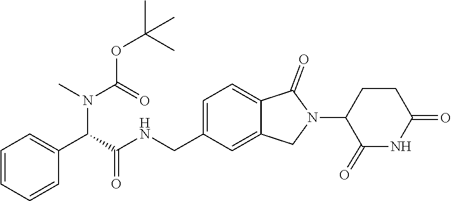 [SMILES: CN(C(=O)OC(C)(C)C)[C@H](C(=O)NCc1ccc2c(c1)CN(C1CCC(=O)NC1=O)C2=O)c1ccccc1] |
| 5-35 | 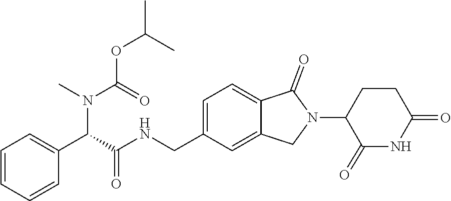 [SMILES: CC(C)OC(=O)N(C)[C@H](C(=O)NCc1ccc2c(c1)CN(C1CCC(=O)NC1=O)C2=O)c1ccccc1] |
| 5-36 | 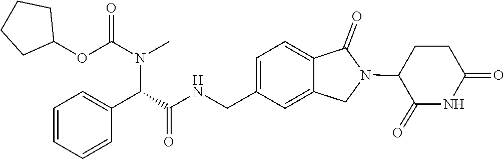 [SMILES: CN(C(=O)OC1CCCC1)[C@H](C(=O)NCc1ccc2c(c1)CN(C1CCC(=O)NC1=O)C2=O)c1ccccc1] |
| 5-40 | 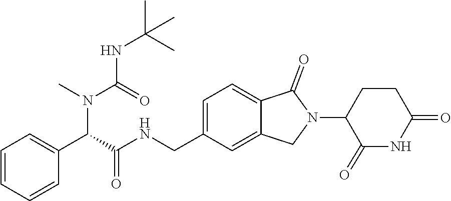 [SMILES: CN(C(=O)NC(C)(C)C)[C@H](C(=O)NCc1ccc2c(c1)CN(C1CCC(=O)NC1=O)C2=O)c1ccccc1] |
| 5-75 | 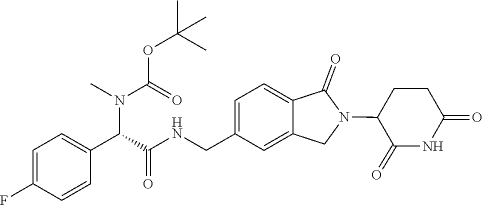 [SMILES: CN(C(=O)OC(C)(C)C)[C@H](C(=O)NCc1ccc2c(c1)CN(C1CCC(=O)NC1=O)C2=O)c1ccc(F)cc1] |
| 5-76 | 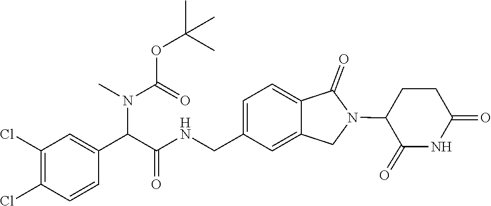 [SMILES: CN(C(=O)OC(C)(C)C)C(C(=O)NCc1ccc2c(c1)CN(C1CCC(=O)NC1=O)C2=O)c1ccc(Cl)c(Cl)c1] |
| 5-77 | 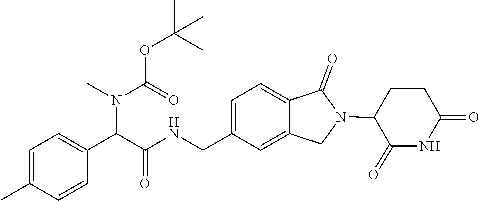 [SMILES: Cc1ccc(C(C(=O)NCc2ccc3c(c2)CN(C2CCC(=O)NC2=O)C3=O)N(C)C(=O)OC(C)(C)C)cc1] |
| 5-78 | 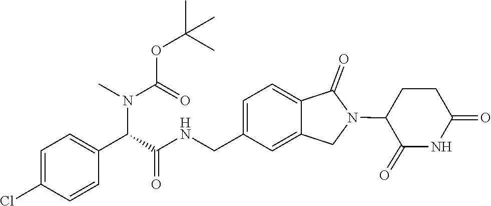 [SMILES: CN(C(=O)OC(C)(C)C)[C@H](C(=O)NCc1ccc2c(c1)CN(C1CCC(=O)NC1=O)C2=O)c1ccc(Cl)cc1] |
| 5-79 | 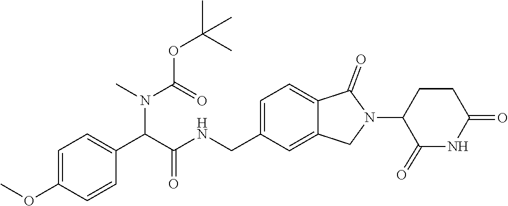 [SMILES: COc1ccc(C(C(=O)NCc2ccc3c(c2)CN(C2CCC(=O)NC2=O)C3=O)N(C)C(=O)OC(C)(C)C)cc1] |
| 1-86 | 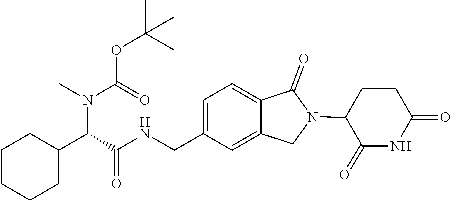 [SMILES: CN(C(=O)OC(C)(C)C)[C@H](C(=O)NCc1ccc2c(c1)CN(C1CCC(=O)NC1=O)C2=O)C1CCCCC1] |
| 1-89 | 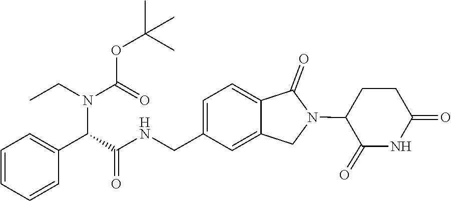 [SMILES: CCN(C(=O)OC(C)(C)C)[C@H](C(=O)NCc1ccc2c(c1)CN(C1CCC(=O)NC1=O)C2=O)c1ccccc1] |
| 5-50 | 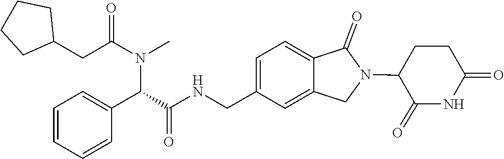 [SMILES: CN(C(=O)CC1CCCC1)[C@H](C(=O)NCc1ccc2c(c1)CN(C1CCC(=O)NC1=O)C2=O)c1ccccc1] |
| 5-49 | 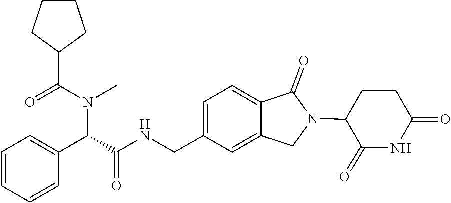 [SMILES: CN(C(=O)C1CCCC1)[C@H](C(=O)NCc1ccc2c(c1)CN(C1CCC(=O)NC1=O)C2=O)c1ccccc1] |
| 5-88 | 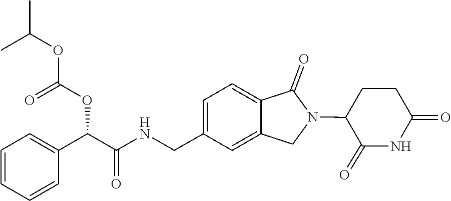 [SMILES: CC(C)OC(=O)O[C@H](C(=O)NCc1ccc2c(c1)CN(C1CCC(=O)NC1=O)C2=O)c1ccccc1] |
| 5-82 | 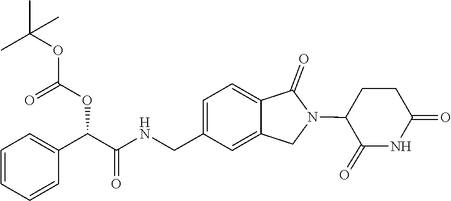 [SMILES: CC(C)(C)OC(=O)O[C@H](C(=O)NCc1ccc2c(c1)CN(C1CCC(=O)NC1=O)C2=O)c1ccccc1] |
| 5-87 | 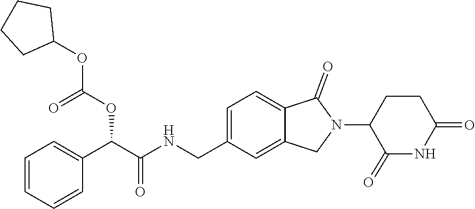 [SMILES: O=C1CCC(N2Cc3cc(CNC(=O)[C@@H](OC(=O)OC4CCCC4)c4ccccc4)ccc3C2=O)C(=O)N1] |
| 5-83 | 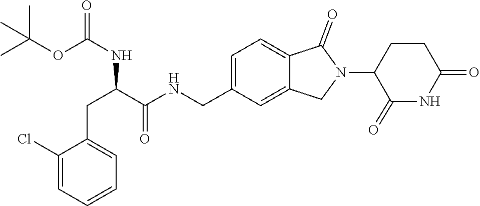 [SMILES: CC(C)(C)OC(=O)N[C@H](Cc1ccccc1Cl)C(=O)NCc1ccc2c(c1)CN(C1CCC(=O)NC1=O)C2=O] |
| 5-85 | 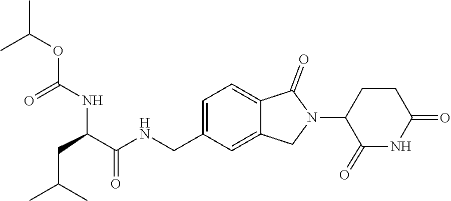 [SMILES: CC(C)C[C@@H](NC(=O)OC(C)C)C(=O)NCc1ccc2c(c1)CN(C1CCC(=O)NC1=O)C2=O] |
| 5-84 | 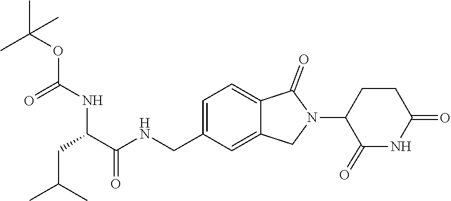 [SMILES: CC(C)C[C@H](NC(=O)OC(C)(C)C)C(=O)NCc1ccc2c(c1)CN(C1CCC(=O)NC1=O)C2=O] |
| 5-92 | 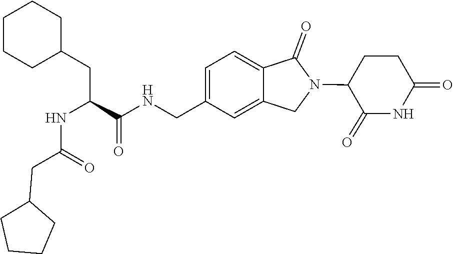 [SMILES: O=C1CCC(N2Cc3cc(CNC(=O)[C@H](CC4CCCCC4)NC(=O)CC4CCCC4)ccc3C2=O)C(=O)N1] |
| 5-93 | 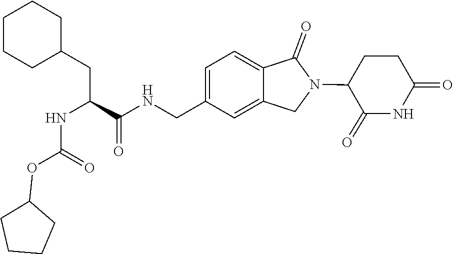 [SMILES: O=C1CCC(N2Cc3cc(CNC(=O)[C@H](CC4CCCCC4)NC(=O)OC4CCCC4)ccc3C2=O)C(=O)N1] |
| 5-94 | 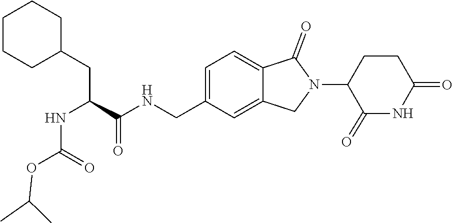 [SMILES: CC(C)OC(=O)N[C@@H](CC1CCCCC1)C(=O)NCc1ccc2c(c1)CN(C1CCC(=O)NC1=O)C2=O] |
| 5-95 | 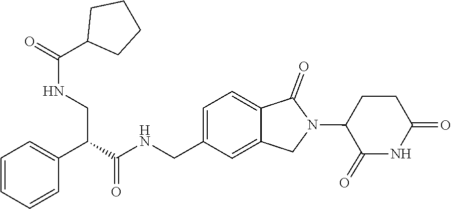 [SMILES: O=C1CCC(N2Cc3cc(CNC(=O)[C@@H](CNC(=O)C4CCCC4)c4ccccc4)ccc3C2=O)C(=O)N1] |
| 5-17b | 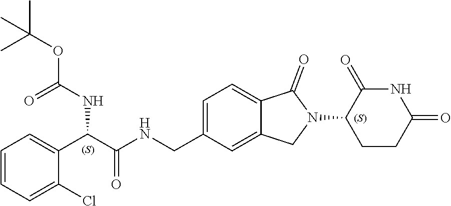 |
| 5-97 | 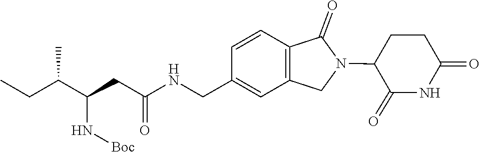 [SMILES: CC[C@H](C)[C@@H](CC(=O)NCc1ccc2c(c1)CN(C1CCC(=O)NC1=O)C2=O)NC(=O)OC(C)(C)C] |
| 5-99 | 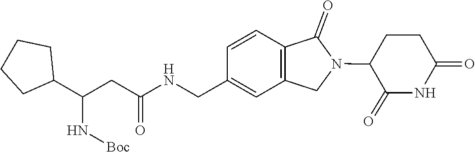 [SMILES: CC(C)(C)OC(=O)NC(CC(=O)NCc1ccc2c(c1)CN(C1CCC(=O)NC1=O)C2=O)C1CCCC1] |
| 6-01 | 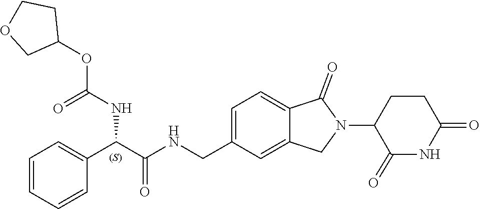 |
| 6-02 | 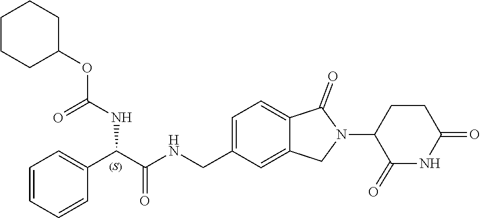 |
| 6-03 | 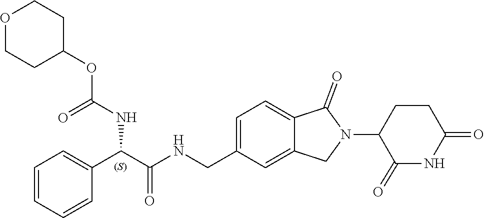 |
| 4-175b | 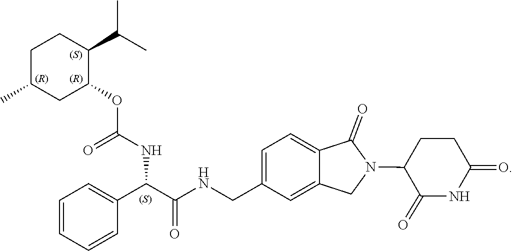 |

11. A pharmaceutical composition, wherein the composition contains an effective amount of the compound of claim 1, or its pharmaceutically acceptable salts, prodrugs, and a pharmaceutically acceptable carrier.

12. Use of the compound of claim 1, wherein it is used for:
(a) Preparation of drugs for treating diseases related to GSPT1 activity or expression levels;
(b) Preparation of GSPT1 targeting inhibitors or degradation agents;
(c) Non-therapeutic inhibition or degradation of GSPT1 in vitro;
(d) Non-therapeutic inhibition of tumor cell proliferation in vitro; and/or
(e) Treating diseases related to GSPT1 activity or expression levels.

13. A method of inhibiting tumor cells in vitro, wherein the method comprises: administering to the tumor cells an inhibitory effective amount of a compound of Formula I of claim 1, or a pharmaceutical composition, wherein the composition contains an effective amount of the compound of Formula I of claim 1, or its pharmaceutical acceptable salts, prodrugs, and a pharmaceutically acceptable carrier.
